(19) United States
(12) Patent Application Publication
(10) Pub. No.: US 2026/0224486 A1
(43) Pub. Date: Aug. 6, 2026

(54) DIANHYDROHEXITOL BASED IONIZABLE LIPIDS FOR NUCLEIC ACID DELIVERY

(71) Applicant: SANOFI PASTEUR INC., Swiftwater, PA (US)

(72) Inventors: Ramesh DASARI, Cambridge, MA (US); Hongfeng DENG, Cambridge, MA (US); Saswata KARMAKAR, Cambridge, MA (US); Shrirang KARVE, Cambridge, MA (US); Amita VAIDYA, Cambridge, MA (US); Kudzai NYAMKONDIWA, Cambridge, MA (US)

(21) Appl. No.: 19/431,288

(22) Filed: Dec. 23, 2025

Related U.S. Application Data
(63) Continuation of application No. PCT/IB2024/000352, filed on Jun. 28, 2024.

(30) Foreign Application Priority Data
Jun. 28, 2023 (EP) 23306049.0

(51) Int. Cl. A61K 9/1271; A61K 9/51; A61K 31/7105; C07D 493/04

(52) U.S. Cl. CPC A61K 9/1271; A61K 9/5123; A61K 31/7105; C07D 493/04

(57) ABSTRACT

The present invention provides, in part, dianhydrohexitol-based cationic lipids of Formula (I), and sub-formulas thereof:

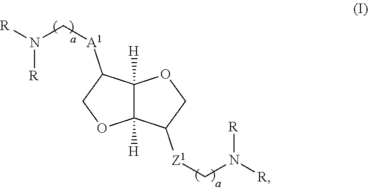

or a pharmaceutically acceptable saft thereof. The present invention also provides, in part, dianhydrohexitol-based cationic lipids of Formula (II), and sub-formulas thereof:

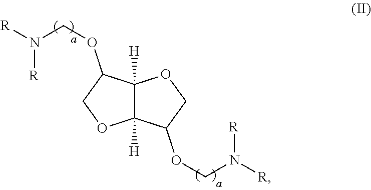

or a pharmaceutically acceptable salt thereof. The compounds provided herein be useful for delivery and expression of mRNA and encoded protein, e.g., as a component of liposomal delivery vehicle, and accordingly can be useful for treating various diseases, disorders and conditions, such as those associated with deficiency of one or more proteins.

### RELATED APPLICATIONS

This application is a continuation of International Patent Application No. PCT/IB2024/000352, filed Jun. 28, 2024, which claims priority to European Patent Application No. 23306049.0, filed Jun. 28, 2023, the entire disclosures of which are hereby incorporated by reference.

### BACKGROUND

Delivery of nucleic acids has been explored extensively as a potential therapeutic option for certain disease states. In particular, messenger RNA (mRNA) therapy has become an increasingly important option for the prevention and treatment of various diseases (e.g. in the use of vaccines).

Efficient delivery of liposome-encapsulated nucleic acids remains an active area of research. Liposome-encapsulated nucleic acids can be administered intramuscularly (IM).

The cationic lipid component of a liposome plays an important role in facilitating effective encapsulation of the nucleic acid during the loading of liposomes. In addition, cationic lipids may play an important role in the efficient release of the nucleic acid cargo from the liposome into the cytoplasm of a target cell. Various cationic lipids suitable for in vivo use have been discovered. However, there remains a need to identify cationic lipids that are effective for intramuscular delivery of mRNA (e.g., in vaccines, such as for Flu or Respiratory Syncytial virus (RSV)). There also remains a need to identify cationic lipids that can be synthesized efficiently and cheaply without the formation of potentially toxic by-products.

### SUMMARY OF THE INVENTION

The present invention provides, among other things, a novel class of cationic lipid compounds for in vivo delivery of therapeutic agents, such as nucleic acids. The inventors of the present invention have surprisingly found that lipid nanoparticles comprising cationic lipids with Dianhydrohexitol based cores (e.g. isosorbide, isomannide and isoidide based cores) are very effective for the intramuscular delivery of mRNA encapsulated in said lipid nanoparticles. Indeed, lipid nanoparticles comprising the cationic lipids of the present invention have demonstrated high levels of peptide or protein expression when delivering mRNA encoding for said peptide or protein by intramuscular delivery. For example, lipid nanoparticles comprising cationic lipids of the present invention and encapsulating human erythropoietin (hEPO) mRNA achieved improved expression of hEPO mRNA when administered to mice by intramuscular delivery to lipid nanoparticles comprising MC3, which is the current gold standard for in vivo delivery of e.g. siRNA (see WO2010/144740).

The cationic lipids of the present invention are also more straightforward to synthesize than other cationic lipids, such as MC3. Indeed, the synthesis of MC3 involves a six-step process and requires handling of a Grignard reagent. In contrast, the present invention provides cationic lipids that can be prepared from readily available and inexpensive starting reagents, such as isosorbide (1,4:3,6-dianhydro-D-glucidol), isomannide (1,4:3,6-dianhydro-D-mannitol) and isoidide (1,4:3,6-dianhydro-L-iditol).

The cationic lipids of the present invention also comprise cleavable groups (e.g., esters, thioesters, disulphides, carbonates, carbamates and thiocarbamates) that are contemplated to improve biodegradability and thus contribute to their favorable safety profile.

It is contemplated that these compounds are capable of highly effective in vivo intramuscular delivery of the therapeutic agents and vaccines (e.g., for Flu or Respiratory Syncytial virus (RSV)). It is also contemplated that lipid nanoparticles comprising these cationic lipid compounds are capable of highly effective in vivo delivery while maintaining a favorable safety profile. It is also contemplated that lipid nanoparticles comprising these cationic lipid compounds may exhibit improved degradation in vivo.

In an aspect, provided herein are cationic lipids having a structure according to Formula (I):

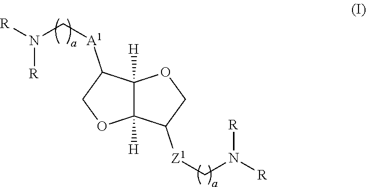

or a pharmaceutically acceptable salt thereof wherein:
A¹ is selected from —C(═O)O—, —C(═O)S—, —C(═O)NH—, —OC(═O)O—, —OC(═O)NH—, —NHC(═O)O—, —SC(═O)NH—, —OCH₂CH₂O—, —OCH₂O—, —OCH(CH₃)O—, —S— and —S—S—, wherein the left hand side of each recited structure is bound to the —(CH₂)ₐ—;
Z¹ is selected from —OC(═O)—, —SC(═O)—, —NHC(═O)—, —OC(═O)O—, —NHC(═O)O—, —OC(═O)NH—, —NHC(═O)S—, —OCH₂CH₂O—, —OCH₂O—, —OCH(CH₃)O—, —S— and —S—S—, wherein the right hand side of each recited structure is bound to the —(CH₂)ₐ—;
each R is independently selected from:

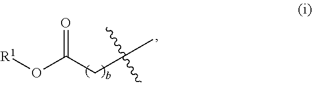

wherein each R¹ is independently selected from optionally substituted alkyl, optionally substituted alkenyl, and optionally substituted alkynyl;

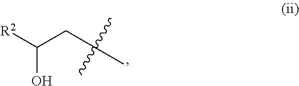

wherein each R² is independently selected from optionally substituted alkyl, optionally substituted alkenyl, optionally substituted alkynyl, and —W¹—X¹,
   wherein each W¹ is independently selected from optionally substituted alkylene and optionally substituted alkenylene, and
   each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;

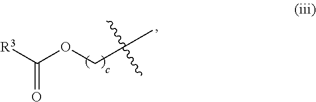

wherein each R³ is independently selected from optionally substituted alkyl, optionally substituted alkenyl, and optionally substituted alkynyl; and

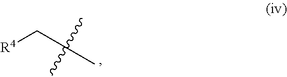

wherein each R⁴ is independently selected from optionally substituted cycloalkyl or optionally substituted heterocycloalkyl;
wherein at least three R are independently selected from

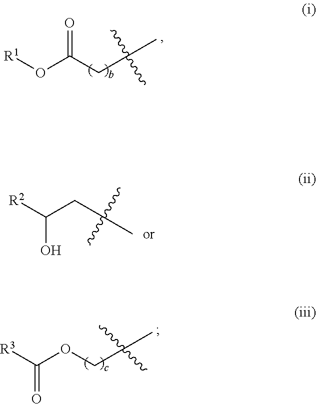

each a is independently selected from 2, 3, 4, and 5;
each b is independently selected from 2, 3, 4, 5, 6, 7, 8, 9 and 10; and
each c is independently selected from 2, 3, 4, 5, 6, 7, 8, 9 and 10.

In an aspect, provided herein are cationic lipids that are pharmaceutically acceptable salts of Formula (I).

In an aspect, provided herein are cationic lipids having a structure according to Formula (II):

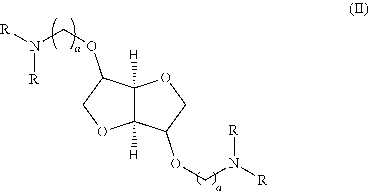

or a pharmaceutically acceptable salt thereof wherein:
each R is independently selected from:

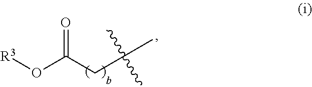

wherein each R¹ is independently selected from optionally substituted alkyl, optionally substituted alkenyl, and optionally substituted alkynyl; and

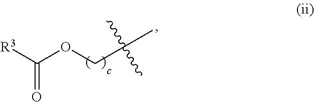

wherein each R³ is independently selected from optionally substituted alkyl, optionally substituted alkenyl, and optionally substituted alkynyl;
each a is independently selected from 2, 3, 4, and 5;
each b is independently selected from 2, 3, 4, 5, 6, and 7; and
each c is independently selected from 2, 3, 4, 5, 6, and 7.

In an aspect, provided herein are cationic lipids that are pharmaceutically acceptable salts of Formula (II).

In an aspect, provided herein are compositions comprising the cationic lipid of the present invention or a pharmaceutically acceptable salt thereof, and further comprising:
(i) one or more non-cationic lipids,
(ii) one or more cholesterol-based lipids and
(iii) one or more PEG-modified lipid.

In an aspect, the composition is a lipid nanoparticle, optionally a liposome.

In an aspect, the compositions comprising the cationic lipids of the present invention may be used in therapy. For example, to treat, prevent or ameliorate Flu or Respiratory Syncytial virus (RSV).

### DETAILED DESCRIPTION OF CERTAIN EMBODIMENTS

### Definitions

In order for the present invention to be more readily understood, certain terms are first defined below. Additional definitions for the following terms and other terms are set forth throughout the specification. The publications and other reference materials cited herein to describe the background of the invention and to provide additional detail regarding its practice are hereby incorporated by reference.

Amino acid: As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an I-amino acid. “Standard amino acid” refers to any of the twenty standard I-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or post-translational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the term “biologically active” refers to a characteristic of any agent that has activity in a biological system, and particularly in an organism. For instance, an agent that, when administered to an organism, has a biological effect on that organism, is considered to be biologically active.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery”).

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalents thereof, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Helper lipid: The term “helper lipid” as used herein refers to any neutral or zwitterionic lipid material including cholesterol. Without wishing to be held to a particular theory, helper lipids may add stability, rigidity, and/or fluidity within lipid bilayers/nanoparticles.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Liposome: As used herein, the term “liposome” refers to any lamellar, multilamellar, or solid nanoparticle vesicle. Typically, a liposome as used herein can be formed by mixing one or more lipids or by mixing one or more lipids and polymer(s). In some embodiments, a liposome suitable for the present invention contains a cationic lipid(s) and optionally further comprises:
(i) non-cationic lipid(s),
(ii) cholesterol-based lipid(s), and/or
(iii) PEG-modified lipid(s).

messenger RNA (mRNA): As used herein, the term “messenger RNA (mRNA)” or “mRNA” refers to a polynucleotide that encodes at least one polypeptide. mRNA as used herein encompasses both modified and unmodified RNA. The term “modified mRNA” related to mRNA comprising at least one chemically modified nucleotide. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C5-propynyl-cytidine, C5-propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. In some embodiments, “nucleic acid” encompasses ribonucleic acids (RNA), including but not limited to any one or more of interference RNAs (RNAi), small interfering RNA (siRNA), short hairpin RNA (shRNA), antisense RNA (aRNA), messenger RNA (mRNA), modified messenger RNA (mmRNA), long non-coding RNA (lncRNA), micro-RNA (miRNA) multimeric coding nucleic acid (MCNA), polymeric coding nucleic acid (PCNA), guide RNA (gRNA) and CRISPR RNA (crRNA). In some embodiments, “nucleic acid” encompasses deoxyribonucleic acid (DNA), including but not limited to any one or more of single-stranded DNA (ssDNA), double-stranded DNA (dsDNA) and complementary DNA (cDNA). In some embodiments, “nucleic acid” encompasses both RNA and DNA. In embodiments, DNA may be in the form of antisense DNA, plasmid DNA, parts of a plasmid DNA, pre-condensed DNA, a product of a polymerase chain reaction (PCR), vectors (e.g., P1, PAC, BAC, YAC, artificial chromosomes), expression cassettes, chimeric sequences, chromosomal DNA, or derivatives of these groups. In embodiments, RNA may be in the form of messenger RNA (mRNA), ribosomal RNA (rRNA), signal recognition particle RNA (7 SL RNA or SRP RNA), transfer RNA (tRNA), transfer-messenger RNA (tmRNA), small nuclear RNA (snRNA), small nucleolar RNA (snoRNA), SmY RNA, small Cajal body-specific RNA (scaRNA), guide RNA (gRNA), ribonuclease P (RNase P), Y RNA, telomerase RNA component (TERC), spliced leader RNA (SL RNA), antisense RNA (aRNA or asRNA), cis-natural antisense transcript (cis-NAT), CRISPR RNA (crRNA), long noncoding RNA (lncRNA), micro-RNA (miRNA), piwi-interacting RNA (piRNA), small interfering RNA (siRNA), transacting siRNA (tasiRNA), repeat associated siRNA (rasiRNA), 73K RNA, retrotransposons, a viral genome, a viroid, satellite RNA, or derivatives of these groups. In some embodiments, a nucleic acid is a mRNA encoding a protein such as an enzyme.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, non-toxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quaternization of an amine using an appropriate electrophile, e.g., an alkyl halide, to form a quarternized alkylated amino salt.

Systemic distribution or delivery: As used herein, the terms “systemic distribution” or “systemic delivery,” or grammatical equivalents thereof, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### Chemical Definitions

Acyl: As used herein, the term “acyl” refers to R^{Z}—(C═O)—, wherein R^{Z} is, for example, any alkyl, alkenyl, alkynyl, heteroalkyl or heteroalkylene.

Aliphatic: As used herein, the term aliphatic refers to (C₁-C₅₀) hydrocarbons and includes both saturated and unsaturated hydrocarbons. An aliphatic may be linear, branched, or cyclic. For example, (C₁-C₂₀) aliphatics can include (C₁-C₂₀) alkyls (e.g., linear or branched (C₁-C₂₀) saturated alkyls), (C₂-C₂₀) alkenyls (e.g., linear or branched (C₁-C₂₀) dienyls, linear or branched (C₆-C₂₀) trienyls, and the like), and (C₂-C₂₀)alkynyls (e.g., linear or branched (C₂-C₂₀) alkynyls). (C₁-C₂₀) aliphatics can include (C₃-C₂₀) cyclic aliphatics (e.g., (C₃-C₂₀)cycloalkyls, (C₁-C₂₀) cycloalkenyls, or (C₈-C₂₀) cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the aliphatic is unsubstituted. In embodiments, the aliphatic does not include any heteroatoms. Alkyl: As used herein, the term “alkyl” means acyclic linear and branched hydrocarbon groups, e.g. “(C₁-C₃₀) alkyl” refers to alkyl groups having 1-30 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the alkyl is substituted (e.g., with 1, 2, 3, 4, 5, or 6 substituent groups as described herein). In embodiments, an alkyl group is substituted with a —OH group and may also be referred to herein as a “hydroxyalkyl” group, where the prefix denotes the —OH group and “alkyl” is as described herein.

As used herein, “alkyl” also refers to a radical of a straight-chain or branched saturated hydrocarbon group having from 1 to 50 carbon atoms (“(C₁-C₅₀) alkyl”). In some embodiments, an alkyl group has 1 to 40 carbon atoms (“(C₁-C₄₀) alkyl”). In some embodiments, an alkyl group has 1 to 30 carbon atoms (“(C₁-C₃₀) alkyl”). In some embodiments, an alkyl group has 1 to 20 carbon atoms (“(C₁-C₂₀) alkyl”). In some embodiments, an alkyl group has 1 to 10 carbon atoms (“(C₁-C₁₀) alkyl”). In some embodiments, an alkyl group has 1 to 9 carbon atoms (“(C₁-C₉) alkyl”). In some embodiments, an alkyl group has 1 to 8 carbon atoms (“(C₁-C₈) alkyl”). In some embodiments, an alkyl group has 1 to 7 carbon atoms (“(C₁-C₇) alkyl”). In some embodiments, an alkyl group has 1 to 6 carbon atoms (“(C₁-C₆) alkyl”). In some embodiments, an alkyl group has 1 to 5 carbon atoms (“(C₁-C₅) alkyl”). In some embodiments, an alkyl group has 1 to 4 carbon atoms (“(C₁-C₄) alkyl”). In some embodiments, an alkyl group has 1 to 3 carbon atoms (“(C₁-C₃) alkyl”). In some embodiments, an alkyl group has 1 to 2 carbon atoms (“(C₁-C₂) alkyl”). In some embodiments, an alkyl group has 1 carbon atom (“C₁ alkyl”). In some embodiments, an alkyl group has 2 to 6 carbon atoms (“(C₂-C₆) alkyl”). Examples of (C₁-C₆) alkyl groups include, without limitation, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted (C₁-C₅₀) alkyl. In certain embodiments, the alkyl group is a substituted (C₁-C₅₀) alkyl.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In certain embodiments, an alkylene, alkenylene, or alkynylene is unsubstituted. In certain embodiments, an alkylene, alkenylene, or alkynylene does not include any heteroatoms. Alkenyl: As used herein, “alkenyl” means any linear or branched hydrocarbon chains having one or more unsaturated carbon-carbon double bonds that may occur in any stable point along the chain, e.g. “(C₂-C₃₀) alkenyl” refers to an alkenyl group having 2-30 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In embodiments, the alkenyl comprises 1, 2, or 3 carbon-carbon double bond. In embodiments, the alkenyl comprises a single carbon-carbon double bond. In embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a-OH group and may also be referred to herein as a “hydroxyalkenyl” group, where the prefix denotes the —OH group and “alkenyl” is as described herein.

As used herein, “alkenyl” also refers to a radical of a straight-chain or branched hydrocarbon group having from 2 to 50 carbon atoms and one or more carbon-carbon double bonds (e.g., 1, 2, 3, or 4 double bonds) (“(C₂-C₅₀) alkenyl”). In some embodiments, an alkenyl group has 2 to 40 carbon atoms (“(C₂-C₄₀) alkenyl”). In some embodiments, an alkenyl group has 2 to 30 carbon atoms (“(C₂-C₃₀) alkenyl”). In some embodiments, an alkenyl group has 2 to 20 carbon atoms (“(C₁-C₂₀) alkenyl”). In some embodiments, an alkenyl group has 2 to 10 carbon atoms (“(C₂-C₁₀) alkenyl”). In some embodiments, an alkenyl group has 2 to 9 carbon atoms (“(C₂-C₉) alkenyl”). In some embodiments, an alkenyl group has 2 to 8 carbon atoms (“(C₂-C₈) alkenyl”). In some embodiments, an alkenyl group has 2 to 7 carbon atoms (“(C₂-C₇) alkenyl”). In some embodiments, an alkenyl group has 2 to 6 carbon atoms (“(C₂-C₆) alkenyl”). In some embodiments, an alkenyl group has 2 to 5 carbon atoms (“(C₂-C₅) alkenyl”). In some embodiments, an alkenyl group has 2 to 4 carbon atoms (“(C₂-C₄) alkenyl”). In some embodiments, an alkenyl group has 2 to 3 carbon atoms (“(C₂-C₃) alkenyl”). In some embodiments, an alkenyl group has 2 carbon atoms (“(C₂) alkenyl”). The one or more carbon-carbon double bonds can be internal (such as in 2-butenyl) or terminal (such as in 1-butenyl). Examples of (C₂-C₆) alkenyl groups include, without limitation, ethenyl (C₂), 1-propenyl (C₃), 2-propenyl (C₃), 1-butenyl (C₄), 2-butenyl (C₄), butadienyl (C₄), and the like. Examples of (C₂-C₆) alkenyl groups include the aforementioned (C₂-C₄) alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted (C₂-C₅₀) alkenyl. In certain embodiments, the alkenyl group is a substituted (C₂-C₅₀) alkenyl.

Alkynyl: As used herein, “alkynyl” means any hydrocarbon chain of either linear or branched configuration, having one or more carbon-carbon triple bonds occurring in any stable point along the chain, e.g., “(Q-C₃₀) alkynyl”, refers to an alkynyl group having 2-30 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the alkynyl is unsubstituted. In embodiments, the alkynyl is substituted (e.g., with 1, 2, 3, 4, 5, or 6 substituent groups as described herein).

As used herein, “alkynyl” also refers to a radical of a straight-chain or branched hydrocarbon group having from 2 to 50 carbon atoms and one or more carbon-carbon triple bonds (e.g., 1, 2, 3, or 4 triple bonds) and optionally one or more double bonds (e.g., 1, 2, 3, or 4 double bonds) (“(C₁-C₅₀) alkynyl”). An alkynyl group that has one or more triple bonds, and one or more double bonds is also referred to as an “ene-yne”. In some embodiments, an alkynyl group has 2 to 40 carbon atoms (“(C₂-C₄₀) alkynyl”). In some embodiments, an alkynyl group has 2 to 30 carbon atoms (“(C₂-C₃₀) alkynyl”). In some embodiments, an alkynyl group has 2 to 20 carbon atoms (“(C₁-C₂₀) alkynyl”). In some embodiments, an alkynyl group has 2 to 10 carbon atoms (“(C₂-C₁₀) alkynyl”). In some embodiments, an alkynyl group has 2 to 9 carbon atoms (“(C₂-C₉) alkynyl”). In some embodiments, an alkynyl group has 2 to 8 carbon atoms (“(C₂-C₈) alkynyl”). In some embodiments, an alkynyl group has 2 to 7 carbon atoms (“(C₂-C₇) alkynyl”). In some embodiments, an alkynyl group has 2 to 6 carbon atoms (“(C₂-C₆) alkynyl”). In some embodiments, an alkynyl group has 2 to 5 carbon atoms (“(C₂-C₅) alkynyl”). In some embodiments, an alkynyl group has 2 to 4 carbon atoms (“(C₂-C₄) alkynyl”). In some embodiments, an alkynyl group has 2 to 3 carbon atoms (“(C₂-C₃) alkynyl”). In some embodiments, an alkynyl group has 2 carbon atoms (“(C₂) alkynyl”). The one or more carbon-carbon triple bonds can be internal (such as in 2-butynyl) or terminal (such as in 1-butynyl). Examples of (C₂-C₄) alkynyl groups include, without limitation, ethynyl (C₂), 1-propynyl (C₃), 2-propynyl (C₃), 1-butynyl (C₄), 2-butynyl (C₄), and the like. Examples of (C₂-C₆) alkenyl groups include the aforementioned (C₂-C₄) alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted (C₂-C₅₀) alkynyl. In certain embodiments, the alkynyl group is a substituted (C₂-C₅₀) alkynyl.

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In embodiments, an aryl group has 6 ring carbon atoms (“(C₆) aryl,” e.g., phenyl). In some embodiments, an aryl group has 10 ring carbon atoms (“(C₁₀) aryl,” e.g., naphthyl such as 1-naphthyl and 2-naphthyl). In some embodiments, an aryl group has 14 ring carbon atoms (“(C₄) aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

As used herein, “aryl” also refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“(C₆-C₁₄) aryl”). In some embodiments, an aryl group has 6 ring carbon atoms (“(C₆) aryl”; e.g., phenyl). In some embodiments, an aryl group has 10 ring carbon atoms (“(C₁₀) aryl”; e.g., naphthyl such as 1-naphthyl and 2-naphthyl). In some embodiments, an aryl group has 14 ring carbon atoms (“(C₁₄) aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted (C₆-C₁₄) aryl. In certain embodiments, the aryl group is a substituted (C₆-C₁₄) aryl.

Arylene: The term “arylene” as used herein refers to an aryl group that is divalent (that is, having two points of attachment to the molecule). Exemplary arylenes include phenylene (e.g., unsubstituted phenylene or substituted phenylene).

Carbocyclyl: As used herein, “carbocyclyl” or “carbocyclic” refers to a radical of a non-aromatic cyclic hydrocarbon group having from 3 to 10 ring carbon atoms (“(C₃-C₁₀) carbocyclyl”) and zero heteroatoms in the non-aromatic ring system. In some embodiments, a carbocyclyl group has 3 to 8 ring carbon atoms (“(C₃-C₈) carbocyclyl”). In some embodiments, a carbocyclyl group has 3 to 7 ring carbon atoms (“(C₃-C₇) carbocyclyl”). In some embodiments, a carbocyclyl group has 3 to 6 ring carbon atoms (“(C₃-C₆) carbocyclyl”). In some embodiments, a carbocyclyl group has 4 to 6 ring carbon atoms (“(C₁-C₆) carbocyclyl”). In some embodiments, a carbocyclyl group has 5 to 6 ring carbon atoms (“(C₅-C₆) carbocyclyl”). In some embodiments, a carbocyclyl group has 5 to 10 ring carbon atoms (“(C₅-C₁₀) carbocyclyl”). Exemplary (C₃-C₆) carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary (C₃-C₈) carbocyclyl groups include, without limitation, the aforementioned (C₃-C₆) carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary (C₃-C₁₀) carbocyclyl groups include, without limitation, the aforementioned (C₃-C₈) carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃-C₁₀ carbocyclyl. In certain embodiments, the carbocyclyl group is a substituted (C₃-C₁₀) carbocyclyl.

In some embodiments, “carbocyclyl” or “carbocyclic” is referred to as a “cycloalkyl”, i.e., a monocyclic, saturated carbocyclyl group having from 3 to 10 ring carbon atoms (“(C₃-C₁₀) cycloalkyl”). In some embodiments, a cycloalkyl group has 3 to 8 ring carbon atoms (“(C₃-C₈) cycloalkyl”). In some embodiments, a cycloalkyl group has 3 to 6 ring carbon atoms (“(C₃-C₆), cycloalkyl”). In some embodiments, a cycloalkyl group has 4 to 6 ring carbon atoms (“(C₄-C₆) cycloalkyl”). In some embodiments, a cycloalkyl group has 5 to 6 ring carbon atoms (“(C₅-C₆) cycloalkyl”). In some embodiments, a cycloalkyl group has 5 to 10 ring carbon atoms (“(C₅-C₁₀) cycloalkyl”). Examples of (C₅-C₆) cycloalkyl groups include cyclopentyl (C₅) and cyclohexyl (C₅). Examples of (C₃-C₆) cycloalkyl groups include the aforementioned (C₅-C₆) cycloalkyl groups as well as cyclopropyl (C₃) and cyclobutyl (C₄). Examples of (C₃-C₈) cycloalkyl groups include the aforementioned (C₃-C₆) cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted (C₃-C₁₀) cycloalkyl. In certain embodiments, the cycloalkyl group is a substituted (C₃-C₁₀) cycloalkyl.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: The term “heteroalkylene,” as used herein, represents a divalent form of a heteroalkyl group as described herein.

Heteroaryl: The term “heteroaryl,” as used herein, is fully unsaturated heteroatom-containing ring wherein at least one ring atom is a heteroatom such as, but not limited to, nitrogen and oxygen.

As used herein, “heteroaryl” also refers to a radical of a 5-14 membered monocyclic or polycyclic (e.g., bicyclic or tricyclic) 4n+2 aromatic ring system (e.g., having 6, 10, or 14 n electrons shared in a cyclic array) having ring carbon atoms and 1 or more (e.g., 1, 2, 3, or 4 ring heteroatoms) ring heteroatoms provided in the aromatic ring system, wherein each heteroatom is independently selected from oxygen, sulfur, nitrogen, boron, silicon, and phosphorus (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-10 membered heteroaryl”). In some embodiments, a heteroaryl group is a 5-8 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-8 membered heteroaryl”). In some embodiments, a heteroaryl group is a 5-6 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-6 membered heteroaryl”). In some embodiments, the 5-6 membered heteroaryl has 1 or more (e.g., 1, 2, or 3) ring heteroatoms selected from oxygen, sulfur, nitrogen, boron, silicon, and phosphorus. In some embodiments, the 5-6 membered heteroaryl has 1 or 2 ring heteroatoms selected from oxygen, sulfur, nitrogen, boron, silicon, and phosphorus. In some embodiments, the 5-6 membered heteroaryl has 1 ring heteroatom selected from oxygen, sulfur, nitrogen, boron, silicon, and phosphorus. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, “heterocyclyl” or “heterocyclic” refers to a radical of a 3- to 14-membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)) and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-10 membered heterocyclyl”). In some embodiments, a heterocyclyl group is a 5-8 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-8 membered heterocyclyl”). In some embodiments, a heterocyclyl group is a 5-6 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-6 membered heterocyclyl”). In some embodiments, the 5-6 membered heterocyclyl has 1 or more (e.g., 1, 2, or 3) ring heteroatoms selected from oxygen, sulfur, nitrogen, boron, silicon, and phosphorus. In some embodiments, the 5-6 membered heterocyclyl has 1 or 2 ring heteroatoms selected from oxygen, sulfur, nitrogen, boron, silicon, and phosphorus. In some embodiments, the 5-6 membered heterocyclyl has 1 ring heteroatom selected from oxygen, sulfur, nitrogen, boron, silicon, and phosphorus.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno [3,2-b]pyridinyl, 1,2,3,4-tetrahydro-1,6-naphthyridinyl, and the like.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. The heterocycloalkyl group can be substituted or unsubstituted.

As understood from the above, alkyl, alkenyl, alkynyl, acyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valences of the heteroatoms and results in the formation of a stable moiety.

Exemplary carbon atom substituents include, but are not limited to, halogen, —CN, —NO₂, —N₃, —SO₂, —SO₃H, —OH, —OR^{aa}, —ON(R^{bb})₂, —N(R^{bb})₂, —N(R^{bb})₃+X⁻, —N(OR^{cc})R^{bb}, —SeH, —SeR^{aa}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃—OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{aa})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), (C₁-C₅₀) alkyl, (C₂-C₅₀) alkenyl, (C₂-C₅₀) alkynyl, (C₃-C₁₄) carbocyclyl, 3-14 membered heterocyclyl, (C₆-C₁₄) aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{bb} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of RV is, independently, selected from hydrogen, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₄) aryl, and 5-14 membered heteroaryl, or two R groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Ra groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ee}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ee})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, (C₁-C₅₀) alkyl, (C₂-C₅₀) alkenyl, (C₂-C₅₀) alkynyl, (C₃-C₁₀) carbocyclyl, 3-10 membered heterocyclyl, (C₆-C₁₀) aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from (C₁-C₅₀) alkyl, (C₂-C₅₀) alkenyl, (C₂-C₅₀) alkynyl, (C₃-C₁₀) carbocyclyl, (C₆-C₁₀) aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, (C₁-C₅₀) alkyl, (C₂-C₅₀) alkenyl, (C₂-C₅₀) alkynyl, (C₃-C₁₀) carbocyclyl, 3-10 membered heterocyclyl, (C₆-C₁₀) aryl and 5-10 membered heteroaryl, or two R^{ff} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —O(C₁-C₅₀) alkyl, —ON((C₁-C₅₀) alkyl)₂, —N((C₁-C₅₀) alkyl)₂, —N((C₁-C₅₀) alkyl)₃+X⁻, —NH((C₁-C₅₀) alkyl)₂+X⁻, —NH₂((C₁-C₅₀) alkyl)+X⁻, —NH₃+X⁻, —N(O(C₁-C₅₀) alkyl)((C₁-C₅₀) alkyl), —N(OH)((C₁-C₅₀) alkyl), —NH(OH), —SH, —S(C₁-C₅₀) alkyl, —SS((C₁-C₅₀) alkyl), —C(═O)((C₁-C₅₀) alkyl), —CO₂H, —CO₂((C₁-C₅₀) alkyl), —OC(═O)((C₁-C₅₀) alkyl), —OCO₂((C₁-C₅₀) alkyl), —C(═O)NH₂, —C(═O)N((C₁-C₅₀) alkyl)₂, —OC(═O)NH((C₁-C₅₀) alkyl), —NHC(═O)((C₁-C₅₀) alkyl), —N((C₁-C₅₀) alkyl)C(═O)((C₁-C₅₀) alkyl), —NHCO₂((C₁-C₅₀) alkyl), —NHC(═O)N((C₁-C₅₀) alkyl)₂, —NHC(═O)NH((C₁-C₅₀) alkyl), —NHC(═O)NH₂, —C(═NH)O((C₁-C₅₀) alkyl), —OC(═NH)((C₁-C₅₀) alkyl), —OC(═NH)O(C₁-C₅₀) alkyl, —C(═NH)N((C₁-C₅₀) alkyl)₂, —C(═NH)NH((C₁-C₅₀) alkyl), —C(═NH)NH₂, —OC(═NH)N((C₁-C₅₀)alkyl)₂, —OC(NH)NH((C₁-C₅₀) alkyl), —OC(NH)NH₂, —NHC(NH)N((C₁-C₅₀) alkyl)₂, —NHC(═NH)NH₂, —NHSO₂((C₁-C₅₀) alkyl), —SO₂N((C₁-C₅₀) alkyl)₂, —SO₂NH((C₁-C₅₀) alkyl), —SO₂NH₂, —SO₂((C₁-C₅₀) alkyl), —SO₂O((C₁-C₅₀) alkyl), —OSO₂((C₁-C₆) alkyl), —SO((C₁-C₆) alkyl), —Si((C₁-C₅₀) alkyl)₃, —OSi((C₁-C₆) alkyl)₃, —C(═S)N((C₁-C₅₀) alkyl)₂, C(═S)NH((C₁-C₅₀) alkyl), C(═S)NH₂, —C(═O)S((C₁-C₆) alkyl), —C(═S)S((C₁-C₆) alkyl), —SC(═S)S((C₁-C₆) alkyl), —P(═O)₂((C₁-C₅₀) alkyl), —P(═O)((C₁-C₅₀) alkyl)₂, —OP(═O)((C₁-C₅₀) alkyl)₂, —OP(═O)(O(C₁-C₅₀) alkyl)₂, (C₁-C₅₀) alkyl, (C₂-C₅₀) alkenyl, (C₂-C₅₀) alkynyl, (C₃-C₁₀) carbocyclyl, (C₆-C₁₀) aryl, 3-10 membered heterocyclyl, 5-10 membered heteroaryl; or two geminal Ra substituents can be joined to form ═O or ═S; wherein X⁻ is a counterion.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quaternary amine in order to maintain electronic neutrality. Exemplary counterions include halide ions (e.g., F⁻, Cl⁻, Br⁻, I⁻), NO₃⁻, CO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}—, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{cc} groups, together with the N atom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-I-cyclopropylmethyl carbamate, 1-methyl-1(3,5-dimethoxyphenyl)ethyl carbamate, 1-methyl-1-(p-phenylazophenyl)ethyl carbamate, 1-methyl-I-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl) phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary sulfur protecting groups include, but are not limited to, alkyl, benzyl, p-methoxybenzyl, 2,4,6-trimethylbenzyl, 2,4,6-trimethoxybenzyl, o-hydroxybenzyl, p-hydroxybenzyl, o-acetoxybenzyl, p-acetoxybenzyl, p-nitrobenzyl, 4-picolyl, 2-quinolinylmethyl, 2-picolyl N-oxido, 9-anthrylmethyl, 9-fluorenylmethyl, xanthenyl, ferrocenylmethyl, diphenylmethyl, bis(4-methoxyphenyl)methyl, 5-dibenzosuberyl, triphenylmethyl, diphenyl-4-pyridylmethyl, phenyl, 2,4-dinitrophenyl, t-butyl, 1-adamantyl, methoxymethyl (MOM), isobutoxymethyl, benzyloxymethyl, 2-tetrahydropyranyl, benzylthiomethyl, phenylthiomethyl, thiazolidino, acetamidomethyl, trimethylacetamidomethyl, benzamidomethyl, allyloxycarbonylaminomethyl, phenylacetamidomethyl, phthalimidomethyl, acetylmethyl, carboxymethyl, cyanomethyl, (2-nitro-1-phenyl)ethyl, 2-(2,4-dinitrophenyl)ethyl, 2-cyanoethyl, 2-(Trimethylsilyl)ethyl, 2,2-bis(carboethoxy)ethyl, (1-m-nitrophenyl-2-benzoyl)othyl, 2-phenylsulfonylethyl, 2-(4-methylphenylsulfonyl)-2-methylprop-2-yl, acetyl, benzoyl, trifluoroacetyl, N-[[(p-biphenylyl)isopropoxy]carbonyl]-N-methyl]-γ-aminothiobutyrate, 2,2,2-trichloroethoxycarbonyl, t-butoxycarbonyl, benzyloxycarbonyl, p-methoxybenzyloxycarbonyl, N-ethyl, N-methoxymethyl, sulfonate, sulfenylthiocarbonate, 3-nitro-2-pyridinesulfenyl sulfide, oxathiolone.

### Compounds of the Invention

Liposomal-based vehicles are considered as an attractive carrier for therapeutic agents and remain subject to continued development efforts. While liposomal-based vehicles that comprise certain lipid components have shown promising results with regard to encapsulation, stability and site localization, there remains a great need for improvement of liposomal-based delivery systems. For example, a significant drawback of liposomal delivery systems relates to the construction of liposomes that have sufficient cell culture or in vivo stability to reach desired target cells and/or intracellular compartments, and the ability of such liposomal delivery systems to efficiently release their encapsulated materials to such target cells.

In particular, there remains a need for cationic lipids that are effective for intramuscular delivery of mRNA (e.g. for treating Flu or Respiratory Syncytial virus (RSV)). There also remains a need for improved lipids compounds that demonstrate improved pharmacokinetic properties, and which are capable of delivering macromolecules, such as nucleic acids, to a wide variety cell types and tissues with enhanced efficiency. Importantly, there also remains a particular need for novel lipid compounds that are characterized as having improved safety profiles and are capable of efficiently delivering encapsulated nucleic acids and polynucleotides to targeted cells, tissues and organs.

Described herein is a novel class of cationic lipid compounds for improved in vivo delivery of therapeutic agents, such as nucleic acids (e.g., for Flu or Respiratory Syncytial virus (RSV)). In particular, a cationic lipid described herein may be used, optionally with other lipids, to formulate a lipid-based nanoparticle (e.g., liposome) for encapsulating therapeutic agents, such as nucleic acids (e.g., DNA, siRNA, mRNA, microRNA) for therapeutic use, such as disease treatment and prevention (vaccine e.g., for Flu or Respiratory Syncytial virus (RSV)) purposes.

In embodiments, compounds of the invention as described herein can provide one or more desired characteristics or properties. That is, in certain embodiments, compounds of the invention as described herein can be characterized as having one or more properties that afford such compounds advantages relative to other similarly classified lipids. For example, compounds disclosed herein can allow for the control and tailoring of the properties of liposomal compositions (e.g., lipid nanoparticles) of which they are a component. In particular, compounds disclosed herein can be characterized by enhanced transfection efficiencies and their ability to provoke specific biological outcomes. Such outcomes can include, for example enhanced cellular uptake, endosomal/lysosomal disruption capabilities and/or promoting the release of encapsulated materials (e.g., polynucleotides) intracellularly. The compounds disclosed herein can also be characterized by achieving high levels of peptide or protein expression when delivering mRNA encoding for said peptide or protein by intravenous, intrathecal, intramuscular, intranasal, sublingual, or by pulmonary delivery, optionally through nebulization. Additionally, the compounds disclosed herein have advantageous pharmacokinetic properties, biodistribution, and efficiency.

The present application demonstrates that the cationic lipids of the present invention are not only synthetically tractable from readily available starting materials, but they also have unexpectedly high encapsulation efficiencies.

Additionally, the cationic lipids of the present invention have cleavable groups such as ester groups. These cleavable groups (e.g. esters, thioesters, disulphides, carbonates, carbamates and thiocarbamates) are contemplated to improve biodegradability and thus contribute to the lipids' favorable safety profiles.

It is contemplated that the cationic lipids of the present invention are capable of highly effective in vivo intramuscular delivery of the therapeutic agents and vaccines (e.g., for Flu or Respiratory Syncytial virus (RSV)). It is also contemplated that lipid nanoparticles comprising the cationic lipids of the present invention are capable of highly effective in vivo delivery while maintaining a favorable safety profile. It is also contemplated that lipid nanoparticles comprising the cationic lipids of the present invention may exhibit improved degradation in vivo.

Provided herein are compounds which are cationic lipids. In embodiments, the cationic lipids of the present invention include compounds having a structure according to Formula (I):

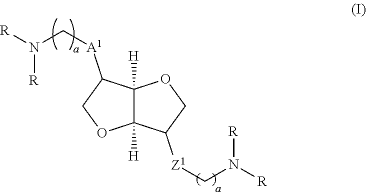

or a pharmaceutically acceptable salt thereof wherein:
A¹ is selected from —C(═O)O—, —C(═O)S—, —C(═O)NH—, —OC(═O)O—, —OC(═O)NH—, —NHC(═O)O—, —SC(═O)NH—, —OCH₂CH₂O—, —OCH₂O—, —OCH(CH₃)O—, —S— and —S—S—, wherein the left hand side of each recited structure is bound to the —(CH₂)ₐ—;
Z¹ is selected from —OC(═O)—, —SC(═O)—, —NHC(═O)—, —OC(═O)O—, —NHC(═O)O—, —OC(═O)NH—, —NHC(═O)S—, —OCH₂CH₂O—, —OCH₂O—, —OCH(CH₃)O—, —S— and —S—S—, wherein the right hand side of each recited structure is bound to the —(CH₂)ₐ—;
each R is independently selected from:

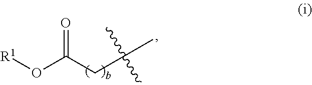

wherein each R¹ is independently selected from optionally substituted alkyl, optionally substituted alkenyl, and optionally substituted alkynyl;

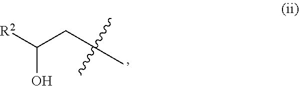

wherein each R² is independently selected from optionally substituted alkyl, optionally substituted alkenyl, optionally substituted alkynyl, and —W¹—X¹,
   wherein each W¹ is independently selected from optionally substituted alkylene and optionally substituted alkenylene, and
   each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;

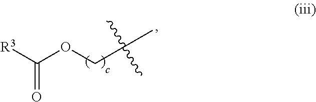

wherein each R³ is independently selected from optionally substituted alkyl, optionally substituted alkenyl, and optionally substituted alkynyl; and

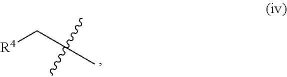

wherein each R⁴ is independently selected from optionally substituted cycloalkyl or optionally substituted heterocycloalkyl;
wherein at least three R are independently selected from

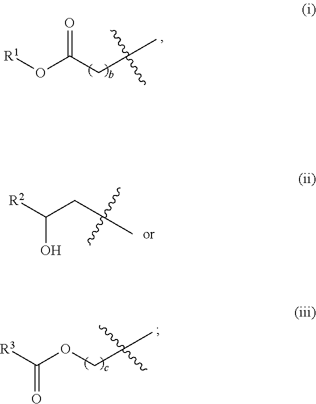

each a is independently selected from 2, 3, 4, and 5;
each b is independently selected from 2, 3, 4, 5, 6, 7, 8, 9 and 10; and
each c is independently selected from 2, 3, 4, 5, 6, 7, 8, 9 and 10.

In embodiments, the cationic lipids of the present invention include compounds having a structure according to Formula (I′):

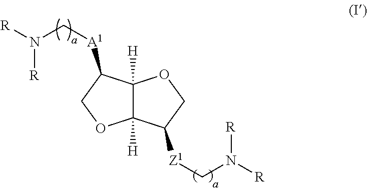

or a pharmaceutically acceptable salt thereof.

In embodiments, the cationic lipids of the present invention include compounds having a structure according to Formula (IA):

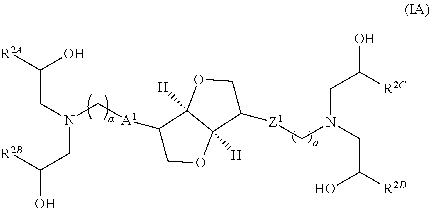

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

In embodiments, the cationic lipids of the present invention include compounds having a structure according to Formula (IE):

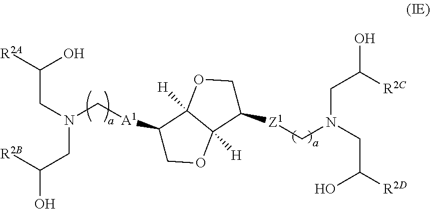

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

In embodiments, the cationic lipids of the present invention include compounds having a structure according to Formula (IB1a):

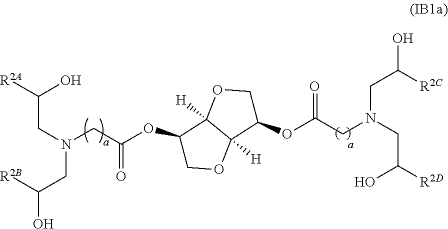

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is independently selected from 3 or 4.

In embodiments, the cationic lipids of the present invention include compounds having a structure according to Formula (IB1b):

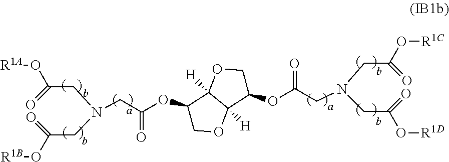

or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl;
optionally wherein i) each a is 2, and/or ii) each b is independently selected from 5 or 7.

In embodiments, the cationic lipids of the present invention include compounds having a structure according to Formula (IB1c):

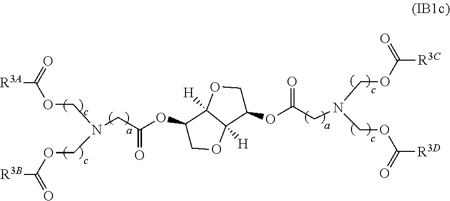

or a pharmaceutically acceptable salt thereof, wherein each R^{3A}, R^{3B}, R^{3C} and R^{3D} is independently selected from optionally substituted alkyl, optionally substituted alkenyl, and optionally substituted alkynyl;
optionally wherein i) each a is 3, and/or ii) each c is 6.

In embodiments, the cationic lipids of the present invention include compounds having a structure according to Formula (IB1d):

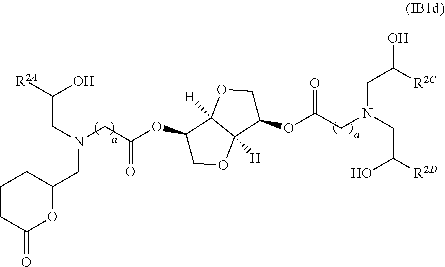

or a pharmaceutically acceptable salt thereof, wherein each R^{2A}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is 3.

In embodiments, the cationic lipids of the present invention include compounds having a structure according to Formula (I″):

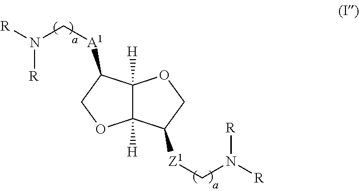

or a pharmaceutically acceptable salt thereof.

In embodiments, the cationic lipids of the present invention include compounds having a structure according to Formula (IB2a):

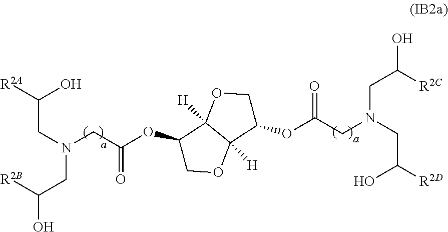

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is independently selected from 3 or 4.

In embodiments, the cationic lipids of the present invention include compounds having a structure according to Formula (IC1a):

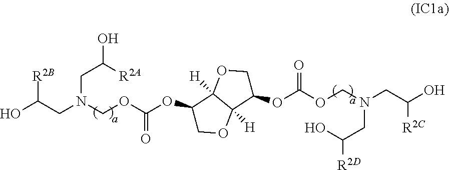

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is independently selected from 3 or 4.

In embodiments, the cationic lipids of the present invention include compounds having a structure according to Formula (IC1b):

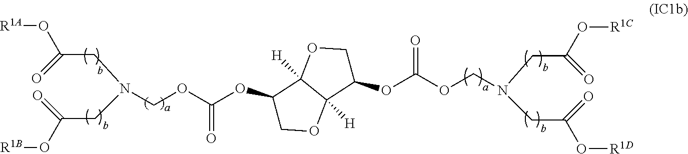

or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl;
optionally wherein each a is 2, and/or ii) each b is independently selected from 5 or 7.

In embodiments, the cationic lipids of the present invention include compounds having a structure according to Formula (IC2a):

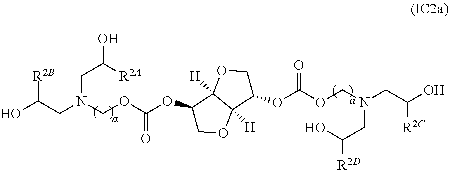

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is 3.

In embodiments, the cationic lipids of the present invention include compounds having a structure according to Formula (ID):

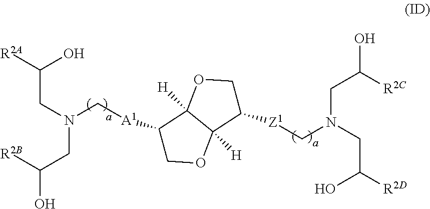

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

In embodiments, A¹ is —C(═O)NH—, wherein the left hand side of the recited structure is bound to the —(CH₂)ₐ—, and Z¹ is —NHC(═O)—, wherein the right hand side of the recited structure is bound to the —(CH₂)ₐ—; optionally wherein each a is 3.

In embodiments, A¹ is —OC(═O)NH—, wherein the left hand side of the recited structure is bound to the —(CH₂)ₐ—, and Z¹ is —NHC(═O)O—, wherein the right hand side of the recited structure is bound to the —(CH₂)ₐ—; optionally wherein each a is 3.

In embodiments, A¹ is —SC(═O)NH—, wherein the left hand side of the recited structure is bound to the —(CH₂)ₐ—, and Z¹ is —NHC(═O)S—, wherein the right hand side of the recited structure is bound to the —(CH₂)ₐ—; optionally wherein each a is 3.

In embodiments, A¹ is —C(═O)S—, wherein the left hand side of the recited structure is bound to the —(CH₂)ₐ—, and Z¹ is —SC(═O)—, wherein the right hand side of the recited structure is bound to the —(CH₂)ₐ—; optionally wherein each a is 3.

In embodiments, A¹ is —S—S—, and Z¹ is —S—S—; optionally wherein each a is 3.

In embodiments, A¹ is —S—, and Z¹ is —S—; optionally wherein each a is 4.

In embodiments, A¹ is —S—S—, and Z¹ is —SC(═O)—, wherein the right hand side of the recited structure is bound to the —(CH₂)ₐ—; optionally wherein each a is 3.

In embodiments, A¹ is —NHC(═O)O—, wherein the left hand side of the recited structure is bound to the —(CH₂)ₐ—, and Z¹ is —OC(═O)NH—, wherein the right hand side of the recited structure is bound to the —(CH₂)ₐ—; optionally wherein each a is 3.

In embodiments, the cationic lipids of the present invention include compounds having a structure according to Formula (II):

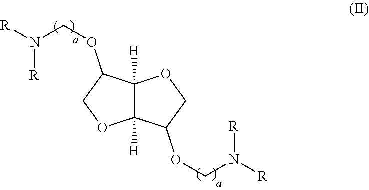

or a pharmaceutically acceptable salt thereof wherein:
each R is independently selected from:

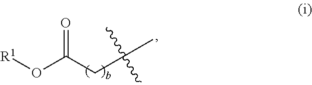

wherein each R¹ is independently selected from optionally substituted alkyl, optionally substituted alkenyl, and optionally substituted alkynyl; and

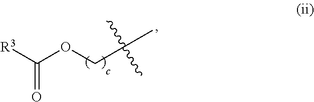

wherein each R³ is independently selected from optionally substituted alkyl, optionally substituted alkenyl, and optionally substituted alkynyl;
each a is independently selected from 2, 3, 4, and 5;
each b is independently selected from 2, 3, 4, 5, 6, and 7; and
each c is independently selected from 2, 3, 4, 5, 6, and 7.

In embodiments, the cationic lipids of the present invention include compounds having a structure according to Formula (II′):

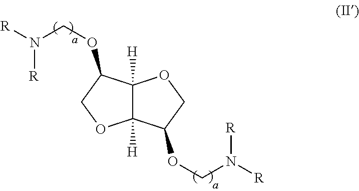

or a pharmaceutically acceptable salt thereof.

In embodiments, the cationic lipids of the present invention include compounds having a structure according to Formula (IIA):

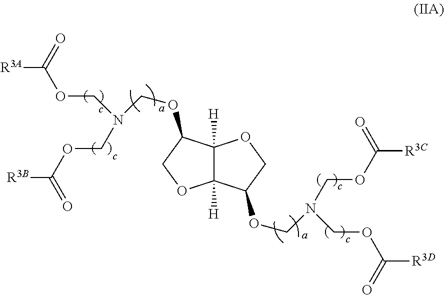

or a pharmaceutically acceptable salt thereof, wherein each R^{3A}, R^{3B}, R^{3C} and R^{3D} is independently selected from optionally substituted alkyl, optionally substituted alkenyl, and optionally substituted alkynyl.

In embodiments, the cationic lipids of the present invention include compounds having a structure according to Formula (IIB):

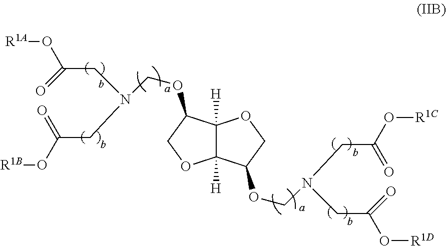

or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl.

In embodiments, the cationic lipids of the present invention include compounds of Formula (I) having a structure according to Formula (ID1):

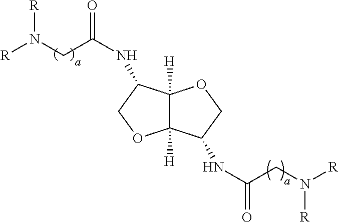

or a pharmaceutically acceptable salt thereof, wherein each R is independently selected from:

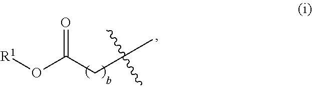

wherein each R¹ is independently selected from optionally substituted alkyl, optionally substituted alkenyl, and optionally substituted alkynyl;

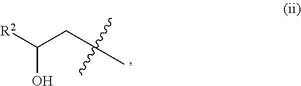

wherein each R² is independently selected from optionally substituted alkyl, optionally substituted alkenyl, optionally substituted alkynyl, and —W¹—X¹, wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;

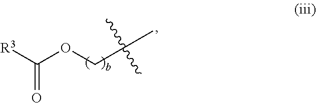

wherein each R³ is independently selected from optionally substituted alkyl, optionally substituted alkenyl, and optionally substituted alkynyl;
optionally wherein (i) each a is 3 or 4 and/or (ii) each b is 5, 6, or 7.

In embodiments, the cationic lipids of the present invention include compounds of Formula (I) having a structure according to Formula (ID2):

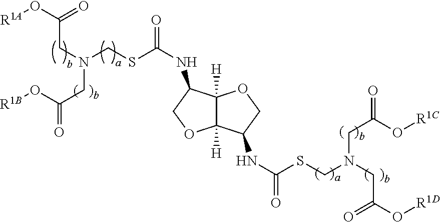

or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl;
optionally wherein (i) each a is 4, and/or (ii) each b is independently selected from 5 or 7.

In embodiments, the cationic lipids of the present invention include compounds of Formula (I) having a structure according to Formula (IE1):

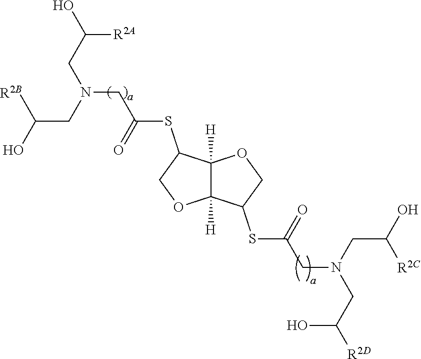

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is 3 or 4.

In embodiments, the cationic lipids of the present invention include compounds of Formula (I) having a structure according to Formula (IE2):

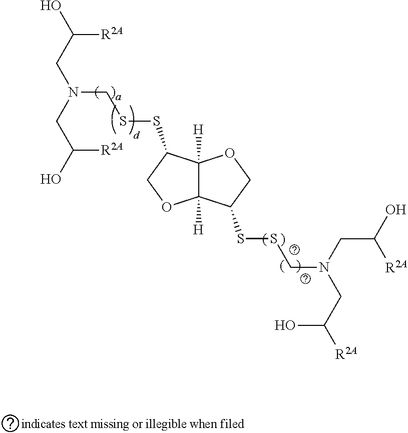

or a pharmaceutically acceptable salt thereof, wherein d is 0 or 1, and
wherein each R^{2A}, R^{2B}, R^{2C} and R^{2D} is independently selected from optionally substituted alkyl, optionally substituted alkenyl, optionally substituted alkynyl, and —W¹—X¹, wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹; optionally wherein each a is 3 or 4.

In embodiments, A¹ and Z¹ are the same. In embodiments, A¹ and Z¹ are different.

In embodiments, A¹ is —C(═O)O—, wherein the left hand side of the recited structure is bound to the —(CH₂)ₐ—. In embodiments, A¹ is —OC(═O)O—.

In embodiments, A¹ is —C(═O)S—, wherein the left hand side of the recited structure is bound to the —(CH₂)ₐ—. In embodiments, A¹ is —C(═O)NH—, wherein the left hand side of the recited structure is bound to the —(CH₂)ₐ—. In embodiments, A¹ is —OC(═O)NH—, wherein the left hand side of the recited structure is bound to the —(CH₂)ₐ—. In embodiments, A¹ is —NHC(═O)O—, wherein the left hand side of the recited structure is bound to the —(CH₂)ₐ—. In embodiments, A¹ is —SC(═O)NH—, wherein the left hand side of the recited structure is bound to the —(CH₂)ₐ—. In embodiments, A¹ is —OCH₂CH₂O—. In embodiments, A¹ is —OCH₂O—. In embodiments, A¹ is —OCH(CH₃)O—. In embodiments, A¹ is —S—. In embodiments, A¹ is —S—S—.

In embodiments, Z¹ is —OC(═O)—, wherein the right hand side of the recited structure is bound to the —(CH₂)ₐ—. In embodiments, Z¹ is —OC(═O)O—.

In embodiments, Z¹ is —SC(═O)—, wherein the right hand side of the recited structure is bound to the —(CH₂)ₐ—. In embodiments, Z¹ is —NHC(═O)—, wherein the right hand side of the recited structure is bound to the —(CH₂)ₐ—. In embodiments, Z¹ is —NHC(═O)O—, wherein the right hand side of the recited structure is bound to the —(CH₂)ₐ—. In embodiments, Z¹ is —OC(═O)NH—, wherein the right hand side of the recited structure is bound to the —(CH₂)ₐ—. In embodiments, Z¹ is —NHC(═O)S—, wherein the right hand side of the recited structure is bound to the —(CH₂)ₐ—. In embodiments, Z¹ is —OCH₂CH₂O—. In embodiments, Z¹ is —OCH₂O—. In embodiments, Z¹ is —OCH(CH₃)O—. In embodiments, Z¹ is —S—. In embodiments, Z¹ is —S—S—.

In embodiments, each a is independently selected from 3 and 4. In embodiments, each a is 2. In embodiments, each a is 3. In embodiments, each a is 4. In embodiments, each a is 5. In embodiments, each a is different. In embodiments, the value for the a on the left hand side of the depicted Formula is 3 and the value for the a on the right hand side of the depicted Formula is 4. In embodiments, the value for the a on the left hand side of the depicted Formula is 4 and the value for the a on the right hand side of the depicted Formula is 3.

In embodiments, the value for the a on the left hand side of the depicted Formula is 2. In embodiments, the value for the a on the left hand side of the depicted Formula is 3. In embodiments, the value for the a on the left hand side of the depicted Formula is 4. In embodiments, the value for the a on the left hand side of the depicted Formula is 5.

In embodiments, the value for the a on the right hand side of the depicted Formula is 2. In embodiments, the value for the a on the right hand side of the depicted Formula is 3. In embodiments, the value for the a on the right hand side of the depicted Formula is 4. In embodiments, the value for the a on the right hand side of the depicted Formula is 5.

In embodiments, each b is independently selected from 5, 6, and 7. In embodiments, each b is independently selected from 5 and 7. In embodiments, each b is 2. In embodiments, each b is 3. In embodiments, each b is 4. In embodiments, each b is 5. In embodiments, each b is 6. In embodiments, each b is 7. In embodiments, each b is 8. In embodiments, each b is 9. In embodiments, each b is 10.

In embodiments, each c is 2. In embodiments, each c is 3. In embodiments, each c is 4. In embodiments, each c is 5. In embodiments, each c is 6. In embodiments, each c is 7. In embodiments, each c is 8. In embodiments, each c is 9. In embodiments, each c is 10.

In embodiments, each R⁴ is optionally substituted cycloalkyl. In embodiments, each R⁴ is optionally substituted heterocycloalkyl. In embodiments, each R⁴ is

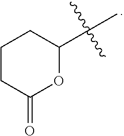 [SMILES: *C1CCCC(=O)O1]

In embodiments, each R is independently selected from:

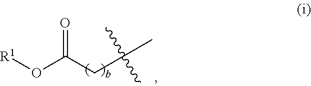

wherein each R¹ is independently selected from optionally substituted alkyl, optionally substituted alkenyl, and optionally substituted alkynyl;

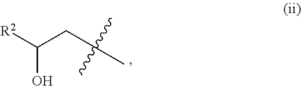

wherein each R² is independently selected from optionally substituted alkyl, optionally substituted alkenyl, optionally substituted alkynyl, and —W¹—X¹,
   wherein each W¹ is independently selected from optionally substituted alkylene and optionally substituted alkenylene, and
   each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹; and

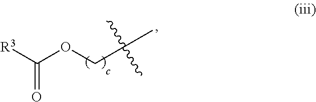

wherein each R³ is independently selected from optionally substituted alkyl, optionally substituted alkenyl, and optionally substituted alkynyl.

In embodiments, each R is independently selected from

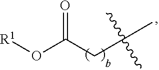

wherein each R¹ is independently selected from optionally substituted alkyl, optionally substituted alkenyl, and optionally substituted alkynyl.

In embodiments, each R¹ is the same. In embodiments, at least one R¹ is different.

In embodiments, R^{1A}, R^{1B}, R^{1C} and R^{1D} are the same. In embodiments, R^{1A} and R^{1B} are the same. In embodiments, R^{1C} and R^{1D} are the same. In embodiments, R^{1A} and R^{1C} are the same. In embodiments, R^{1B} and R^{1D} are the same.

In embodiments, R^{1A} and R^{1B} are the same and R^{1C} and R^{1D} are the same, but wherein R^{1A} and R^{1B} are different to R^{1C} and R^{1D}. In embodiments, R^{1A} and R^{1C} are the same and R^{1B} and R^{1D} are the same, but wherein R^{1A} and R^{1C} are different to R^{1B} and R^{1D}.

In embodiments, each R¹ or each R^{1A}, R^{1B}, R^{1C} and R^{1D}, when present, is independently selected from optionally substituted (C₅-C₂₅) alkyl, optionally substituted (C₅-C₂₅) alkenyl, and optionally substituted (C₅-C₂₅) alkynyl.

In embodiments, each R¹ or each R^{1A}, R^{1B}, R^{1C} and R^{1D}, when present, is independently selected from optionally substituted alkyl. In embodiments, each R¹ or each R^{1A}, R^{1B}, R^{1C} and R^{1D}, when present, is independently selected from optionally substituted (C₅-C₂₅) alkyl. In embodiments, each R¹ or each R^{1A}, R^{1B}, R^{1C} and R^{1D}, when present, is independently selected from optionally substituted (C₁₀-C₂₀) alkyl.

In embodiments, each R^{1A}, when present, is optionally substituted alkyl. In embodiments, each R^{1A}, when present, is optionally substituted (C₅-C₂₅) alkyl. In embodiments, each R^{1A}, when present, is optionally substituted (C₁₀-C₂₀) alkyl.

In embodiments, each R^{1B}, when present, is optionally substituted alkyl. In embodiments, each R^{1B}, when present, is optionally substituted (C₅-C₂₅) alkyl. In embodiments, each R^{1B}, when present, is optionally substituted (C₁₀-C₂₀) alkyl.

In embodiments, each R^{1C}, when present, is optionally substituted alkyl. In embodiments, each R^{1C}, when present, is optionally substituted (C₅-C₂₅) alkyl. In embodiments, each R^{1C}, when present, is optionally substituted (C₁₀-C₂₀) alkyl.

In embodiments, each R^{1D}, when present, is optionally substituted alkyl. In embodiments, each R^{1D}, when present, is optionally substituted (C₅-C₂₅) alkyl. In embodiments, each R^{1D}, when present, is optionally substituted (C₁₀-C₂₀) alkyl.

In embodiments, each R¹ or each R^{1A}, R^{1B}, R^{1B} and R^{1D}, when present, is independently selected from optionally substituted alkenyl. In embodiments, each R¹ or each R^{1A}, R^{1B}, R^{1C} and R^{1D}, when present, is independently selected from optionally substituted (C₅-C₂₅) alkenyl. In embodiments, each R¹ or each R^{1A}, R^{1B}, R^{1C} and R^{1D}, when present, is independently selected from optionally substituted (C₁₀-C₂₀) alkenyl.

In embodiments, each R^{1A}, when present, is optionally substituted alkenyl. In embodiments, each R^{1A}, when present, is optionally substituted (C₅-C₂₅) alkenyl. In embodiments, each R^{1A}, when present, is optionally substituted (C₁₀-C₂₀) alkenyl.

In embodiments, each R^{1B}, when present, is optionally substituted alkenyl. In embodiments, each R^{1B}, when present, is optionally substituted (C₅-C₂₅) alkenyl. In embodiments, each R^{1B}, when present, is optionally substituted (C₁₀-C₂₀) alkenyl.

In embodiments, each R^{1C}, when present, is optionally substituted alkenyl. In embodiments, each R^{1C}, when present, is optionally substituted (C₅-C₂₅) alkenyl. In embodiments, each R^{1C}, when present, is optionally substituted (C₁₀-C₂₀) alkenyl.

In embodiments, each R^{1D}, when present, is optionally substituted alkenyl. In embodiments, each R^{1D}, when present, is optionally substituted (C₅-C₂₅) alkenyl. In embodiments, each R^{1D}, when present, is optionally substituted (C₁₀-C₂₀) alkenyl.

In embodiments, each R¹ or each R^{1A}, R^{1B}, R^{1C} and R^{1D}, when present, is independently selected from optionally substituted alkynyl. In embodiments, each R¹ or each R^{1A}, R^{1B}, R^{1C} and R^{1D}, when present, is independently selected from optionally substituted (C₅-C₂₅) alkynyl. In embodiments, each R¹ or each R^{1A}, R^{1B}, R^{1C} and R^{1D}, when present, is independently selected from optionally substituted (C₁₀-C₂₀) alkynyl.

In embodiments, each R^{1A}, when present, is optionally substituted alkynyl. In embodiments, each R^{1A}, when present, is optionally substituted (C₅-C₂₅) alkynyl. In embodiments, each R^{1A}, when present, is optionally substituted (C₁₀-C₂₀) alkynyl.

In embodiments, each R^{1B}, when present, is optionally substituted alkynyl. In embodiments, each R^{1B}, when present, is optionally substituted (C₅-C₂₅) alkynyl. In embodiments, each R^{1B}, when present, is optionally substituted (C₁₀-C₂₀) alkynyl.

In embodiments, each R^{1C}, when present, is optionally substituted alkynyl. In embodiments, each R^{1C}, when present, is optionally substituted (C₅-C₂₅) alkynyl. In embodiments, each R^{1C}, when present, is optionally substituted (C₁₀-C₂₀) alkynyl.

In embodiments, each R^{1D}, when present, is optionally substituted alkynyl. In embodiments, each R^{1D}, when present, is optionally substituted (C₅-C₂₅) alkynyl. In embodiments, each R^{1D}, when present, is optionally substituted (C₁₀-C₂₀) alkynyl.

In embodiments, each R¹ or each R^{1A}, R^{1B}, R^{1C} and R^{1D}, when present, is independently selected from:

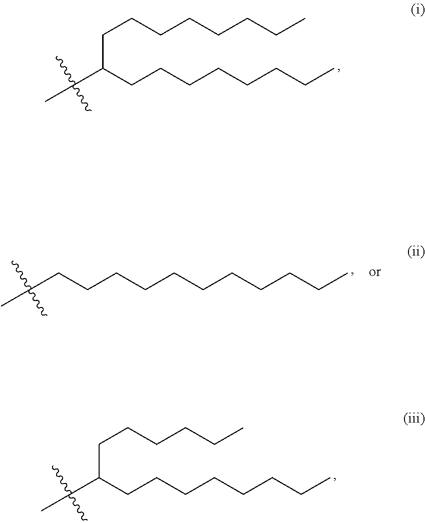 [SMILES: *C(CCCCCCCC)CCCCCCCC ; *CCCCCCCCCCC ; *C(CCCCCC)CCCCCCCC]

optionally wherein each R¹ or each R^{1A}, R^{1B}, R^{1C} and R^{1D}, when present, is independently selected from options (i) and (ii).

In embodiments, each R¹ or each R^{1A}, R^{1B}, R^{1C} and R^{1D}, when present, is

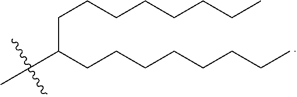 [SMILES: *C(CCCCCCCC)CCCCCCCC]

In embodiments, each R¹ or each R^{1A}, R^{1B}, R^{1C} and R^{1D}, when present, is

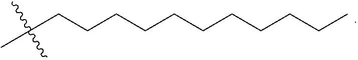 [SMILES: *CCCCCCCCCCC]

In embodiments, each R¹ or each R^{1A}, R^{1B}, R^{1C} and R^{1D}, when present, is

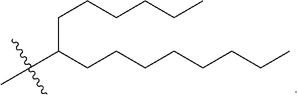 [SMILES: *C(CCCCCC)CCCCCCCC]

In embodiments, each R^{1A}, when present, is

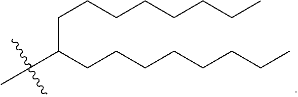 [SMILES: *C(CCCCCCCC)CCCCCCCC]

In embodiments, each R^{1A}, when present, is

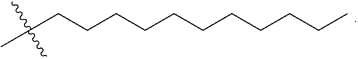 [SMILES: *CCCCCCCCCCC]

In embodiments each R^{1A}, when present, is

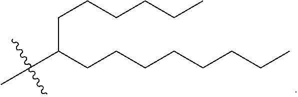 [SMILES: *C(CCCCCC)CCCCCCCC]

In embodiments, each R^{1B}, when present,

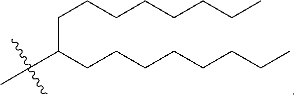 [SMILES: *C(CCCCCCCC)CCCCCCCC]

In embodiments, each R^{1B}, when present, is

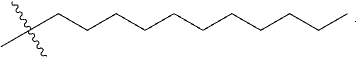 [SMILES: *CCCCCCCCCCC]

In embodiments, each R^{1B}, when present, is

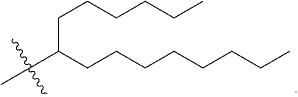 [SMILES: *C(CCCCCC)CCCCCCCC]

In embodiments, each R^{1C}, when present, is

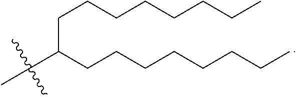 [SMILES: *C(CCCCCCCC)CCCCCCCC]

In embodiments, reach R^{1C}, when present, is

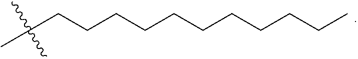 [SMILES: *CCCCCCCCCCC]

In embodiments, each R^{1C}, when present, is

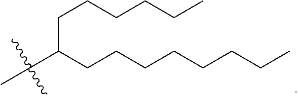 [SMILES: *C(CCCCCC)CCCCCCCC]

In embodiments, each R^{1D}, when present is

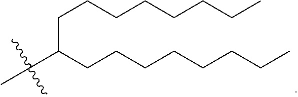 [SMILES: *C(CCCCCCCC)CCCCCCCC]

In each embodiments, each R^{1D}, when present, is

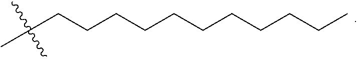 [SMILES: *CCCCCCCCCCC]

In embodiments, each R^{1D}, when present, is

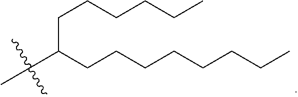 [SMILES: *C(CCCCCC)CCCCCCCC]

In embodiments, each R is independently selected from

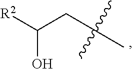

wherein each R² is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

In embodiments, each R² is the same. In embodiments, at least one R² is different.

In embodiments, R^{2A}, R^{2B}, R^{2C} and R^{2D} are the same. In embodiments, R^{2A} and R^{2B} are the same. In embodiments, R^{2C} and R^{2D} are the same. In embodiments, R^{2A} and R^{2C} are the same. In embodiments, R^{2B} and R^{2D} are the same.

In embodiments, R^{2A} and R^{2B} are the same and R^{2C} and R^{2D} are the same, but wherein R^{2A} and R^{2B} are different to R^{2C} and R^{2D}. In embodiments, R^{2A} and R^{2C} are the same and R^{2B} and R^{2D} are the same, but wherein R^{2A} and R^{2C} are different to R^{2B} and R^{2D}.

In embodiments, each R² or each R^{2A}, R^{2B}, R^{2C} and R^{2D}, when present, is independently selected from optionally substituted (C₅-C₂₅) alkyl, optionally substituted (C₅-C₂₅) alkenyl, optionally substituted (C₅-C₂₅) alkynyl, and —W¹—X¹,
wherein each W¹ is independently selected from optionally substituted (C₁-C₁₀) alkylene and optionally substituted (C₂-C₁₀) alkenylene, and
each X¹ is independently selected from —*O—(C═O)-optionally substituted (C₅-C₂₅) alkyl, —(*C═O)—O-optionally substituted (C₅-C₂₅) alkyl, —*O—(C═O)-optionally substituted (C₅-C₂₅) alkenyl, and —(*C═O)—O-optionally substituted (C₅-C₂₅) alkenyl, wherein the atom marked with a * is connected to W¹.

In embodiments, each R² or each R^{2A}, R^{2B}, R^{2C} and R^{2D}, when present, is independently selected from optionally substituted (C₅-C₂₅) alkyl, for example optionally substituted (C₅-C₂₀) alkyl, and —W¹—X¹,
wherein each W¹ is independently selected from optionally substituted (C₁-C₁₀) alkylene, for example optionally substituted (C₂-C₆) alkylene, and optionally substituted (C₂-C₁₀) alkenylene, for example optionally substituted (C₂-C₆) alkenylene, and
each X¹ is independently selected from —*O—(C═O)-optionally substituted (C₅-C₂₅) alkyl, for example —*O—(C═O)-optionally substituted (C₈-C₂₀) alkyl, —(*C═O)—O-optionally substituted (C₅-C₂₅) alkyl, for example —(*C═O)—O-optionally substituted (C₈-C₂₀) alkyl, —*O—(C═O)-optionally substituted (C₅-C₂₅) alkenyl, for example —*O—(C═O)-optionally substituted (C₈-C₂₀) alkenyl, and —(*C═O)—O-optionally substituted (C₅-C₂₅) alkenyl, for example —(*C═O)—O-optionally substituted (C₈-C₂₀) alkenyl, wherein the atom marked with a * is connected to W¹.

In embodiments, each R² or each R^{2A}, R^{2B}, R^{2C} and R^{2D}, when present, is independently selected from optionally substituted alkyl and —W¹—X¹, optionally wherein
each W¹ is independently selected from optionally substituted alkylene, and
each X¹ is independently selected from —*O—(C═O)-optionally substituted alkyl, —(*C═O)—O-optionally substituted alkyl, and —(*C═O)—O-optionally substituted alkenyl, wherein the atom marked with a * is connected to W¹.

In embodiments, each R² or each R^{2A}, R^{2B}, R^{2C} and R^{2D}, when present, is independently selected from optionally substituted alkyl. In embodiments, each R² or each R^{2A}, R^{2B}, R^{2C} and R^{2D}, when present, is independently selected from optionally substituted (C₅-C₂₅) alkyl. In embodiments, each R² or each R^{2A}, R^{2B}, R^{2C} and R^{2D}, when present, is independently selected from optionally substituted (C₅-C₂₀) alkyl.

In embodiments, each R^{2A}, when present, is optionally substituted alkyl. In embodiments, each R^{2A}, when present, is optionally substituted (C₅-C₂₅) alkyl. In embodiments, each R^{2A}, when present, is optionally substituted (C₅-C₂₀) alkyl.

In embodiments, each R^{2B}, when present, is optionally substituted alkyl. In embodiments, each R^{2B}, when present, is optionally substituted (C₅-C₂₅) alkyl. In embodiments, each R^{2B}, when present, is optionally substituted (C₅-C₂₀) alkyl.

In embodiments, each R^{2C}, when present, is optionally substituted alkyl. In embodiments, each R^{2C}, when present, is optionally substituted (C₅-C₂₅) alkyl. In embodiments, each R^{2C}, when present, is optionally substituted (C₅-C₂₀) alkyl.

In embodiments, each R^{2D}, when present, is optionally substituted alkyl. In embodiments, each R^{2D}, when present, is optionally substituted (C₅-C₂₅) alkyl. In embodiments, each R^{2D}, when present, is optionally substituted (C₅-C₂₀) alkyl.

In embodiments, each R² or each R^{2A}, R^{2B}, R^{2C} and R^{2D}, when present, is independently selected from optionally substituted alkenyl. In embodiments, each R² or each R^{2A}, R^{2B}, R^{2C} and R^{2D}, when present, is independently selected from optionally substituted (C₅-C₂₅) alkenyl.

In embodiments, each R^{2A}, when present, is optionally substituted alkenyl. In embodiments, each R^{2A}, when present, is optionally substituted (C₅-C₂₅) alkenyl.

In embodiments, each R^{2B}, when present, is optionally substituted alkenyl. In embodiments, each R^{2B}, when present, is optionally substituted (C₅-C₂₅) alkenyl.

In embodiments, each R^{2C}, when present, is optionally substituted alkenyl. In embodiments, each R^{2C}, when present, is optionally substituted (C₅-C₂₅) alkenyl.

In embodiments, each R^{2D}, when present, is optionally substituted alkenyl. In embodiments, each R^{2D}, when present, is optionally substituted (C₅-C₂₅) alkenyl.

In embodiments, each R² or each R^{2A}, R^{2B}, R^{2C} and R^{D}, when present, is independently selected from optionally substituted alkynyl. In embodiments, each R² or each R^{2A}, R^{2B}, R^{2C} and R^{2D}, when present, is independently selected from optionally substituted (C₅-C₂₅) alkynyl.

In embodiments, each R^{2A}, when present, is optionally substituted alkynyl. In embodiments, each R^{2A}, when present, is optionally substituted (C₅-C₂₅) alkynyl.

In embodiments, each R^{2B}, when present, is optionally substituted alkynyl. In embodiments, each R^{2B}, when present, is optionally substituted (C₅-C₂₅) alkynyl.

In embodiments, each R^{2C}, when present, is optionally substituted alkynyl. In embodiments, each R^{2C}, when present, is optionally substituted (C₅-C₂₅) alkynyl.

In embodiments, each R^{2D}, when present, is optionally substituted alkynyl. In embodiments, each R^{2D}, when present, is optionally substituted (C₅-C₂₅) alkynyl.

In embodiments, each R² or each R^{2A}, R^{2B}, R^{2C} and R^{2D}, when present, is independently selected from —W¹—X¹.

In embodiments, each R² or each R^{2A}, R^{2B}, R^{2C} and R^{2D}, when present, is independently selected from —W¹—X¹, wherein
each W¹ is independently selected from optionally substituted alkylene, and
each X¹ is independently selected from —*O—(C═O)-optionally substituted alkyl, —(*C═O)—O-optionally substituted alkyl, and —(*C═O)—O-optionally substituted alkenyl, wherein the atom marked with a * is connected to W¹.

In embodiments, each R² or each R^{2A}, R^{2B}, R^{2C} and R^{2D}, when present, is independently selected from —W¹—X¹,
wherein each W¹ is independently selected from optionally substituted (C₁-C₁₀) alkylene and optionally substituted (C₂-C₁₀) alkenylene, and
each X¹ is independently selected from —*O—(C═O)-optionally substituted (C₅-C₂₅) alkyl, —(*C═O)—O-optionally substituted (C₅-C₂₅) alkyl, —*O—(C═O)-optionally substituted (C₅-C₂₅) alkenyl, and —(*C═O)—O-optionally substituted (C₅-C₂₅) alkenyl, wherein the atom marked with a * is connected to W¹.

In embodiments, each R² or each R^{2A}, R^{2B}, R^{2C} and R^{2D}, when present, is independently selected from —W¹—X¹,
wherein each W¹ is independently selected from optionally substituted (C₁-C₁₀) alkylene, for example optionally substituted (C₂-C₆) alkylene, and optionally substituted (C₂-C₁₀) alkenylene, for example optionally substituted (C₂-C₆) alkenylene, and
each X¹ is independently selected from —*O—(C═O)-optionally substituted (C₅-C₂₅) alkyl, for example —*O—(C═O)-optionally substituted (C₈-C₂₀) alkyl, —(*C═O)—O-optionally substituted (C₅-C₂₅) alkyl, for example —(*C═O)—O-optionally substituted (C₈-C₂₀) alkyl, —*O—(C═O)-optionally substituted (C₅-C₂₅) alkenyl, for example —*O—(C═O)-optionally substituted (C₈-C₂₀) alkenyl, and —(*C═O)—O-optionally substituted (C₅-C₂₅) alkenyl, for example —(*C═O)—O-optionally substituted (C₈-C₂₀) alkenyl, wherein the atom marked with a * is connected to W¹.

In embodiments, each R^{2A}, when present, is —W¹—X¹.

In embodiments, each R^{2B}, when present, is —W¹—X¹.

In embodiments, each R^{2C}, when present, is —W¹—X¹.

In embodiments, each R^{2D}, when present, is —W¹—X¹.

In embodiments, each W¹ is independently selected from optionally substituted alkylene. In embodiments, each W¹ is independently selected from optionally substituted (C₁-C₁₀) alkylene. In embodiments, each W¹ is independently selected from optionally substituted (C₂-C₆) alkylene.

In embodiments, each W¹ is independently selected from optionally substituted alkenylene. In embodiments, each W¹ is independently selected from optionally substituted (C₂-C₁₀) alkenylene. In embodiments, each W¹ is independently selected from optionally substituted (C₂-C₆) alkenylene.

In embodiments, each X¹ is independently selected from —*O—(C═O)-optionally substituted alkyl, wherein the atom marked with a * is connected to W¹. In embodiments, each X¹ is independently selected from —*O—(C═O)-optionally substituted (C₅-C₂₅) alkyl, wherein the atom marked with a * is connected to W¹. In embodiments, each X¹ is independently selected from —*0-(C═O)-optionally substituted (C₅-C₂₀) alkyl, wherein the atom marked with a * is connected to W¹.

In embodiments, each X¹ is independently selected from —(*C═O)—O-optionally substituted alkyl, wherein the atom marked with a * is connected to W¹. In embodiments, each X¹ is independently selected from —(*C═O)—O-optionally substituted (C₅-C₂₅) alkyl, wherein the atom marked with a * is connected to W¹. In embodiments, each X¹ is independently selected from —(*C═O)—O-optionally substituted (C₈-C₂₀) alkyl, wherein the atom marked with a * is connected to W¹.

In embodiments, each X¹ is independently selected from —*O—(C═O)-optionally substituted alkenyl, wherein the atom marked with a * is connected to W¹. In embodiments, each X¹ is independently selected from —*O—(C═O)-optionally substituted (C₅-C₂₅) alkenyl, wherein the atom marked with a * is connected to W¹. In embodiments, each X¹ is independently selected from —*0-(C═O)-optionally substituted (C₈-C₂₀) alkenyl, wherein the atom marked with a * is connected to W¹.

In embodiments, each X¹ is independently selected from —(*C═O)—O-optionally substituted alkenyl, wherein the atom marked with a * is connected to W¹. In embodiments, each X¹ is independently selected from —(*C═O)—O-optionally substituted (C₅-C₂₅) alkenyl, wherein the atom marked with a * is connected to W¹. In embodiments, each X¹ is independently selected from —(*C═O)—O-optionally substituted (C₈-C₂₀) alkenyl, wherein the atom marked with a * is connected to W¹.

In embodiments, each R² or each R^{2A}, R^{2B}, R^{2C} and R^{2D}, when present, is independently selected from:

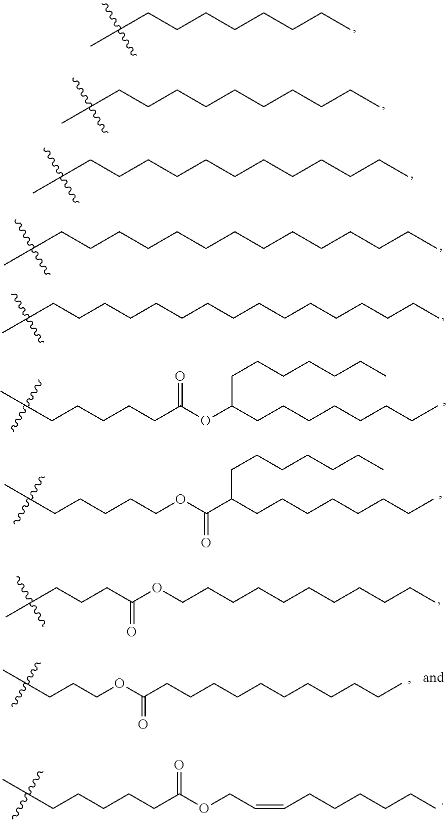

In embodiments, each R² or each R^{2A}, R^{2B}, R^{2C} and R^{2D}, when present, is

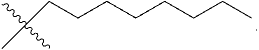 [SMILES: *CCCCCCCC]

In embodiments, each R² or each R^{2A}, R^{2B}, R^{2C} and R^{2D}, when present, is

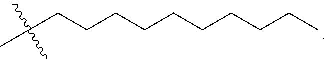 [SMILES: *CCCCCCCCCC]

In embodiments, each R² or each R^{2A}, R^{2B}, R^{2C} and R^{2D}, when present, is

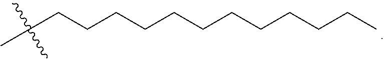 [SMILES: *CCCCCCCCCCCC]

In embodiments, each R² or each R^{2A}, R^{2B}, R^{2C} and R^{2D}, when present, is

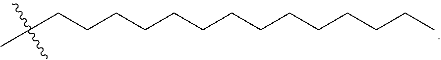 [SMILES: *CCCCCCCCCCCCCC]

In embodiments, each R² or each R^{2A}, R^{2B}, R^{2C} and R^{2D}, when present, is

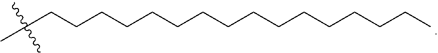 [SMILES: *CCCCCCCCCCCCCCCC]

In embodiments, each R² or each R^{2A}, R^{2B}, R^{2C} and R^{2D}, when present, is

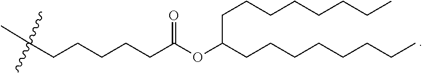 [SMILES: *CCCCCC(=O)OC(CCCCCCCC)CCCCCCCC]

In embodiments, each R² or each R^{2A}, R^{2B}, R^{2C} and R^{2D}, when present, is

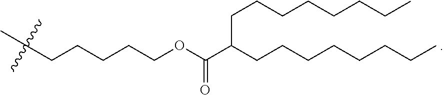 [SMILES: *CCCCCOC(=O)C(CCCCCCCC)CCCCCCCC]

In embodiments, each R² or each R^{2A}, R^{2B}, R^{2C} and R^{2D}, when present, is

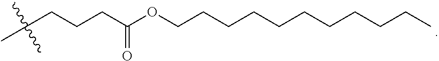 [SMILES: *CCCC(=O)OCCCCCCCCCCC]

In embodiments, each R² or each R^{2A}, R^{2B}, R^{2C} and R^{2D}, when present, is

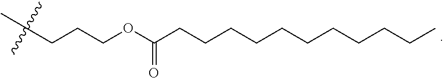 [SMILES: *CCCOC(=O)CCCCCCCCCCC]

In embodiments, each R² or each R^{2A}, R^{2B}, R^{2C} and R^{2D}, when present, is

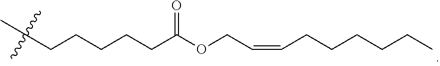 [SMILES: *CCCCCC(=O)OC/C=C\CCCCCC]

In embodiments, each R^{2A}, when present, is

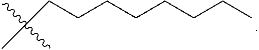 [SMILES: *CCCCCCCC]

In embodiments, each R^{2A}, when present, is

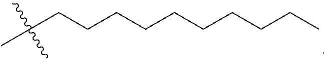 [SMILES: *CCCCCCCCCC]

In embodiments, each R^{2A}, when present, is

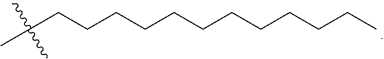 [SMILES: *CCCCCCCCCCCC]

In embodiments, each R^{2A}, when present, is

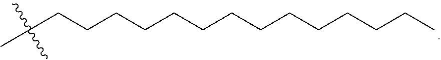 [SMILES: *CCCCCCCCCCCCCC]

In embodiments, each R^{2A}, when present, is

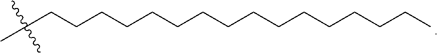 [SMILES: *CCCCCCCCCCCCCCCC]

In embodiments, each R^{2A}, when present, is

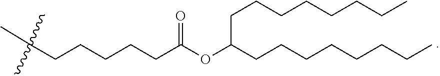 [SMILES: *CCCCCC(=O)OC(CCCCCCCC)CCCCCCCC]

In embodiments, each R^{2A}, when present, is

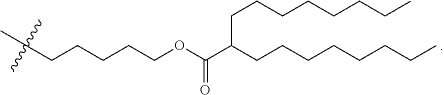 [SMILES: *CCCCCOC(=O)C(CCCCCCCC)CCCCCCCC]

In embodiments, each R^{2A}, when present, is

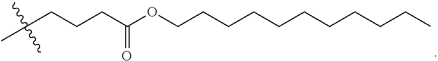 [SMILES: *CCCC(=O)OCCCCCCCCCCC]

In embodiments, each R^{2A}, when

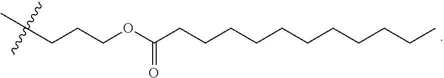 [SMILES: *CCCOC(=O)CCCCCCCCCCC]

In embodiments, each R^{2A}, when present, is

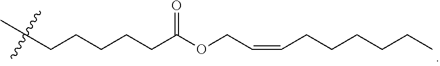 [SMILES: *CCCCCC(=O)OC/C=C\CCCCCC]

In embodiments, each R^{2B}, when present, is

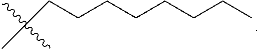 [SMILES: *CCCCCCCC]

In embodiments, each R^{2B}, when present, is

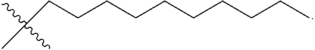 [SMILES: *CCCCCCCCCC]

In embodiments, each R^{2B}, when present, is

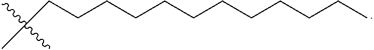 [SMILES: *CCCCCCCCCCCC]

In embodiments, each R^{2B}, when present, is

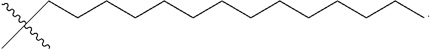 [SMILES: *CCCCCCCCCCCCCC]

In embodiments, each R^{2B}, when present, is

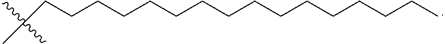 [SMILES: *CCCCCCCCCCCCCCCC]

In embodiments, each R^{2B}, when present, is

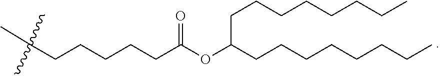 [SMILES: *CCCCCC(=O)OC(CCCCCCCC)CCCCCCCC]

In embodiments, each R^{2B}, when present, is

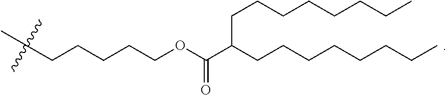 [SMILES: *CCCCCOC(=O)C(CCCCCCCC)CCCCCCCC]

In embodiments, each R^{2B}, when present, is

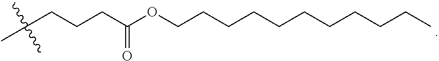 [SMILES: *CCCC(=O)OCCCCCCCCCCC]

In embodiments, each R^{2B}, when present, is

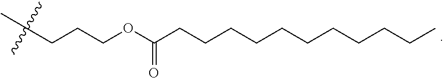 [SMILES: *CCCOC(=O)CCCCCCCCCCC]

In embodiments, each R^{2B}, when present, is

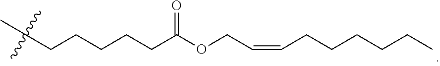 [SMILES: *CCCCCC(=O)OC/C=C\CCCCCC]

In embodiments, each R^{2C}, when present, is

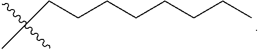 [SMILES: *CCCCCCCC]

In embodiments, each R^{2C}, when present, is

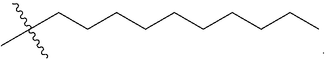 [SMILES: *CCCCCCCCCC]

In embodiments, each R^{2C}, when present, is

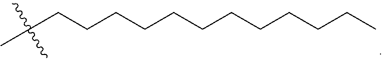 [SMILES: *CCCCCCCCCCCC]

In embodiments, each R^{2C}, when present, is

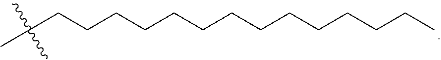 [SMILES: *CCCCCCCCCCCCCC]

In embodiments, each R^{2C}, when present, is

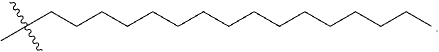 [SMILES: *CCCCCCCCCCCCCCCC]

In embodiments, each R^{2C}, when present, is

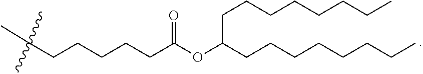 [SMILES: *CCCCCC(=O)OC(CCCCCCCC)CCCCCCCC]

In embodiments, each R^{2C}, when present, is

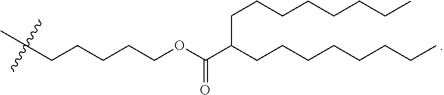 [SMILES: *CCCCCOC(=O)C(CCCCCCCC)CCCCCCCC]

In embodiments, each R^{2C}, when

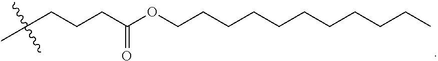 [SMILES: *CCCC(=O)OCCCCCCCCCCC]

In embodiments, each R^{2C}, when

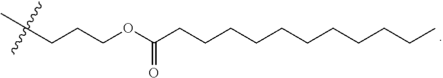 [SMILES: *CCCOC(=O)CCCCCCCCCCC]

In embodiments, each R^{2C}, when present, is

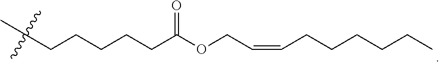 [SMILES: *CCCCCC(=O)OC/C=C\CCCCCC]

In embodiments, each R^{2D}, when present, is

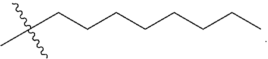 [SMILES: *CCCCCCCC]

In embodiments, each R^{2D}, when present, is

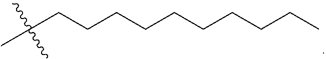 [SMILES: *CCCCCCCCCC]

In embodiments, each R^{2D}, when present, is

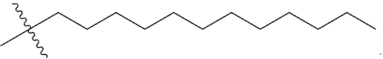 [SMILES: *CCCCCCCCCCCC]

In embodiments, each R^{2D}, when present, is

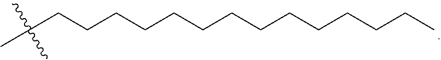 [SMILES: *CCCCCCCCCCCCCC]

In embodiments, each R^{2D}, when

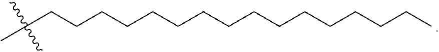 [SMILES: *CCCCCCCCCCCCCCCC]

In embodiments, each R^{2D}, when present, is

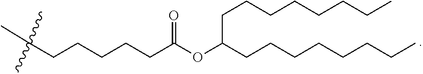 [SMILES: *CCCCCC(=O)OC(CCCCCCCC)CCCCCCCC]

In embodiments, each R^{2D}, when present, is

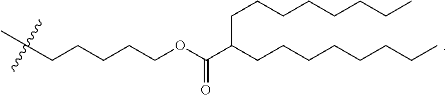 [SMILES: *CCCCCOC(=O)C(CCCCCCCC)CCCCCCCC]

In embodiments, each R^{2D}, when present, is

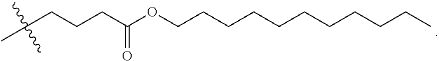 [SMILES: *CCCC(=O)OCCCCCCCCCCC]

In embodiments, each R^{2D}, when present, is

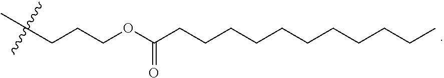 [SMILES: *CCCOC(=O)CCCCCCCCCCC]

In embodiments, each R^{2D}, when present, is

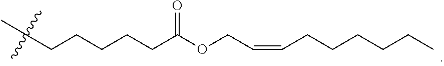 [SMILES: *CCCCCC(=O)OC/C=C\CCCCCC]

In embodiments, each R is independently selected from

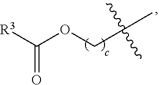

wherein each R³ is independently selected from optionally substituted alkyl, optionally substituted alkenyl, and optionally substituted alkynyl.

In embodiments, each R³ is the same. In embodiments, at least one R³ is different.

In embodiments, R^{3A}, R^{3B}, R^{3C} and R^{3D} are the same. In embodiments, R^{3A} and R^{3B} are the same. In embodiments, R^{3C} and R^{3D} are the same. In embodiments, R^{3A} and R^{3C} are the same. In embodiments, R^{3B} and R^{3D} are the same.

In embodiments, R^{3A} and R^{3B} are the same and R^{3C} and R^{3D} are the same, but wherein R^{3A} and R^{3B} are different to R^{3C} and R^{3D}. In embodiments, R^{3A} and R^{3C} are the same and R^{3B} and R^{3D} are the same, but wherein R^{3A} and R^{3C} are different to R^{3B} and R^{3D}.

In embodiments, each R³ or each R^{3A}, R^{3B}, R^{3C} and R^{3D}, when present, is independently selected from optionally substituted (C₅-C₂₅) alkyl, optionally substituted (C₅-C₂₅) alkenyl, and optionally substituted (C₅-C₂₅) alkynyl.

In embodiments, each R³ or each R^{3A}, R^{3B}, R^{3C} and R^{3D}, when present, is independently selected from optionally substituted alkyl. In embodiments, each R³ or each R^{3A}, R^{3B}, R^{3C} and R^{3D}, when present, is independently selected from optionally substituted (C₅-C₂₅) alkyl. In embodiments, each R³ or each R^{3A}, R^{3B}, R^{3C} and R^{3D}, when present, is independently selected from optionally substituted (C₁₀-C₂₀) alkyl.

In embodiments, each R^{3A}, when present, is optionally substituted alkyl. In embodiments, each R^{3A}, when present, is optionally substituted (C₅-C₂₅) alkyl. In embodiments, each R^{3A}, when present, is optionally substituted (C₁₀-C₂₀) alkyl.

In embodiments, each R^{3B}, when present, is optionally substituted alkyl. In embodiments, each R^{3B}, when present, is optionally substituted (C₅-C₂₅) alkyl. In embodiments, each R^{3B}, when present, is optionally substituted (C₁₀-C₂₀) alkyl.

In embodiments, each R^{3C}, when present, is optionally substituted alkyl. In embodiments, each R^{3C}, when present, is optionally substituted (C₅-C₂₅) alkyl. In embodiments, each R^{3C}, when present, is optionally substituted (C₁₀-C₂₀) alkyl.

In embodiments, each R^{3D}, when present, is optionally substituted alkyl. In embodiments, each R^{3D}, when present, is optionally substituted (C₅-C₂₅) alkyl. In embodiments, each R^{3D}, when present, is optionally substituted (C₁₀-C₂₀) alkyl.

In embodiments, each R³ or each R^{3A}, R^{3B}, R^{3C} and R^{3D}, when present, is independently selected from optionally substituted alkenyl. In embodiments, each R³ or each R^{3A}, R^{3B}, R^{3C} and R^{3D}, when present, is independently selected from optionally substituted (C₅-C₂₅) alkenyl. In embodiments, each R³ or each R^{3A}, R^{3B}, R^{3C} and R^{3D}, when present, is independently selected from optionally substituted (C₁₀-C₂₀) alkenyl.

In embodiments, each R^{3A}, when present, is optionally substituted alkenyl. In embodiments, each R^{3A}, when present, is optionally substituted (C₅-C₂₅) alkenyl. In embodiments, each R^{3A}, when present, is optionally substituted (C₁₀-C₂₀) alkenyl.

In embodiments, each R^{3B}, when present, is optionally substituted alkenyl. In embodiments, each R^{3B}, when present, is optionally substituted (C₅-C₂₅) alkenyl. In embodiments, each R^{3B}, when present, is optionally substituted (C₁₀-C₂₀) alkenyl.

In embodiments, each R^{3C}, when present, is optionally substituted alkenyl. In embodiments, each R^{3C}, when present, is optionally substituted (C₅-C₂₅) alkenyl. In embodiments, each R^{3C}, when present, is optionally substituted (C₁₀-C₂₀) alkenyl.

In embodiments, each R^{3D}, when present, is optionally substituted alkenyl. In embodiments, each R^{3D}, when present, is optionally substituted (C₅-C₂₅) alkenyl. In embodiments, each R^{3D}, when present, is optionally substituted (C₁₀-C₂₀) alkenyl.

In embodiments, each R³ or each R^{3A}, R^{3B}, R^{3C} and R^{3D}, when present, is independently selected from optionally substituted alkynyl. In embodiments, each R³ or each R^{3A}, R^{3B}, R^{3C} and R^{3D}, when present, is independently selected from optionally substituted (C₅-C₂₅) alkynyl. In embodiments, each R³ or each R^{3A}, R^{3B}, R^{3C} and R³_{A}, when present, is independently selected from optionally substituted (C₁₀-C₂₀) alkynyl.

In embodiments, each R^{3A}, when present, is optionally substituted alkynyl. In embodiments, each R^{3A}, when present, is optionally substituted (C₅-C₂₅) alkynyl. In embodiments, each R^{3A}, when present, is optionally substituted (C₁₀-C₂₀) alkynyl.

In embodiments, each R^{3B}, when present, is optionally substituted alkynyl. In embodiments, each R^{3B}, when present, is optionally substituted (C₅-C₂₅) alkynyl. In embodiments, each R^{3B}, when present, is optionally substituted (C₁₀-C₂₀) alkynyl.

In embodiments, each R^{3C}, when present, is optionally substituted alkynyl. In embodiments, each R^{3C}, when present, is optionally substituted (C₅-C₂₅) alkynyl. In embodiments, each R^{3C}, when present, is optionally substituted (C₁₀-C₂₀) alkynyl.

In embodiments, each R^{3D}, when present, is optionally substituted alkynyl. In embodiments, each R^{3D}, when present, is optionally substituted (C₅-C₂₅) alkynyl. In embodiments, each R^{3D}, when present, is optionally substituted (C₁₀-C₂₀) alkynyl.

In embodiments, each R³ or each R^{3A}, R^{3B}, R^{3C} and R^{3D}, when present, is independently selected from:

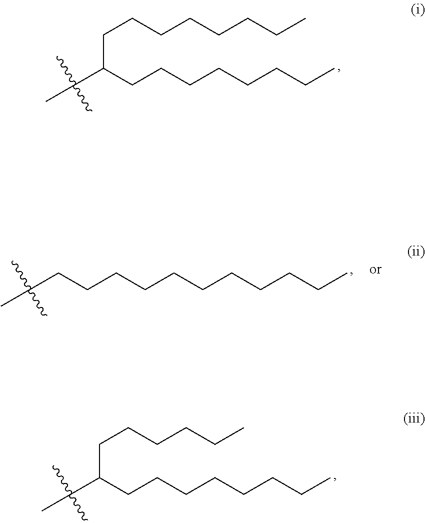 [SMILES: *C(CCCCCCCC)CCCCCCCC ; *CCCCCCCCCCC ; *C(CCCCCC)CCCCCCCC]

optionally wherein each R³ or each R^{3A}, R^{3B}, R^{3C} and R^{3D}, when present, is option (iii).

In embodiments, each R³ or each R^{3A}, R^{3B}, R^{3C} and R^{3D}, when present, is

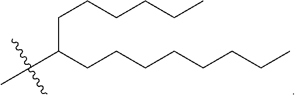 [SMILES: *C(CCCCCC)CCCCCCCC]

In embodiments, each R³ or each R^{3A}, R^{3B}, R^{3C} and R^{3D}, when present, is

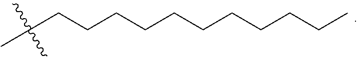 [SMILES: *CCCCCCCCCCC]

In embodiments, each R³ or each R^{3A}, R^{3B}, R^{3C} and R^{3D}, when present, is

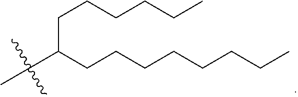 [SMILES: *C(CCCCCC)CCCCCCCC]

In embodiments, each R^{3A}, when present, is

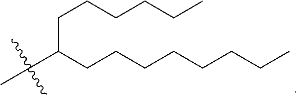 [SMILES: *C(CCCCCC)CCCCCCCC]

In embodiments, each R^{3A}, when present, is

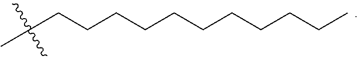 [SMILES: *CCCCCCCCCCC]

In embodiments, each R^{3A}, when present, is

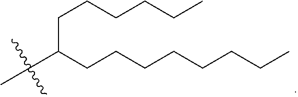 [SMILES: *C(CCCCCC)CCCCCCCC]

In embodiments, each R^{3B}, when present, is

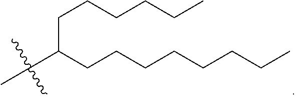 [SMILES: *C(CCCCCC)CCCCCCCC]

In embodiments, each R^{3B}, when present, is

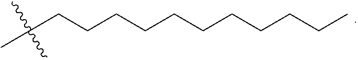 [SMILES: *CCCCCCCCCCC]

In embodiments, each R^{3B}, when present, is

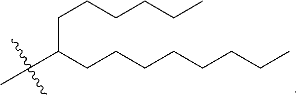 [SMILES: *C(CCCCCC)CCCCCCCC]

In embodiments, each R^{3C}, when present, is

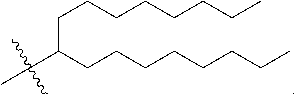 [SMILES: *C(CCCCCCCC)CCCCCCCC]

In embodiments, each R^{3C}, when present, is

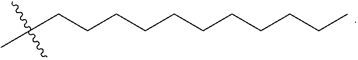 [SMILES: *CCCCCCCCCCC]

In embodiments, each R^{3C}, when present, is

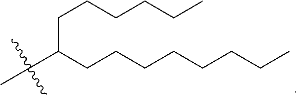 [SMILES: *C(CCCCCC)CCCCCCCC]

In embodiments, each R^{3D}, when present, is

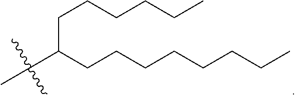 [SMILES: *C(CCCCCC)CCCCCCCC]

In embodiments, each R^{3D}, when present, is

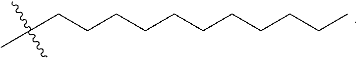 [SMILES: *CCCCCCCCCCC]

In embodiments, each R^{3D}, when present, is

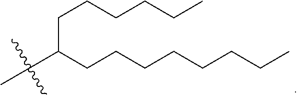 [SMILES: *C(CCCCCC)CCCCCCCC]

In embodiments, the substituents are not optionally substituted.

In embodiments, the cationic lipids of the present invention have any one of the structures in Tables A or B, or a pharmaceutically acceptable salt thereof.

In embodiments, provided herein is a composition comprising a cationic lipid of the present invention, and further comprising:
(i) one or more non-cationic lipids,
(ii) one or more cholesterol-based lipids and
(iii) one or more PEG-modified lipids.

In embodiments, this composition is a lipid nanoparticle, optionally a liposome. In embodiments, the one or more cationic lipid(s) constitute(s) about 30 mol %-60 mol % of the lipid nanoparticle. In embodiments, the one or more non-cationic lipid(s) constitute(s) about 10 mol %-50 mol % of the lipid nanoparticle. In embodiments, the one or more PEG-modified lipid(s) constitute(s) about 1 mol %-10 mol % of the lipid nanoparticle. In embodiments, the cholesterol-based lipid constitutes about 10 mol %-50 mol % of the lipid nanoparticle.

In embodiments, the lipid nanoparticle encapsulates a nucleic acid, optionally an mRNA encoding a peptide or protein. In embodiments, the lipid nanoparticle encapsulates an mRNA encoding a peptide or protein, optionally for use in a vaccine. In embodiments, the peptide is an antigen. As used herein, the phrase “encapsulation percentage” refers to the fraction of therapeutic agent (e.g. mRNA) that is effectively encapsulated within a liposomal-based vehicle (e.g. a lipid nanoparticle) relative to the initial fraction of therapeutic agent present in the lipid phase. In embodiments, the lipid nanoparticles have an encapsulation percentage for mRNA of at least 50%. In embodiments, the lipid nanoparticles have an encapsulation percentage for mRNA of at least 55%. In embodiments, the lipid nanoparticles have an encapsulation percentage for mRNA of at least 60%. In embodiments, the lipid nanoparticles have an encapsulation percentage for mRNA of at least 65%. In embodiments, the lipid nanoparticles have an encapsulation percentage for mRNA of at least 70%. In embodiments, the lipid nanoparticles have an encapsulation percentage for mRNA of at least 75%. In embodiments, the lipid nanoparticles have an encapsulation percentage for mRNA of at least 80%. In embodiments, the lipid nanoparticles have an encapsulation percentage for mRNA of at least 85%. In embodiments, the lipid nanoparticles have an encapsulation percentage for mRNA of at least 90%. In embodiments, the lipid nanoparticles have an encapsulation percentage for mRNA of at least 95%. In embodiments, the encapsulation percentage is calculated by performing the Ribogreen assay (Invitrogen) with and without the presence of 0.1% Triton-X 100.

In embodiments, the composition of the present invention is for use in therapy.

In embodiments, the composition of the present invention is for use in a method of treating or preventing a disease amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

In embodiments, a method for treating or preventing a disease is provided, wherein said method comprises administering to a subject in need thereof a composition of the present invention and wherein the disease is amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

In embodiments, the composition is administered intravenously, intrathecally, intramuscularly, intranasally, sublingually, or by pulmonary delivery, optionally through nebulization. In embodiments, the composition is administered intramuscularly.

### Exemplary Compounds

In embodiments, the cationic lipids of the present invention include compounds selected from those depicted in Tables A or B, or a pharmaceutically acceptable salt thereof.

Exemplary compounds include those described in Table A and Table B, or a pharmaceutically acceptable salt thereof.

TABLE A

| Compound # | Compound structure |
|---|---|
| Formula I | 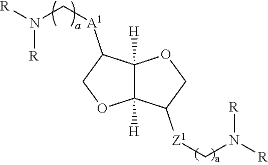 |
| Formula IA | 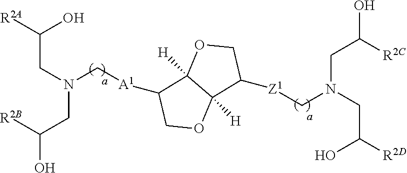 |
| Formula IB1a | 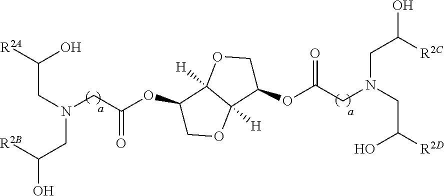 |
| | optionally wherein each a is independently selected from 3 or 4. |
| 1 | 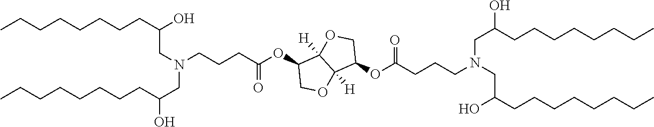 [SMILES: CCCCCCCCC(O)CN(CCCC(=O)O[C@@H]1CO[C@H]2[C@@H]1OC[C@H]2OC(=O)CCCN(CC(O)CCCCCCCC)CC(O)CCCCCCCC)CC(O)CCCCCCCC] |
| 2 | 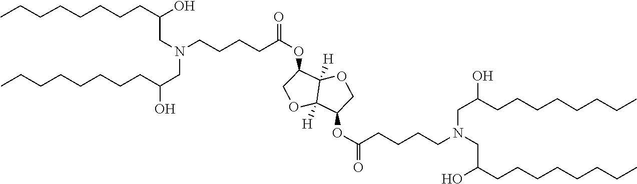 [SMILES: CCCCCCCCC(O)CN(CCCCC(=O)O[C@@H]1CO[C@H]2[C@@H]1OC[C@H]2OC(=O)CCCCN(CC(O)CCCCCCCC)CC(O)CCCCCCCC)CC(O)CCCCCCCC] |
| 3 | 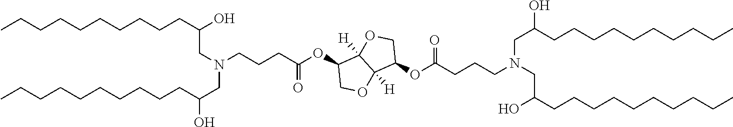 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC] |
| 4 | 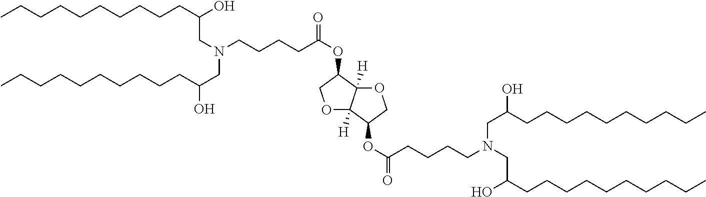 [SMILES: CCCCCCCCCCC(O)CN(CCCCC(=O)O[C@@H]1CO[C@H]2[C@@H]1OC[C@H]2OC(=O)CCCCN(CC(O)CCCCCCCCCC)CC(O)CCCCCCCCCC)CC(O)CCCCCCCCCC] |
| 5 | 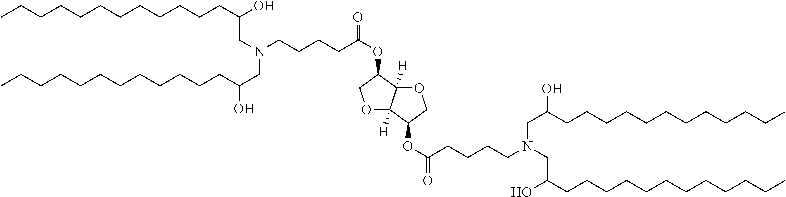 [SMILES: CCCCCCCCCCCCC(O)CN(CCCCC(=O)O[C@@H]1CO[C@H]2[C@@H]1OC[C@H]2OC(=O)CCCCN(CC(O)CCCCCCCCCCCC)CC(O)CCCCCCCCCCCC)CC(O)CCCCCCCCCCCC] |
| 6 | 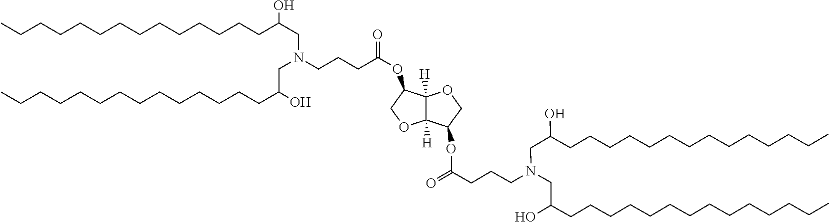 [SMILES: CCCCCCCCCCCCCCC(O)CN(CCCC(=O)O[C@@H]1CO[C@H]2[C@@H]1OC[C@H]2OC(=O)CCCN(CC(O)CCCCCCCCCCCCCC)CC(O)CCCCCCCCCCCCCC)CC(O)CCCCCCCCCCCCCC] |
| 7 | 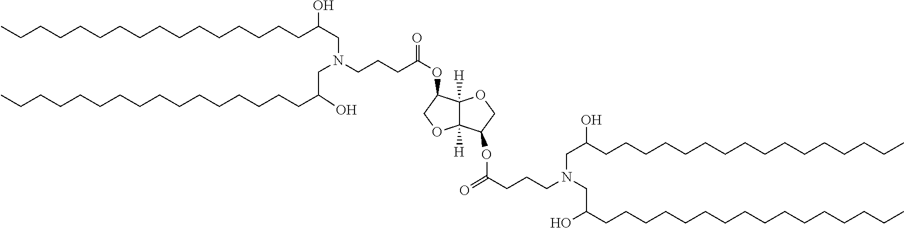 [SMILES: CCCCCCCCCCCCCCCCC(O)CN(CCCC(=O)O[C@@H]1CO[C@H]2[C@@H]1OC[C@H]2OC(=O)CCCN(CC(O)CCCCCCCCCCCCCCCC)CC(O)CCCCCCCCCCCCCCCC)CC(O)CCCCCCCCCCCCCCCC] |
| 8 | 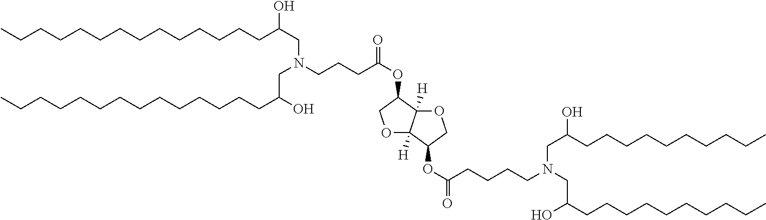 [SMILES: CCCCCCCCCCCCCCC(O)CN(CCCC(=O)O[C@@H]1CO[C@H]2[C@@H]1OC[C@H]2OC(=O)CCCCN(CC(O)CCCCCCCCCC)CC(O)CCCCCCCCCC)CC(O)CCCCCCCCCCCCCC] |
| 9 | 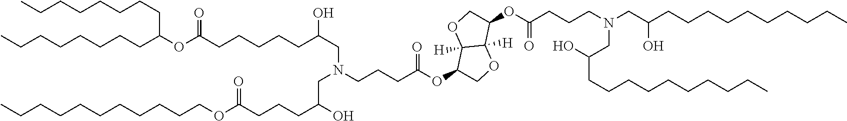 [SMILES: CCCCCCCCCCCOC(=O)CCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCC(=O)OC(CCCCCCCC)CCCCCCCC] |
| 10 | 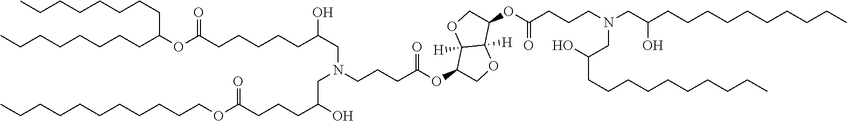 [SMILES: CCCCCCCCCCCOC(=O)CCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCC(=O)OC(CCCCCCCC)CCCCCCCC] |
| 11 | 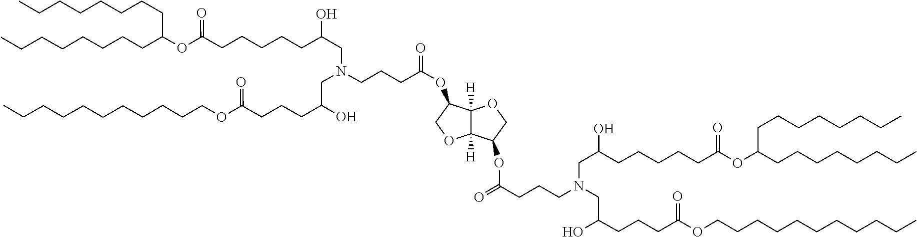 [SMILES: CCCCCCCCCCCOC(=O)CCCC(O)CN(CCCC(=O)O[C@@H]1CO[C@H]2[C@@H]1OC[C@H]2OC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| 12 | 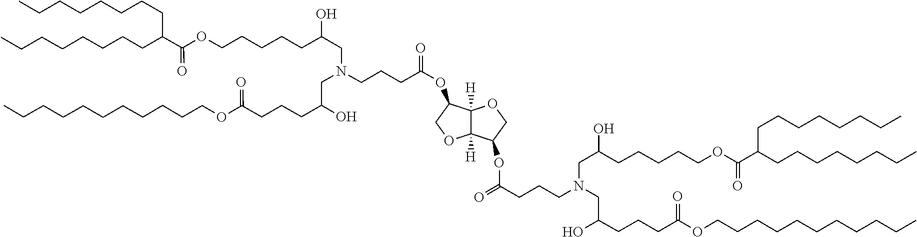 [SMILES: CCCCCCCCCCCOC(=O)CCCC(O)CN(CCCC(=O)O[C@@H]1CO[C@H]2[C@@H]1OC[C@H]2OC(=O)CCCN(CC(O)CCCCCOC(=O)C(CCCCCCCC)CCCCCCCC)CC(O)CCCC(=O)OCCCCCCCCCCC)CC(O)CCCCCOC(=O)C(CCCCCCCC)CCCCCCCC] |
| 13 | 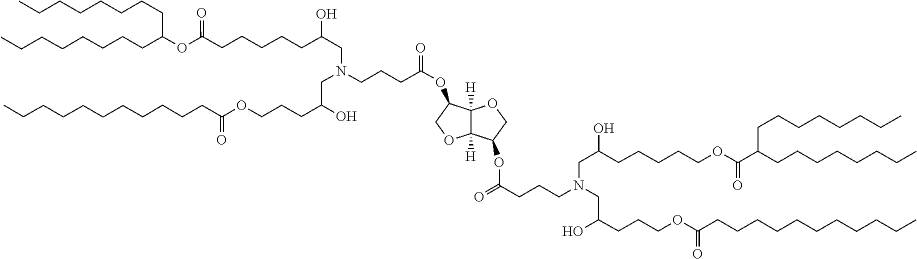 [SMILES: CCCCCCCCCCCC(=O)OCCCC(O)CN(CCCC(=O)O[C@@H]1CO[C@H]2[C@@H]1OC[C@H]2OC(=O)CCCN(CC(O)CCCCCC(=O)OC(CCCCCCCC)CCCCCCCC)CC(O)CCCOC(=O)CCCCCCCCCCC)CC(O)CCCCCOC(=O)C(CCCCCCCC)CCCCCCCC] |
| 14 | 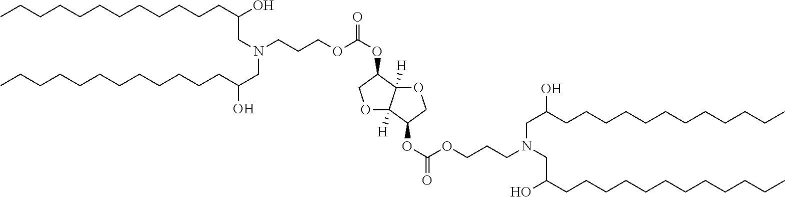 [SMILES: CCCCCCCCCCCCC(O)CN(CCCOC(=O)O[C@@H]1CO[C@H]2[C@@H]1OC[C@H]2OC(=O)OCCCN(CC(O)CCCCCCCCCCCC)CC(O)CCCCCCCCCCCC)CC(O)CCCCCCCCCCCC] |
| 15 | 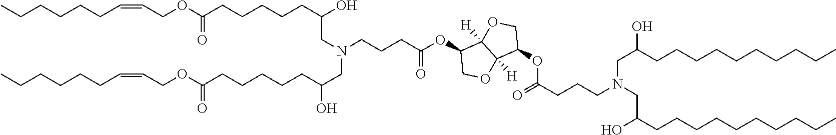 [SMILES: CCCCCC/C=C\COC(=O)CCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCC(=O)OC/C=C\CCCCCC] |
| 16 | 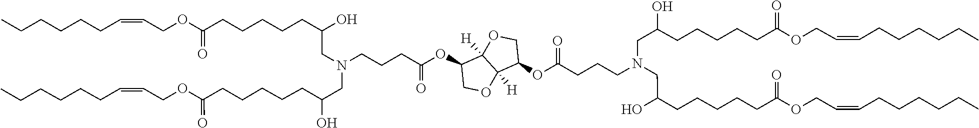 [SMILES: CCCCCC/C=C\COC(=O)CCCCCC(O)CN(CCCC(=O)O[C@@H]1CO[C@H]2[C@@H]1OC[C@H]2OC(=O)CCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |
| Formula IB1b | 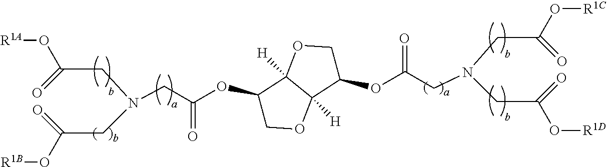 |
| | optionally wherein i) each a is 2; and/or ii) each b is independently selected from 5 or 7. |
| 17 | 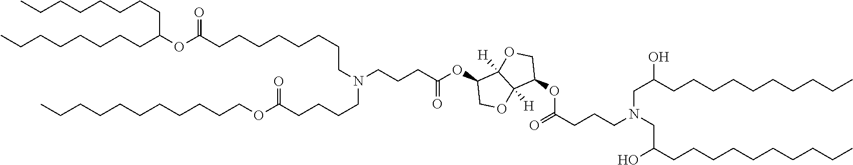 [SMILES: CCCCCCCCCCCOC(=O)CCCCN(CCCCCCCCC(=O)OC(CCCCCCCC)CCCCCCCC)CCCC(=O)O[C@@H]1CO[C@H]2[C@@H]1OC[C@H]2OC(=O)CCCN(CC(O)CCCCCCCCCC)CC(O)CCCCCCCCCC] |
| Formula IB1c | 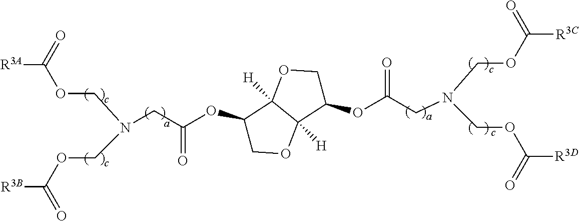 |
| | optionally wherein i) each a is 3; and/or ii) each c is 6. |
| 18 | 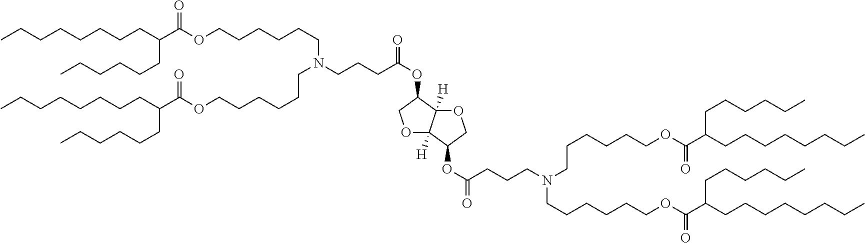 [SMILES: CCCCCCCCC(CCCCCC)C(=O)OCCCCCCN(CCCCCCOC(=O)C(CCCCCC)CCCCCCCC)CCCC(=O)O[C@@H]1CO[C@H]2[C@@H]1OC[C@H]2OC(=O)CCCN(CCCCCCOC(=O)C(CCCCCC)CCCCCCCC)CCCCCCOC(=O)C(CCCCCC)CCCCCCCC] |
| Formula IB1d | 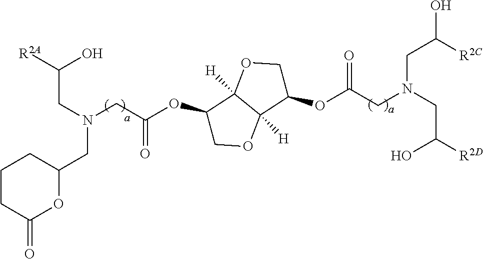 |
| | optionally wherein each a is 3. |
| 19 | 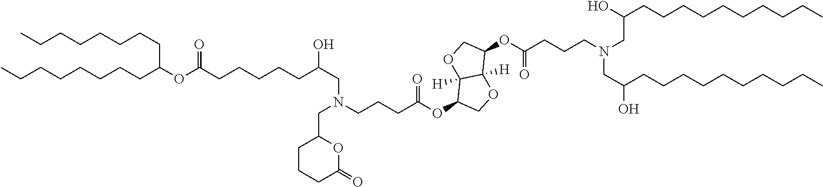 [SMILES: CCCCCCCCCCC(O)CN(CCCC(=O)O[C@@H]1CO[C@H]2[C@@H]1OC[C@H]2OC(=O)CCCN(CC(O)CCCCCC(=O)OC(CCCCCCCC)CCCCCCCC)CC1CCCC(=O)O1)CC(O)CCCCCCCCCC] |
| Formula IB2a | 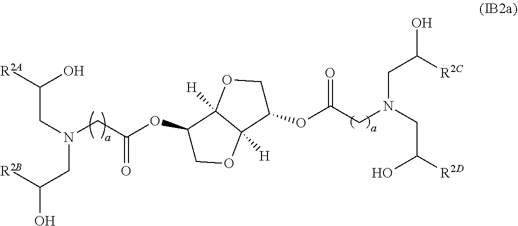 |
| | optionally wherein each a is independently selected from 3 or 4. |
| 20 | 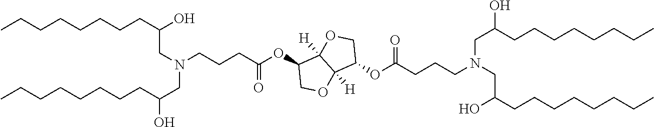 [SMILES: CCCCCCCCC(O)CN(CCCC(=O)O[C@H]1CO[C@H]2[C@@H]1OC[C@H]2OC(=O)CCCN(CC(O)CCCCCCCC)CC(O)CCCCCCCC)CC(O)CCCCCCCC] |
| 21 | 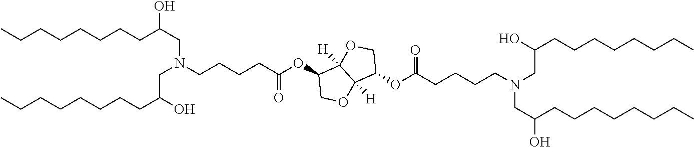 [SMILES: CCCCCCCCC(O)CN(CCCCC(=O)O[C@H]1CO[C@H]2[C@@H]1OC[C@H]2OC(=O)CCCCN(CC(O)CCCCCCCC)CC(O)CCCCCCCC)CC(O)CCCCCCCC] |
| 22 | 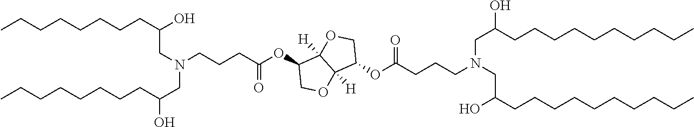 [SMILES: CCCCCCCCCCC(O)CN(CCCC(=O)O[C@H]1CO[C@H]2[C@@H]1OC[C@H]2OC(=O)CCCN(CC(O)CCCCCCCC)CC(O)CCCCCCCC)CC(O)CCCCCCCCCC] |
| 23 | 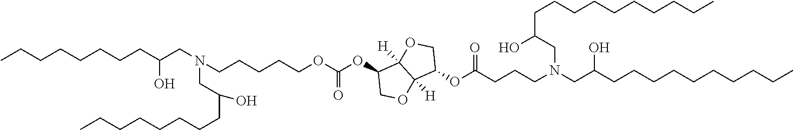 [SMILES: CCCCCCCCCCC(O)CN(CCCC(=O)O[C@H]1CO[C@H]2[C@@H]1OC[C@H]2OC(=O)OCCCCCN(CC(O)CCCCCCCC)CC(O)CCCCCCCC)CC(O)CCCCCCCCCC] |
| 24 | 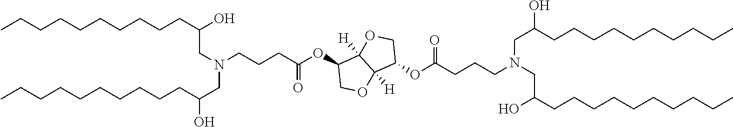 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC] |
| 25 | 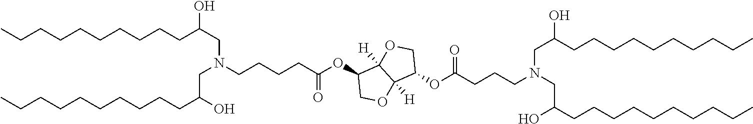 [SMILES: CCCCCCCCCCC(O)CN(CCCCC(=O)O[C@@H]1CO[C@H]2[C@@H]1OC[C@@H]2OC(=O)CCCN(CC(O)CCCCCCCCCC)CC(O)CCCCCCCCCC)CC(O)CCCCCCCCCC] |
| 26 | 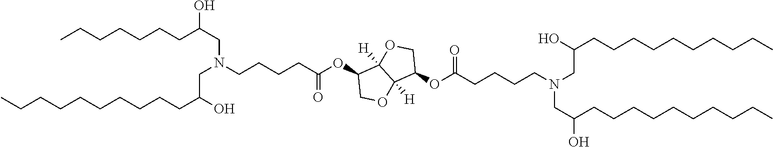 [SMILES: CCCCCCCCCCC(O)CN(CCCCC(=O)O[C@@H]1CO[C@H]2[C@@H]1OC[C@H]2OC(=O)CCCCN(CC(O)CCCCCCCCCC)CC(O)CCCCCCCCCC)CC(O)CCCCCCC] |
| 27 | 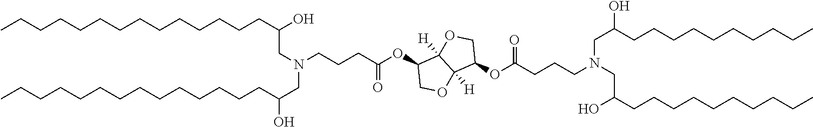 [SMILES: CCCCCCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCCCCCC] |
| 28 | 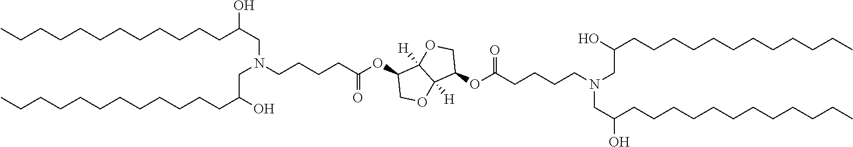 [SMILES: CCCCCCCCCCCCC(O)CN(CCCCC(=O)O[C@@H]1CO[C@H]2[C@@H]1OC[C@H]2OC(=O)CCCCN(CC(O)CCCCCCCCCCCC)CC(O)CCCCCCCCCCCC)CC(O)CCCCCCCCCCCC] |
| 29 | 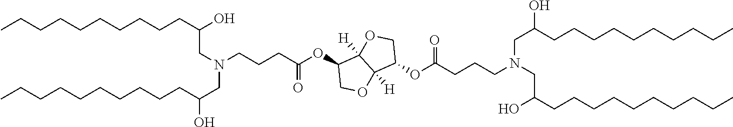 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC] |
| 30 | 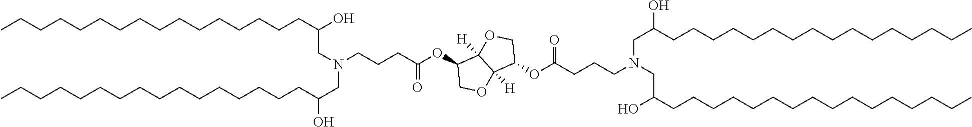 [SMILES: CCCCCCCCCCCCCCCCC(O)CN(CCCC(=O)O[C@H]1CO[C@H]2[C@@H]1OC[C@H]2OC(=O)CCCN(CC(O)CCCCCCCCCCCCCCCC)CC(O)CCCCCCCCCCCCCCCC)CC(O)CCCCCCCCCCCCCCCC] |
| 31 | 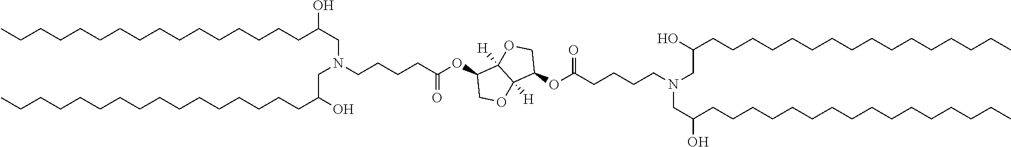 [SMILES: CCCCCCCCCCCCCCCCC(O)CN(CCCCC(=O)O[C@@H]1CO[C@H]2[C@@H]1OC[C@H]2OC(=O)CCCCN(CC(O)CCCCCCCCCCCCCCCC)CC(O)CCCCCCCCCCCCCCCC)CC(O)CCCCCCCCCCCCCCCC] |
| 32 | 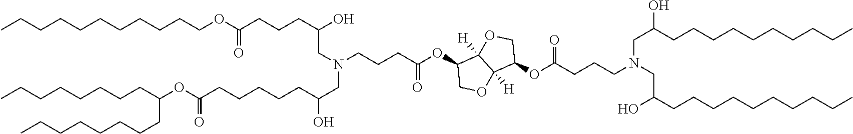 [SMILES: CCCCCCCCCCCOC(=O)CCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCC(=O)OC(CCCCCCCC)CCCCCCCC] |
| 33 | 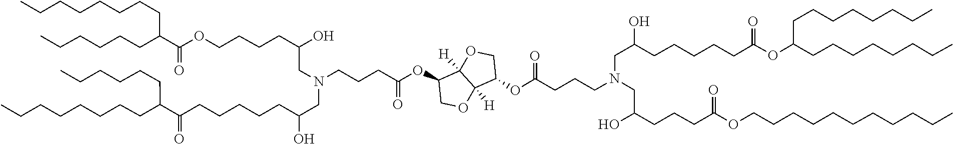 [SMILES: CCCCCCCCCCCOC(=O)CCCC(O)CN(CCCC(=O)O[C@H]1CO[C@H]2[C@@H]1OC[C@H]2OC(=O)CCCN(CC(O)CCCCCC(=O)C(CCCCCC)CCCCCCCC)CC(O)CCCCOC(=O)C(CCCCCC)CCCCCCCC)CC(O)CCCCCC(=O)OC(CCCCCCCC)CCCCCCCC] |
| 34 | 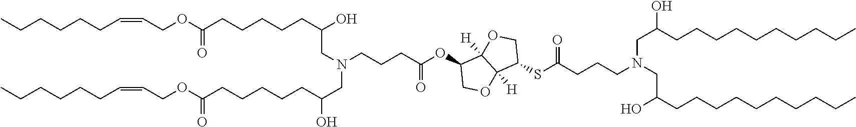 [SMILES: CCCCCC/C=C\COC(=O)CCCCCC(O)CN(CCCC(=O)O[C@@H]1CO[C@H]2[C@@H]1OC[C@@H]2SC(=O)CCCN(CC(O)CCCCCCCCCC)CC(O)CCCCCCCCCC)CC(O)CCCCCC(=O)OC/C=C\CCCCCC] |
| 35 | 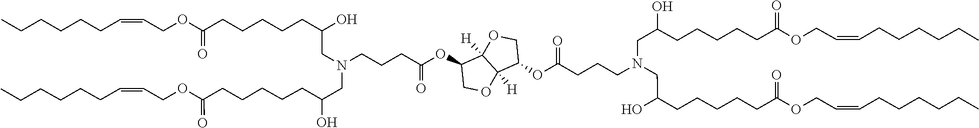 [SMILES: CCCCCC/C=C\COC(=O)CCCCCC(O)CN(CCCC(=O)O[C@H]1CO[C@H]2[C@@H]1OC[C@H]2OC(=O)CCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |
| Formula IC1a | 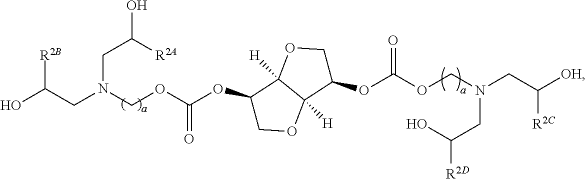 |
| | optionally wherein each a independently selected from 3 or 4 |
| 36 | 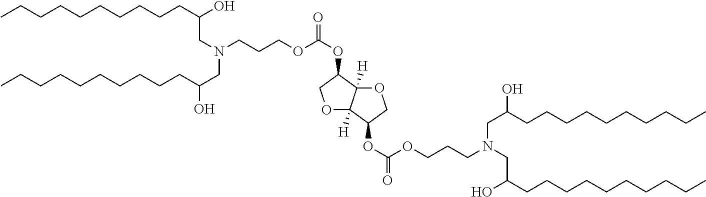 [SMILES: CCCCCCCCCCC(O)CN(CCCOC(=O)O[C@@H]1CO[C@H]2[C@@H]1OC[C@H]2OC(=O)OCCCN(CC(O)CCCCCCCCCC)CC(O)CCCCCCCCCC)CC(O)CCCCCCCCCC] |
| 37 | 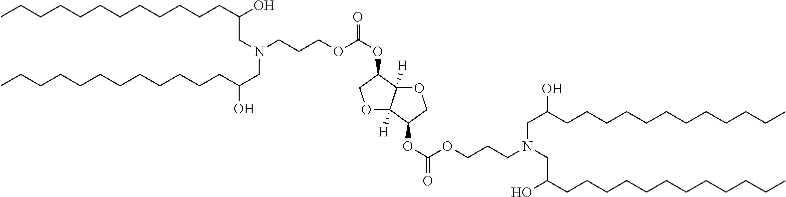 [SMILES: CCCCCCCCCCCCC(O)CN(CCCOC(=O)O[C@@H]1CO[C@H]2[C@@H]1OC[C@H]2OC(=O)OCCCN(CC(O)CCCCCCCCCCCC)CC(O)CCCCCCCCCCCC)CC(O)CCCCCCCCCCCC] |
| 38 | 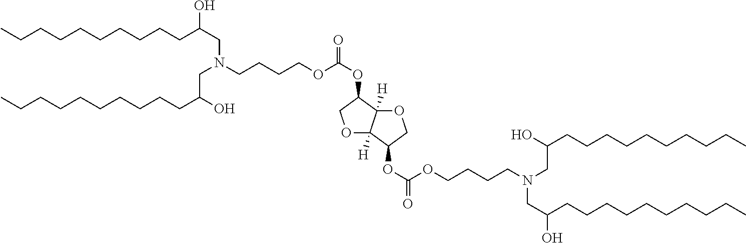 [SMILES: CCCCCCCCCCC(O)CN(CCCCOC(=O)O[C@@H]1CO[C@H]2[C@@H]1OC[C@H]2OC(=O)OCCCCN(CC(O)CCCCCCCCCC)CC(O)CCCCCCCCCC)CC(O)CCCCCCCCCC] |
| Formula IC1b | 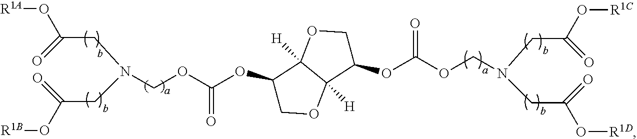 |
| | optionally wherein each a is 2; and/or ii) each b is independently selected from 5 or 7. |
| 39 | 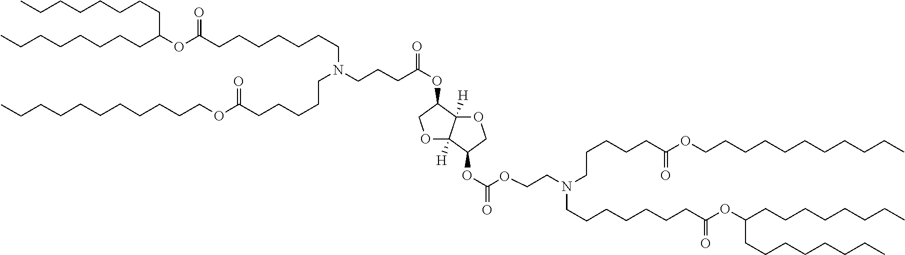 [SMILES: CCCCCCCCCCCOC(=O)CCCCCN(CCCCCCCC(=O)OC(CCCCCCCC)CCCCCCCC)CCCC(=O)O[C@@H]1CO[C@H]2[C@@H]1OC[C@H]2OC(=O)OCCN(CCCCCCCC(=O)OC(CCCCCCCC)CCCCCCCC)CCCCCC(=O)OCCCCCCCCCCC] |
| Formula IC2a | 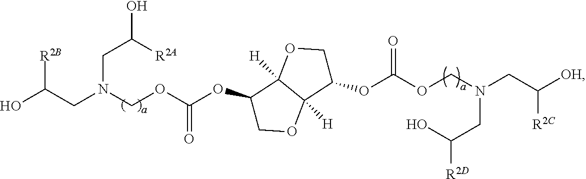 |
| | optionally wherein each a is 3. |
| 40 | 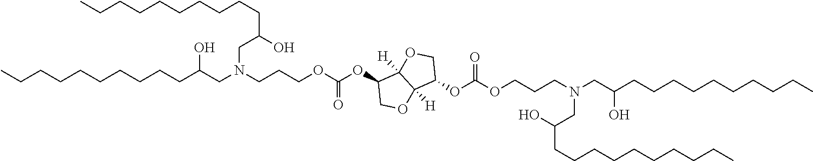 [SMILES: CCCCCCCCCCC(O)CN(CCCOC(=O)O[C@H]1CO[C@H]2[C@@H]1OC[C@H]2OC(=O)OCCCN(CC(O)CCCCCCCCCC)CC(O)CCCCCCCCCC)CC(O)CCCCCCCCCC] |
| Formula ID | 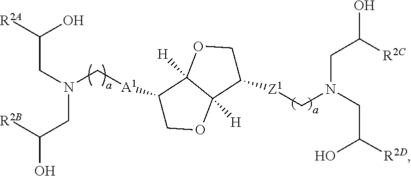 |
| | optionally wherein each a is independently selected from 3 or 4. |
| 41 | 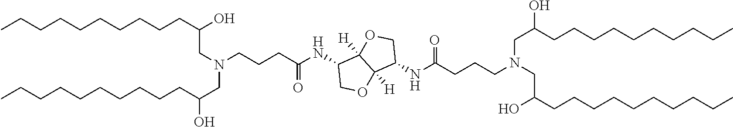 [SMILES: CCCCCCCCCCC(O)CN(CCCC(=O)N[C@H]1CO[C@H]2[C@@H]1OC[C@@H]2NC(=O)CCCN(CC(O)CCCCCCCCCC)CC(O)CCCCCCCCCC)CC(O)CCCCCCCCCC] |
| 42 | 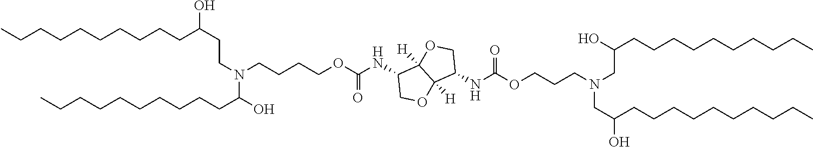 [SMILES: CCCCCCCCCCC(O)CCN(CCCCOC(=O)N[C@H]1CO[C@H]2[C@@H]1OC[C@@H]2NC(=O)OCCCN(CC(O)CCCCCCCCCC)CC(O)CCCCCCCCCC)C(O)CCCCCCCCCC] |
| 43 | 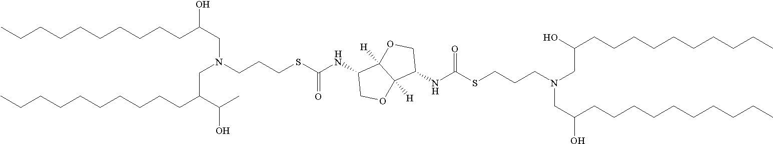 [SMILES: CCCCCCCCCCC(O)CN(CCCSC(=O)N[C@H]1CO[C@H]2[C@@H]1OC[C@@H]2NC(=O)SCCCN(CC(O)CCCCCCCCCC)CC(CCCCCCCCCC)C(C)O)CC(O)CCCCCCCCCC] |
| 44 | 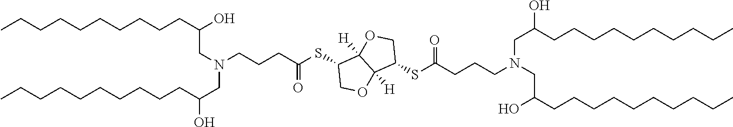 [SMILES: CCCCCCCCCCC(O)CN(CCCC(=O)S[C@H]1CO[C@H]2[C@@H]1OC[C@@H]2SC(=O)CCCN(CC(O)CCCCCCCCCC)CC(O)CCCCCCCCCC)CC(O)CCCCCCCCCC] |
| 45 | 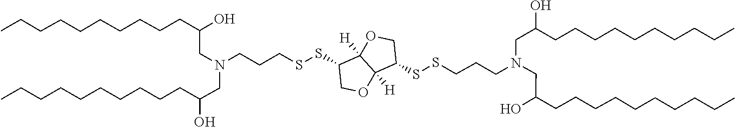 |
| 46 | 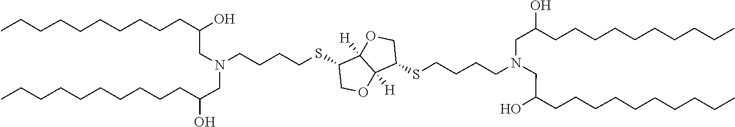 |
| 47 | 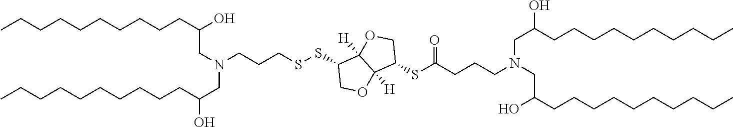 [SMILES: CCCCCCCCCCC(O)CN(CCCSS[C@H]1CO[C@H]2[C@@H]1OC[C@@H]2SC(=O)CCCN(CC(O)CCCCCCCCCC)CC(O)CCCCCCCCCC)CC(O)CCCCCCCCCC] |
| 51 | 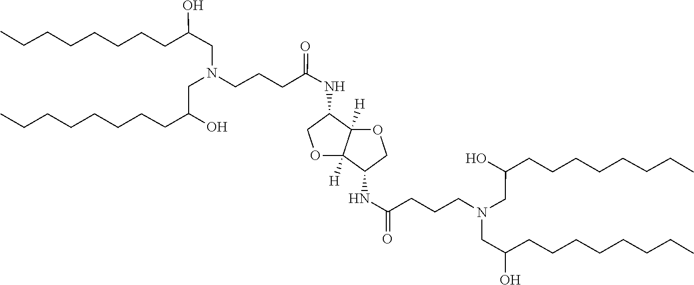 [SMILES: CCCCCCCCC(O)CN(CCCC(=O)N[C@H]1CO[C@H]2[C@@H]1OC[C@@H]2NC(=O)CCCN(CC(O)CCCCCCCC)CC(O)CCCCCCCC)CC(O)CCCCCCCC] |
| 52 | 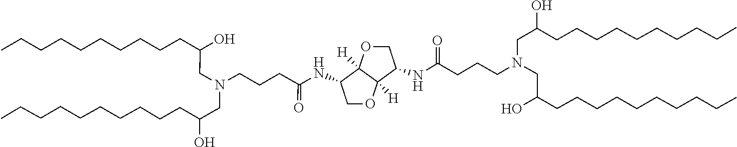 [SMILES: CCCCCCCCCCC(O)CN(CCCC(=O)N[C@H]1CO[C@H]2[C@@H]1OC[C@@H]2NC(=O)CCCN(CC(O)CCCCCCCCCC)CC(O)CCCCCCCCCC)CC(O)CCCCCCCCCC] |
| 53 | 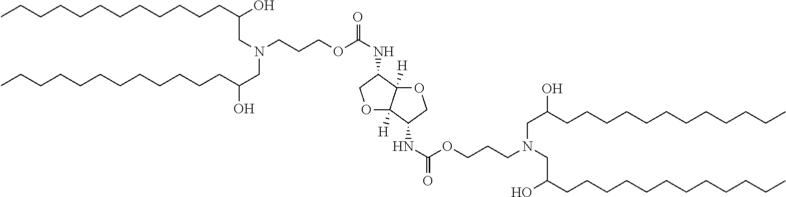 [SMILES: CCCCCCCCCCCCC(O)CN(CCCOC(=O)N[C@H]1CO[C@H]2[C@@H]1OC[C@@H]2NC(=O)OCCCN(CC(O)CCCCCCCCCCCC)CC(O)CCCCCCCCCCCC)CC(O)CCCCCCCCCCCC] |
| 54 | 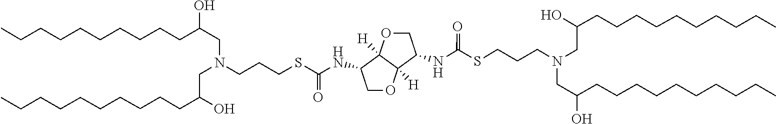 [SMILES: CCCCCCCCCCC(O)CN(CCCSC(=O)N[C@H]1CO[C@H]2[C@@H]1OC[C@@H]2NC(=O)SCCCN(CC(O)CCCCCCCCCC)CC(O)CCCCCCCCCC)CC(O)CCCCCCCCCC] |
| 55 | 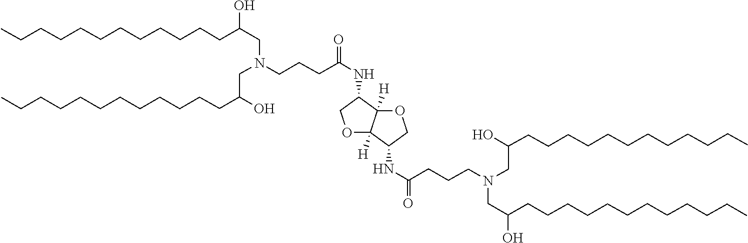 [SMILES: CCCCCCCCCCCCC(O)CN(CCCC(=O)N[C@H]1CO[C@H]2[C@@H]1OC[C@@H]2NC(=O)CCCN(CC(O)CCCCCCCCCCCC)CC(O)CCCCCCCCCCCC)CC(O)CCCCCCCCCCCC] |
| Formula IE | 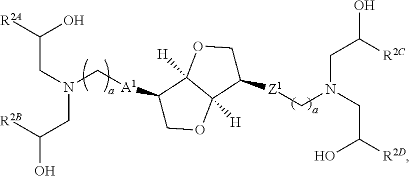 |
| | optionally wherein each a is 3. |
| 48 | 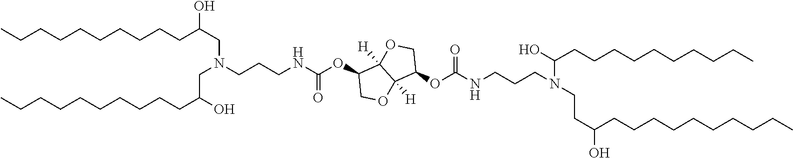 [SMILES: CCCCCCCCCCC(O)CCN(CCCNC(=O)O[C@@H]1CO[C@H]2[C@@H]1OC[C@H]2OC(=O)NCCCN(CC(O)CCCCCCCCCC)CC(O)CCCCCCCCCC)C(O)CCCCCCCCCC] |
| Formula II | 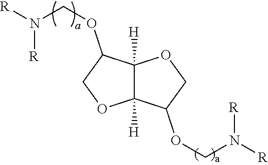 |
| Formula IIA | 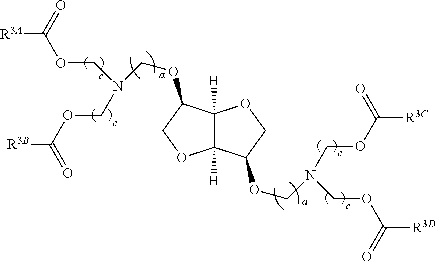 |
| | optionally wherein each a is 4; and/or ii) each c is 6. |
| 49 | 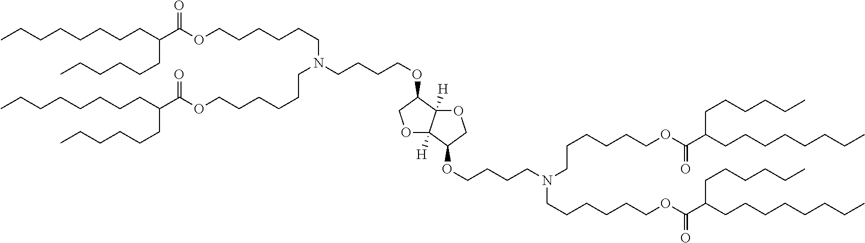 [SMILES: CCCCCCCCC(CCCCCC)C(=O)OCCCCCCN(CCCCCCOC(=O)C(CCCCCC)CCCCCCCC)CCCCO[C@@H]1CO[C@H]2[C@@H]1OC[C@H]2OCCCCN(CCCCCCOC(=O)C(CCCCCC)CCCCCCCC)CCCCCCOC(=O)C(CCCCCC)CCCCCCCC] |
| Formula IIB | 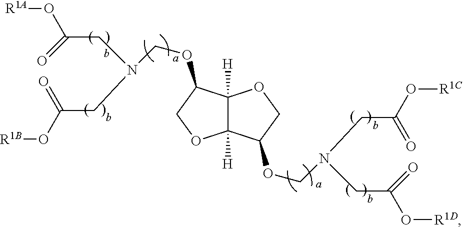 |
| | optionally wherein each a is 2; and/or ii) each b is independently selected from 5 or 7. |
| 50 | 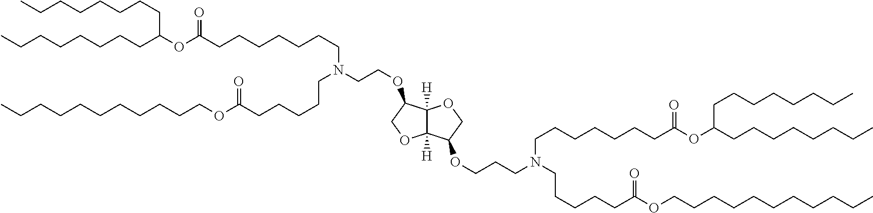 [SMILES: CCCCCCCCCCCOC(=O)CCCCCN(CCCCCCCC(=O)OC(CCCCCCCC)CCCCCCCC)CCCO[C@@H]1CO[C@H]2[C@@H]1OC[C@H]2OCCN(CCCCCCCC(=O)OC(CCCCCCCC)CCCCCCCC)CCCCCC(=O)OCCCCCCCCCCC] |

TABLE B

| Compound | |
|---|---|
| # | Compound structure |
| 56 | 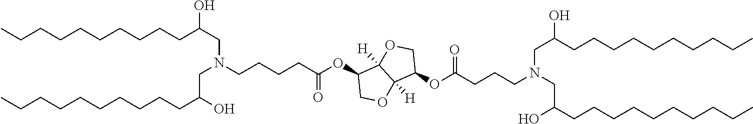 [SMILES: CCCCCCCCCCC(O)CN(CCCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC] |
| 57 | 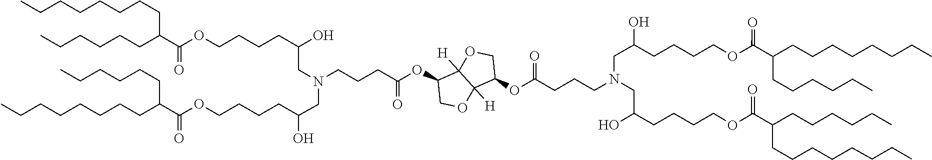 [SMILES: CCCCCCCCC(CCCCCC)C(=O)OCCCCC(O)CN(CCCC(=O)O[C@@H]1COC2C1OC[C@H]2OC(=O)CCCN(CC(O)CCCCOC(=O)C(CCCCCC)CCCCCCCC)CC(O)CCCCOC(=O)C(CCCCCC)CCCCCCCC)CC(O)CCCCOC(=O)C(CCCCCC)CCCCCCCC] |
| 58 | 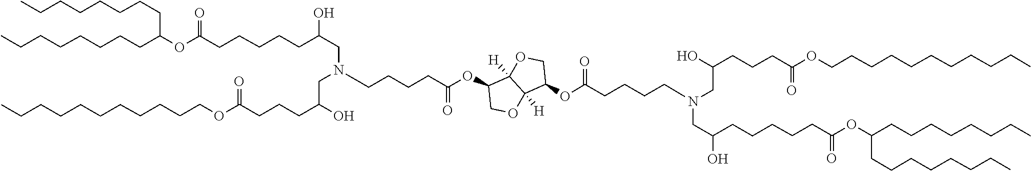 [SMILES: CCCCCCCCCCCOC(=O)CCCC(O)CN(CCCCC(=O)O[C@@H]1CO[C@H]2[C@@H]1OC[C@H]2OC(=O)CCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| 59 | 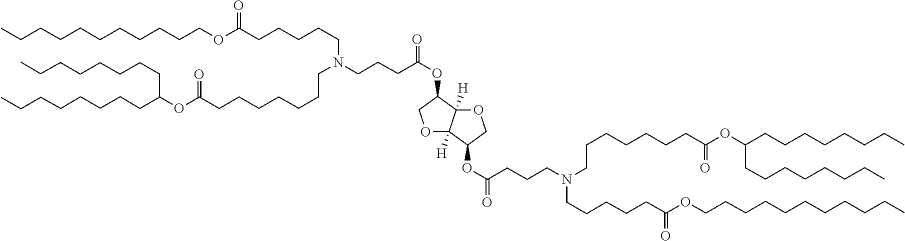 [SMILES: CCCCCCCCCCCOC(=O)CCCCCN(CCCCCCCC(=O)OC(CCCCCCCC)CCCCCCCC)CCCC(=O)O[C@@H]1CO[C@H]2[C@@H]1OC[C@H]2OC(=O)CCCN(CCCCCCCC(=O)OC(CCCCCCCC)CCCCCCCC)CCCCCC(=O)OCCCCCCCCCCC] |
| 60 | 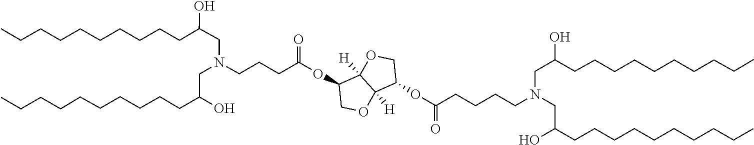 [SMILES: CCCCCCCCCCC(O)CN(CCCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC] |
| 61 | 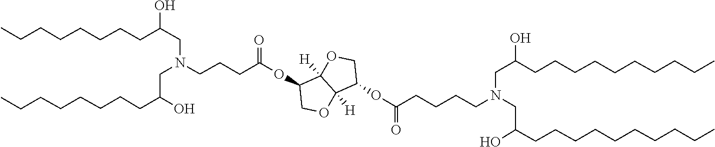 [SMILES: CCCCCCCCCCC(O)CN(CCCCC(=O)O[C@H]1CO[C@H]2[C@@H]1OC[C@H]2OC(=O)CCCN(CC(O)CCCCCCCC)CC(O)CCCCCCCC)CC(O)CCCCCCCCCC] |
| 62 | 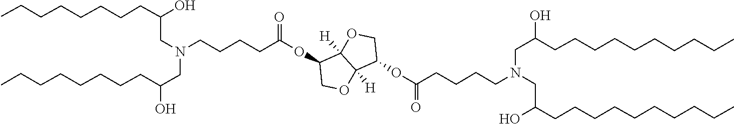 [SMILES: CCCCCCCCCCC(O)CN(CCCCC(=O)O[C@H]1CO[C@H]2[C@@H]1OC[C@H]2OC(=O)CCCCN(CC(O)CCCCCCCC)CC(O)CCCCCCCC)CC(O)CCCCCCCCCC] |
| 63 | 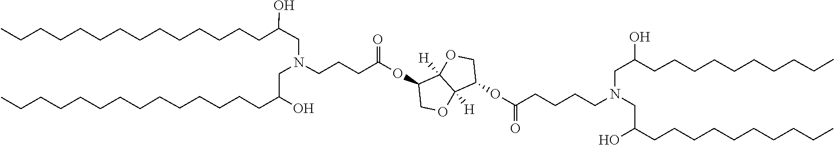 [SMILES: CCCCCCCCCCCCCCC(O)CN(CCCC(=O)O[C@@H]1CO[C@H]2[C@@H]1OC[C@@H]2OC(=O)CCCCN(CC(O)CCCCCCCCCC)CC(O)CCCCCCCCCC)CC(O)CCCCCCCCCCCCCC] |
| 64 | 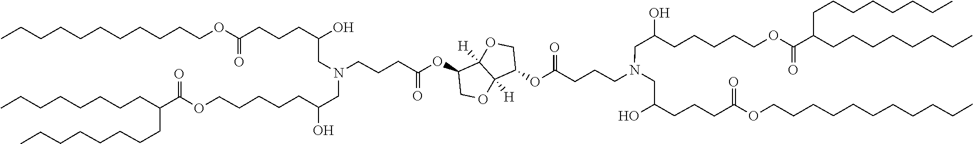 [SMILES: CCCCCCCCCCCOC(=O)CCCC(O)CN(CCCC(=O)O[C@H]1CO[C@H]2[C@@H]1OC[C@H]2OC(=O)CCCN(CC(O)CCCCCOC(=O)C(CCCCCCCC)CCCCCCCC)CC(O)CCCC(=O)OCCCCCCCCCCC)CC(O)CCCCCOC(=O)C(CCCCCCCC)CCCCCCCC] |
| 65 | 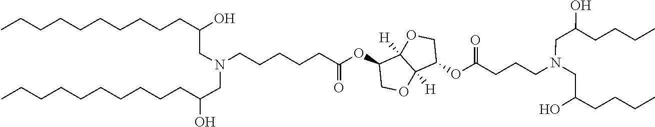 [SMILES: CCCCCCCCCCC(O)CN(CCCCCC(=O)O[C@@H]1CO[C@H]2[C@@H]1OC[C@@H]2OC(=O)CCCN(CC(O)CCCC)CC(O)CCCC)CC(O)CCCCCCCCCC] |
| 66 | 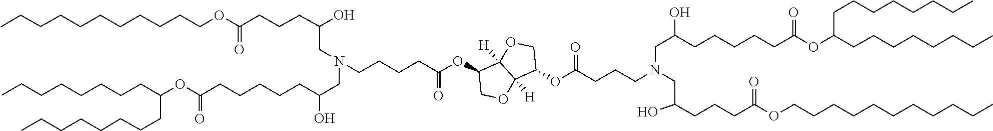 [SMILES: CCCCCCCCCCCOC(=O)CCCC(O)CN(CCCCC(=O)O[C@@H]1CO[C@H]2[C@@H]1OC[C@@H]2OC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| 67 | 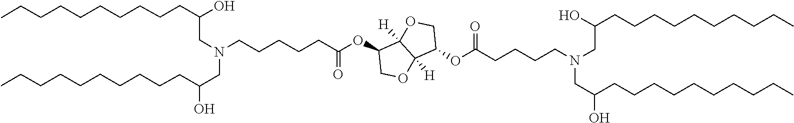 [SMILES: CCCCCCCCCCC(O)CN(CCCCCC(=O)O[C@@H]1CO[C@H]2[C@@H]1OC[C@@H]2OC(=O)CCCCN(CC(O)CCCCCCCCCC)CC(O)CCCCCCCCCC)CC(O)CCCCCCCCCC] |
| 68 | 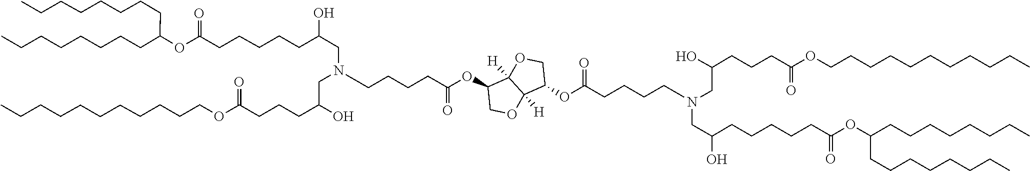 [SMILES: CCCCCCCCCCCOC(=O)CCCC(O)CN(CCCCC(=O)O[C@H]1CO[C@H]2[C@@H]1OC[C@H]2OC(=O)CCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| 69 | 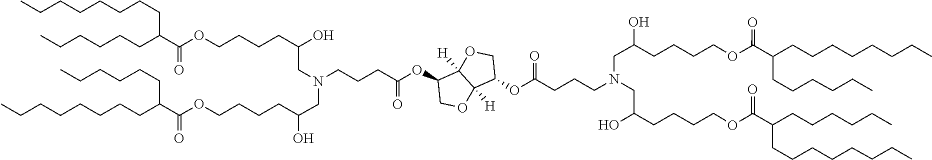 [SMILES: CCCCCCCCC(CCCCCC)C(=O)OCCCCC(O)CN(CCCC(=O)O[C@H]1CO[C@H]2[C@@H]1OC[C@H]2OC(=O)CCCN(CC(O)CCCCOC(=O)C(CCCCCC)CCCCCCCC)CC(O)CCCCOC(=O)C(CCCCCC)CCCCCCCC)CC(O)CCCCOC(=O)C(CCCCCC)CCCCCCCC] |
| 70 | 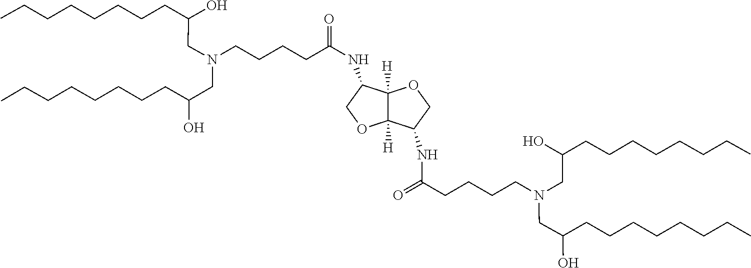 [SMILES: CCCCCCCCC(O)CN(CCCCC(=O)N[C@H]1CO[C@H]2[C@@H]1OC[C@@H]2NC(=O)CCCCN(CC(O)CCCCCCCC)CC(O)CCCCCCCC)CC(O)CCCCCCCC] |
| 71 | 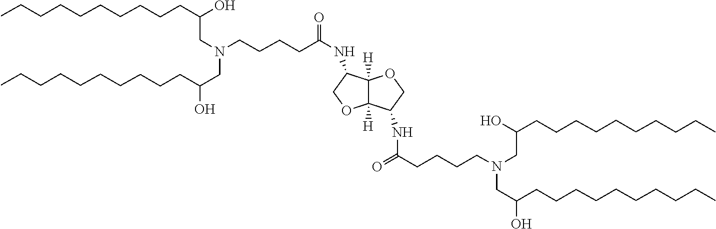 [SMILES: CCCCCCCCCCC(O)CN(CCCCC(=O)N[C@H]1CO[C@H]2[C@@H]1OC[C@@H]2NC(=O)CCCCN(CC(O)CCCCCCCCCC)CC(O)CCCCCCCCCC)CC(O)CCCCCCCCCC] |
| 72 | 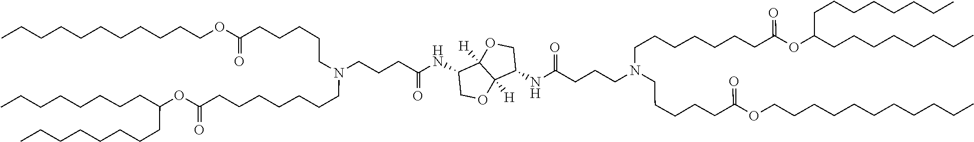 [SMILES: CCCCCCCCCCCOC(=O)CCCCCN(CCCCCCCC(=O)OC(CCCCCCCC)CCCCCCCC)CCCC(=O)N[C@H]1CO[C@H]2[C@@H]1OC[C@@H]2NC(=O)CCCN(CCCCCCCC(=O)OC(CCCCCCCC)CCCCCCCC)CCCCCC(=O)OCCCCCCCCCCC] |
| 73 | 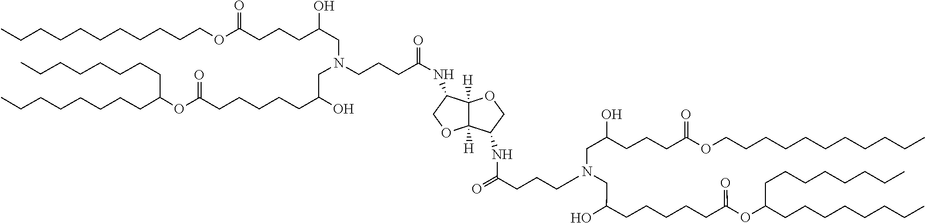 [SMILES: CCCCCCCCCCCOC(=O)CCCC(O)CN(CCCC(=O)N[C@H]1CO[C@H]2[C@@H]1OC[C@@H]2NC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| 74 | 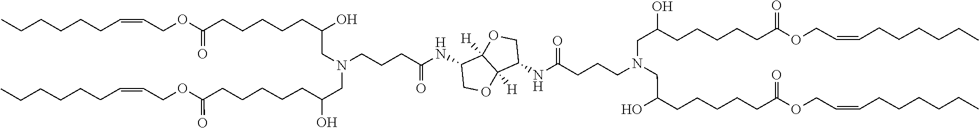 [SMILES: CCCCCC/C=C\COC(=O)CCCCCC(O)CN(CCCC(=O)N[C@H]1CO[C@H]2[C@@H]1OC[C@@H]2NC(=O)CCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC] |
| 75 | 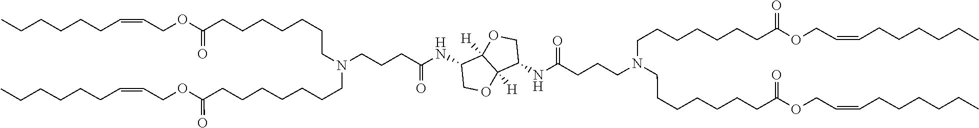 [SMILES: CCCCCC/C=C\COC(=O)CCCCCCCN(CCCCCCCC(=O)OC/C=C\CCCCCC)CCCC(=O)N[C@H]1CO[C@H]2[C@@H]1OC[C@@H]2NC(=O)CCCN(CCCCCCCC(=O)OC/C=C\CCCCCC)CCCCCCCC(=O)OC/C=C\CCCCCC] |
| 76 | 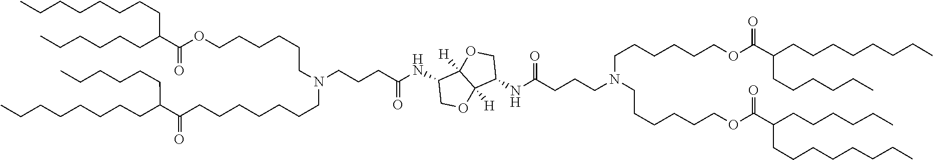 [SMILES: CCCCCCCCC(CCCCCC)C(=O)CCCCCCCN(CCCCCCOC(=O)C(CCCCCC)CCCCCCCC)CCCC(=O)N[C@H]1CO[C@H]2[C@@H]1OC[C@@H]2NC(=O)CCCN(CCCCCCOC(=O)C(CCCCCC)CCCCCCCC)CCCCCCOC(=O)C(CCCCCC)CCCCCCCC] |
| 77 | 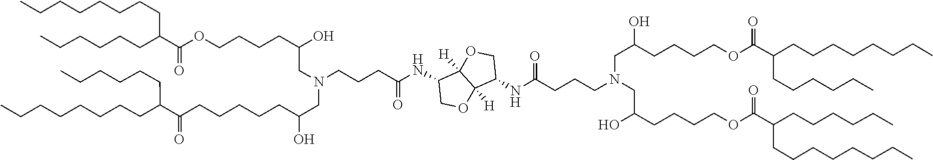 [SMILES: CCCCCCCCC(CCCCCC)C(=O)CCCCCC(O)CN(CCCC(=O)N[C@H]1CO[C@H]2[C@@H]1OC[C@@H]2NC(=O)CCCN(CC(O)CCCCOC(=O)C(CCCCCC)CCCCCCCC)CC(O)CCCCOC(=O)C(CCCCCC)CCCCCCCC)CC(O)CCCCOC(=O)C(CCCCCC)CCCCCCCC] |
| 78 | 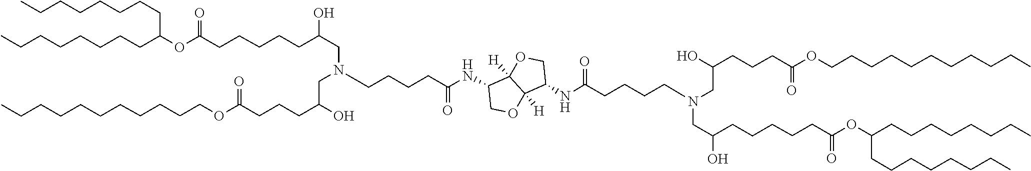 [SMILES: CCCCCCCCCCCOC(=O)CCCC(O)CN(CCCCC(=O)N[C@H]1CO[C@H]2[C@@H]1OC[C@@H]2NC(=O)CCCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC] |
| 79 | 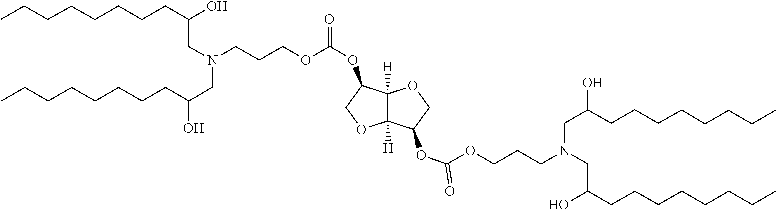 [SMILES: CCCCCCCCC(O)CN(CCCOC(=O)O[C@@H]1CO[C@H]2[C@@H]1OC[C@H]2OC(=O)OCCCN(CC(O)CCCCCCCC)CC(O)CCCCCCCC)CC(O)CCCCCCCC] |
| 80 | 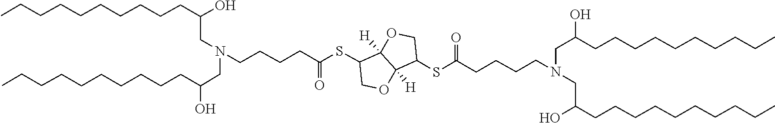 [SMILES: CCCCCCCCCCC(O)CN(CCCCC(=O)SC1CO[C@@H]2C(SC(=O)CCCCN(CC(O)CCCCCCCCCC)CC(O)CCCCCCCCCC)CO[C@H]12)CC(O)CCCCCCCCCC] |
| 81 | 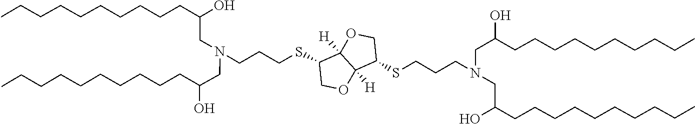 [SMILES: CCCCCCCCCCC(O)CN(CCCS[C@H]1CO[C@H]2[C@@H]1OC[C@@H]2SCCCN(CC(O)CCCCCCCCCC)CC(O)CCCCCCCCCC)CC(O)CCCCCCCCCC] |
| 82 | 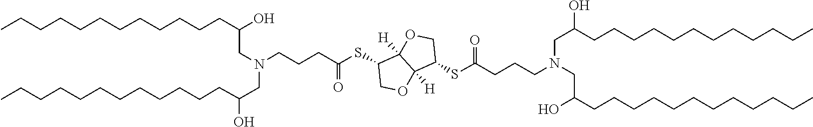 [SMILES: CCCCCCCCCCCCC(O)CN(CCCC(=O)S[C@H]1CO[C@H]2[C@@H]1OC[C@@H]2SC(=O)CCCN(CC(O)CCCCCCCCCCCC)CC(O)CCCCCCCCCCCC)CC(O)CCCCCCCCCCCC] |
| 83 | 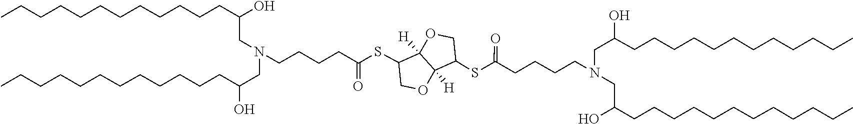 [SMILES: CCCCCCCCCCCCC(O)CN(CCCCC(=O)SC1CO[C@@H]2C(SC(=O)CCCCN(CC(O)CCCCCCCCCCCC)CC(O)CCCCCCCCCCCC)CO[C@H]12)CC(O)CCCCCCCCCCCC] |
| 84 | 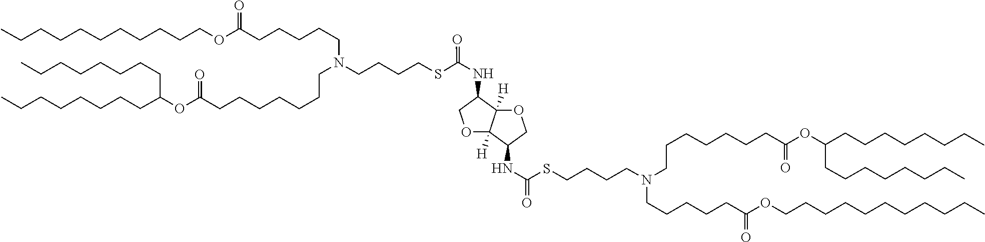 [SMILES: CCCCCCCCCCCOC(=O)CCCCCN(CCCCCCCC(=O)OC(CCCCCCCC)CCCCCCCC)CCCCSC(=O)N[C@@H]1CO[C@H]2[C@@H]1OC[C@H]2NC(=O)SCCCCN(CCCCCCCC(=O)OC(CCCCCCCC)CCCCCCCC)CCCCCC(=O)OCCCCCCCCCCC] |
| 85 | 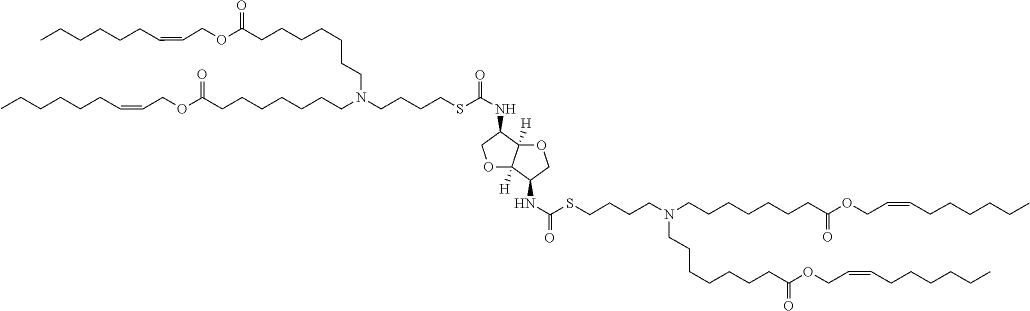 [SMILES: CCCCCC/C=C\COC(=O)CCCCCCCN(CCCCCCCC(=O)OC/C=C\CCCCCC)CCCCSC(=O)N[C@@H]1CO[C@H]2[C@@H]1OC[C@H]2NC(=O)SCCCCN(CCCCCCCC(=O)OC/C=C\CCCCCC)CCCCCCCC(=O)OC/C=C\CCCCCC] |
| 86 | 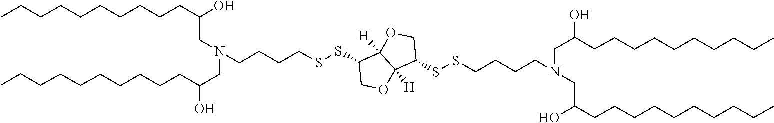 [SMILES: CCCCCCCCCCC(O)CN(CCCCSS[C@H]1CO[C@H]2[C@@H]1OC[C@@H]2SSCCCCN(CC(O)CCCCCCCCCC)CC(O)CCCCCCCCCC)CC(O)CCCCCCCCCC] |

Any of the compounds (1-86) identified in Tables A or B above may be provided in the form of a pharmaceutically acceptable salt and such salts are intended to be encompassed by the present invention.

The compounds of the invention as described herein can be prepared according to methods known in the art, including the exemplary syntheses of the Examples provided herein.

### Nucleic Acids

The compounds of the invention as described herein can be used to prepare compositions useful for the delivery of nucleic acids.

### Synthesis of Nucleic Acids

Nucleic acids according to the present invention may be synthesized according to any known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, mutated T7 or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7, mutated T7 or SP6 promoter, for in vitro transcription, followed by desired nucleotide sequence for desired mRNA and a termination signal.

Desired mRNA sequence(s) according to the invention may be determined and incorporated into a DNA template using standard methods. For example, starting from a desired amino acid sequence (e.g., an enzyme sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

Modified mRNA

In some embodiments, mRNA according to the present invention may be synthesized as unmodified or modified mRNA. Modified mRNA comprises nucleotide modifications in the RNA. A modified mRNA according to the invention can thus include nucleotide modification that are, for example, backbone modifications, sugar modifications or base modifications. In some embodiments, mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g., 1-methyl-adenine, 2-methyl-adenine, 2-methylthio-N-6-isopentenyl-adenine, N-6-methyl-adenine, N-6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queuosine, beta-D-mannosyl-queuosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

### Pharmaceutical Formulations of Cationic Lipids and Nucleic Acids

In certain embodiments, the compounds of the invention as described herein, as well as pharmaceutical and liposomal compositions comprising such lipids, can be used in formulations to facilitate the delivery of encapsulated materials (e.g., one or more polynucleotides such as mRNA) to, and subsequent transfection of one or more target cells. For example, in certain embodiments cationic lipids described herein (and compositions such as liposomal compositions comprising such lipids) are characterized as resulting in one or more of receptor-mediated endocytosis, clathrin-mediated and caveolae-mediated endocytosis, phagocytosis and macropinocytosis, fusogenicity, endosomal or lysosomal disruption and/or releasable properties that afford such compounds advantages relative other similarly classified lipids.

According to the present invention, a nucleic acid, e.g., mRNA encoding a protein (e.g., a full length, fragment or portion of a protein) as described herein may be delivered via a delivery vehicle comprising a compound of the invention as described herein.

As used herein, the terms “delivery vehicle,” “transfer vehicle,” “nanoparticle,” or grammatical equivalents thereof, are used interchangeably.

For example, the present invention provides a composition (e.g., a pharmaceutical composition) comprising a compound described herein and one or more polynucleotides. A composition (e.g., a pharmaceutical composition) may further comprise
(i) one or more cationic lipids,
(ii) one or more non-cationic lipids,
(iii) one or more cholesterol-based lipids and/or
(iv) one or more PEG-modified lipids.

In certain embodiments a composition exhibits an enhanced (e.g., increased) ability to transfect one or more target cells. Accordingly, also provided herein are methods of transfecting one or more target cells. Such methods generally comprise the step of contacting the one or more target cells with the cationic lipids and/or pharmaceutical compositions disclosed herein (e.g., a liposomal formulation comprising a compound described herein encapsulating one or more polynucleotides) such that the one or more target cells are transfected with the materials encapsulated therein (e.g., one or more polynucleotides). As used herein, the terms “transfect” or “transfection” refer to the intracellular introduction of one or more encapsulated materials (e.g., nucleic acids and/or polynucleotides) into a cell (e.g., into a target cell). The introduced polynucleotide may be stably or transiently maintained in the target cell. The term “transfection efficiency” refers to the relative amount of such encapsulated material (e.g., polynucleotides) up-taken by, introduced into, and/or expressed by the target cell which is subject to transfection. In practice, transfection efficiency may be estimated by the amount of a reporter polynucleotide product produced by the target cells following transfection. In certain embodiments, the compounds and pharmaceutical compositions described herein demonstrate high transfection efficiencies thereby improving the likelihood that appropriate dosages of the encapsulated materials (e.g., one or more polynucleotides) will be delivered to the site of pathology and subsequently expressed, while at the same time minimizing potential systemic adverse effects or toxicity associated with the compound or their encapsulated contents.

Following transfection of one or more target cells by, for example, the polynucleotides encapsulated in the one or more lipid nanoparticles comprising the pharmaceutical or liposomal compositions disclosed herein, the production of the product (e.g., a polypeptide or protein) encoded by such polynucleotide may be stimulated and the capability of such target cells to express the polynucleotide and produce, for example, a polypeptide or protein of interest is enhanced. For example, transfection of a target cell by one or more compounds or pharmaceutical compositions encapsulating mRNA will enhance (i.e., increase) the production of the protein or enzyme encoded by such mRNA.

Further, delivery vehicles described herein (e.g., liposomal delivery vehicles) may be prepared to preferentially distribute to other target tissues, cells or organs, such as the heart, lungs, kidneys, spleen. In embodiments, the delivery vehicles described herein (e.g., liposomal delivery vehicles) may be prepared to preferentially distribute to the lungs. In embodiments, the lipid nanoparticles of the present invention may be prepared to achieve enhanced delivery to the target cells and tissues. For example, polynucleotides (e.g., mRNA) encapsulated in one or more of the compounds or pharmaceutical and liposomal compositions described herein can be delivered to and/or transfect targeted cells or tissues. In some embodiments, the encapsulated polynucleotides (e.g., mRNA) are capable of being expressed and functional polypeptide products produced (and in some instances excreted) by the target cell, thereby conferring a beneficial property to, for example the target cells or tissues. Such encapsulated polynucleotides (e.g., mRNA) may encode, for example, an antigen hormone, enzyme, receptor, polypeptide, peptide or other protein of interest.

### Liposomal Delivery Vehicles

In some embodiments, a composition is a suitable delivery vehicle. In embodiments, a composition is a liposomal delivery vehicle, e.g., a lipid nanoparticle.

The terms “liposomal delivery vehicle” and “liposomal composition” are used interchangeably.

Enriching liposomal compositions with one or more of the cationic lipids disclosed herein may be used as a means of improving the safety profile or otherwise conferring one or more desired properties to such enriched liposomal composition (e.g., improved delivery of the encapsulated polynucleotides to one or more target cells and/or reduced in vivo toxicity of a liposomal composition). Accordingly, also contemplated are pharmaceutical compositions, and in particular liposomal compositions, that comprise one or more of the cationic lipids disclosed herein.

Thus, in certain embodiments, the compounds of the invention as described herein may be used as a component of a liposomal composition to facilitate or enhance the delivery and release of encapsulated materials (e.g., one or more therapeutic agents) to one or more target cells (e.g., by permeating or fusing with the lipid membranes of such target cells).

As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphophilic polymers and surfactants (e.g., polymerosomes, niosomes, etc.). In the context of the present invention, a liposomal delivery vehicle typically serves to transport a desired mRNA to a target cell or tissue.

In certain embodiments, such compositions (e.g., liposomal compositions) are loaded with or otherwise encapsulate materials, such as for example, one or more biologically-active polynucleotides (e.g., mRNA).

In embodiments, a composition (e.g., a pharmaceutical composition) comprises an mRNA encoding a peptide or protein, encapsulated within a liposome. In embodiments, a liposome comprises:
(i) one or more cationic lipids,
(ii) one or more non-cationic lipids,
(iii) one or more cholesterol-based lipids and
(iv) one or more PEG-modified lipids, wherein at least one cationic lipid is a compound of the invention as described herein.

In embodiments, a composition comprises an mRNA encoding for a peptide or protein (e.g., any peptide or protein described herein). In embodiments, a composition comprises an mRNA encoding for a peptide (e.g., any peptide described herein). In embodiments, a composition comprises an mRNA encoding for a protein (e.g., any protein described herein).

In embodiments, a composition (e.g., a pharmaceutical composition) comprises a nucleic acid encapsulated within a liposome, wherein the liposome comprises a compound described herein.

In embodiments, a nucleic acid is an mRNA encoding a peptide or protein. In embodiments, an mRNA encodes a peptide or protein for use in the delivery to or treatment of the lung of a subject or a lung cell. In embodiments, an mRNA encodes a peptide or protein for use in the delivery to or treatment of the liver of a subject or a liver cell. In embodiments, an mRNA encodes a peptide or protein for use in the delivery to or treatment of a muscle cell. In embodiments, an mRNA encodes a peptide or protein for use in the delivery to or treatment of an immune cell. Still other exemplary mRNAs are described herein.

In embodiments, a liposomal delivery vehicle (e.g., a lipid nanoparticle) can have a net positive charge.

In embodiments, a liposomal delivery vehicle (e.g., a lipid nanoparticle) can have a net negative charge.

In embodiments, a liposomal delivery vehicle (e.g., a lipid nanoparticle) can have a net neutral charge.

In embodiments, a lipid nanoparticle that encapsulates a nucleic acid (e.g., mRNA encoding a peptide or protein) comprises one or more compounds of the invention as described herein.

For example, the amount of a compound of the invention as described herein in a composition can be described as a percentage (“wt %”) of the combined dry weight of all lipids of a composition (e.g., the combined dry weight of all lipids present in a liposomal composition).

In embodiments of the pharmaceutical compositions described herein, a compound of the invention as described herein is present in an amount that is about 0.5 wt % to about 30 wt % (e.g., about 0.5 wt % to about 20 wt %) of the combined dry weight of all lipids present in a composition (e.g., a liposomal composition).

In embodiments, a compound of the invention as described herein is present in an amount that is about 1 wt % to about 30 wt %, about 1 wt % to about 20 wt %, about 1 wt % to about 15 wt %, about 1 wt % to about 10 wt %, or about 5 wt % to about 25 wt % of the combined dry weight of all lipids present in a composition (e.g., a liposomal composition). In embodiments, a compound of the invention as described herein is present in an amount that is about 0.5 wt % to about 5 wt %, about 1 wt % to about 10 wt %, about 5 wt % to about 20 wt %, or about 10 wt % to about 20 wt % of the combined dry weight of all lipids present in a composition such as a liposomal delivery vehicle.

In embodiments, the amount of a compound of the invention as described herein is present in an amount that is at least about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, the amount of a compound of the invention as described herein is present in an amount that is no more than about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, a composition (e.g., a liposomal delivery vehicle such as a lipid nanoparticle) comprises about 0.1 wt % to about 20 wt % (e.g., about 0.1 wt % to about 15 wt %) of a compound described herein. In embodiments, a delivery vehicle (e.g., a liposomal delivery vehicle such as a lipid nanoparticle) comprises about 0.5 wt %, about 1 wt %, about 3 wt %, about 5 wt %, or about 10 wt % of a compound described herein. In embodiments, a delivery vehicle (e.g., a liposomal delivery vehicle such as a lipid nanoparticle) comprises up to about 0.5 wt %, about 1 wt %, about 3 wt %, about 5 wt %, about 10 wt %, about 15 wt %, or about 20 wt % of a compound described herein. In embodiments, the percentage results in an improved beneficial effect (e.g., improved delivery to targeted tissues such as the liver or the lung).

The amount of a compound of the invention as described herein in a composition also can be described as a percentage (“mol %”) of the combined molar amounts of total lipids of a composition (e.g., the combined molar amounts of all lipids present in a liposomal delivery vehicle).

In embodiments of pharmaceutical compositions described herein, a compound of the invention as described herein is present in an amount that is about 0.5 mol % to about 50 mol % (e.g., about 0.5 mol % to about 20 mol %) of the combined molar amounts of all lipids present in a composition such as a liposomal delivery vehicle.

In embodiments, a compound of the invention as described herein is present in an amount that is about 0.5 mol % to about 5 mol %, about 1 mol % to about 10 mol %, about 5 mol % to about 20 mol %, about 10 mol % to about 20 mol %, about 15 mol % to about 30 mol %, about 20 mol % to about 35 mol %, about 25 mol % to about 40 mol %, about 30 mol % to about 45 mol %, about 35 mol % to about 50 mol %, about 40 mol % to about 55 mol %, or about 45 mol % to about 60 mol % of the combined molar amounts of all lipids present in a composition such as a liposomal delivery vehicle. In embodiments, a compound of the invention as described herein is present in an amount that is about 1 mol % to about 60 mol %, 1 mol % to about 50 mol %, 1 mol % to about 40 mol %, 1 mol % to about 30 mol %, about 1 mol % to about 20 mol %, about 1 mol % to about 15 mol %, about 1 mol % to about 10 mol %, about 5 mol % to about 55 mol %, about 5 mol % to about 45 mol %, about 5 mol % to about 35 mol % or about 5 mol % to about 25 mol % of the combined molar amounts of all lipids present in a composition such as a liposomal delivery vehicle.

In certain embodiments, a compound of the invention as described herein can comprise from about 0.1 mol % to about 50 mol %, or from 0.5 mol % to about 50 mol %, or from about 1 mol % to about 50 mol %, or from about 5 mol % to about 50 mol %, or from about 10 mol % to about 50 mol %, or from about 15 mol % to about 50 mol %, or from about 20 mol % to about 50 mol %, or from about 25 mol % to about 50 mol %, or from about 30 mol % to about 50 mol %, of the total amount of lipids in a composition (e.g., a liposomal delivery vehicle).

In certain embodiments, a compound of the invention as described herein can comprise greater than about 0.1 mol %, or greater than about 0.5 mol %, or greater than about 1 mol %, greater than about 5 mol %, greater than about 10 mol %, greater than about 20 mol %, greater than about 30 mol %, or greater than about 40 mol % of the total amount of lipids in the lipid nanoparticle.

In certain embodiments, a compound as described can comprise less than about 60 mol %, or less than about 55 mol %, or less than about 50 mol %, or less than about 45 mol %, or less than about 40 mol %, or less than about 35 mol %, less than about 30 mol %, or less than about 25 mol %, or less than about 10 mol %, or less than about 5 mol %, or less than about 1 mol % of the total amount of lipids in a composition (e.g., a liposomal delivery vehicle).

In embodiments, the amount of a compound of the invention as described herein is present in an amount that is at least about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined molar amounts of total lipids in a composition (e.g., a liposomal composition).

In embodiments, the amount of a compound of the invention as described herein is present in an amount that is no more than about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined molar amounts of total lipids in a composition (e.g., a liposomal composition).

In embodiments, the percentage results in an improved beneficial effect (e.g., improved delivery to targeted tissues such as the liver, the lung or muscle).

In a typical embodiment, a composition of the invention (e.g., a liposomal composition) comprises:
(i) one or more cationic lipids,
(ii) one or more non-cationic lipids,
(iii) one or more cholesterol-based lipids, and
(iv) one or more PEG-modified lipids, wherein at least one cationic lipid is a compound of the invention as described herein.

For example, a composition suitable for practicing the invention has four lipid components comprising a compound of the invention as described herein as the cationic lipid component, and further comprising:
(i) a non-cationic lipid,
(ii) a cholesterol-based lipid and
(iii) a PEG-modified lipid.

The non-cationic lipid may be DOPE or DEPE. The cholesterol-based lipid may be cholesterol. The PEG-modified lipid may be DMG-PEG2K.

In further embodiments, pharmaceutical (e.g., liposomal) compositions comprise one or more of a PEG-modified lipid, a non-cationic lipid and a cholesterol lipid. In other embodiments, such pharmaceutical (e.g., liposomal) compositions comprise: one or more PEG-modified lipids; one or more non-cationic lipids; and one or more cholesterol lipids. In yet further embodiments, such pharmaceutical (e.g., liposomal) compositions comprise: one or more PEG-modified lipids and one or more cholesterol lipids.

In embodiments, a composition (e.g., lipid nanoparticle) that encapsulates a nucleic acid (e.g., mRNA encoding a peptide or protein) comprises one or more compounds of the invention as described herein, and one or more lipids selected from the group consisting of a cationic lipid, a non-cationic lipid, and a PEGylated lipid.

In embodiments, a composition (e.g., lipid nanoparticle) that encapsulates a nucleic acid (e.g., mRNA encoding a peptide or protein) comprises one or more compound of the invention as described herein; one or more lipids selected from the group consisting of a cationic lipid, a non-cationic lipid, and a PEGylated lipid; and further comprises a cholesterol-based lipid. Typically, such a composition has four lipid components comprising a compound of the invention as described herein as the cationic lipid component, and further comprising:
(i) a non-cationic lipid (e.g., DOPE),
(ii) a cholesterol-based lipid (e.g., cholesterol) and
(iii) a PEG-modified lipid (e.g., DMG-PEG2K).

In embodiments, a lipid nanoparticle that encapsulates a nucleic acid (e.g., mRNA encoding a peptide or protein) comprises one or more compounds of the invention as described herein, as well as one or more lipids selected from the group consisting of:
(i) a cationic lipid,
(ii) a non-cationic lipid,
(iii) a PEGylated lipid, and
(iv) a cholesterol-based lipid.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). Thus, the molar ratios may be adjusted accordingly.

### Cationic Lipids

In addition to any of the compounds of the invention as described herein, a composition may comprise one or more additional cationic lipids.

In some embodiments, liposomes may comprise one or more additional cationic lipids. As used herein, the phrase “cationic lipid” refers to any of a number of lipid species that have a net positive charge at a selected pH, such as physiological pH. Several cationic lipids have been described in the literature, many of which are commercially available.

Suitable additional cationic lipids for use in the compositions include the cationic lipids as described in the literature.

### Helper Lipids

Compositions (e.g., liposomal compositions) may also comprise one or more helper lipids. Such helper lipids include non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), 1,2-Dierucoyl-sn-glycero-3-phosphoethanolamine (DEPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof. A non-cationic or helper lipid suitable for practicing the invention is dioleoylphosphatidylethanolamine (DOPE). Alternatively, 1,2-Dierucoyl-sn-glycero-3-phosphoethanolamine (DEPE) can be used as a non-cationic or helper lipid.

In some embodiments, a non-cationic lipid is a neutral lipid, i.e., a lipid that does not carry a net charge in the conditions under which the composition is formulated and/or administered.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 mol %, greater than about 10 mol %, greater than about 20 mol %, greater than about 30 mol %, or greater than about 40 mol %. In some embodiments, the percentage total non-cationic lipids in a liposome may be greater than about 5 mol %, greater than about 10 mol %, greater than about 20 mol %, greater than about 30 mol %, or greater than about 40 mol %. In some embodiments, the percentage of non-cationic lipid in a liposome is no more than about 5 mol %, no more than about 10 mol %, no more than about 20 mol %, no more than about 30 mol %, or no more than about 40 mol %. In some embodiments, the percentage total non-cationic lipids in a liposome may be no more than about 5 mol %, no more than about 10 mol %, no more than about 20 mol %, no more than about 30 mol %, or no more than about 40 mol %.

In some embodiments, a non-cationic lipid may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 wt %, greater than about 10 wt %, greater than about 20 wt %, greater than about 30 wt %, or greater than about 40 wt %. In some embodiments, the percentage total non-cationic lipids in a liposome may be greater than about 5 wt %, greater than about 10 wt %, greater than about 20 wt %, greater than about 30 wt %, or greater than about 40 wt %. In some embodiments, the percentage of non-cationic lipid in a liposome is no more than about 5 wt %, no more than about 10 wt %, no more than about 20 wt %, no more than about 30 wt %, or no more than about 40 wt %. In some embodiments, the percentage total non-cationic lipids in a liposome may be no more than about 5 wt %, no more than about 10 wt %, no more than about 20 wt %, no more than about 30 wt %, or no more than about 40 wt %.

### Cholesterol-Based Lipids

In some embodiments, a composition (e.g., a liposomal composition) comprising a cationic lipid of the present invention further comprises one or more cholesterol-based lipids. For example, a suitable cholesterol-based lipid for practicing the invention is cholesterol. Other suitable cholesterol-based lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), beta-sitosterol, or imidazole cholesterol ester (ICE), which has the following structure,

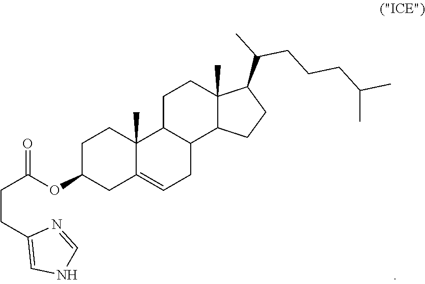 [SMILES: CC(C)CCCC(C)[C@H]1CCC2C3CC=C4C[C@@H](OC(=O)CCc5c[nH]cn5)CC[C@]4(C)C3CC[C@@]21C]

In some embodiments, a cholesterol-based lipid may be present in a molar ratio (mol %) of about 1% to about 30%, or about 5% to about 20% of the total lipids present in a liposome. In some embodiments, the percentage of cholesterol-based lipid in the lipid nanoparticle may be greater than about 5 mol %, greater than about 10 mol %, greater than about 20 mol %, greater than about 30 mol %, or greater than about 40 mol %. In some embodiments, the percentage of cholesterol-based lipid in the lipid nanoparticle may be no more than about 5 mol %, no more than about 10 mol %, no more than about 20 mol %, no more than about 30 mol %, or no more than about 40 mol %.

In some embodiments, a cholesterol-based lipid may be present in a weight ratio (wt %) of about 1% to about 30%, or about 5% to about 20% of the total lipids present in a liposome. In some embodiments, the percentage of cholesterol-based lipid in the lipid nanoparticle may be greater than about 5 wt %, greater than about 10 wt %, greater than about 20 wt %, greater than about 30 wt %, or greater than about 40 wt %. In some embodiments, the percentage of cholesterol-based lipid in the lipid nanoparticle may be no more than about 5 wt %, no more than about 10 wt %, no more than about 20 wt %, no more than about 30 wt %, or no more than about 40 wt %.

### PEGylated Lipids

In some embodiments, a composition (e.g., a liposomal composition) comprises one or more further PEGylated lipids. A suitable PEG-modified or PEGylated lipid for practicing the invention is 1,2-dimyristoyl-rac-glycero-3-methoxypolyethylene glycol-2000 (DMG-PEG2K).

For example, the use of polyethylene glycol (PEG)-modified phospholipids and derivatized lipids such as derivatized ceramides (PEG-CER), including N-octanoyl-sphingosine-1-[succinyl(methoxy polyethylene glycol)-2000](C8 PEG-2000 ceramide) is also contemplated by the present invention in combination with one or more of compounds of the invention as described herein and, in some embodiments, other lipids together which comprise the liposome. In some embodiments, particularly useful exchangeable lipids are PEG-ceramides having shorter acyl chains (e.g., (C₁₄) or (C₁₈)).

Contemplated further PEG-modified lipids (also referred to herein as a PEGylated lipid, which term is interchangeable with PEG-modified lipid) include, but are not limited to, a polyethylene glycol chain of up to 5 kDa in length covalently attached to a lipid with alkyl chain(s) of (C₆-C₂₀) length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613).

Further PEG-modified phospholipid and derivatized lipids of the present invention may be present in a molar ratio (mol %) from about 0% to about 10%, about 0.5% to about 10%, about 1% to about 10%, about 2% to about 10%, about 3% to about 5%, about 1% to about 5%, or about 1.5% to about 3% of the total lipid present in the composition (e.g., a liposomal composition).

### Pharmaceutical Formulations and Therapeutic Uses

Compounds of the invention as described herein may be used in the preparation of compositions (e.g., to construct liposomal compositions) that facilitate or enhance the delivery and release of encapsulated materials (e.g., one or more therapeutic polynucleotides) to one or more target cells (e.g., by permeating or fusing with the lipid membranes of such target cells).

For example, when a liposomal composition (e.g., a lipid nanoparticle) comprises or is otherwise enriched with one or more of the compounds disclosed herein, the phase transition in the lipid bilayer of the one or more target cells may facilitate the delivery of the encapsulated materials (e.g., one or more therapeutic polynucleotides encapsulated in a lipid nanoparticle) into the one or more target cells.

Similarly, in certain embodiments compounds of the invention as described herein may be used to prepare liposomal vehicles that are characterized by their reduced toxicity in vivo. In certain embodiments, the reduced toxicity is a function of the high transfection efficiencies associated with the compositions disclosed herein, such that a reduced quantity of such composition may be administered to the subject to achieve a desired therapeutic response or outcome.

In certain embodiments, compounds of the invention as described herein may be used to prepare liposomal vehicles that are characterized by effective intranasal delivery of mRNA. In certain embodiments, compounds of the invention as described herein may be used to prepare liposomal vehicles that are characterized by effective pulmonary delivery of mRNA. In certain embodiments, compounds of the invention as described herein may be used to prepare liposomal vehicles that are characterized by achieving high levels of peptide or protein expression when delivering mRNA encoding for said peptide or protein by intravenous, intrathecal, intramuscular, intranasal, sublingual, or by pulmonary delivery, optionally through nebulization. In certain embodiments, compounds of the invention as described herein may be used to prepare liposomal vehicles that are characterized by achieving high levels of peptide or protein expression when delivering mRNA encoding for said peptide or protein by intramuscular delivery.

Thus, pharmaceutical formulations comprising a compound described and nucleic acids provided by the present invention may be used for various therapeutic disease and/or disease prevention purposes. To facilitate delivery of nucleic acids in vivo, a compound described herein and nucleic acids can be formulated in combination with one or more additional pharmaceutical carriers, targeting ligands or stabilizing reagents. In some embodiments, a compound described herein can be formulated via pre-mixed lipid solution. In other embodiments, a composition comprising a compound described herein can be formulated using post-insertion techniques into the lipid membrane of the nanoparticles. Techniques for formulation and administration of drugs may be found in “Remington's Pharmaceutical Sciences,” Mack Publishing Co., Easton, Pa., latest edition.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In embodiments, the route of administration is selected from intravenous, intrathecal, intramuscular, intranasal, sublingual, or by pulmonary delivery, optionally through nebulization. In embodiments, the route of administration is intramuscular. In particular embodiments, the intramuscular administration is to a muscle selected from the group consisting of skeletal muscle, smooth muscle and cardiac muscle. In some embodiments the administration results in delivery of the nucleic acids to a muscle cell. In some embodiments the administration results in delivery of the nucleic acids to a hepatocyte (i.e., liver cell).

A common route for administering a liposomal composition of the invention may be intravenous delivery, in particular when treating metabolic disorders, especially those affecting the liver (e.g., ornithine transcarbamylase (OTC) deficiency). Alternatively, depending on the disease or disorder to be treated, the liposomal composition may be administered via pulmonary delivery (e.g., for the treatment of cystic fibrosis). For vaccination, a liposomal composition of the invention is typically administered intramuscularly. Alternatively, a liposomal composition of the invention may be administered intranasally for vaccination. Diseases or disorders affecting the eye may be treated by administering a liposomal composition of the invention intravitreally.

Alternatively or additionally, pharmaceutical formulations of the invention may be administered in a local rather than systemic manner, for example, via injection of the pharmaceutical formulation directly into a targeted tissue (e.g., in a sustained release formulation). Local delivery can be affected in various ways, depending on the tissue to be targeted. Exemplary tissues in which mRNA may be delivered and/or expressed include, but are not limited to the liver, kidney, heart, spleen, serum, brain, skeletal muscle, lymph nodes, skin, and/or cerebrospinal fluid. In embodiments, the tissue to be targeted in the liver. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection.

Alternatively or additionally, pharmaceutical formulations of the invention may be administered intranasally. For example, the pharmaceutical formulations of the invention may be administered via nasal spray. Exemplary tissues in which mRNA may be delivered and/or expressed include, but are not limited to the lungs, heart, liver, spleen and muscle. In embodiments, the tissue to be targeted is in the lungs. In embodiments, the tissue to be targeted is in muscle.

Alternatively or additionally, pharmaceutical formulations of the invention may be administered by pulmonary delivery, optionally through nebulization or dry powder inhalation. In embodiments, the pharmaceutical formulations of the invention are administered by pulmonary delivery through nebulization. In embodiments, the pharmaceutical formulations of the invention are administered by pulmonary delivery through dry powder inhalation. Exemplary tissues in which mRNA may be delivered and/or expressed include, but are not limited to the lungs, heart, liver, spleen and muscle. In embodiments, the tissue to be targeted is in the lungs. In embodiments, the tissue to be targeted is in muscle.

Compositions described herein can comprise mRNA encoding peptides including those described herein (e.g., a polypeptide such as a protein).

In embodiments, a mRNA encodes a polypeptide. In embodiments, a mRNA encodes a peptide. In embodiments, the peptide is an antigen. In embodiments, a mRNA encodes a peptide for treating Flu. In embodiments, a mRNA encodes a peptide for treating Respiratory Syncytial virus (RSV).

An antigen encoded by a nucleic acid of the disclosure may be suitable to be used for the treatment or prevention of various diseases that may affect humans or animals other than humans.

An antigen can be from bacteria, viruses, parasites or from cancer cells.

Viral antigens may be selected from the group of viruses consisting of: poliovirus, rabies virus, hepatitis A, hepatitis B, hepatitis C, yellow fever virus, varicella zoster virus (VZV), measles virus, mumps virus, rubella virus, Japanese encephalitis, influenza virus, norovirus, rhinovirus, respiratory syncytial virus (RSV), human metapneumovirus (hMPV), sars-cov-1, sars-cov-2, herpes simplex virus, papilloma virus, cytomegalovirus virus, rotavirus, West Nile virus, dengue virus, chikungunya virus, HIV (AIDS), and combinations thereof.

Bacterial antigens may be selected from the group of bacteria consisting of: Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus Thuringiensis, Bacteroides fragilis, Bacteroides gingivalis, Bacteroides melaninogenicus, Bartonella henselae, Bartonella Quintana, Bordetella bronchiseptica, Bordetella pertussis, Borrelia burgdorferi. Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Cutibacterium acnes, Cutibacterium avidum, Cutibacterium granulosum, Cutibacterium namnetense, Cutibacterium humerusii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica, Propionibacterium acnes, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Viridans streptococci, Wolbachia, Yersinia enterocolitica, Yersinia pestis, Yersinia pseudotuberculosis, and combinations thereof.

Parasitic antigens may be selected from the group of parasites consisting of: Plasmodium spp., Leishmania spp., Trypanosoma spp., Schistosome spp.

Cancer antigens are molecules expressed on the surface of cancer cells or secreted into the bloodstream which can be recognized and targeted by the immune system. These antigens are often absent or present at much lower levels on healthy cells.

Cancer antigens may be selected from the group consisting of: HER2/neu, EGFR (Epidermal Growth Factor Receptor), BRAF, carcinoembryonic antigen (CEA), MAGE-A, NY-ESO-1.

In embodiments, a mRNA encodes a protein. In embodiments, a mRNA encodes a protein for treating Flu. In embodiments, a mRNA encodes a protein for treating Respiratory Syncytial virus (RSV).

The present invention provides methods for delivering a composition having full-length mRNA molecules encoding a peptide or protein of interest for use in the treatment of a subject, e.g., a human subject or a cell of a human subject or a cell that is treated and delivered to a human subject.

### Delivery Methods

The route of delivery used in the methods of the invention allows for non-invasive, self-administration of the compounds of the invention. In some embodiments, the methods involve intranasal, intratracheal or pulmonary administration by aerosolization, nebulization, or instillation of a composition comprising mRNA encoding a therapeutic peptide or protein in a suitable transfection or lipid carrier vehicles as described above. In some embodiments, the methods involve intranasal, intratracheal or pulmonary administration by intravenous, intrathecal, intramuscular, intranasal, sublingual, or by pulmonary delivery, optionally through nebulization of a composition comprising mRNA encoding a therapeutic peptide or protein in a suitable transfection or lipid carrier vehicles as described above. In some embodiments, the peptide or protein is encapsulated with a liposome. In some embodiments, the liposome comprises a lipid, which is a compound of the invention. As used herein below, administration of a compound of the invention includes administration of a composition comprising a compound of the invention.

Although the local cells and tissues of the lung represent a potential target capable of functioning as a biological depot or reservoir for production and secretion of the protein encoded by the mRNA, applicants have discovered that administration of the compounds of the invention to the lung via aerosolization, nebulization, or instillation results in the distribution of even non-secreted proteins outside the lung cells. Without wishing to be bound by any particular theory, it is contemplated that nanoparticle compositions of the invention pass, through the lung airway-blood barrier, resulting in translation of the intact nanoparticle to non-lung cells and tissues, such as, e.g., the heart, the liver, the spleen, where it results in the production of the encoded peptide or protein in these non-lung tissues. Thus, the utility of the compounds of the invention and methods of the invention extend beyond production of therapeutic protein in lung cells and tissues of the lung and can be used to delivery to non-lung target cells and/or tissues. They are useful in the management and treatment of a large number of diseases. In certain embodiments, the compounds of the invention, used in the methods of the invention result in the distribution of the mRNA encapsulated nanoparticles and production of the encoded peptide or protein in the liver, spleen, heart, and/or other non-lung cells. For example, administration of the compounds of the invention, by aerosolization, nebulization, or instillation to the lung will result in the composition itself and its peptide or protein product (e.g., an antigen or functional protein) will be detectable in both the local cells and tissues of the lung, as well as in peripheral target cells, tissues and organs as a result of translocation of the mRNA and delivery vehicle to non-lung cells.

In certain embodiments, the compounds of the invention may be employed in the methods of the invention to specifically target peripheral cells or tissues. Following the pulmonary delivery, it is contemplated the compounds of the invention cross the lung airway-blood barrier and distribute into cells other than the local lung cells. Accordingly, the compounds disclosed herein may be administered to a subject by way of the pulmonary route of administration, using a variety of approach known by those skilled in the art (e.g., by inhalation), and distribute to both the local target cells and tissues of the lung, as well as in peripheral non-lung cells and tissues (e.g., cells of the liver, spleen, kidneys, heart, skeletal muscle, lymph nodes, brain, cerebrospinal fluid, and plasma). As a result, both the local cells of the lung and the peripheral non-lung cells can serve as biological reservoirs or depots capable of producing and/or secreting a translation product encoded by one or more polynucleotides. Accordingly, the present invention is not limited to the treatment of lung diseases or conditions, but rather can be used as a non-invasive means of facilitating the delivery of polynucleotides, or the production of peptides or proteins encoded thereby, in peripheral organs, tissues and cells (e.g., hepatocytes) which would otherwise be achieved only by systemic administration. Exemplary peripheral non-lung cells include, but are not limited to, hepatocytes, epithelial cells, hematopoietic cells, epithelial cells, endothelial cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

Following administration of the composition to the subject, the peptide or protein product encoded by the mRNA (e.g., a functional protein or enzyme) is detectable in the peripheral target tissues for at least about one to seven days or longer following administration of the compound to the subject. The amount of peptide or protein product necessary to achieve a therapeutic effect will vary depending on the condition being treated, the peptide or protein encoded, and the condition of the patient. For example, the peptide or protein product may be detectable in the peripheral target tissues at a concentration (e.g., a therapeutic concentration) of at least 0.025-1.5 μg/ml (e.g., at least 0.050 μg/ml, at least 0.075 μg/ml, at least 0.1 μg/ml, at least 0.2 μg/ml, at least 0.3 μg/ml, at least 0.4 μg/ml, at least 0.5 μg/ml, at least 0.6 μg/ml, at least 0.7 μg/ml, at least 0.8 μg/ml, at least 0.9 μg/ml, at least 1.0 μg/ml, at least 1.1 μg/ml, at least 1.2 μg/ml, at least 1.3 μg/ml, at least 1.4 μg/ml, or at least 1.5 μg/ml), for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 days or longer following administration of the compound to the subject.

It has been demonstrated that nucleic acids can be delivered to the lungs by intratracheal administration of a liquid suspension of the compound and inhalation of an aerosol mist produced by a liquid nebulizer or the use of a dry powder apparatus such as that described in U.S. Pat. No. 5,780,014, incorporated herein by reference.

In certain embodiments, the compounds of the invention may be formulated such that they may be aerosolized or otherwise delivered as a particulate liquid or solid prior to or upon administration to the subject. Such compounds may be administered with the assistance of one or more suitable devices for administering such solid or liquid particulate compositions (such as, e.g., an aerosolized aqueous solution or suspension) to generate particles that are easily respirable or inhalable by the subject. In some embodiments, such devices (e.g., a metered dose inhaler, jet-nebulizer, ultrasonic nebulizer, dry-powder-inhalers, propellant-based inhaler or an insufflator) facilitate the administration of a predetermined mass, volume or dose of the compositions (e.g., about 0.5 mg/kg of mRNA per dose) to the subject. For example, in certain embodiments, the compounds of the invention are administered to a subject using a metered dose inhaler containing a suspension or solution comprising the compound and a suitable propellant. In certain embodiments, the compounds of the invention may be formulated as a particulate powder (e.g., respirable dry particles) intended for inhalation. In certain embodiments, compositions of the invention formulated as respirable particles are appropriately sized such that they may be respirable by the subject or delivered using a suitable device (e.g., a mean D50 or D90 particle size less than about 500 μm, 400 μm, 300 μm, 250 μm, 200 μm, 150 μm, 100 μm, 75 μm, 50 μm, 25 μm, 20 μm, 15 μm, 12.5 μm, 10 μm, 5 μm, 2.5 μm or smaller). In yet other embodiments, the compounds of the invention are formulated to include one or more pulmonary surfactants (e.g., lamellar bodies). In some embodiments, the compounds of the invention are administered to a subject such that a concentration of at least 0.05 mg/kg, at least 0.1 mg/kg, at least 0.5 mg/kg, at least 1.0 mg/kg, at least 2.0 mg/kg, at least 3.0 mg/kg, at least 4.0 mg/kg, at least 5.0 mg/kg, at least 6.0 mg/kg, at least 7.0 mg/kg, at least 8.0 mg/kg, at least 9.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight is administered in a single dose. In some embodiments, the compounds of the invention are administered to a subject such that a total amount of at least 0.1 mg, at least 0.5 mg, at least 1.0 mg, at least 2.0 mg, at least 3.0 mg, at least 4.0 mg, at least 5.0 mg, at least 6.0 mg, at least 7.0 mg, at least 8.0 mg, at least 9.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg or at least 100 mg mRNA is administered in one or more doses.

### EXAMPLES

While certain compounds, compositions and methods of the present invention have been described with specificity in accordance with certain embodiments, the following examples serve only to illustrate the compounds of the invention and are not intended to limit the same.

### List of Abbreviations

DCM: Dichloromethane
DIPEA: N,N-Diisopropylethylamine
DMAP: 4-Dimethylaminopyridine
EDC: 1-Ethyl-3-(3-dimethylaminopropyl)carbodiimide
EtOAc: Ethyl acetate
NaHCO₃: Sodium hydrogencarbonate
Py: Pyridine
Na₂SO₄: Sodium Sulfate
TEA: Triethylamine
TFA: Trifluoroacetic Acid
MS: Mass spectrometry
ESI-MS: Electrospray ionization mass spectrometry
TLC: Thin Layer Chromatography

### Example 1: Synthesis of Compounds of the Present Invention

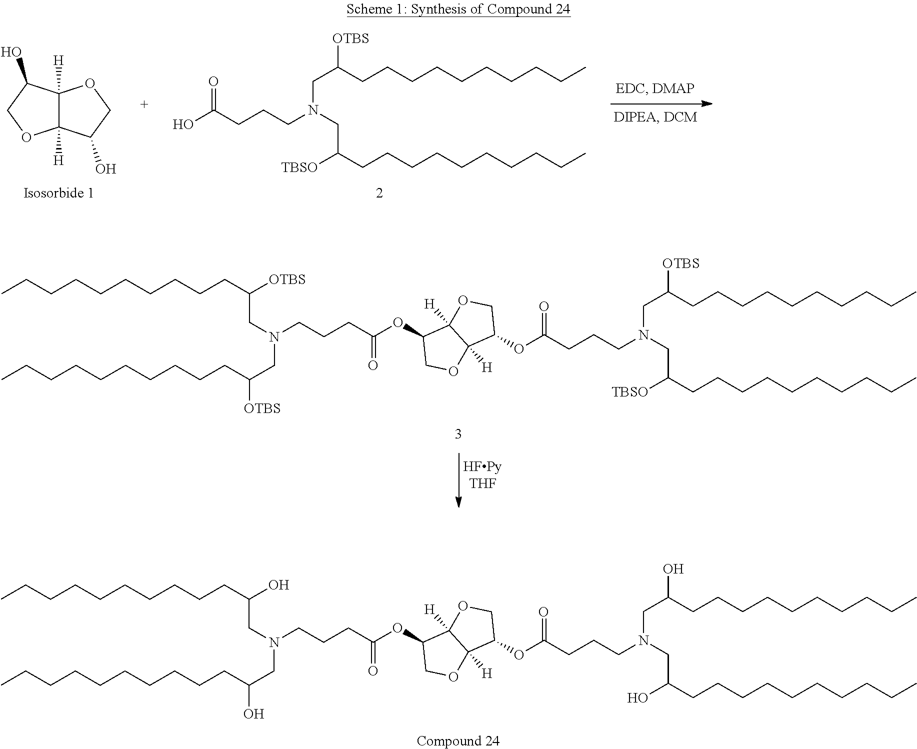

### Step 1: Synthesis of Intermediate (3)

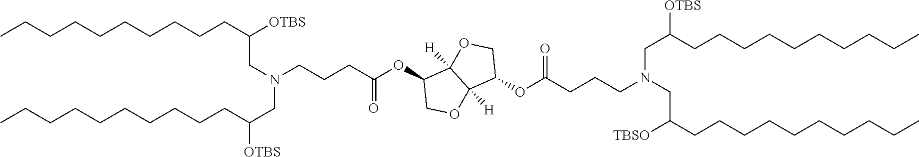 [SMILES: CCCCCCCCCCC(CN(CCCC(=O)O[C@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 1: To a solution of acid (2) (1.2 g, 1.71 mmol) and isosorbide (1) (0.100 g, 0.68 mmol) in dichloromethane (10 mL) were added DIPEA (0.95 mL, 5.47 mmol), DMAP (0.084 g, 0.68 mmol) and EDC (0.393 g, 2.05 mmol). The resulting mixture was stirred at room temperature for overnight. After 16 h, MS and TLC (30% EtOAc in hexanes) analysis indicated completion of the reaction. The reaction mixture was diluted with dichloromethane and washed with saturated NaHCO₃ solution, water and brine solution. The organic layer was dried over anhydrous Na₂SO₄ and concentrated. The crude residue was purified, and the desired product was eluted at 6% EtOAc in hexanes. The product containing fractions were concentrated to obtain 0.72 g (69%) of pure product.

### Results:

ESI-MS: Calculated C₈₆H₁₇₇N₂O₁₀Si₄, [M+H⁺]=1510.25, Observed=1510.3 and 755.4 [M/2+H⁺]

### Step 2: Synthesis of Compound 24

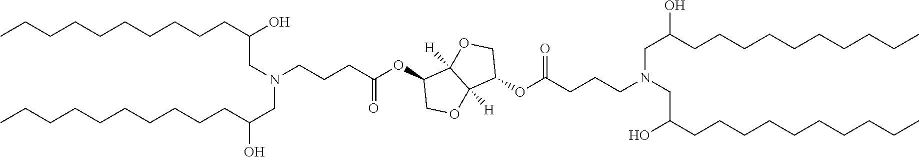 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

As depicted in Scheme 1: To a solution of Intermediate (3) (0.72 g, 0.476 mmol) in tetrahydrofuran (4 mL) was added hydrogen fluoride (70% HF.py complex, 2 mL, 14.298 mmol) at 0° C. and stirred at the same temperature for 5 minutes. Then reaction mixture was warmed to room temperature and stirred for 16 h. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water and brine. Then dried over anhydrous Na₂SO₄ and concentrated. The crude residue was purified, and the desired product was eluted at 65% EtOAc in hexanes. The purest fractions were concentrated to obtain 0.120 g (24%) of pure product.

### Results:

¹H NMR (400 MHz, CDCl₃) δ 5.30-5.00 (m, 2H), 4.97-4.68 (m, 2H), 4.55-3.71 (m, 8H), 3.57-2.92 (m, 8H), 2.84-2.04 (m, 8H), 1.99-1.01 (m, 76H), 0.88 (t, J=6.8 Hz, 12H).

ESI-MS: Calculated C₆₂H₁₂₁N₂O₁₀, [M+H⁺]=1053.90, Observed=1053.2 and 527.3 [M/2+H⁺]

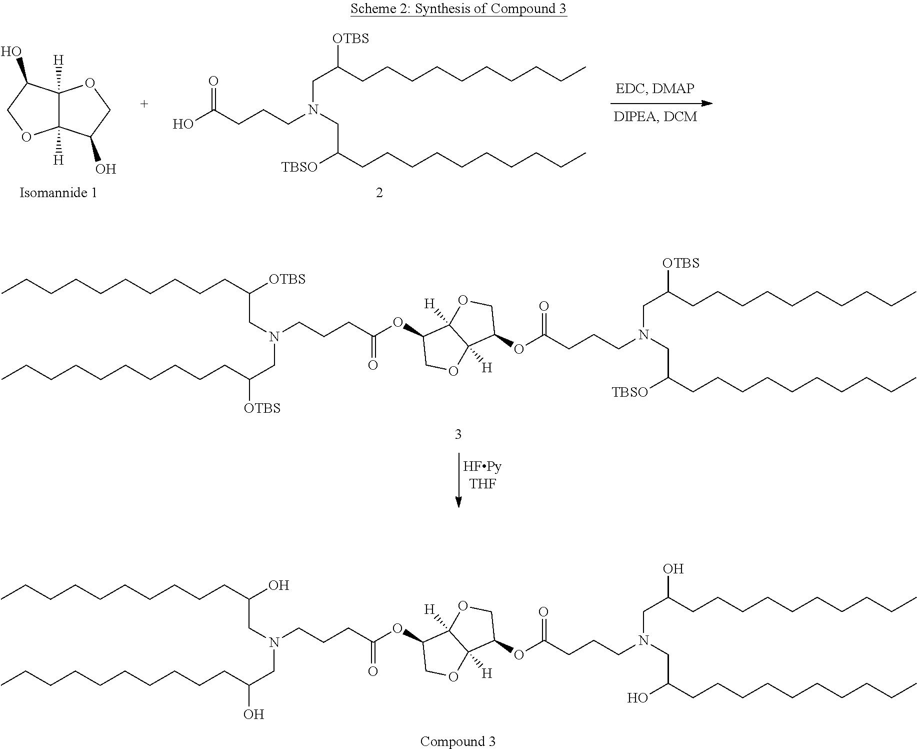

### Step 1: Synthesis Intermediate (3)

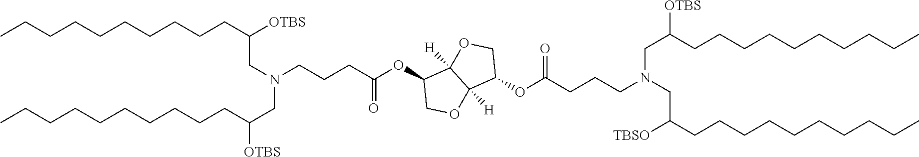 [SMILES: CCCCCCCCCCC(CN(CCCC(=O)O[C@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 2: To a solution of acid (2) (4.58 g, 6.55 mmol) and isomannide (1) (0.38 g, 2.62 mmol) in dichloromethane (40 mL) were added DIPEA (3.65 mL, 20.96 mmol), DMAP (0.32 g, 2.62 mmol) and EDC (1.5 g, 7.86 mmol). The resulting mixture was stirred at room temperature for overnight. After 16 h, MS and TLC (30% EtOAc in hexanes) analysis indicated completion of the reaction. The reaction mixture was diluted with dichloromethane and washed with saturated NaHCO₃ solution, water and brine solution. The organic layer was dried over anhydrous Na₂SO₄ and concentrated. The crude residue was purified, and the desired product was eluted at 6% EtOAc in hexanes. The product containing fractions were concentrated to obtain 2.58 g (65%) of pure product.

### Results:

ESI-MS: Calculated C₈₆H₁₇₇N₂O₁₀Si₄, [M+H⁺]=1510.25, Observed=1510.3

### Step 2: Synthesis of Compound 3

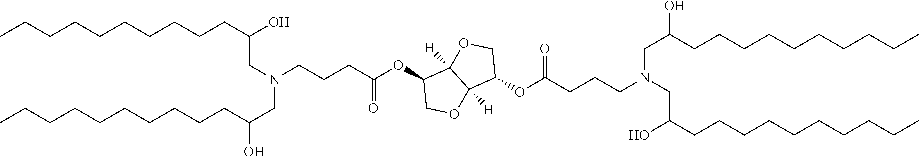 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

As depicted in Scheme 2: To a solution of Intermediate (3) (2.58 g, 1.70 mmol) in tetrahydrofuran (14 mL) was added hydrogen fluoride (70% HF.py complex, 7 mL, 51.23 mmol) at 0° C. and stirred at the same temperature for 5 minutes. Then reaction mixture was warmed to room temperature and stirred for 16 h. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water and brine. Then dried over anhydrous Na₂SO₄ and concentrated. The crude residue was purified, and the desired product was eluted at 67% EtOAc in hexanes. The purest fractions were concentrated to obtain 1.1 g (61%) of pure product.

### Results:

¹H NMR (400 MHz, CDCl₃) δ 5.13-5.03 (m, 2H), 4.73-4.65 (m, 2H), 4.29-3.83 (m, 8H), 3.52-2.98 (m, 12H), 2.69-2.49 (m, 4H), 2.32-2.09 (m, 4H), 1.73-1.12 (m, 72H), 0.88 (t, J=6.6 Hz, 12H).

ESI-MS: Calculated C₆₂H₁₂₁N₂O₁₀, [M+H⁺]=1053.90, Observed=1053.2 and 527.2 [M/2+H⁺]

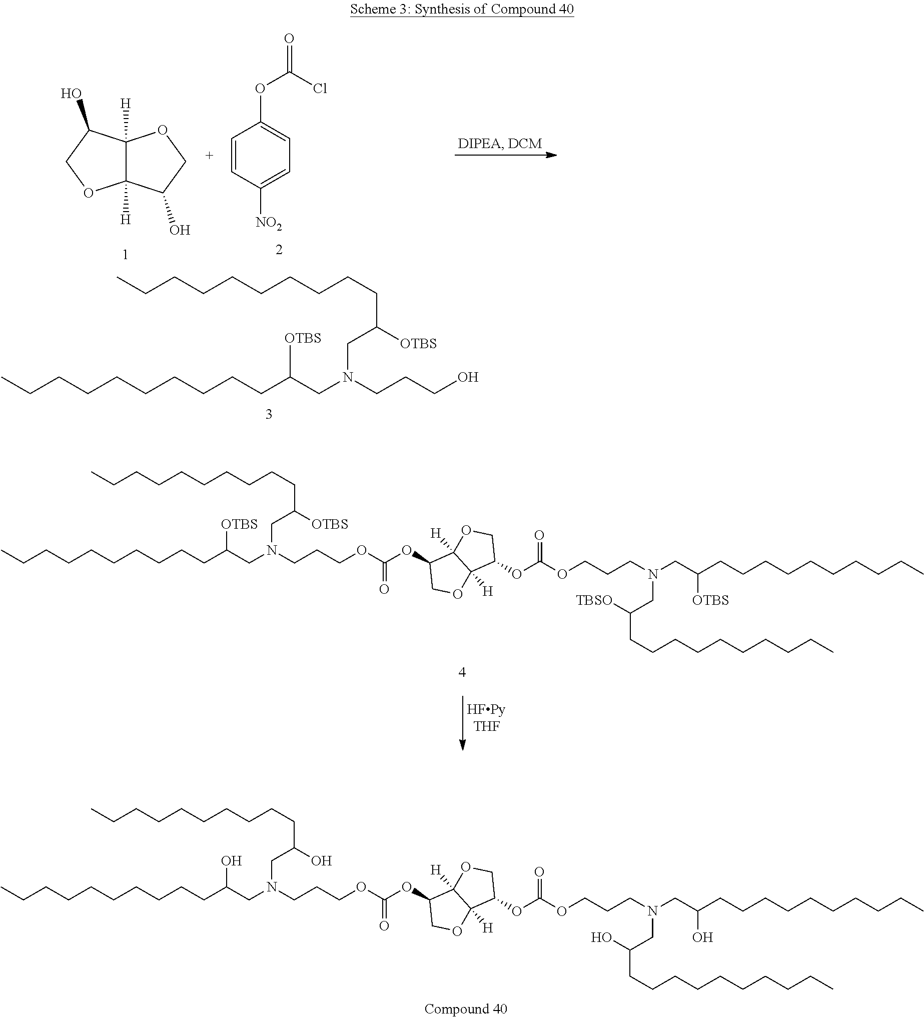

### Step 1: Synthesis of Intermediate (4)

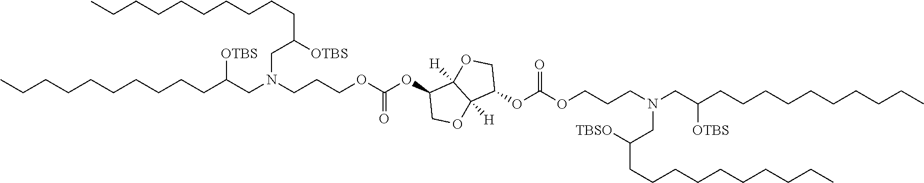 [SMILES: CCCCCCCCCCC(CN(CCCOC(=O)O[C@H]1CO[C@H]2[C@@H]1OC[C@H]2OC(=O)OCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 3: To a solution of isosorbide (1) (0.100 g, 0.68 mmol) in anhydrous DCM (3 mL) was added DMAP (0.033 g, 0.027 mmol) and TEA (0.67 mL, 4.79 mmol). To the resultant mixture (2) (0.33 g, 1.64 mmol) was added and stirred for 20 minutes. To that alcohol (3) (1.15 g, 1.71 mmol) in DCM (5 mL) was added and stirred at room temperature for 20 h. MS (no ionization) and TLC analysis indicated formation of the product. The reaction mixture was diluted with DCM and washed sat. NaHCO₃ solution, water and brine solution. The organic layer was dried over anhydrous Na₂SO₄ and concentrated. The crude residue was purified (SiO₂: 8% ethyl acetate in hexanes) to get Intermediate (4) (1.05 g, quantitative yield).

### Results:

ESI-MS: Calculated C₈₆H₁₇₇N₂O₁₂Si₄, [M+H⁺]=1542.24, Observed=771.5 [M/2+H⁺]

### Step 2: Synthesis of Compound 40

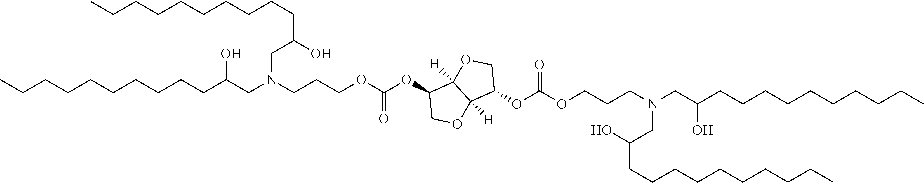 [SMILES: CCCCCCCCCCC(O)CN(CCCOC(=O)O[C@H]1CO[C@H]2[C@@H]1OC[C@H]2OC(=O)OCCCN(CC(O)CCCCCCCCCC)CC(O)CCCCCCCCCC)CC(O)CCCCCCCCCC]

As depicted in Scheme 3: To a solution of Intermediate (4) (1.05 g, 0.68 mmol) in tetrahydrofuran (4 mL) was added hydrogen fluoride (70% HF.py complex, 1 mL, 6.80 mmol) at 0° C. and stirred at the same temperature for 5 minutes. Then reaction mixture was warmed to room temperature and stirred for 16 h. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water and brine. Then dried over anhydrous Na₂SO₄ and concentrated. The crude residue was purified, and the purest fractions were concentrated to obtain 0.154 g (20%) of pure product.

### Results:

ESI-MS: Calculated C₆₂H₁₂₀N₂O₁₂, [M+H⁺]=1085.89, Observed=1085.1, and 543.2 [M/2+H⁺]

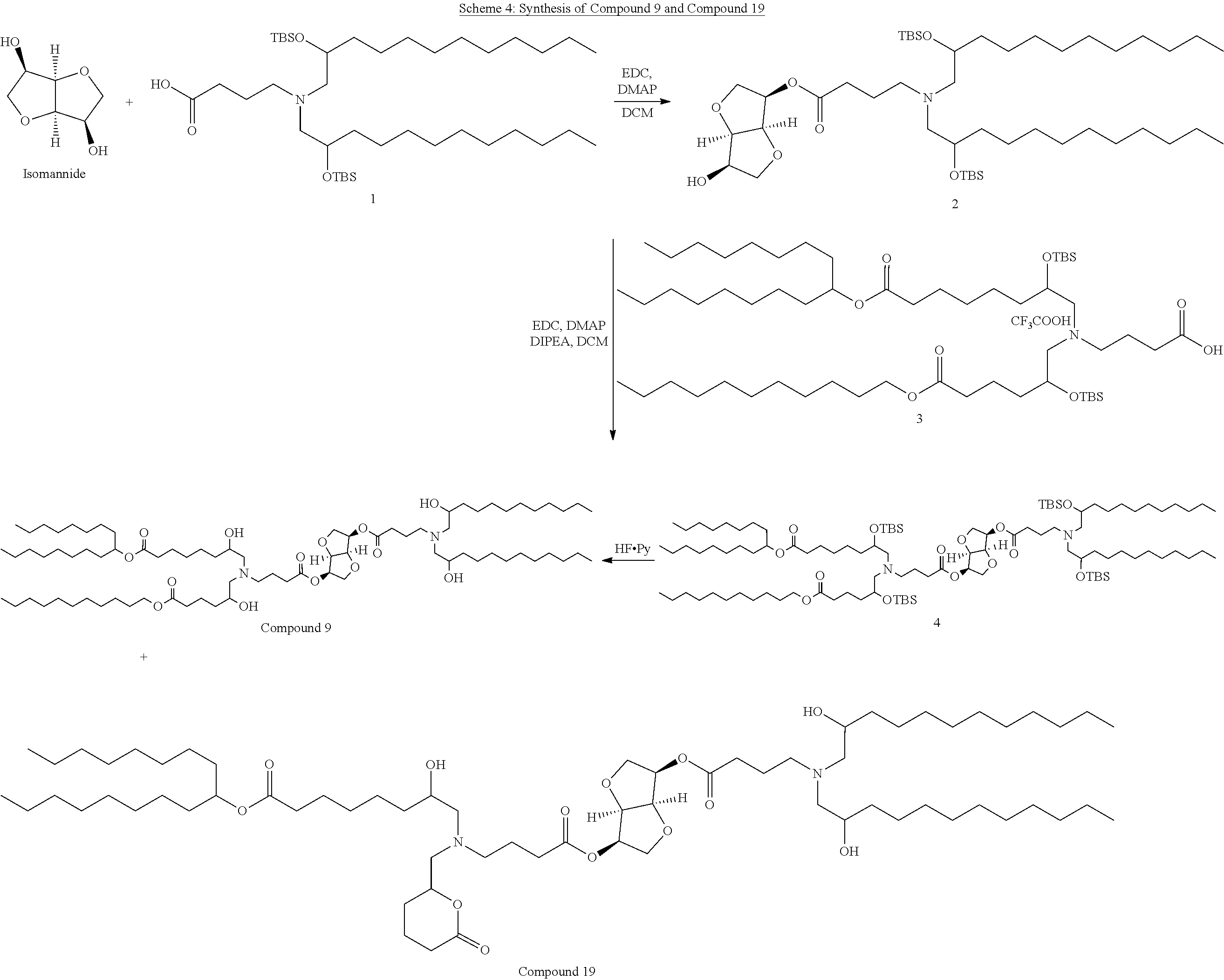

### Step 1: Synthesis of Intermediate (2)

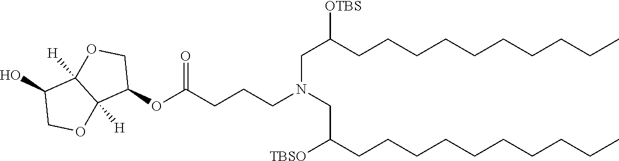 [SMILES: CCCCCCCCCCC(CN(CCCC(=O)O[C@@H]1CO[C@H]2[C@@H]1OC[C@H]2O)CC(CCCCCCCCCC)O[Si](C)(C)C(C)(C)C)O[Si](C)(C)C(C)(C)C]

As depicted in Scheme 4: To a solution of acid (1) (750 mg, 1.07 mmol) in 8 mL of DCM was added isomannide (144 mg, 0.985 mmol), DMAP (120 mg, 0.978 mmol), and EDC (244 mg, 1.27 mmol). The resulting mixture was stirred at room temperature for overnight. MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (10% ethyl acetate in hexanes) to get Intermediate (2) (455 mg, 55%).

### Results:

ESI-MS: Calculated C₄₆H₉₄NO₇Si₂, [M+H⁺]=828.66, Observed=828.6.

### Step 2: Synthesis of Intermediate (4)

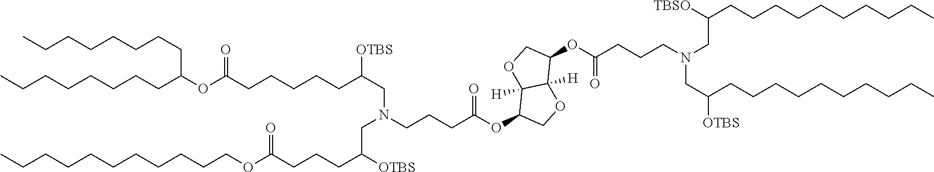 [SMILES: CCCCCCCCCCCOC(=O)CCCC(CN(CCCC(=O)O[C@@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 4: To a solution of acid (with TFA salt) (3) (0.335 g, 0.298 mmol) and isomannide mono ester (2) (0.19 g, 0.229 mmol) in dichloromethane (5 mL) were added DIPEA (0.16 mL, 0.917 mmol), DMAP (0.028 g, 0.229 mmol) and EDC (0.132 g, 0.688 mmol). The resulting mixture was stirred at room temperature for overnight. After 16 h, MS and TLC (30% EtOAc in hexanes) analysis indicated completion of the reaction. The reaction mixture was diluted with dichloromethane and washed with saturated NaHCO₃ solution, water and brine solution. The organic layer was dried over anhydrous Na₂SO₄ and concentrated. The crude residue was purified, and the desired product was eluted at 7% EtOAc in hexanes. The product containing fractions were concentrated to obtain Intermediate (4) 0.255 g (61%) of pure product.

### Results:

ESI-MS: Calculated C₁₀₄H₂₀₉N₂O₁₄Si₄, [M+H⁺]=1822.48, Observed=911.5 [M/2+H⁺]

### Step 3: Synthesis of Compound 9 and Compound 19

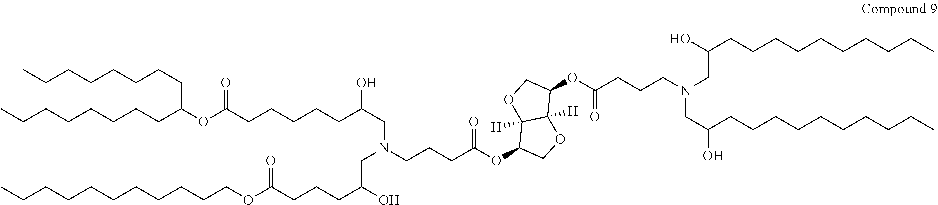 [SMILES: CCCCCCCCCCCOC(=O)CCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCC(=O)OC(CCCCCCCC)CCCCCCCC]

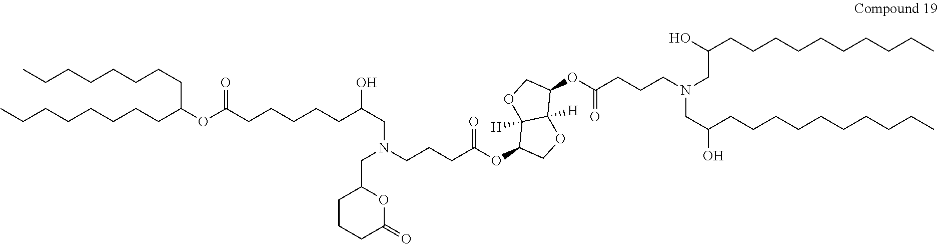 [SMILES: CCCCCCCCCCC(O)CN(CCCC(=O)O[C@@H]1CO[C@H]2[C@@H]1OC[C@H]2OC(=O)CCCN(CC(O)CCCCCC(=O)OC(CCCCCCCC)CCCCCCCC)CC1CCCC(=O)O1)CC(O)CCCCCCCCCC]

As depicted in Scheme 4: To a solution of Intermediate (4) (0.255 g, 0.139 mmol) in tetrahydrofuran (2 mL) was added hydrogen fluoride (70% HF.py complex, 2 mL, 6.993 mmol) at 0° C. and stirred at the same temperature for 5 minutes. Then reaction mixture was warmed to room temperature and stirred for 16 h. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water and brine. Then dried over anhydrous Na₂SO₄ and concentrated. The crude residue was purified, and the desired product Compound 9 was eluted between 70-97% EtOAc in hexanes and the cyclized product Compound 19 was eluted between 97-100% EtOAc in hexanes. The purest fractions were concentrated to obtain 50 mg (26%) of Compound 9 and 46 mg (27%) of Compound 19.

### Results:

ESI-MS for Compound 9: Calculated C₈₀H₁₅₃N₂O₁₄, [M+H⁺]=1366.13, Observed=1366.1 and 683.6 [M/2+H⁺]

ESI-MS for Compound 19: Calculated C₈₀H₁₅₃N₂O₁₄, [M+H⁺]=1193.95, Observed=1193.1 and 597.2 [M/2+H⁺]

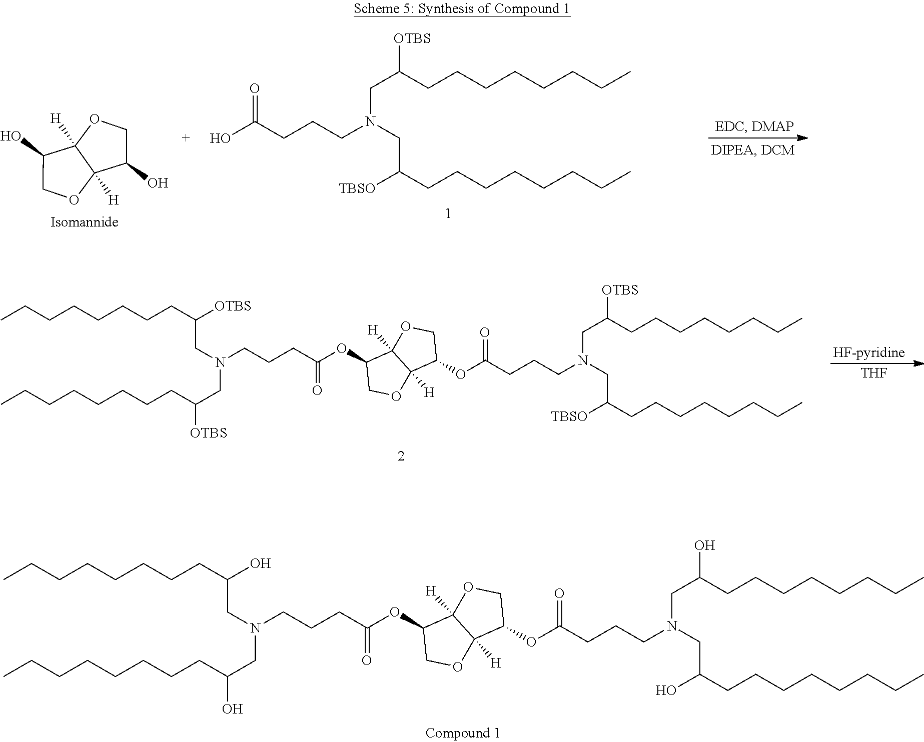

### Step 1: Synthesis of Intermediate (2)

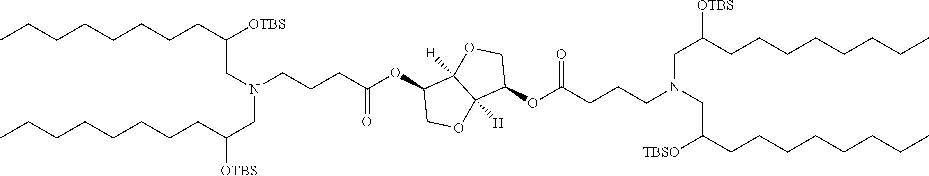 [SMILES: CCCCCCCCC(CN(CCCC(=O)O[C@@H]1CO[C@H]2[C@@H]1OC[C@H]2OC(=O)CCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 5: To a solution of acid (1) (3.306 g, 5.132 mmol) in 25 mL of anhydrous dichloromethane (DCM) was added isomannide (0.250 g, 1.711 mmol), 4-Dimethylaminopyridine (DMAP) (0.210 g, 1.711 mmol), Diisopropylethylamine (DIPEA) (2.4 mL, 14 mmol), and 1-Ethyl-3-(3-dimethylaminopropyl) carbodiimide (EDC) (1.312 g, 6.844 mmol). The resulting mixture was stirred at room temperature overnight. Mass spectrometry (MS) analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (1.493 g, 62%).

### Results:

ESI-MS: Calculated C₇₈H₁₆₀N₂O₁₀Si₄, [M+H⁺]=1398.12, Observed=1398.2, and 699.1[M/2+H⁺].

### Step 2: Synthesis of Compound 1

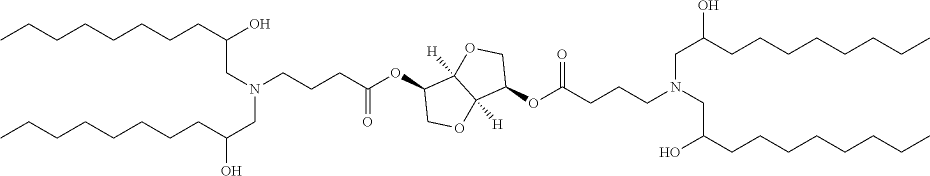 [SMILES: CCCCCCCCC(O)CN(CCCC(=O)O[C@@H]1CO[C@H]2[C@@H]1OC[C@H]2OC(=O)CCCN(CC(O)CCCCCCCC)CC(O)CCCCCCCC)CC(O)CCCCCCCC]

As depicted in Scheme 5: To a solution of Intermediate (2) (2.471 g, 1.767 mmol) in 16 mL of anhydrous tetrahydrofuran at 0° C. was added hydrogen fluoride pyridine (70% HF, 8.0 mL, 62 mmol). The reaction mixture was warmed to room temperature and stirred for 16 hours. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 47% ethyl acetate in hexanes) to obtain Compound 1 (720 mg, 43%).

### Results:

¹H NMR (400 MHz, CDCl₃) δ 5.12-5.02 (m, 2H), 4.74-4.66 (m, 2H), 4.28-4.11 (m, 2H), 4.10-3.98 (m, 4H), 3.97-3.71 (m, 2H), 3.43-2.90 (m, 12H), 2.78-2.49 (m, 4H), 2.45-2.08 (m, 4H), 1.69-1.19 (m, 56H), 0.88 (t, J=6.6 Hz, 12H).

ESI-MS: Calculated C₅₄H₁₀₄N₂O₁₀, [M+H⁺]=941.78, Observed=941.2, and 471.1 [M/2+H⁺]

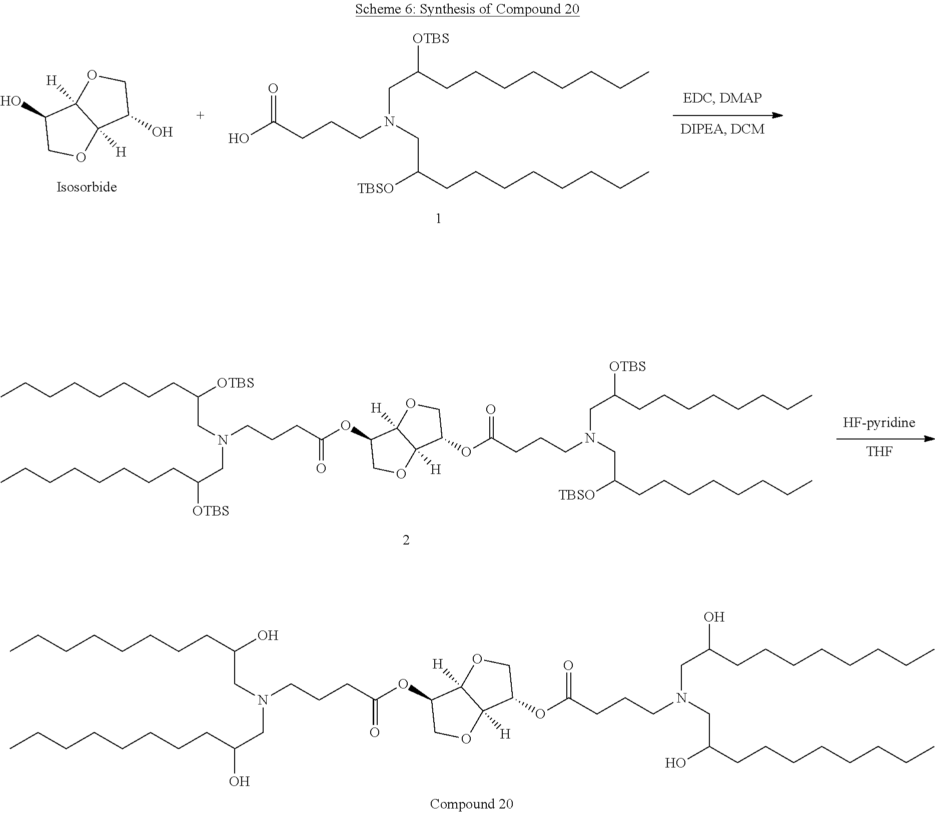

### Step 1: Synthesis of Intermediate (2)

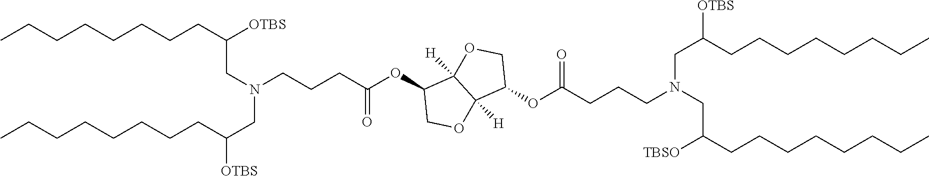 [SMILES: CCCCCCCCC(CN(CCCC(=O)O[C@H]1CO[C@H]2[C@@H]1OC[C@H]2OC(=O)CCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 6: To a solution of acid (1) (3.306 g, 5.132 mmol) in 25 mL of anhydrous dichloromethane was added isosorbide (0.250 g, 1.711 mmol), DMAP (0.210 g, 1.711 mmol), DIPEA (2.4 mL, 14 mmol), and EDC (1.312 g, 6.844 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (1.402 g, 59%).

### Results:

ESI-MS: Calculated C₇₈H₁₆₀N₂O₁₀Si₄, [M+H⁺]=1398.12, Observed=1398.2, and 699.2 [M/2+H⁺].

### Step 2: Synthesis of Compound 20

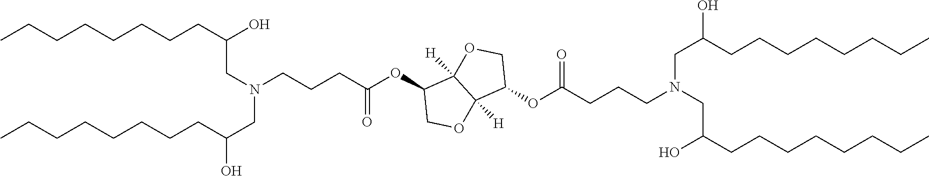 [SMILES: CCCCCCCCC(O)CN(CCCC(=O)O[C@H]1CO[C@H]2[C@@H]1OC[C@H]2OC(=O)CCCN(CC(O)CCCCCCCC)CC(O)CCCCCCCC)CC(O)CCCCCCCC]

As depicted in Scheme 6: To a solution of Intermediate (2) (2.250 g, 1.609 mmol) in 16 mL of anhydrous tetrahydrofuran at 0° C. was added hydrogen fluoride pyridine (70% HF, 8.0 mL, 62 mmol). The reaction mixture was warmed to room temperature and stirred for 16 hours. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 37% ethyl acetate in hexanes) to obtain Compound 20 (366 mg, 24%).

### Results:

¹H NMR (400 MHz, CDCl₃) δ 5.22-5.13 (m, 2H), 4.89-4.82 (m, 2H), 4.52-4.45 (m, 2H), 4.24-3.77 (m, 8H), 3.41-2.57 (m, 12H), 2.58-2.27 (m, 4H), 2.22-1.95 (m, 2H), 1.70-1.16 (m, 56H), 0.88 (t, 12H).

ESI-MS: Calculated C₅₄H₁₀₄N₂O₁₀, [M+H⁺]=941.78, Observed=941.1, and 471.1 [M/2+H⁺]

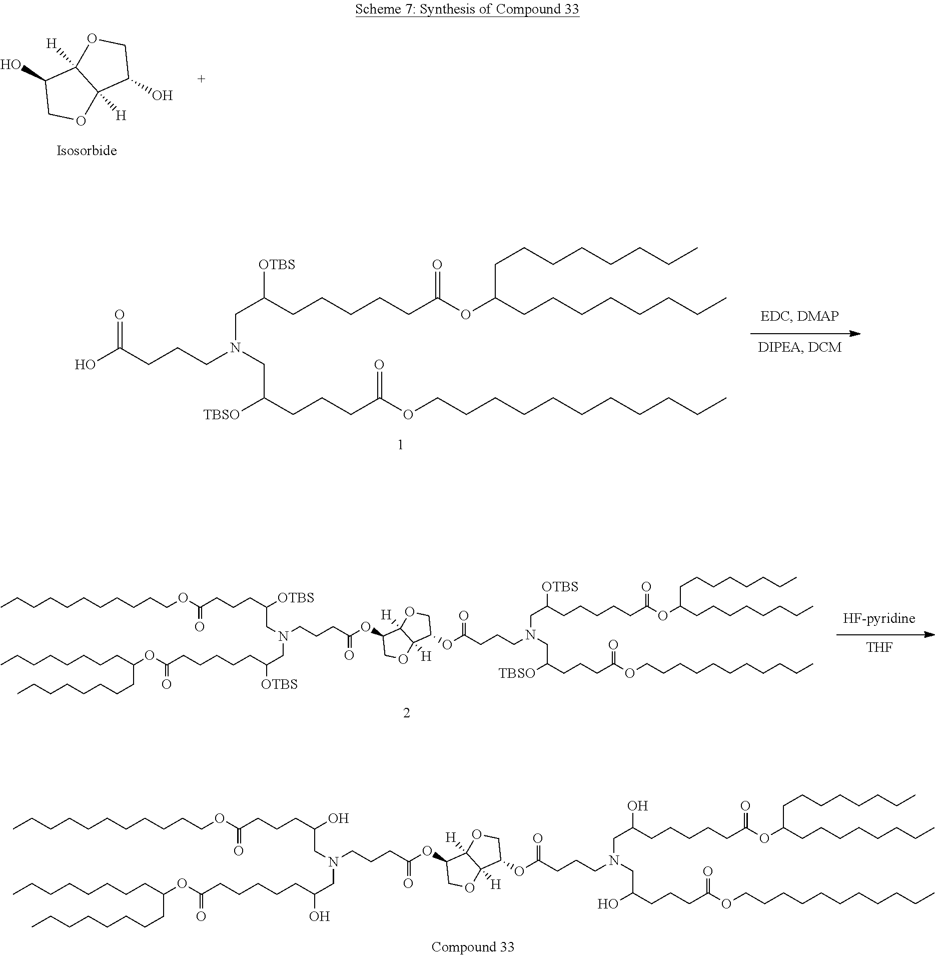

### Step 1: Synthesis of Intermediate (2)

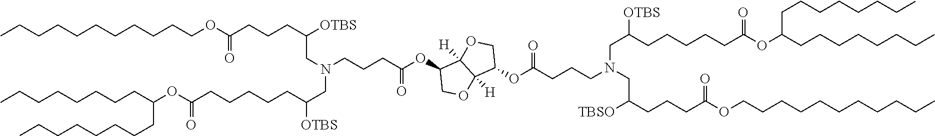 [SMILES: CCCCCCCCCCCOC(=O)CCCC(CN(CCCC(=O)O[C@H]1CO[C@H]2[C@@H]1OC[C@H]2OC(=O)CCCN(CC(CCCCCC(=O)OC(CCCCCCCC)CCCCCCCC)O[Si](C)(C)C(C)(C)C)CC(CCCC(=O)OCCCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 7: To a solution of acid (1) (751 mg, 0.742 mmol) in 7.5 mL of anhydrous dichloromethane was added isosorbide (38 mg, 0.26 mmol), DMAP (30 mg, 0.24 mmol), DIPEA (0.35 mL, 2.0 mmol), and EDC (190 mg, 0.991 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (266 mg, 48%).

### Results:

ESI-MS analysis: Calculated C₁₂₂H₂₄₀N₂O₁₈Si₄, [M+H⁺]=2134.71, Observed=1067.3 [M/2+H⁺].

### Step 2: Synthesis of Compound 33

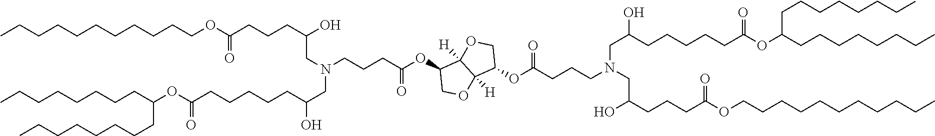 [SMILES: CCCCCCCCCCCOC(=O)CCCC(O)CN(CCCC(=O)O[C@H]1CO[C@H]2[C@@H]1OC[C@H]2OC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC]

As depicted in Scheme 7: To a solution of Intermediate (2) (583 mg, 0.264 mmol) in 1.5 mL of anhydrous tetrahydrofuran at 0° C. was added hydrogen fluoride pyridine (70% HF, 0.05 mL, 0.4 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 53% ethyl acetate in hexanes) to obtain Compound 33 (119 mg, 27%).

### Results:

¹H NMR (400 MHz, CDCl₃) δ 5.21-5.17 (m, 2H), 4.90-4.79 (m, 2H), 4.51-4.46 (m, 6H), 4.29-4.23 (m, 2H), 4.09-4.01 (m, 4H), 3.97-3.88 (m, 4H), 3.29-3.24 (m, 6H), 3.15-3.10 (m, 4H), 2.54-2.50 (m, 6H), 2.39-2.24 (m, 12H), 2.22-2.18 (m, 6H), 1.84-1.79 (m, 4H), 1.65-1.55 (m, 12H), 1.55-1.45 (m, 12H), 1.41-1.33 (m, 4H), 1.33-1.27 (m, 18H), 1.27-1.05 (m, 58H), 0.87 (t, J=6.6 Hz, 18H).

ESI-MS: Calculated C₉₈H₁₈₄N₂O₁₈, [M+H⁺]=1678.36, Observed=1678.2 and 839.3 [M/2+H⁺].

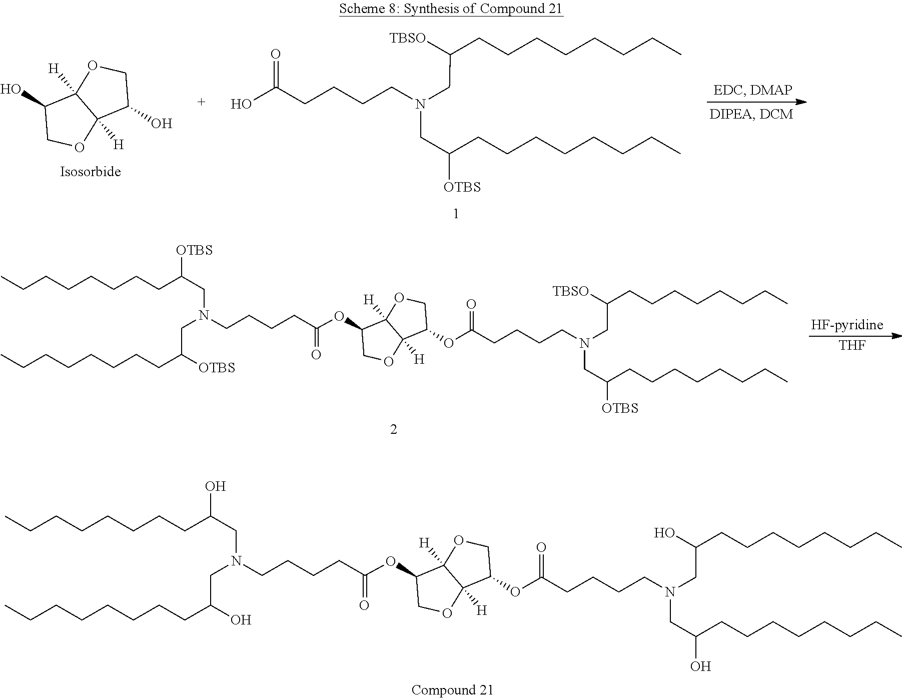

### Step 1: Synthesis of Intermediate (2)

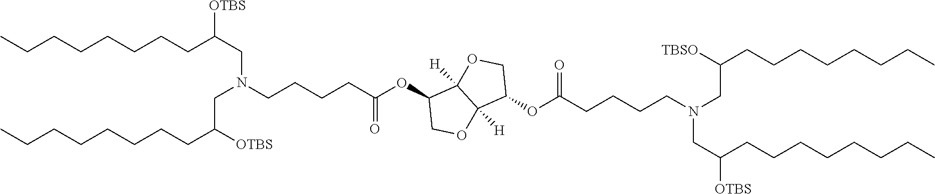 [SMILES: CCCCCCCCC(CN(CCCCC(=O)O[C@H]1CO[C@H]2[C@@H]1OC[C@H]2OC(=O)CCCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 8: To a solution of acid (1) (500 mg, 0.760 mmol) in 5 mL of anhydrous dichloromethane was added isosorbide (44 mg, 0.30 mmol), DMAP (37 mg, 0.30 mmol), DIPEA (0.423 mL, 2.43 mmol), and EDC (233 mg, 1.22 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (265 mg, 24%).

### Results:

ESI-MS analysis: Calculated C₈₀H₁₆₄N₂O₁₀Si₄, [M+H⁺]=1426.15, Observed=714.0 [M/2+H⁺].

### Step 2: Synthesis of Compound 21

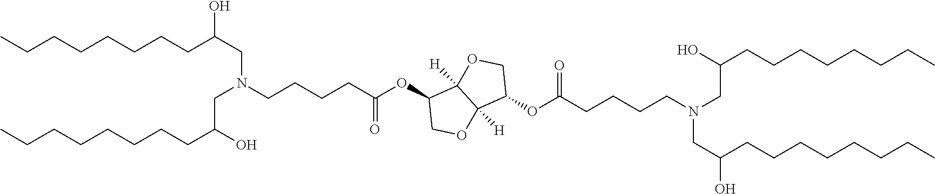 [SMILES: CCCCCCCCC(O)CN(CCCCC(=O)O[C@H]1CO[C@H]2[C@@H]1OC[C@H]2OC(=O)CCCCN(CC(O)CCCCCCCC)CC(O)CCCCCCCC)CC(O)CCCCCCCC]

As depicted in Scheme 8: To a solution of Intermediate (2) (265 mg, 0.186 mmol) in 1 mL of anhydrous tetrahydrofuran at 0° C. was added hydrogen fluoride pyridine (70% HF, 0.75 mL, 5.8 mmol). The reaction mixture was warmed to room temperature and stirred for 2 hours. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 47% ethyl acetate in hexanes) to obtain Compound 21(64 mg, 36%).

### Results:

ESI-MS: Calculated C₅₆H₁₀₈N₂O₁₀, [M+H⁺]=969.81, Observed=969.2, and 485.2 [M/2+H⁺].

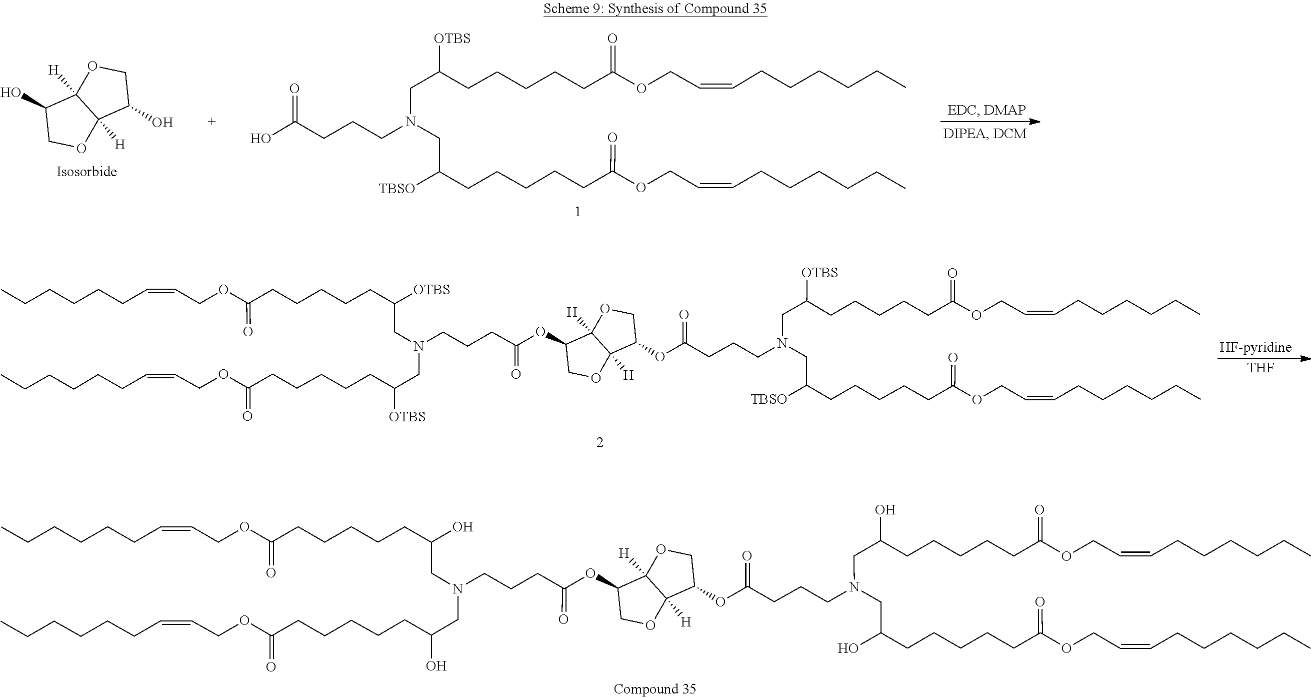

### Step 1: Synthesis of Intermediate (2)

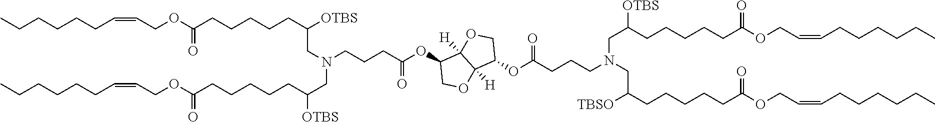 [SMILES: CCCCCC/C=C\COC(=O)CCCCCC(CN(CCCC(=O)O[C@H]1CO[C@H]2[C@@H]1OC[C@H]2OC(=O)CCCN(CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)O[Si](C)(C)C(C)(C)C]

As depicted in Scheme 9: To a solution of acid (1) (750 mg, 0.837 mmol) in 7.5 mL of anhydrous dichloromethane was added isosorbide (42.1 mg, 0.288 mmol), DMAP (35 mg, 0.29 mmol), DIPEA (0.39 mL, 2.2 mmol), and EDC (215 mg, 1.12 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 7% ethyl acetate in hexanes) to get Intermediate (2) (364 mg, 66%).

### Results:

ESI-MS analysis: Calculated C₁₀₆H₂₀₀N₂O₁₈Si₄, [M+H⁺]=1902.40, Observed=951.2 [M/2+H⁺].

### Step 2: Synthesis of Compound 35

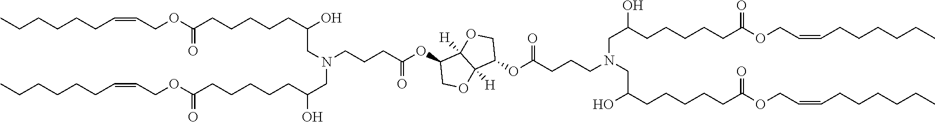 [SMILES: CCCCCC/C=C\COC(=O)CCCCCC(O)CN(CCCC(=O)O[C@H]1CO[C@H]2[C@@H]1OC[C@H]2OC(=O)CCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC]

As depicted in Scheme 9: To a solution of Intermediate (2) (364 mg, 0.191 mmol) in 2.3 mL of anhydrous tetrahydrofuran at 0° C. was added hydrogen fluoride pyridine (70% HF, 0.08 mL, 0.6 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 84% ethyl acetate in hexanes) to obtain Compound 35 (94 mg, 34%).

### Results:

¹H NMR (400 MHz, CDCl₃) δ 5.69-5.57 (m, 4H), 5.57-5.38 (m, 4H), 5.25-4.94 (m, 2H), 4.94-4.74 (m, 2H), 4.74-4.56 (m, 8H), 4.56-4.42 (m, 1H), 4.42-4.32 (m, 1H), 4.32-4.00 (m, 4H), 4.00-3.65 (m, 2H), 2.86-2.00 (m, 32H), 1.96-1.81 (m, 2H), 1.81-1.00 (m, 66H), 0.99-0.76 (m, 12H).

ESI-MS: Calculated C₈₂H₁₄₄N₂O₁₈, [M+H⁺]=1446.05, Observed=1446.1, and 723.5 [M/2+H⁺]

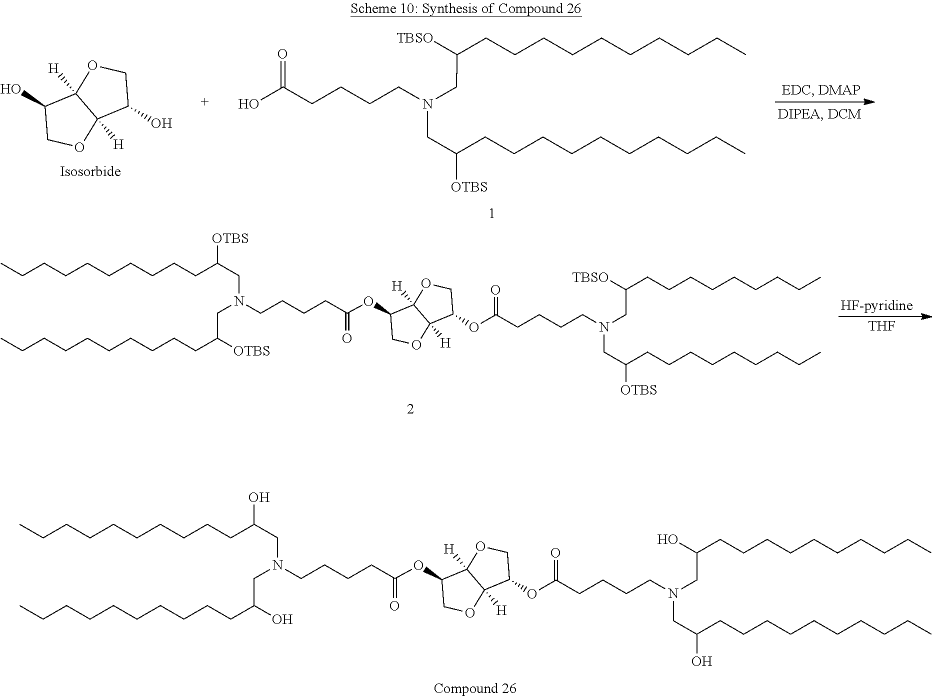

### Step 1: Synthesis of Intermediate (2)

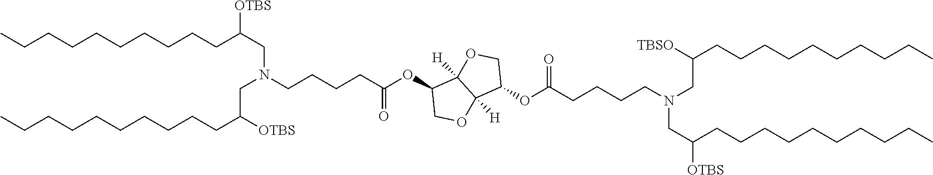 [SMILES: CCCCCCCCCCC(CN(CCCCC(=O)O[C@H]1CO[C@H]2[C@@H]1OC[C@H]2OC(=O)CCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 10: To a solution of acid (1) (750 mg, 1.05 mmol) in 7.5 mL of anhydrous dichloromethane was added isosorbide (51 mg, 0.35 mmol), DMAP (44 mg, 0.36 mmol), DIPEA (0.50 mL, 3.0 mmol), and EDC (268 mg, 1.40 mmol). The resulting mixture was stirred at room temperature overnight. MS analysis showed the presence of monoester. Additional EDC (134 mg, 0.699 mmol) was added to the reaction and the reaction was run for an additional 90 minutes. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (166 mg, 31%).

### Results:

ESI-MS analysis: Calculated C₈₈H₁₈₀N₂O₁₀Si₄, [M+H⁺]=1538.28, Observed=1538.3, and 769.6 [M/2+H⁺].

### Step 2: Synthesis of Compound 26

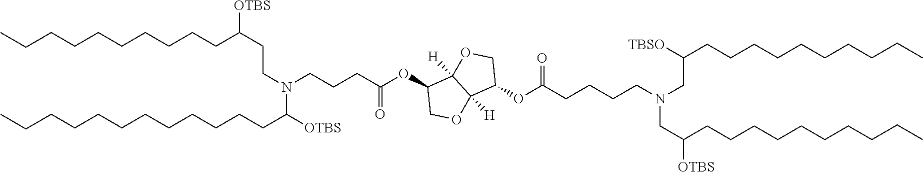 [SMILES: CCCCCCCCCCCCC(O[Si](C)(C)C(C)(C)C)N(CCCC(=O)O[C@@H]1CO[C@H]2[C@@H]1OC[C@@H]2OC(=O)CCCCN(CC(CCCCCCCCCC)O[Si](C)(C)C(C)(C)C)CC(CCCCCCCCCC)O[Si](C)(C)C(C)(C)C)CCC(CCCCCCCCCC)O[Si](C)(C)C(C)(C)C]

As depicted in Scheme 10: To a solution of Intermediate (2) (306 mg, 0.200 mmol) in 3 mL of anhydrous tetrahydrofuran at 0° C. was added hydrogen fluoride pyridine (70% HF, 0.1 mL, 0.8 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 60% ethyl acetate in hexanes) to obtain Compound 26 (74 mg, 34%).

### Results:

¹H NMR (400 MHz, CDCl₃) δ 5.26-5.10 (m, 2H), 4.92-4.76 (m, 2H), 4.32-4.15 (m, 2H), 3.98-3.79 (m, 2H), 3.35-2.99 (m, 16H), 2.67-2.17 (m, 4H), 2.06-1.90 (m, 4H), 1.79-1.18 (m, 76H), 0.88 (t, J=6.7 Hz, 12H).

ESI-MS: Calculated C₆₄H₁₂₄N₂O₁₀, [M+H⁺]=1081.93, Observed=1081.3, and 541.2 [M/2+H⁺]

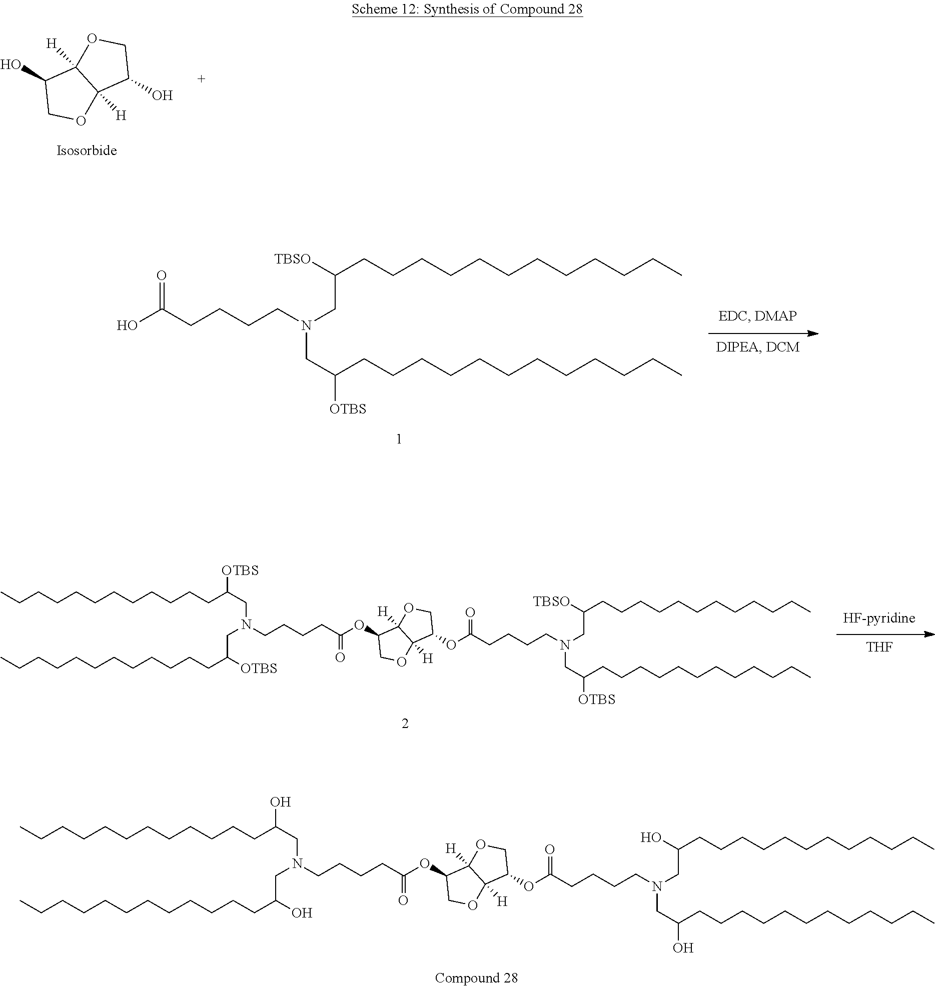

### Step 1: Synthesis of Intermediate (2)

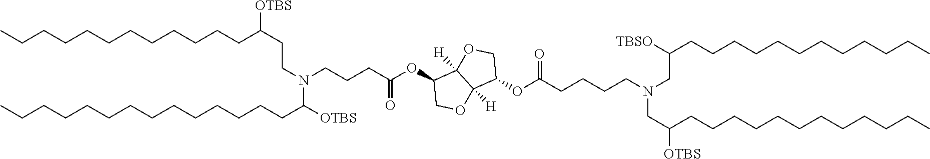 [SMILES: CCCCCCCCCCCCCCC(O[Si](C)(C)C(C)(C)C)N(CCCC(=O)O[C@@H]1CO[C@H]2[C@@H]1OC[C@@H]2OC(=O)CCCCN(CC(CCCCCCCCCCCC)O[Si](C)(C)C(C)(C)C)CC(CCCCCCCCCCCC)O[Si](C)(C)C(C)(C)C)CCC(CCCCCCCCCCCC)O[Si](C)(C)C(C)(C)C]

As depicted in Scheme 12: To a solution of acid (1) (755 mg, 0.980 mmol) in 7.5 mL of anhydrous dichloromethane was added isosorbide (48 mg, 0.33 mmol), DMAP (41 mg, 0.33 mmol), DIPEA (0.45 mL, 2.6 mmol), and EDC (250 mg, 1.30 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (381 mg, 70%).

### Results:

ESI-MS analysis: Calculated C₉₆H₁₉₆N₂O₁₀Si₄, [M+H⁺]=1650.40, Observed=825.8 [M/2+H⁺].

### Step 2: Synthesis of Compound 28

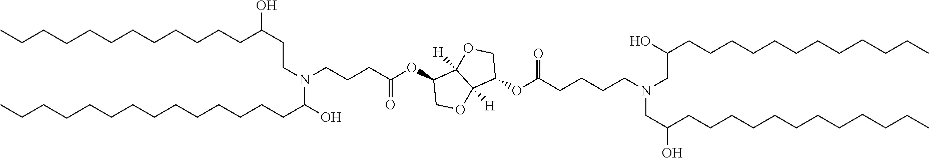 [SMILES: CCCCCCCCCCCCCCC(O)N(CCCC(=O)O[C@@H]1CO[C@H]2[C@@H]1OC[C@@H]2OC(=O)CCCCN(CC(O)CCCCCCCCCCCC)CC(O)CCCCCCCCCCCC)CCC(O)CCCCCCCCCCCC]

As depicted in Scheme 12: To a solution of Intermediate (2) (381 mg, 0.231 mmol) in 2 mL of anhydrous tetrahydrofuran at 0° C. was added hydrogen fluoride pyridine (70% HF, 0.60 mL, 5.0 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 65% ethyl acetate in hexanes) to obtain Compound 28 (174 mg, 63%).

### Results:

¹H NMR (400 MHz, CDCl₃) δ 5.26-5.02 (m, 2H), 4.96-4.75 (m, 2H), 4.31-4.14 (m, 2H), 3.93 (d, J=15.6 Hz, 2H), 3.37-2.96 (m, 16H), 2.50-2.26 (m, 4H), 2.03-1.85 (m, 4H), 1.79-1.17 (m, 92H), 0.91-0.81 (m, 12H).

ESI-MS: Calculated C₇₂H₁₄₀N₂O₁₀, [M+H⁺]=1194.0, Observed=1193.3, and 597.3 [M/2+H⁺].

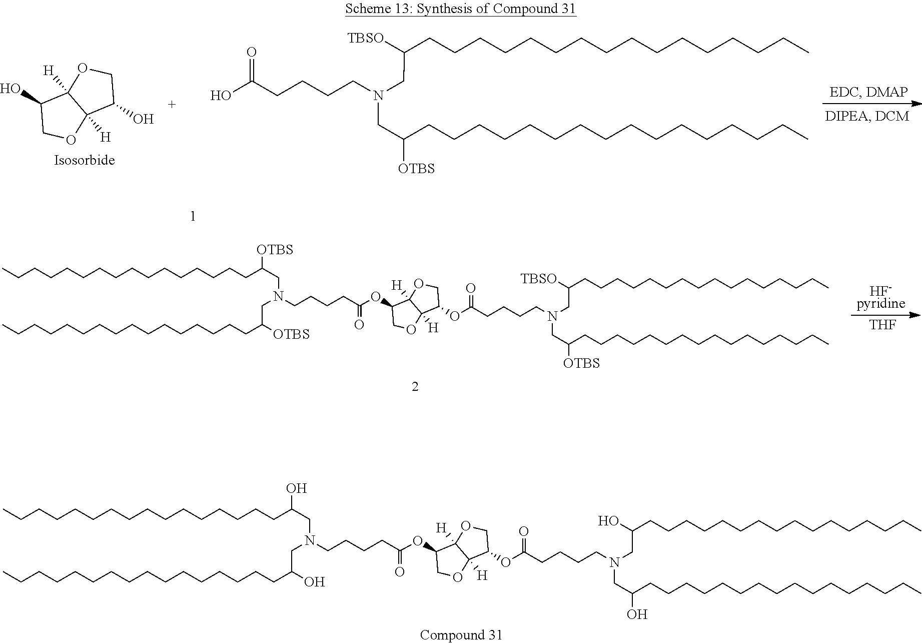

### Step 1: Synthesis of Intermediate (2)

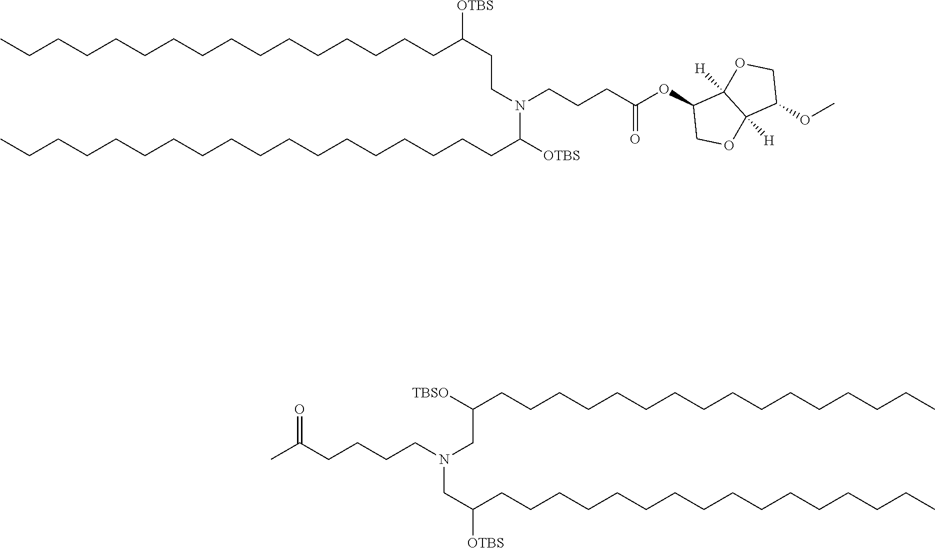

As depicted in Scheme 13: To a solution of acid (1) (755 mg, 0.855 mmol) in 7.5 mL of anhydrous dichloromethane was added isosorbide (43 mg, 0.29 mmol), DMAP (36 mg, 0.29 mmol), DIPEA 0.40 mL, 2.0 mmol), and EDC (221 mg, 1.15 mmol). The resulting mixture was stirred at room temperature overnight. MS analysis showed the presence of monoester. Additional EDC (127 mg, 0.66 mmol) was added to the reaction and the reaction was run for an additional 90 minutes. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (375 mg, 68%).

### Results:

ESI-MS analysis: Calculated C₁₁₂H₂₂₈N₂O₁₀Si₄, [M+H⁺]=1874.65, Observed=937.8 [M/2+H⁺].

### Step 2: Synthesis of Compound 31

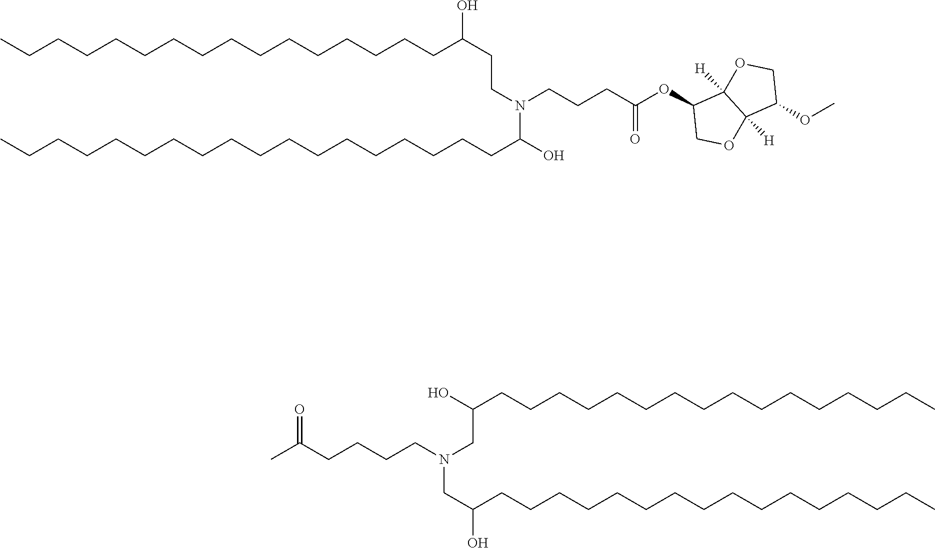

As depicted in Scheme 13: To a solution of Intermediate (2) (375 mg, 0.200 mmol) in 2 mL of anhydrous tetrahydrofuran at 0° C. was added hydrogen fluoride pyridine (70% HF, 0.55 mL, 4.3 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 40% ethyl acetate in hexanes) to obtain Compound 31 (172 mg, 61%).

### Results:

¹H NMR (400 MHz, CDCl₃) δ 5.25-5.18 (m, 2H), 4.51-4.45 (m, 2H), 4.28-4.16 (m, 2H), 3.95-3.81 (m, 2H), 3.34-3.01 (m, 16H), 2.50-2.34 (m, 4H), 2.02-1.87 (m, 4H), 1.85-1.16 (m, 124H), 0.87 (t, 12H).

ESI-MS: Calculated C₈₈H₁₇₂N₂O₁₀, [M+H⁺]=1418.31, Observed=1418.3, and 709.3 [M/2+H⁺]

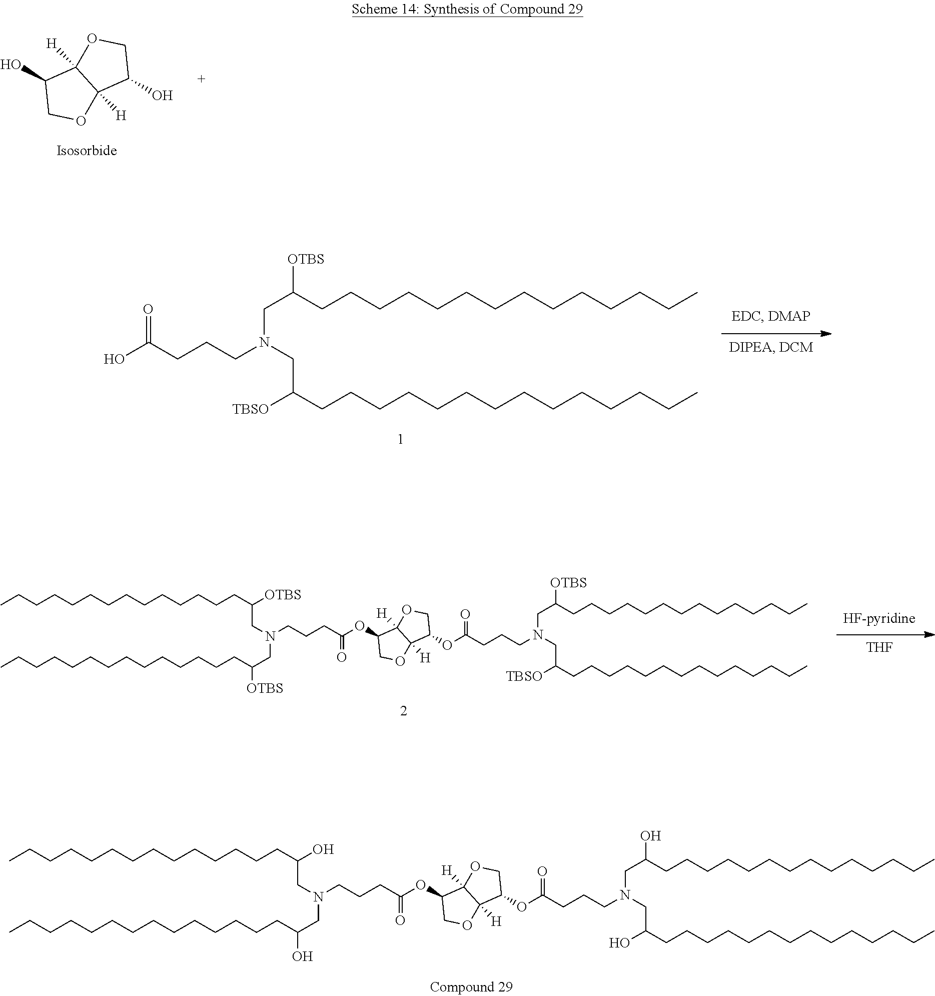

### Step 1: Synthesis of Intermediate (2)

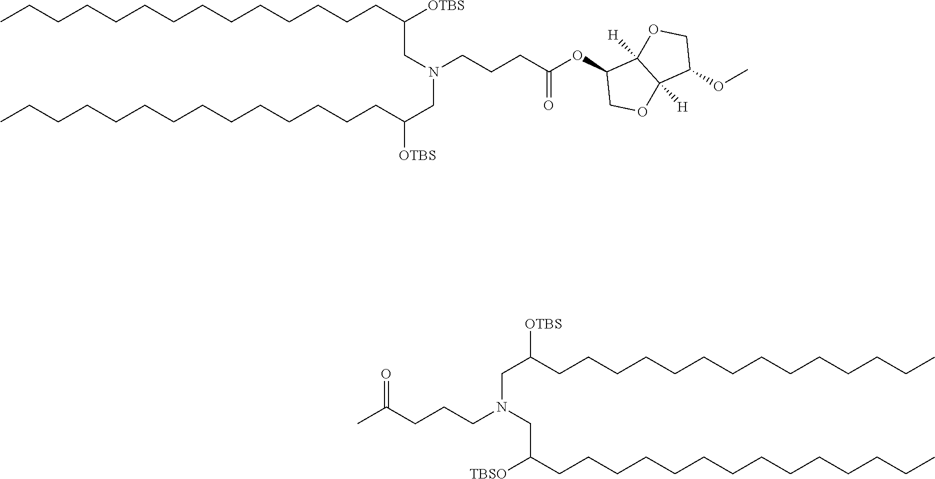

As depicted in Scheme 14: To a solution of acid (1) (758 mg, 0.933 mmol) in 7.5 mL of anhydrous dichloromethane was added isosorbide (45 mg, 0.31 mmol), DMAP (40 mg, 0.33 mmol), DIPEA (0.50 mL, 3.0 mmol), and EDC (237 mg, 1.24 mmol). The resulting mixture was stirred at room temperature overnight. MS analysis showed the presence of monoester. Additional DMAP (19 mg, 0.15 mmol) was added to the reaction and the reaction was run for an additional 2 hours. Mass spec confirmed that less monoester was present. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (503 mg, 94%).

### Results:

ESI-MS analysis: Calculated C₁₀₂H₂₀₈N₂O₁₀Si₄, [M+H⁺]=1734.50, Observed=867.7 [M/2+H⁺].

### Step 2: Synthesis of Compound 29

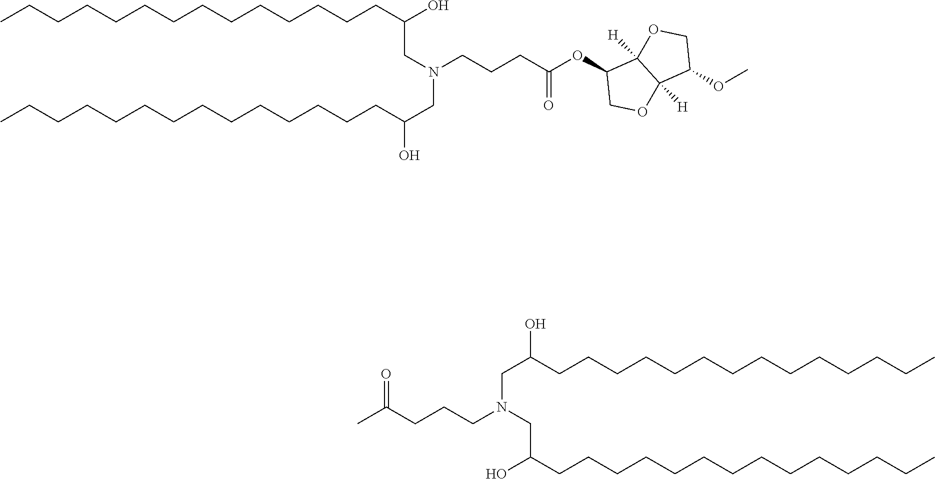

As depicted in Scheme 14: To a solution of Intermediate (2) (503 mg, 0.290 mmol) in 3.5 mL of anhydrous tetrahydrofuran at 0° C. was added hydrogen fluoride pyridine (70% HF, 1.7 mL, 13 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 72% ethyl acetate in hexanes) to obtain Compound 29 (229 mg, 62%).

### Results:

¹H NMR (400 MHz, CDCl₃) δ 5.25-5.10 (m, 2H), 4.90-4.80 (m, 2H), 4.51-4.44 (m, 2H), 4.32-4.14 (m, 2H), 4.00-3.82 (m, 4H), 3.43-3.01 (m, 10H), 2.62-2.13 (m, 6H), 1.74-1.12 (m, 108H), 0.88 (t, 12H).

ESI-MS: Calculated C₇₈H₁₅₂N₂O₁₀, [M+H⁺]=1278.15, Observed=1278.3, and 639.3 [M/2+H⁺]

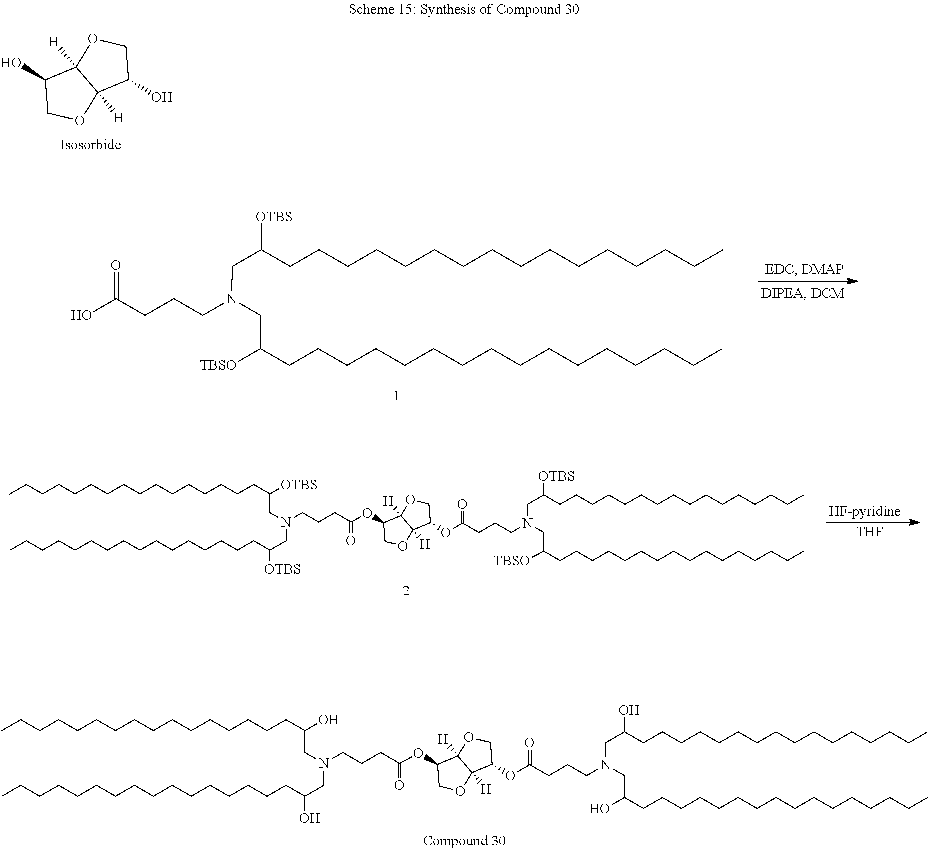

### Step 1: Synthesis of Intermediate (2)

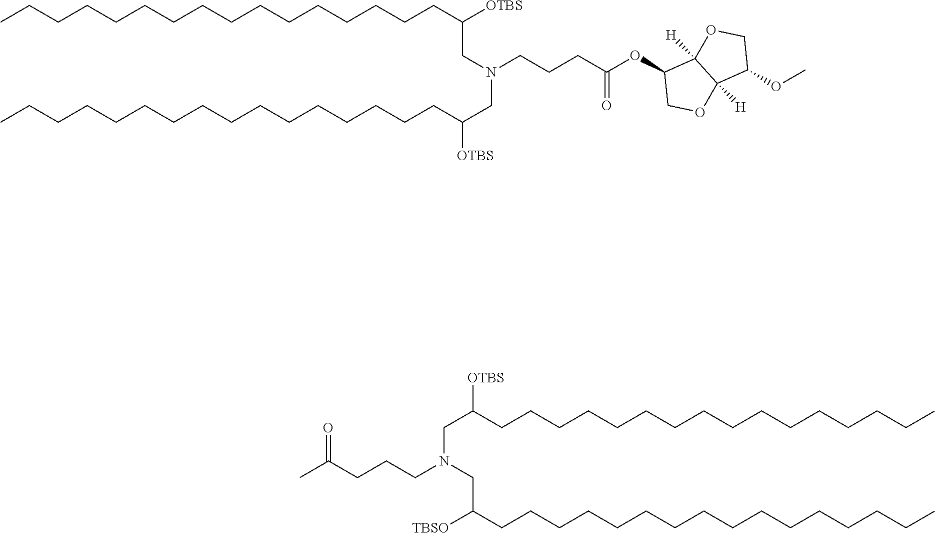

As depicted in Scheme 15: To a solution of acid (1) (751 mg, 0.865 mmol) in 7.5 mL of anhydrous dichloromethane was added isosorbide (43 mg, 0.29 mmol), DMAP (39 mg, 0.32 mmol), DIPEA (0.40 mL, 2.0 mmol), and EDC (223 mg, 1.16 mmol). The resulting mixture was stirred at room temperature overnight. MS analysis showed the presence of monoester. Additional EDC (110 mg, 0.574 mmol) was added to the reaction and the reaction was run for an additional 2 hours. Mass spec confirmed that less monoester was present. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (214 mg, 39%).

### Results:

ESI-MS analysis: Calculated C₁₁₀H₂₂₄N₂O₁₀Si₄, [M+H⁺]=1846.62, Observed=924.3 [M/2+H⁺].

### Step 2: Synthesis of Compound 30

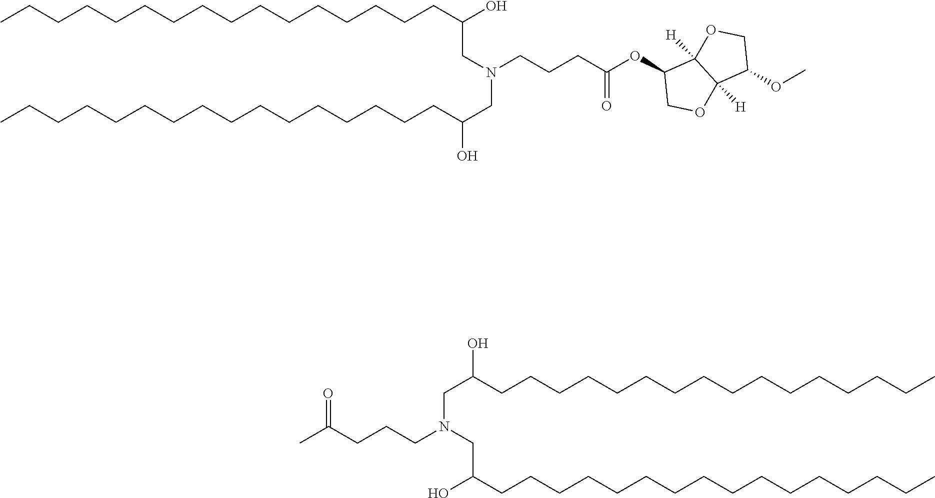

As depicted in Scheme 15: To a solution of Intermediate (2) (214 mg, 0.116 mmol) in 1.8 mL of anhydrous tetrahydrofuran at 0° C. was added hydrogen fluoride pyridine (70% HF, 0.90 mL, 7.0 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 61% ethyl acetate in hexanes) to obtain Compound 30 (87 mg, 54%).

### Results:

¹H NMR (400 MHz, CDCl₃) δ 5.18-5.04 (m, 2H), 4.85-4.73 (m, 2H), 4.48-4.37 (m, 2H), 4.19-4.13 (m, 2H), 3.88-3.78 (m, 4H), 3.28-3.00 (m, 10H), 2.60-2.36 (m, 6H), 2.30-2.05 (m, 4H), 1.79-1.11 (m, 120H), 0.81 (t, J=6.6 Hz, 12H).

ESI-MS: Calculated C₈₆H₁₆₈N₂O₁₀, [M+H⁺]=1390.28, Observed=695.3 [M/2+H⁺]

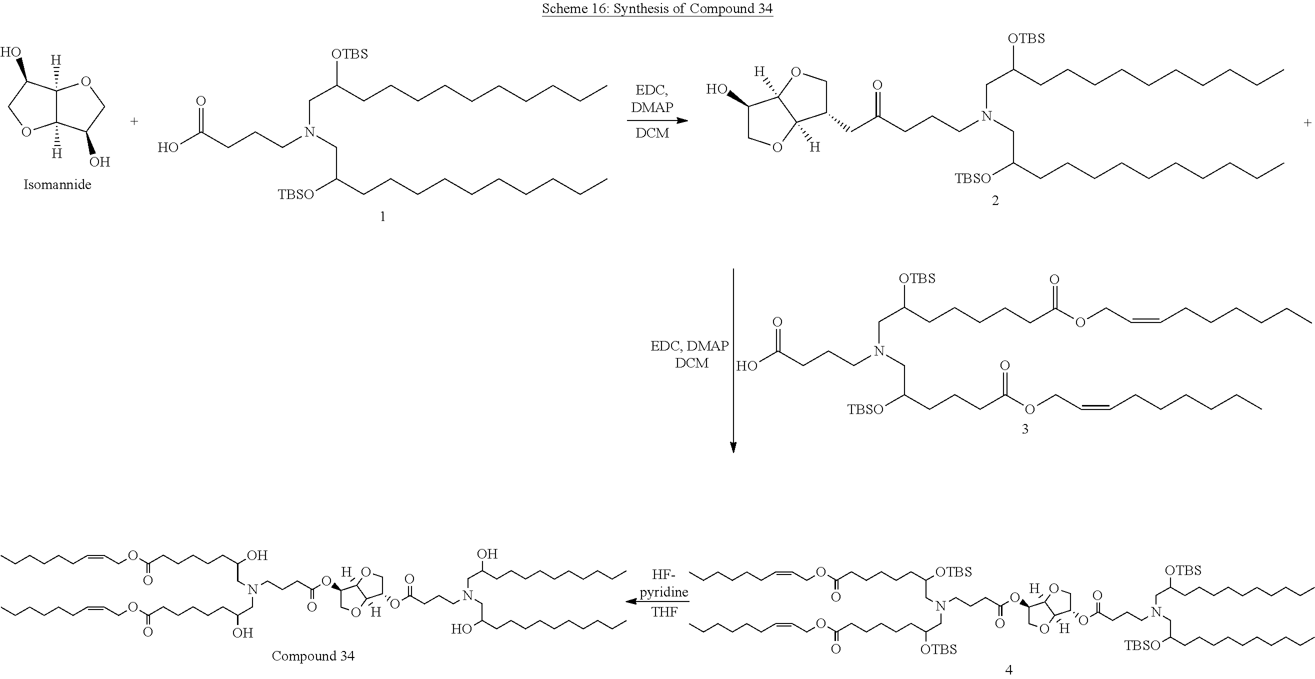

### Step 1: Synthesis of Intermediate (2)

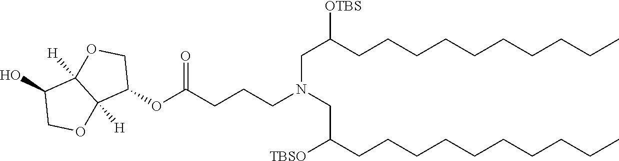 [SMILES: CCCCCCCCCCC(CN(CCCC(=O)O[C@H]1CO[C@H]2[C@@H]1OC[C@H]2O)CC(CCCCCCCCCC)O[Si](C)(C)C(C)(C)C)O[Si](C)(C)C(C)(C)C]

As depicted in Scheme 16: To a solution of acid (1) (750 mg, 1.07 mmol) in 7.5 mL of anhydrous dichloromethane was added isosorbide (144 mg, 0.985 mmol), DMAP (120 mg, 0.978 mmol), and EDC (244 mg, 1.27 mmol). The resulting mixture was stirred at room temperature overnight. MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (SiO₂: 10% ethyl acetate in hexanes) to get Intermediate (2) (368 mg, 45%) (Čekovic, Ź.; Tokic, Z. Selective esterification of 1, 4: 3, 6′-dianhydro-D-glucitol. Synthesis 1989, 8, 610-612).

### Results:

¹H NMR (400 MHz, CDCl₃) δ 5.72-5.63 (m, 2H), 5.59-5.48 (m, 2H), 5.23-5.15 (m, 2H), 4.92-4.80 (m, 1H), 4.67-4.61 (m, 4H), 4.54-4.45 (m, 1H), 4.45-4.30 (m, 1H), 4.30-4.01 (m, 3H), 4.01-3.76 (m, 4H), 3.51-2.90 (m, 18H), 2.90-2.42 (m, 4H), 2.41-2.27 (m, 4H), 2.27-2.02 (m, 6H), 2.01-1.18 (m, 64H), 0.89 (d, J=6.7 Hz, 12H).

ESI-MS analysis: Calculated C₄₆H₉₃NO₇Si₂, [M+H⁺]=828.66, Observed=828.6.

### Step 2: Synthesis of Intermediate (4)

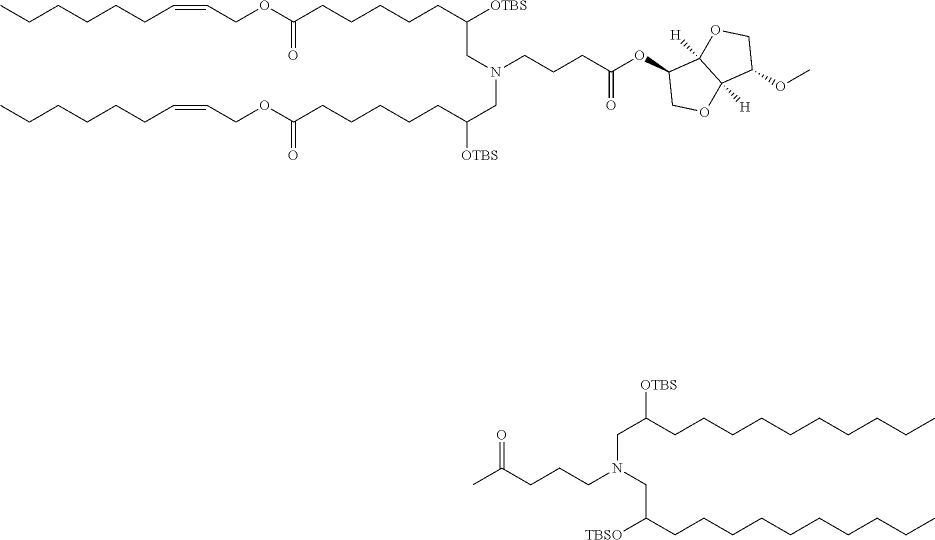

As depicted in Scheme 16: To a solution of acid (3) (460 mg, 0.513 mmol) in 6 mL of anhydrous dichloromethane was added Intermediate (2) (368 mg, 0.444 mmol), DMAP (54 mg, 0.44 mmol), and EDC (110 mg, 0.574 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 5% ethyl acetate in hexanes) to get Intermediate (4) (482 mg, 55%).

### Results:

ESI-MS analysis: Calculated C₉₆H₁₈₈N₂O₁₄Si₄, [M+H⁺]=1706.32, Observed=854.1 [M/2+H⁺].

### Step 3: Synthesis of Compound 34

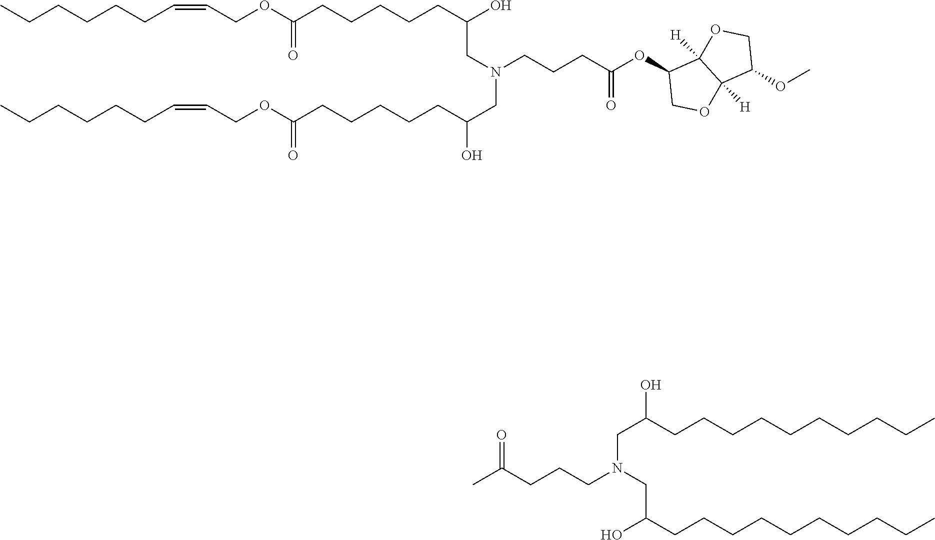

As depicted in Scheme 16: To a solution of Intermediate (4) (482 mg, 0.282 mmol) in 3.5 mL of anhydrous tetrahydrofuran at 0° C. was added hydrogen fluoride pyridine (70% HF, 0.05 mL, 0.4 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 65% ethyl acetate in hexanes) to obtain Compound 34 (220 mg, 62%).

### Results:

¹H NMR (400 MHz, CDCl₃) δ 5.22-5.13 (m, 2H), 4.88-4.79 (m, 2H), 4.50-4.45 (m, 2H), 4.30-4.20 (m, 4H), 4.09-4.01 (m, 2H), 3.97-3.94 (m, OH), 3.94-3.90 (m, 4H), 3.03-2.98 (m, 4H), 2.51-2.46 (m, 6H), 2.39-2.24 (m, 10H), 2.11-2.06 (m, 8H), 1.78-1.68 (m, 1H), 1.68-1.57 (m, 3H), 1.53-1.37 (m, 8H), 1.37-1.27 (m, 25H), 1.27-1.23 (m, 35H), 0.99-0.91 (m, 1H), 0.94-0.80 (m, 11H).

ESI-MS: Calculated C₇₂H₁₃₂N₂O₁₄, [M+H⁺]=1249.98, Observed=1249.8, and 625.5 [M/2+H⁺]

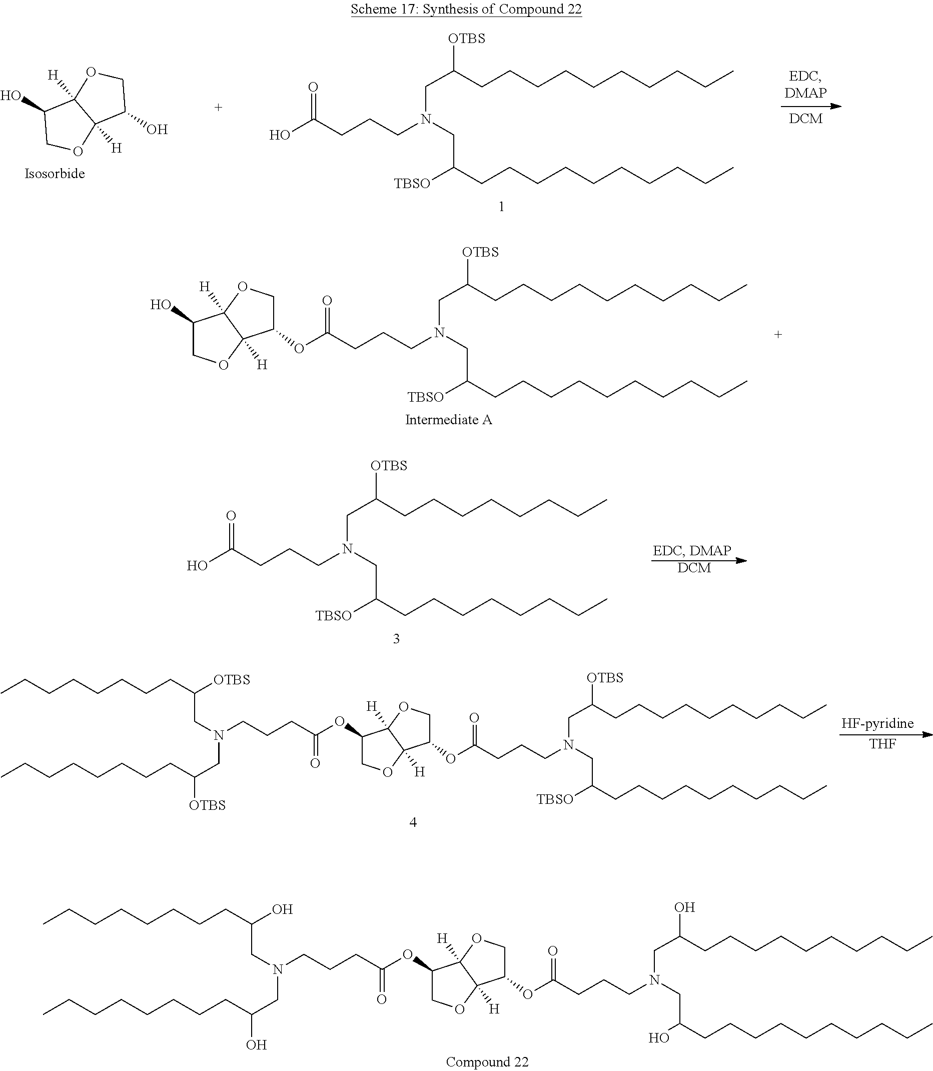

### Step 1: Synthesis of Intermediate A (Scale-Up for Asymmetric Ester Synthesis)

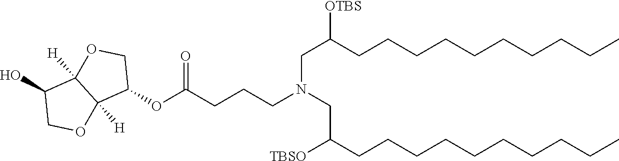 [SMILES: CCCCCCCCCCC(CN(CCCC(=O)O[C@H]1CO[C@H]2[C@@H]1OC[C@H]2O)CC(CCCCCCCCCC)O[Si](C)(C)C(C)(C)C)O[Si](C)(C)C(C)(C)C]

As depicted in Scheme 17: To a solution of acid (1) (4.005 g, 5.719 mmol) in 40 mL of anhydrous dichloromethane was added isosorbide (761 mg, 5.21 mmol), DMAP (638 mg, 5.20 mmol), and EDC (1.296 g, 6.761 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was 4.627 g. This compound was used to create Compound 22, Compound 32, Compound 27, and Compound 25. It is referred to as crude Intermediate A in subsequent procedures. At a later stage, the crude Intermediate A was purified (SiO₂: 10% ethyl acetate in hexanes) and used to create Compound 23.

### Results:

ESI-MS analysis: Calculated C₄₆H₉₃NO₇Si₂, [M+H⁺]=828.66, Observed=828.6.

### Step 2: Synthesis of Intermediate (4)

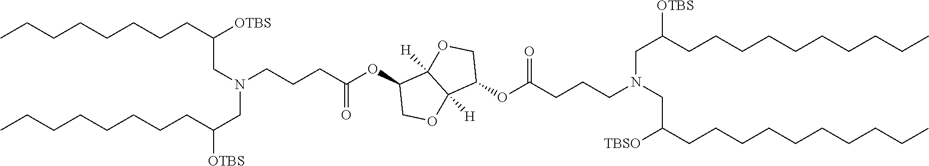 [SMILES: CCCCCCCCCCC(CN(CCCC(=O)O[C@H]1CO[C@H]2[C@@H]1OC[C@H]2OC(=O)CCCN(CC(CCCCCCCC)O[Si](C)(C)C(C)(C)C)CC(CCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 17: To a solution of acid (3) (260 mg, 0.404 mmol) in 6 mL of anhydrous dichloromethane was added crude Intermediate (2) (305 mg, 0.368 mmol), DMAP (46 mg, 0.37 mmol), and EDC (91 mg, 0.47 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (4) (202 mg, 34%).

### Results:

ESI-MS analysis: Calculated C₈₂H₁₆₈N₂O₁₀Si₄, [M+H⁺]=1454.19, Observed=1454.1, and 727.8 [M/2+H⁺].

### Step 3: Synthesis of Compound 22

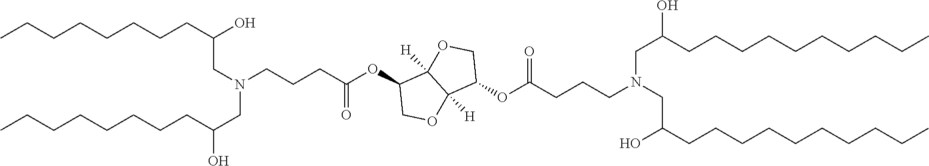 [SMILES: CCCCCCCCCCC(O)CN(CCCC(=O)O[C@H]1CO[C@H]2[C@@H]1OC[C@H]2OC(=O)CCCN(CC(O)CCCCCCCC)CC(O)CCCCCCCC)CC(O)CCCCCCCCCC]

As depicted in Scheme 17: To a solution of Intermediate (4) (202 mg, 0.139 mmol) in 1.4 mL of anhydrous tetrahydrofuran at 0° C. was added hydrogen fluoride pyridine (70% HF, 0.68 mL, 5.3 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 77% ethyl acetate in hexanes) to obtain Compound 22 (78 mg, 56%).

### Results:

¹H NMR (400 MHz, CDCl₃) δ 5.22-5.09 (m, 2H), 4.90-4.79 (m, 1H), 4.51-4.45 (m, 1H), 4.22-3.76 (m, 8H), 3.20-2.54 (m, 12H), 2.54-2.28 (m, 4H), 2.19-1.95 (m, 4H), 1.56-1.20 (m, 64H), 0.88 (t, J=6.7 Hz, 12H).

ESI-MS: Calculated C₅₈H₁₁₂N₂O₁₀, [M+H⁺]=997.84, Observed=997.8, and 499.5 [M/2+H⁺]

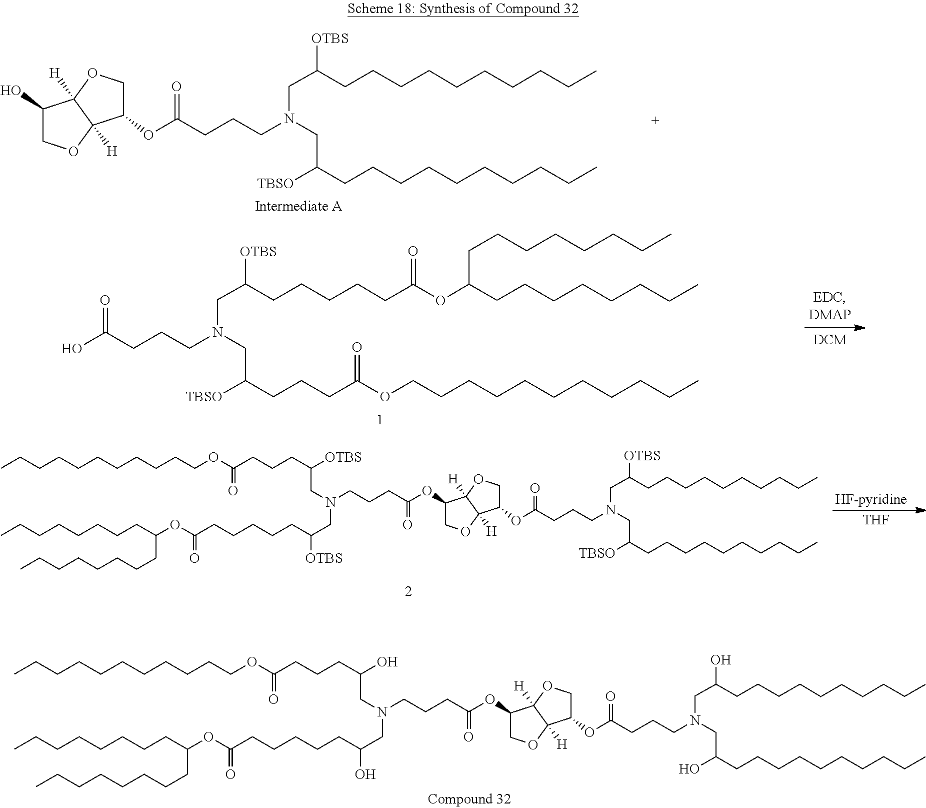

### Step 1: Synthesis of Intermediate (2)

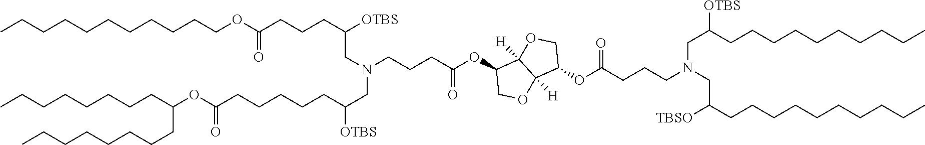 [SMILES: CCCCCCCCCCCOC(=O)CCCC(CN(CCCC(=O)O[C@@H]1CO[C@H]2[C@@H]1OC[C@@H]2OC(=O)CCCN(CC(CCCCCCCCCC)O[Si](C)(C)C(C)(C)C)CC(CCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 18: To a solution of acid (1) (416 mg, 0.411 mmol) in 6 mL of anhydrous dichloromethane was added crude Intermediate A (as prepared in Scheme 17, Step 1) (305 mg, 0.368 mmol), DMAP (47 mg, 0.38 mmol), and EDC (93 mg, 0.49 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (318 mg, 42%).

### Results:

ESI-MS analysis: Calculated C₁₀₄H₂₀₈N₂O₁₄Si₄, [M+H⁺]=1822.48, Observed=912.2 [M/2+H⁺].

### Step 2: Synthesis of Compound 32

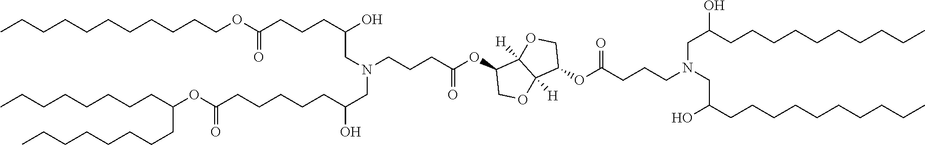 [SMILES: CCCCCCCCCCCOC(=O)CCCC(O)CN(CCCC(=O)O[C@@H]1CO[C@H]2[C@@H]1OC[C@@H]2OC(=O)CCCN(CC(O)CCCCCCCCCC)CC(O)CCCCCCCCCC)CC(O)CCCCCC(=O)OC(CCCCCCCC)CCCCCCCC]

As depicted in Scheme 18: To a solution of Intermediate (2) (318 mg, 0.174 mmol) in 2.1 mL of anhydrous tetrahydrofuran at 0° C. was added hydrogen fluoride pyridine (70% HF, 0.16 mL, 1.2 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 65% ethyl acetate in hexanes) to obtain Compound 32 (88 mg, 37%).

### Results:

¹H NMR (400 MHz, CDCl₃) δ 5.21-5.11 (m, 2H), 4.88-4.79 (m, 2H), 4.50-4.30 (m, 3H), 4.30-4.19 (m, 2H), 4.19-3.66 (m, 8H), 3.31-2.57 (m, 6H), 2.55-2.39 (m, 4H), 2.39-2.24 (m, 4H), 2.18-1.97 (m, 4H), 1.91-1.00 (m, 98H), 0.88 (t, 15H).

ESI-MS: Calculated C₈₀H₁₅₂N₂O₁₄, [M+H⁺]=1366.13, Observed=1365.9, and 683.7 [M/2+H⁺]

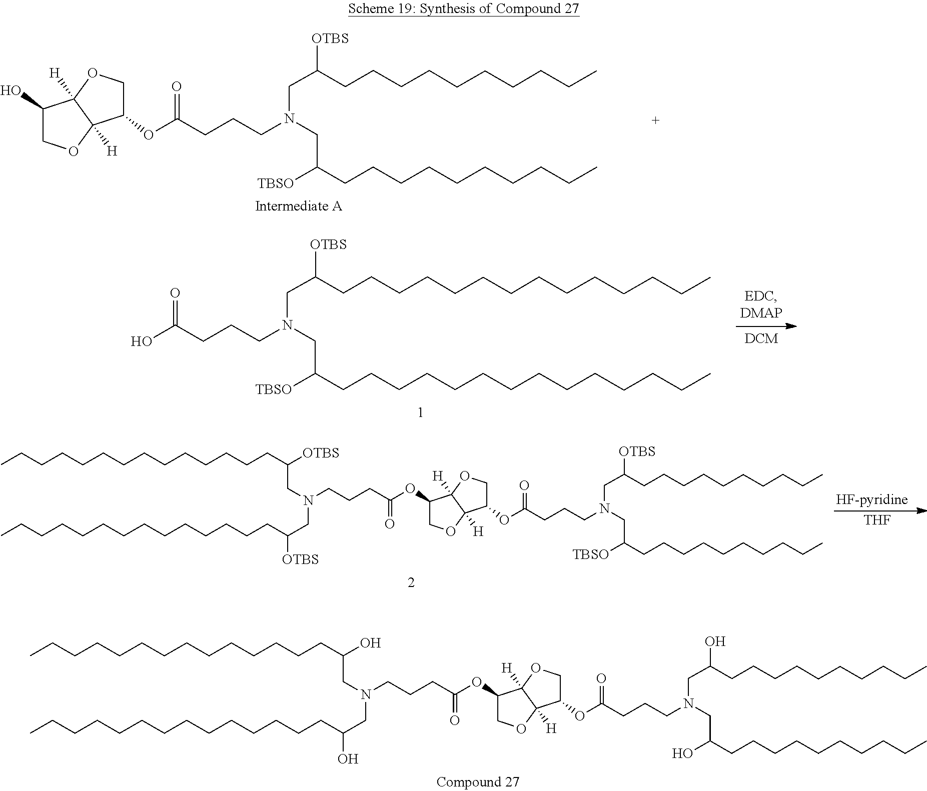

### Step 1: Synthesis of Intermediate (2)

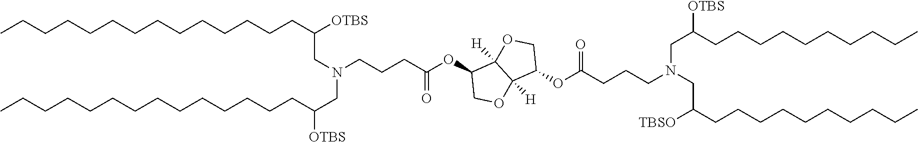 [SMILES: CCCCCCCCCCCCCCC(CN(CCCC(=O)O[C@@H]1CO[C@H]2[C@@H]1OC[C@@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCCCCCC)O[Si](C)(C)C(C)(C)C)O[Si](C)(C)C(C)(C)C]

As depicted in Scheme 19: To a solution of acid (1) (392 mg, 0.483 mmol) in 6 mL of anhydrous dichloromethane was added crude Intermediate A (as prepared in Scheme 17, Step 1) (354 mg, 0.427 mmol), DMAP (52 mg, 0.42 mmol), and EDC (106 mg, 0.553 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (453 mg, 58%).

### Results:

ESI-MS analysis: Calculated C₉₄H₁₉₂N₂O₁₀Si₄, [M+H⁺]=1622.37, Observed=812.6 [M/2+H⁺].

### Step 2: Synthesis of Compound 27

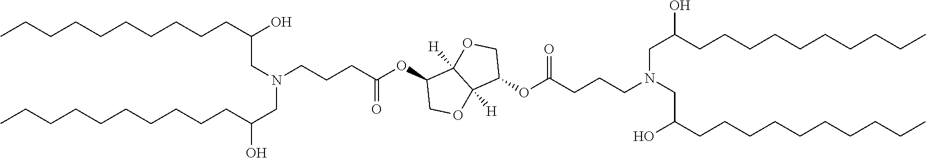 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

As depicted in Scheme 19: To a solution of Intermediate (2) (453 mg, 0.279 mmol) in 3 mL of anhydrous tetrahydrofuran at 0° C. was added hydrogen fluoride pyridine (70% HF, 1.5 mL, 12 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 54% ethyl acetate in hexanes) to obtain Compound 27 (132 mg, 41%).

### Results:

¹H NMR (400 MHz, CDCl₃) δ 5.22-5.13 (m, 2H), 4.92-4.82 (m, 1H), 4.51-4.44 (m, 1H), 4.22-3.76 (m, 8H), 3.34-2.78 (m, 12H), 2.72-2.32 (m, 4H), 2.27-1.96 (m, 4H), 1.58-1.15 (m, 88H), 0.88 (t, J=6.6 Hz, 12H).

ESI-MS: Calculated C₇₀H₁₃₆N₂O₁₀, [M+H⁺]=1166.03, Observed=1165.9, and 583.6 [M/2+H⁺]

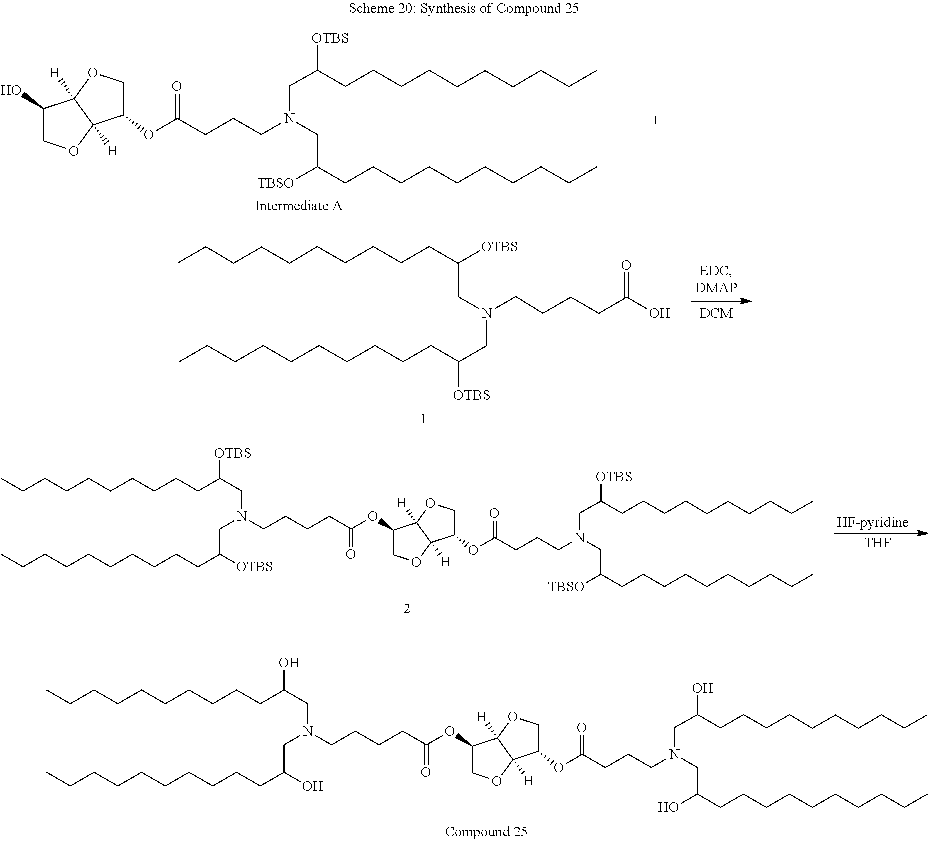

### Step 1: Synthesis of Intermediate (2)

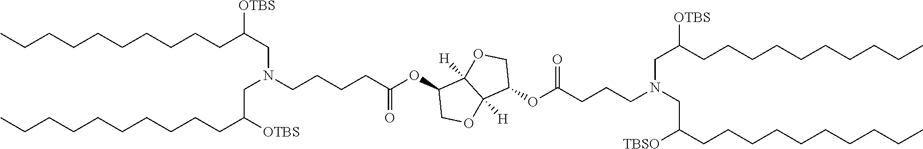 [SMILES: CCCCCCCCCCC(CN(CCCCC(=O)O[C@@H]1CO[C@H]2[C@@H]1OC[C@@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 20: To a solution of acid (1) (349 mg, 0.489 mmol) in 6 mL of anhydrous dichloromethane was added crude Intermediate A (as prepared in Scheme 17, Step 1) (354 mg, 0.427 mmol), DMAP (53 mg, 0.43 mmol), and EDC (105 mg, 0.548 mmol). The resulting mixture was stirred at room temperature overnight. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (429 mg, 58%).

### Results:

ESI-MS analysis: Calculated C₈₇H₁₇₈N₂O₁₀Si₄, [M+H⁺]=1524.26, Observed=762.2 [M/2+H⁺].

### Step 2: Synthesis of Compound 25

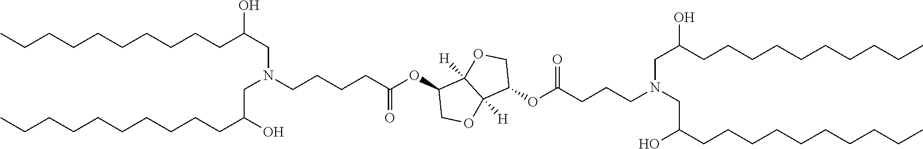 [SMILES: CCCCCCCCCCC(O)CN(CCCCC(=O)O[C@@H]1CO[C@H]2[C@@H]1OC[C@@H]2OC(=O)CCCN(CC(O)CCCCCCCCCC)CC(O)CCCCCCCCCC)CC(O)CCCCCCCCCC]

As depicted in Scheme 20: To a solution of Intermediate (2) (429 mg, 0.281 mmol) in 3 mL of anhydrous tetrahydrofuran at 0° C. was added hydrogen fluoride pyridine (70% HF, 1.5 mL, 12 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 61% ethyl acetate in hexanes) to obtain Compound 25 (87 mg, 29%).

### Results:

¹H NMR (400 MHz, CDCl₃) δ 5.25-5.07 (m, 2H), 4.92-4.73 (m, 1H), 4.54-4.41 (m, 1H), 4.32-4.13 (m, 2H), 4.05-3.83 (m, 2H), 3.39-3.00 (m, 12H), 2.83-2.30 (m, 8H), 2.28-2.11 (m, 2H), 2.07-1.88 (m, 2H), 1.81-1.15 (m, 74H), 0.88 (t, J=6.6 Hz, 12H).

ESI-MS: Calculated C₆₃H₁₂₂N₂O₁₀, [M+H⁺]=1067.92, Observed=1067.8, and 534.5 [M/2+H⁺]

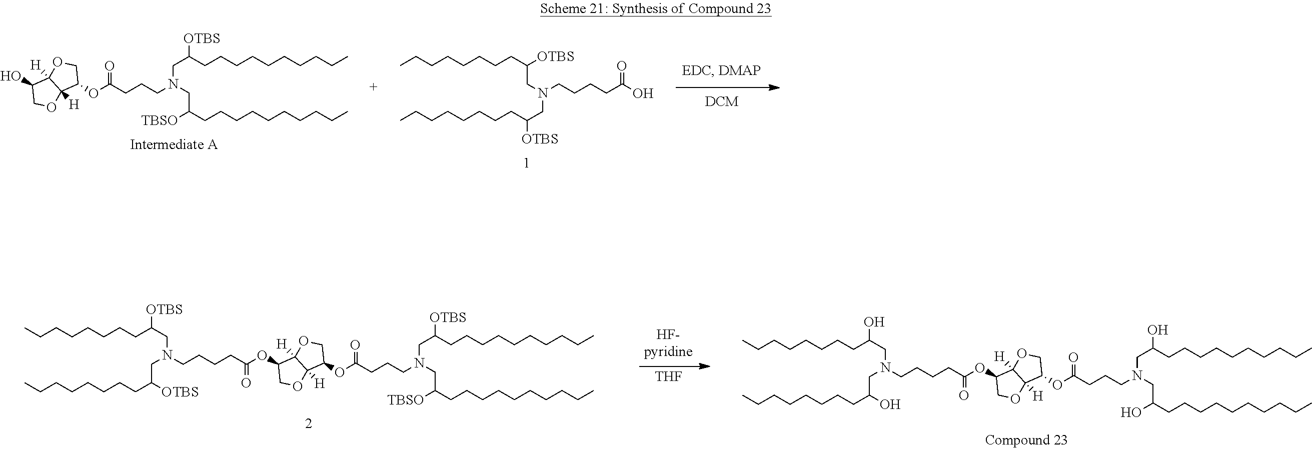

### Step 1: Synthesis of Intermediate (2)

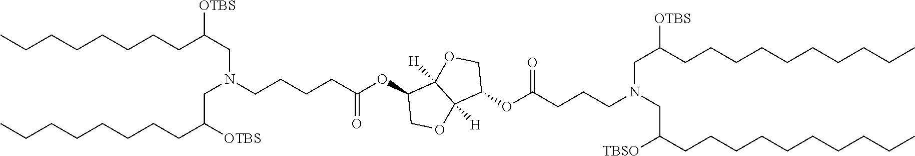 [SMILES: CCCCCCCCCCC(CN(CCCC(=O)O[C@H]1CO[C@H]2[C@@H]1OC[C@H]2OC(=O)CCCCN(CC(CCCCCCCC)O[Si](C)(C)C(C)(C)C)CC(CCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 21: To a solution of acid (1) (460 mg, 0.699 mmol) in 6 mL of anhydrous dichloromethane was added Intermediate A (as prepared in Scheme 17, Step 1) (368 mg, 0.444 mmol), DMAP (54 mg, 0.44 mmol), and EDC (110 mg, 0.574 mmol). The resulting mixture was stirred at room temperature overnight. MS analysis showed the presence of Intermediate (2) in addition to Intermediate A. DIPEA (0.25 mL, 1.4 mmol) was added and the reaction ran for 24 more hours. MS analysis indicated completion of the reaction. The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (482 mg, 47%).

### Results:

ESI-MS analysis: Calculated C₈₃H₁₇₀N₂O₁₀Si₄, [M+H⁺]=1468.20, Observed=1468.1, and 734.8 [M/2+H⁺].

### Step 2: Synthesis of Compound 23

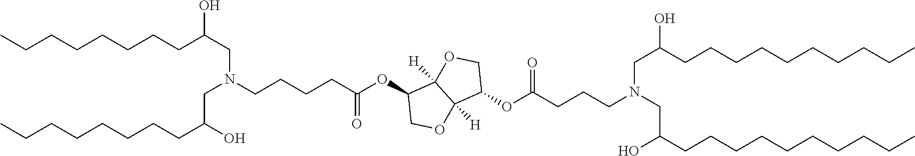 [SMILES: CCCCCCCCCCC(O)CN(CCCC(=O)O[C@H]1CO[C@H]2[C@@H]1OC[C@H]2OC(=O)CCCCN(CC(O)CCCCCCCC)CC(O)CCCCCCCC)CC(O)CCCCCCCCCC]

As depicted in Scheme 21: To a solution of Intermediate (2) (694 mg, 0.473 mmol) in 3.5 mL of anhydrous tetrahydrofuran at 0° C. was added hydrogen fluoride pyridine (70% HF, 1.8 mL, 14 mmol). The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude residue was purified (SiO₂: 45% ethyl acetate in hexanes) to obtain Compound 23 (349 mg, 73%).

### Results:

¹H NMR (400 MHz, CDCl₃) δ 5.21-5.11 (m, 2H), 4.88-4.75 (m, 2H), 4.31-4.20 (m, 2H), 3.99-3.89 (m, 2H), 3.84-3.56 (m, 4H), 2.94-2.17 (m, 16H), 1.89-1.14 (m, 70H), 0.94-0.80 (m, 12H).

ESI-MS: Calculated C₅₉H₁₁₄N₂O₁₀, [M+H⁺]=1011.86, Observed=1011.8, and 506.5 [M/2+H⁺]

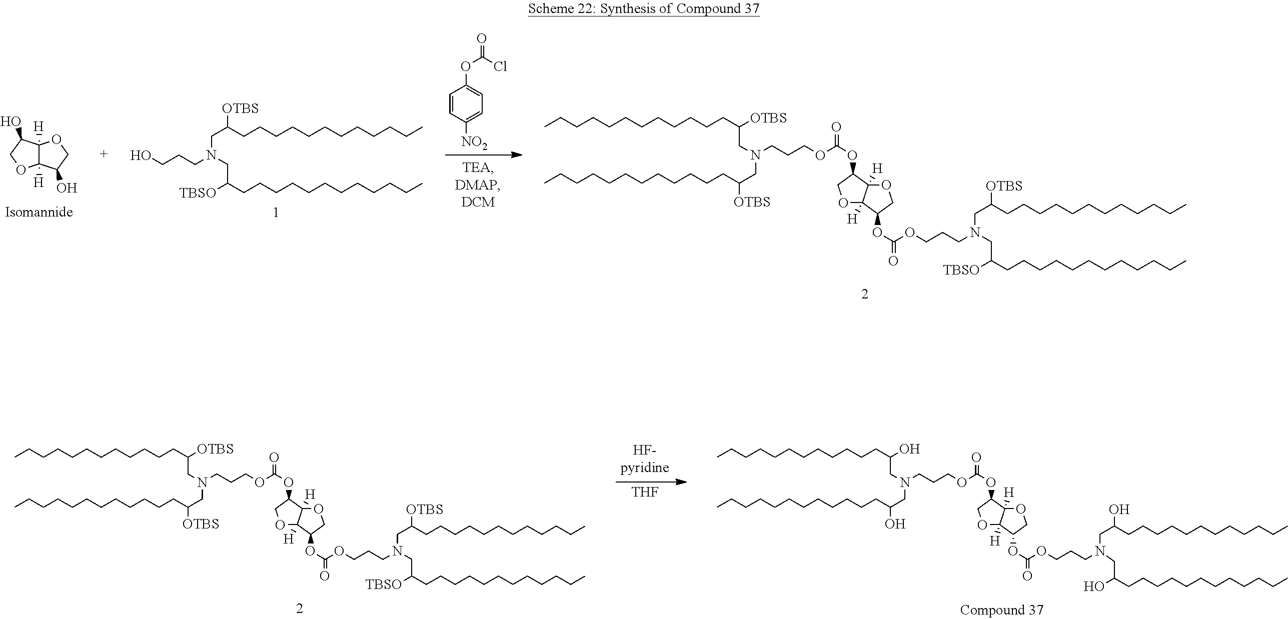

### Step 1: Synthesis of Intermediate (2)

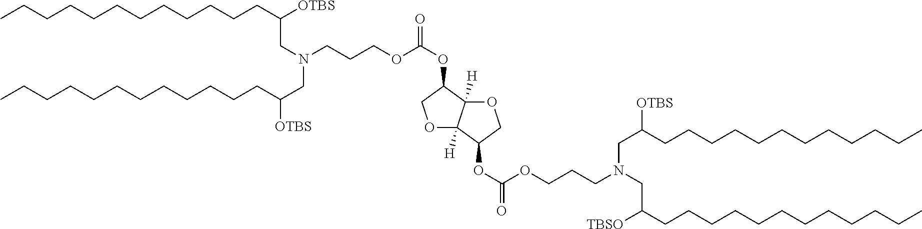 [SMILES: CCCCCCCCCCCCC(CN(CCCOC(=O)O[C@@H]1CO[C@H]2[C@@H]1OC[C@H]2OC(=O)OCCCN(CC(CCCCCCCCCCCC)O[Si](C)(C)C(C)(C)C)CC(CCCCCCCCCCCC)O[Si](C)(C)C(C)(C)C)CC(CCCCCCCCCCCC)O[Si](C)(C)C(C)(C)C)O[Si](C)(C)C(C)(C)C]

As depicted in Scheme 22: To a solution of isomannide (34 mg, 0.23 mmol) in 5.5 mL of anhydrous DCM was added DMAP (6 mg, 0.05 mmol) and triethylamine (0.2 mL, 1.0 mmol). To the resultant mixture, NO₂PhOCOCl (100 mg, 0.496 mmol) was added and stirred for 20 minutes at room temperature. Alcohol (1) (376 mg, 0.516 mmol) was added, and the reaction mixture was stirred at room temperature overnight. MS showed starting material (alcohol (1)) and a small peak for the desired product (poor ionization). The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (225 mg, 58%).

### Results:

ESI-MS analysis: Calculated C₉₄H₁₉₂N₂O₁₂Si₄, [M+H⁺]=1654.36, Observed=827.6 [M/2+H⁺].

### Step 2: Synthesis of Compound 37

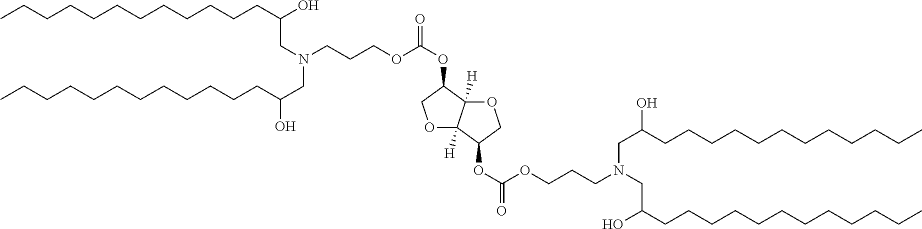 [SMILES: CCCCCCCCCCCCC(O)CN(CCCOC(=O)O[C@@H]1CO[C@H]2[C@@H]1OC[C@H]2OC(=O)OCCCN(CC(O)CCCCCCCCCCCC)CC(O)CCCCCCCCCCCC)CC(O)CCCCCCCCCCCC]

As depicted in Scheme 22: To a solution of Intermediate (2) (225 mg, 0.136 mmol) in 1.5 mL anhydrous tetrahydrofuran at 0° C., hydrogen fluoride pyridine (HF 70%, 0.75 mL, 5.8 mmol) was added. The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then that organic layer was dried over anhydrous Na₂SO₄ and concentrated. The crude residue was purified (SiO₂: 8% methanol in DCM) to obtain Compound 37 (35 mg, 21%).

### Results:

¹H NMR (400 MHz, CDCl₃) δ 5.04-4.98 (m, 2H), 4.77-4.72 (m, 2H), 4.32-4.19 (m, 4H), 4.11-4.02 (m, 2H), 3.92-3.82 (m, 2H), 3.76-3.71 (m, 4H), 2.90-2.42 (m, 12H), 2.02-1.85 (m, 4H), 1.54-1.16 (m, 88H), 0.88 (t, J=6.7 Hz, 12H).

ESI-MS: Calculated C₇₀H₁₃₆N₂O₁₂, [M+H⁺]=1198.01, Observed=1197.9, and 599.5[M/2+H⁺]

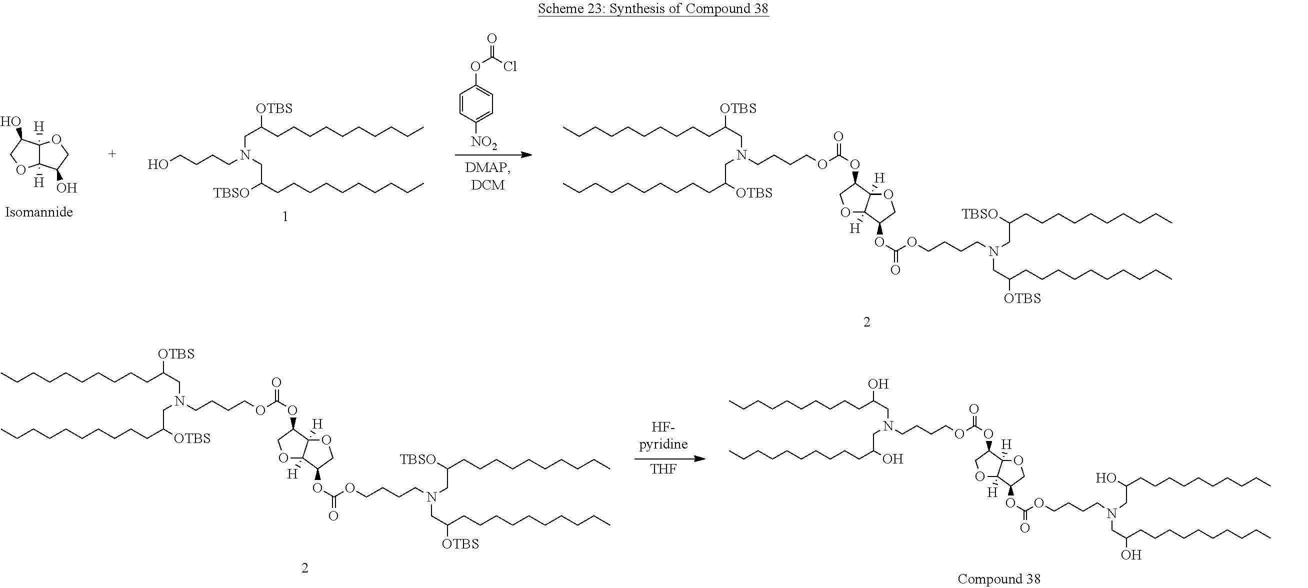

### Step 1: Synthesis of Intermediate (2)

As depicted in Scheme 23: To a solution of isomannide (30 mg, 0.21 mmol) in 3 mL of anhydrous DCM was added DMAP (5 mg, 0.04 mmol) and triethylamine (0.2 mL, 1.0 mmol). To the resultant mixture, NO₂PhOCOCl (100 mg, 0.496 mmol) was added and stirred for 20 minutes at room temperature. Alcohol (1) (374 mg, 0.545 mmol) was added, and the reaction mixture was stirred at room temperature overnight. MS showed starting material (1) and a small peak for the desired product (poor ionization). The reaction mixture was diluted with DCM and washed sat. NaHCO₃ solution, water, and brine solution. The organic layer was dried over anhydrous Na₂SO₄ and concentrated. The crude residue was purified (SiO₂: 4% ethyl acetate in hexanes) to get Intermediate (2) (270 mg, 84%).

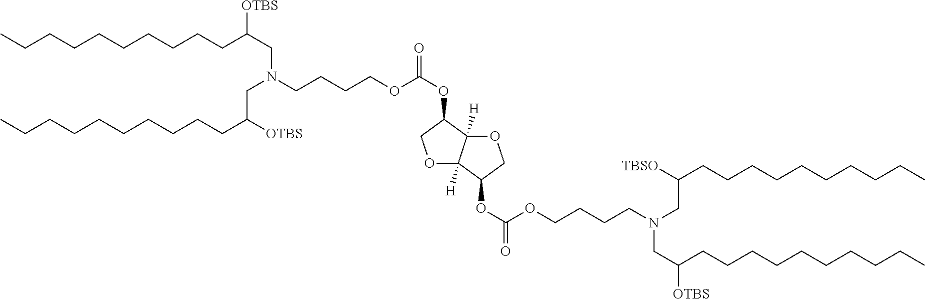 [SMILES: CCCCCCCCCCC(CN(CCCCOC(=O)O[C@@H]1CO[C@H]2[C@@H]1OC[C@H]2OC(=O)OCCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

### Results:

ESI-MS: Calculated C₈₈H₁₈₀N₂O₁₂Si₄, [M+H⁺]=1570.26, Observed=786.1 [M/2+H⁺].

### Step 2: Synthesis of Compound 38

As depicted in Scheme 23: To a solution of Intermediate (2) (270 mg, 0.172 mmol) in 2 mL anhydrous tetrahydrofuran at 0° C., hydrogen fluoride pyridine (HF 70%, 0.90 mL, 7.0 mmol) was added. The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then that organic layer was dried over anhydrous Na₂SO₄ and concentrated. The crude residue was purified (SiO₂: 8% methanol in DCM) to obtain Compound 38 (35 mg, 18%).

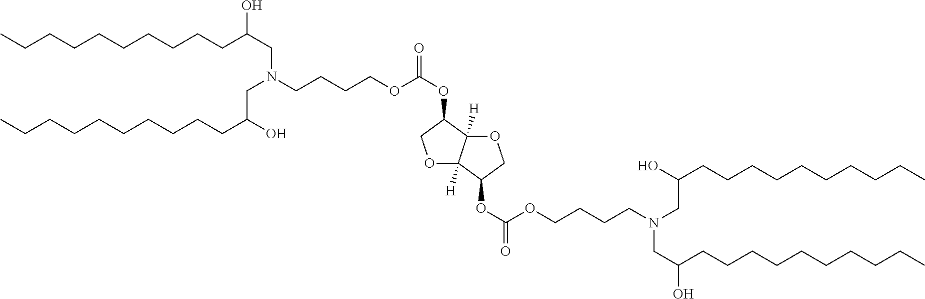 [SMILES: CCCCCCCCCCC(O)CN(CCCCOC(=O)O[C@@H]1CO[C@H]2[C@@H]1OC[C@H]2OC(=O)OCCCCN(CC(O)CCCCCCCCCC)CC(O)CCCCCCCCCC)CC(O)CCCCCCCCCC]

### Results:

¹H NMR (400 MHz, CDCl₃) δ 5.06-4.97 (m, 2H), 4.77-4.71 (m, 2H), 4.22-4.12 (m, 4H), 4.12-3.97 (m, 2H), 3.94-3.83 (m, 2H), 3.83-3.64 (m, 4H), 2.85-2.47 (m, 12H), 1.87-1.57 (m, 8H), 1.57-1.16 (m, 72H), 0.92-0.80 (m, 12H).

ESI-MS: Calculated C₆₄H₁₂₄N₂O₁₂, [M+H⁺]=1113.92, Observed=1113.8, and 557.5[M/2+H⁺]

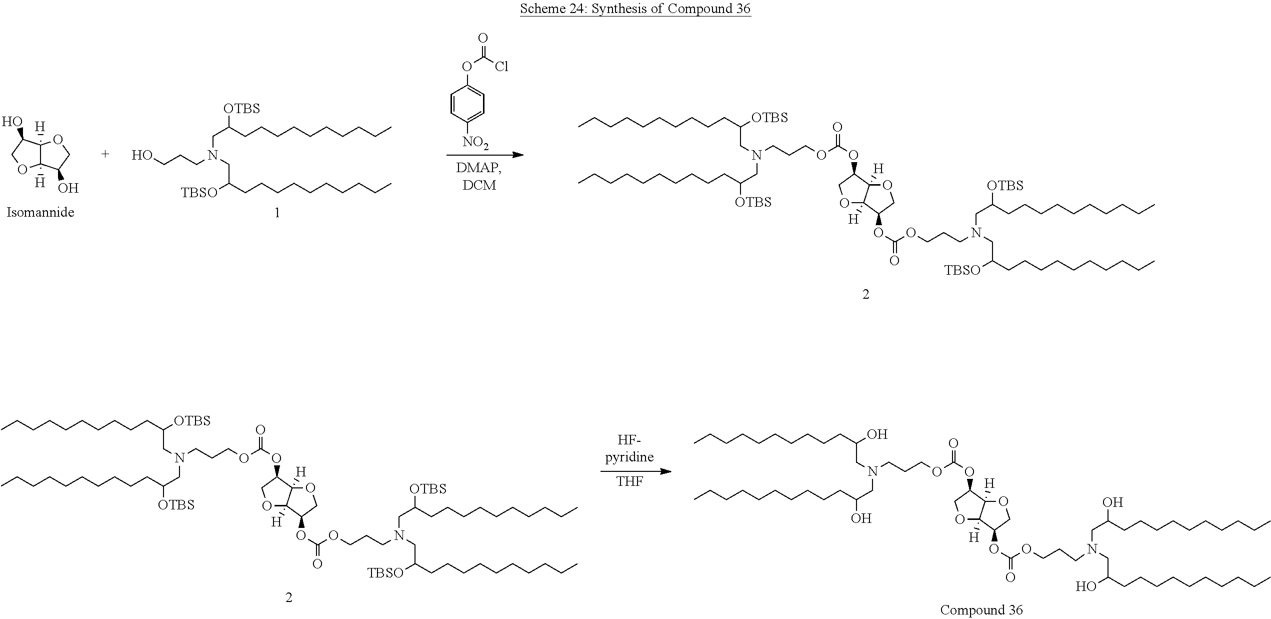

### Step 1: Synthesis of Intermediate (2)

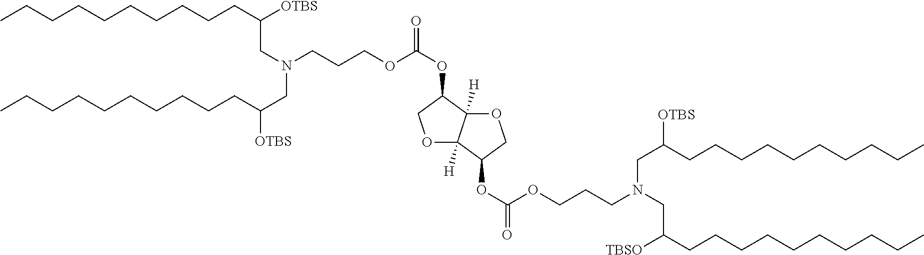 [SMILES: CCCCCCCCCCC(CN(CCCOC(=O)O[C@@H]1CO[C@H]2[C@@H]1OC[C@H]2OC(=O)OCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 24: To a solution of isomannide (66 mg, 0.41 mmol) in 6 mL of anhydrous DCM was added DMAP (12 mg, 0.098 mmol) and triethylamine (0.45 mL, 3.2 mmol). To the resultant mixture, NO₂PhOCOCl (215 mg, 1.07 mmol) was added and stirred for 90 minutes at room temperature. Alcohol (1) (749 mg, 1.11 mmol) was added, and the reaction mixture was stirred at room temperature overnight. MS showed starting material (alcohol (1)) and a small peak for the desired product (poor ionization). The reaction mixture was diluted with DCM and washed sat. NaHCO₃ solution, water, and brine solution. The organic layer was dried over anhydrous Na₂SO₄ and concentrated. The crude residue was purified (SiO₂: 5% ethyl acetate in hexanes) to get Intermediate (2) (630 mg, quantitative yield).

### Results:

ESI-MS analysis: Calculated C₈₆H₁₇₆N₂O₁₂Si₄, [M+H⁺]=1542.23, Observed=772.0 [M/2+H⁺].

### Step 2: Synthesis of Compound 36

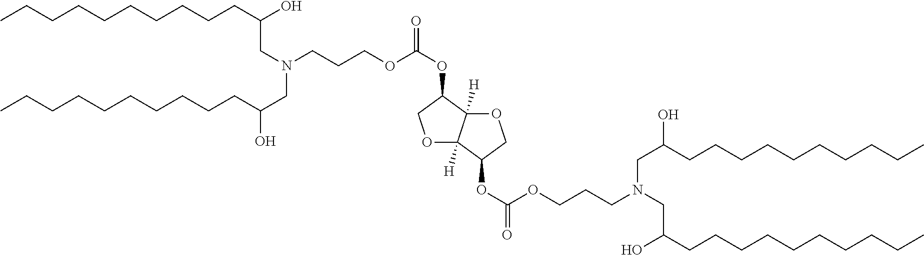 [SMILES: CCCCCCCCCCC(O)CN(CCCOC(=O)O[C@@H]1CO[C@H]2[C@@H]1OC[C@H]2OC(=O)OCCCN(CC(O)CCCCCCCCCC)CC(O)CCCCCCCCCC)CC(O)CCCCCCCCCC]

As depicted in Scheme 24: To a solution of Intermediate (2) (630 mg, 0.408 mmol) in 6 mL anhydrous tetrahydrofuran at 0° C., hydrogen fluoride pyridine (HF 70%, 0.37 mL, 2.9 mmol) was added. The reaction mixture was warmed to room temperature and stirred overnight. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then that organic layer was dried over anhydrous Na₂SO₄ and concentrated. The crude residue was purified (SiO₂: 10% methanol in a solution of 1% triethylamine in DCM) to obtain Compound 36 (73 mg, 16%).

### Results:

¹H NMR (400 MHz, CDCl₃) δ 5.03-4.99 (m, 2H), 4.77-4.72 (m, 2H), 4.30-4.23 (m, 4H), 4.08-4.04 (m, 2H), 3.91-3.86 (m, 2H), 3.67-3.58 (m, 4H), 2.98-2.43 (m, 12H), 2.00-1.93 (m, 4H), 1.49-1.22 (m, 72H), 0.87 (t, J=6.4 Hz, 12H).

ESI-MS: Calculated C₆₂H₁₂₀N₂O₁₂, [M+H⁺]=1085.88, Observed=1085.8, and 543.5 [M/2+H⁺]

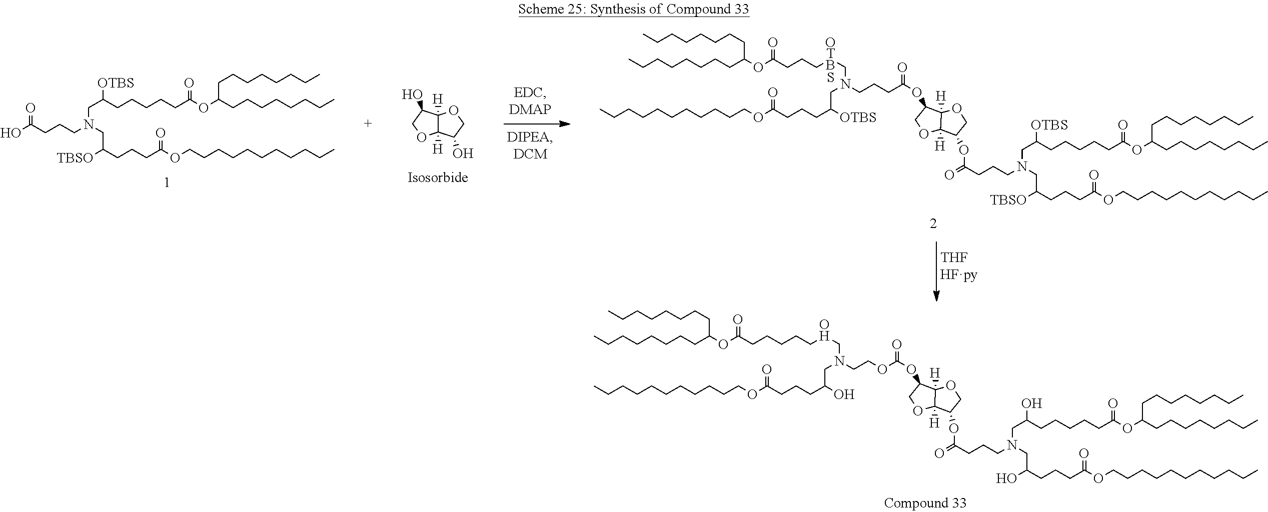

### Step 1: Synthesis of Intermediate (2)

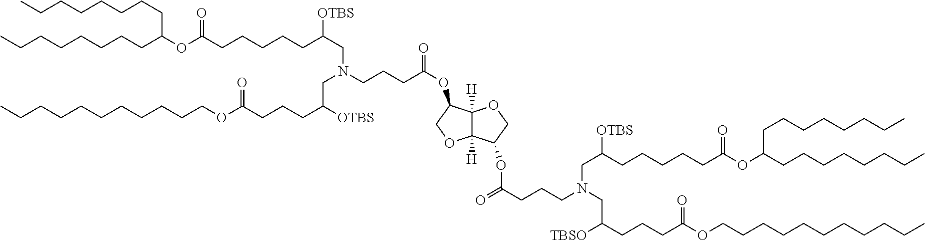 [SMILES: CCCCCCCCCCCOC(=O)CCCC(CN(CCCC(=O)O[C@H]1CO[C@H]2[C@@H]1OC[C@H]2OC(=O)CCCN(CC(CCCCCC(=O)OC(CCCCCCCC)CCCCCCCC)O[Si](C)(C)C(C)(C)C)CC(CCCC(=O)OCCCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 25: In a 20 mL vial was added isosorbide (28.86 mg, 0.2 mmol), acid (1) (500 mg, 0.49 mmol) into 5 mL of DCM. To that solution was DMAP (419.7 mg, 3.421 mmol), DIPEA (0.275 mL, 1.58 mmol), and EDC (151.42 mg, 0.79 mmol) were added, and the resulting mixture was stirred at room temperature overnight. After 16 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (24 g silica column, 7% ethyl acetate-hexane gradient) and obtained 334 mg (79% yield) of Intermediate (2).

### Results

ESI-MS: Calculated C₁₂₂H₂₄₀N₂O₁₈Si₄, [M+H⁺]=2135.71, Observed=1068.2 [M/2+H⁺].

### Step 2: Synthesis of Compound 33

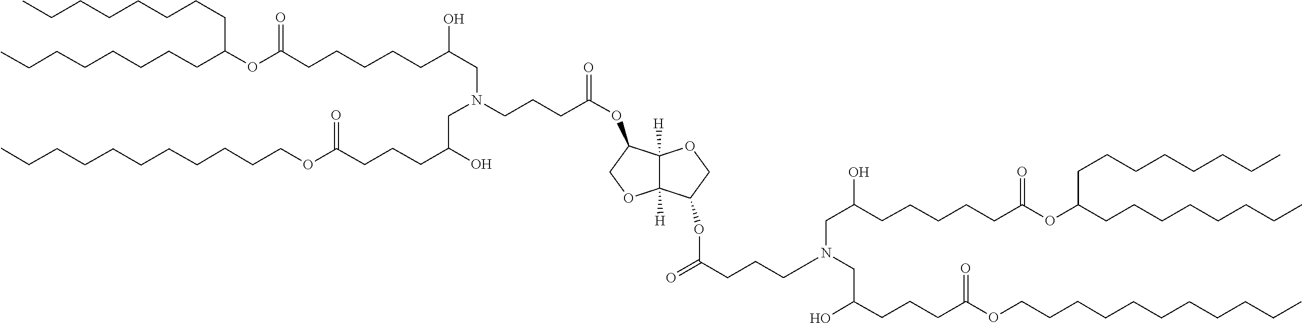 [SMILES: CCCCCCCCCCCOC(=O)CCCC(O)CN(CCCC(=O)O[C@H]1CO[C@H]2[C@@H]1OC[C@H]2OC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC]

As depicted in Scheme 25: To a solution of Intermediate (2) (334 mg, 712.7 mmol) in 2 mL of anhydrous tetrahydrofuran at 0° C. was added hydrogen fluoride pyridine (70% HF.py complex, 1 mL). The reaction mixture was warmed to room temperature and stirred for 16 h. MS analysis indicated completion of the reaction. Reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (12 g, 75% ethyl acetate-hexane gradient) to obtain Compound 33 (10.5 mg, 4%).

### Results

ESI-MS: Calculated C₉₈H₁₈₄N₂O₁₈, [M+H⁺]=1678.36, Observed=839.9 [M/2+H⁺].

¹H NMR (400 MHz, CDCl₃) δ 5.16-4.99 (m, 2H), 4.94-4.79 (m, 2H), 4.77-4.66 (m, 2H), 4.31-4.20 (m, 2H), 4.04 (t, J=6.9 Hz, 8H), 3.20 (d, J=54.9 Hz, 10H), 2.71-2.48 (m, 4H), 2.47-2.08 (m, 24H), 1.91-1.51 (m, 20H), 1.51-1.03 (m, 88H), 0.87 (t, J=6.7 Hz, 18H).

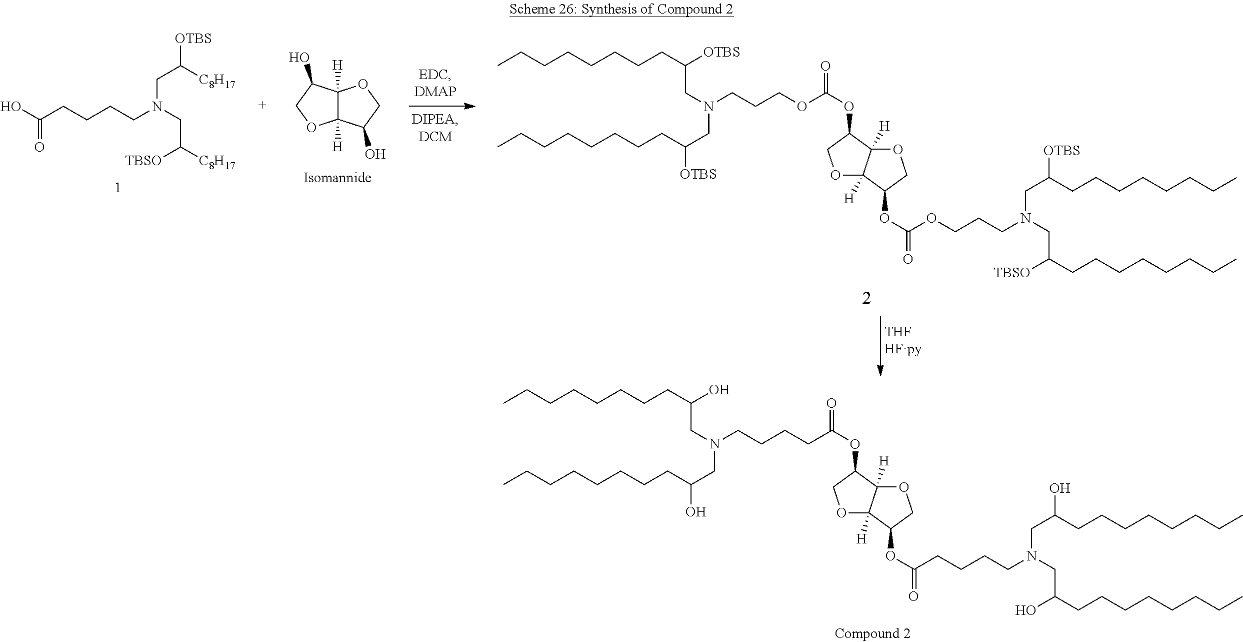

### Step 1: Synthesis of Intermediate (2)

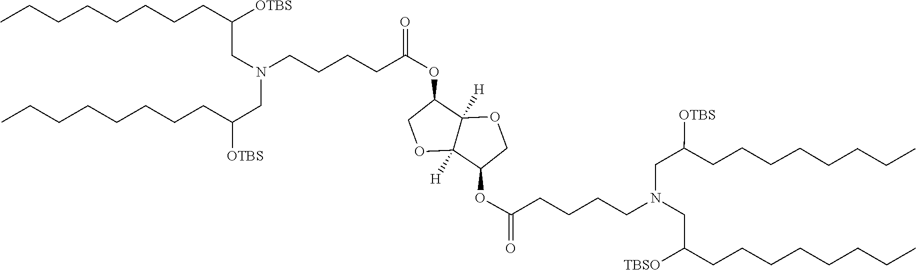 [SMILES: CCCCCCCCC(CN(CCCCC(=O)O[C@@H]1CO[C@H]2[C@@H]1OC[C@H]2OC(=O)CCCCN(CC(CCCCCCCC)O[Si](C)(C)C(C)(C)C)CC(CCCCCCCC)O[Si](C)(C)C(C)(C)C)CC(CCCCCCCC)O[Si](C)(C)C(C)(C)C)O[Si](C)(C)C(C)(C)C]

As depicted in Scheme 26: In a 20 mL vial was added isomannide (44 mg, 0.303 mmol), acid (1) (500 mg, 0.76 mmol) into 5 mL of DCM. To that solution was DMAP (37 mg, 0.303 mmol), DIPEA (0.423 mL, 2.431 mmol) and EDC (233 mg, 1.215 mmol) were added, and the resulting mixture was stirred at room temperature overnight. After 16 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (12 g silica column, 4% ethyl acetate-hexane) and obtained 113 mg (48% yield) of Intermediate (2).

### Results

ESI-MS: Calculated C₈₀H₁₆₄N₂O₁₀Si₄, [M+H⁺]=1426.16, Observed=713.3 [M/2+H⁺].

### Step 2: Synthesis of Compound 2

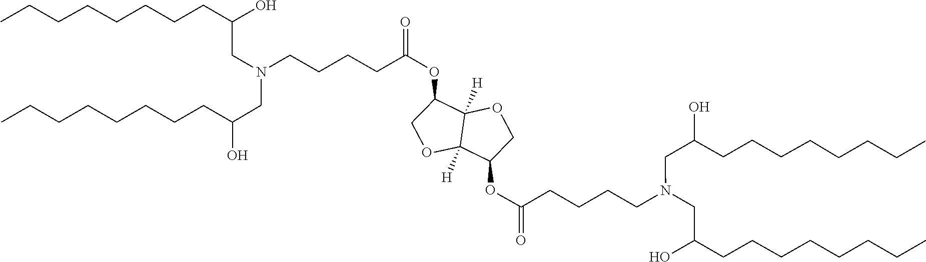 [SMILES: CCCCCCCCC(O)CN(CCCCC(=O)O[C@@H]1CO[C@H]2[C@@H]1OC[C@H]2OC(=O)CCCCN(CC(O)CCCCCCCC)CC(O)CCCCCCCC)CC(O)CCCCCCCC]

As depicted in Scheme 26: To a solution of Intermediate (2) (402.6 mg, 0.282 mmol) in 3 mL of anhydrous tetrahydrofuran at 0° C. was added hydrogen fluoride pyridine (70% HF.py complex, 1.5 mL). The reaction mixture was warmed to room temperature and stirred for 16 h. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (12 g, 45% ethyl acetate-hexane gradient) to obtain Compound 2 (130.4 mg, 37%).

### Results

ESI-MS: Calculated C₅₆H₁₀₈N₂O₁₀, [M+H⁺]=969.81, Observed=969.2 [M+H⁺], 485.1 [M/2+H⁺].

¹H NMR (400 MHz, CDCl₃) δ 5.13-5.04 (m, 2H), 4.73-4.67 (m, 2H), 4.04 (dd, 4H), 4.01-3.66 (m, 4H), 3.17-2.85 (m, 8H), 2.50-2.37 (m, 4H), 1.71 (p, J=7.5 Hz, 4H), 1.57-1.34 (m, 12H), 1.42-1.09 (m, 52H), 0.87 (t, J=6.6 Hz, 12H).

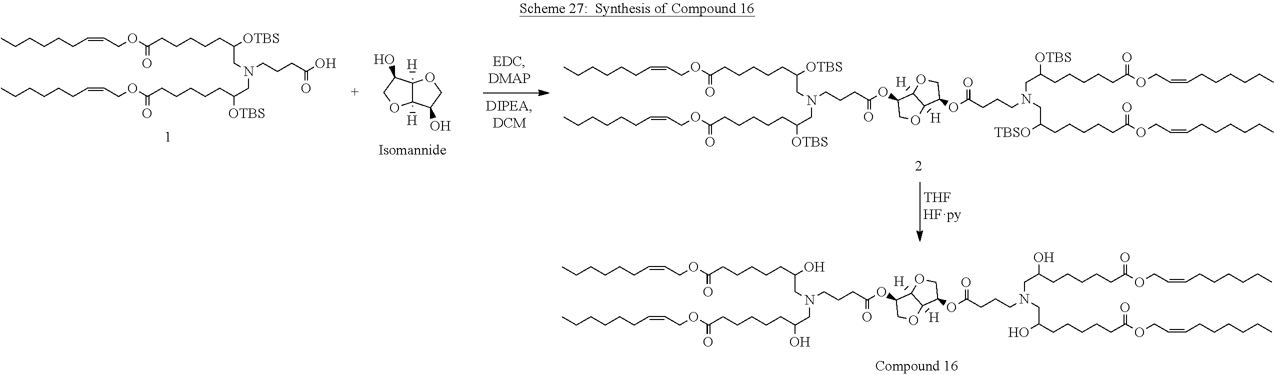

### Step 1: Synthesis of Intermediate (2)

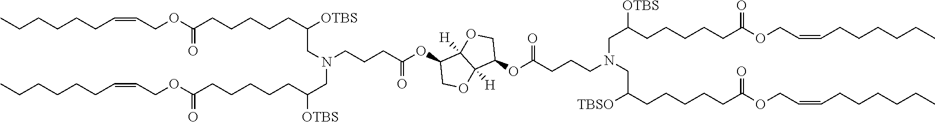 [SMILES: CCCCCC/C=C\COC(=O)CCCCCC(CN(CCCC(=O)O[C@@H]1CO[C@H]2[C@@H]1OC[C@H]2OC(=O)CCCN(CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)CC(CCCCCC(=O)OC/C=C\CCCCCC)O[Si](C)(C)C(C)(C)C)O[Si](C)(C)C(C)(C)C]

As depicted in Scheme 27: In a 20 mL vial was added isomannide (40.74 mg, 0.279 mmol), acid (1) (750 mg, 0.837 mmol) into 7.5 mL of DCM. To that solution was DMAP (34.22 mg, 0.279 mmol), DIPEA (0.389 mL, 2.231 mmol), and EDC (213.83 mg, 1,115 mmol) were added, and the resulting mixture was stirred at room temperature overnight. After 16 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (24 g silica column, 8% ethyl acetate-hexane gradient) to obtain Intermediate (2) (229 mg, 44%).

### Results

ESI-MS: Calculated C₁₀₆H₂₀₀N₂O₁₈Si₄, [M+H⁺]=1902.40, Observed=951.7 [M/2+H⁺].

### Step 2: Synthesis of Compound 16

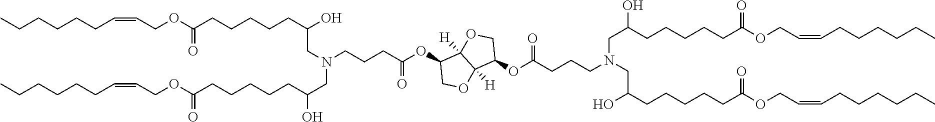 [SMILES: CCCCCC/C=C\COC(=O)CCCCCC(O)CN(CCCC(=O)O[C@@H]1CO[C@H]2[C@@H]1OC[C@H]2OC(=O)CCCN(CC(O)CCCCCC(=O)OC/C=C\CCCCCC)CC(O)CCCCCC(=O)OC/C=C\CCCCCC)CC(O)CCCCCC(=O)OC/C=C\CCCCCC]

As depicted in Scheme 27: To a solution of Intermediate (2) (229 mg, 0.12 mmol) in 1.5 mL of anhydrous tetrahydrofuran at 0° C. was added hydrogen fluoride pyridine (70% HF.py complex, 0.054 mL). The reaction mixture was warmed to room temperature and stirred for 16 h. MS analysis indicated completion of the reaction. The reaction mixtures were combined, and diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (12 g, ethyl acetate in hexane gradient) to obtain Compound 16 (95 mg, 55%).

### Results

ESI-MS: Calculated C₈₂H₁₄₄N₂O₁₈, [M+H⁺]=1446.05, Observed=724.3 [M/2+H⁺].

¹H NMR (400 MHz, CDCl₃) δ 5.79-5.27 (m, 8H), 5.27-4.96 (m, 2H), 4.71 (s, 2H), 4.62 (d, J=6.8 Hz, 4H), 4.54-3.60 (m, 14H), 3.58-2.90 (m, 6H), 2.70-2.43 (m, 2H), 2.44-2.14 (m, 8H), 2.13-2.01 (m, 2H), 1.89-1.47 (m, 24H), 1.40-1.06 (m, 56H), 0.89 (t, J=4.9 Hz, 12H).

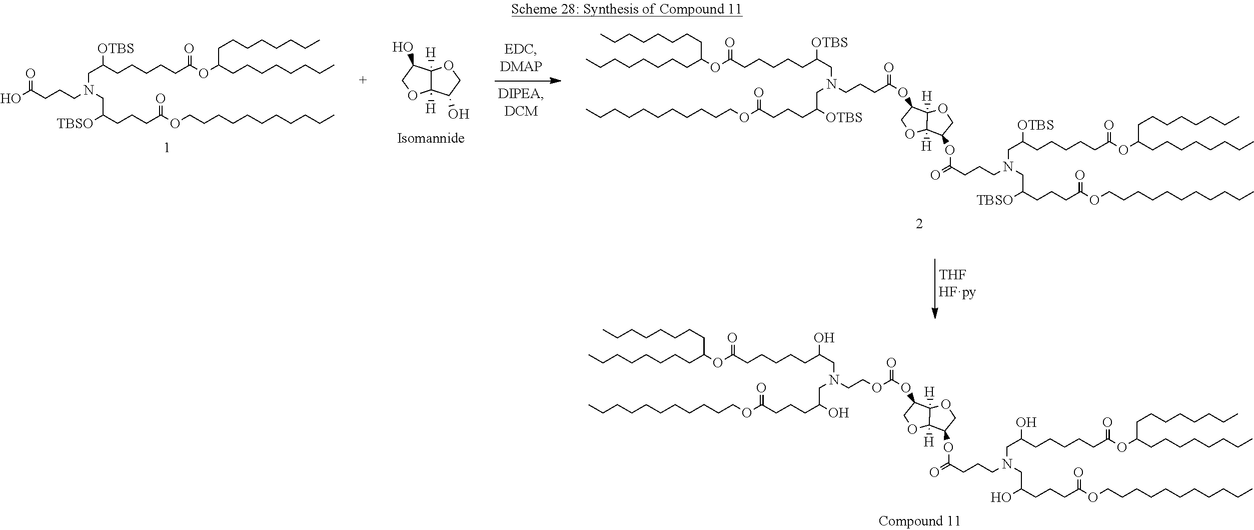

### Step 1: Synthesis of Intermediate (2)

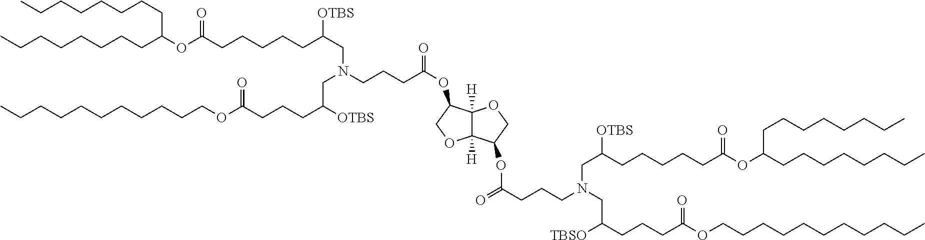 [SMILES: CCCCCCCCCCCOC(=O)CCCC(CN(CCCC(=O)O[C@@H]1CO[C@H]2[C@@H]1OC[C@H]2OC(=O)CCCN(CC(CCCCCC(=O)OC(CCCCCCCC)CCCCCCCC)O[Si](C)(C)C(C)(C)C)CC(CCCC(=O)OCCCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 28: In a 20 mL vial was added isomannide (36.07 mg, 0.2469 mmol), acid (1) (750 mg, 0.741 mmol) into 7.5 mL of DCM. To that solution was DMAP (30.289 mg, 0.2469 mmol), DIPEA (0.344 mL, 1.975 mmol), and EDC (189.29 mg, 0.9874 mmol) were added, and the resulting mixture was stirred at room temperature overnight. After 16 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (24 g silica column, 7% ethyl acetate-hexane gradient) to obtain Intermediate (2) (389 mg, 74%).

### Results

ESI-MS: Calculated C₁₂₂H₂₄₀N₂O₁₈Si₄, [M+H⁺]=2134.71, Observed=1068.0 [M/2+H⁺].

### Step 2: Synthesis of Compound 11

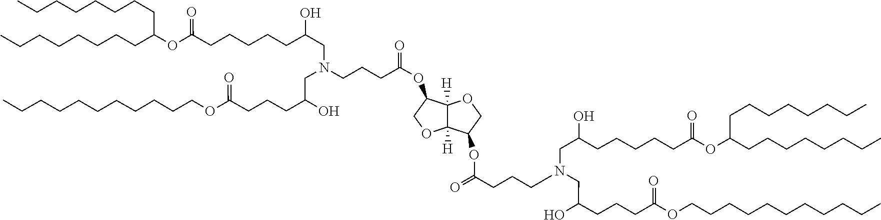 [SMILES: CCCCCCCCCCCOC(=O)CCCC(O)CN(CCCC(=O)O[C@@H]1CO[C@H]2[C@@H]1OC[C@H]2OC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC]

As depicted in Scheme 28: To a solution of Intermediate (2) (389.9 mg, 0.182 mmol) in 3 mL of anhydrous tetrahydrofuran at 0° C. was added hydrogen fluoride pyridine (70% HF.py complex, 0.082 mL). The reaction mixture was warmed to room temperature and stirred for 17 h. MS analysis indicated completion of the reaction. The reaction mixtures were combined, and diluted with ethyl acetate, quenched by slow addition of solid NaHCO₃ at 0° C., followed by saturated NaHCO₃ solution. The organic layer was washed with sat. NaHCO₃ solution, water and brine. Then dried over anhydrous Na₂SO₄ and concentrated. The crude was purified by flash chromatography on silica gel (12 g, ethyl acetate in hexane gradient) to obtain Compound 11 (157 mg, 51%).

### Results

ESI-MS: Calculated C₉₈H₁₈₄N₂O₁₈, [M+H⁺]=1678.36, Observed=1678.3 [M+H⁺], 839.3 [M/2+H⁺].

¹H NMR (400 MHz, CDCl₃) δ 5.16-4.99 (m, 2H), 4.94-4.79 (m, 2H), 4.77-4.66 (m, 2H), 4.31-4.20 (m, 2H), 4.04 (t, J=6.9 Hz, 8H), 3.20 (d, J=54.9 Hz, 10H), 2.71-2.48 (m, 4H), 2.47-2.08 (m, 24H), 1.91-1.51 (m, 20H), 1.51-1.03 (m, 88H), 0.87 (t, J=6.7 Hz, 18H).

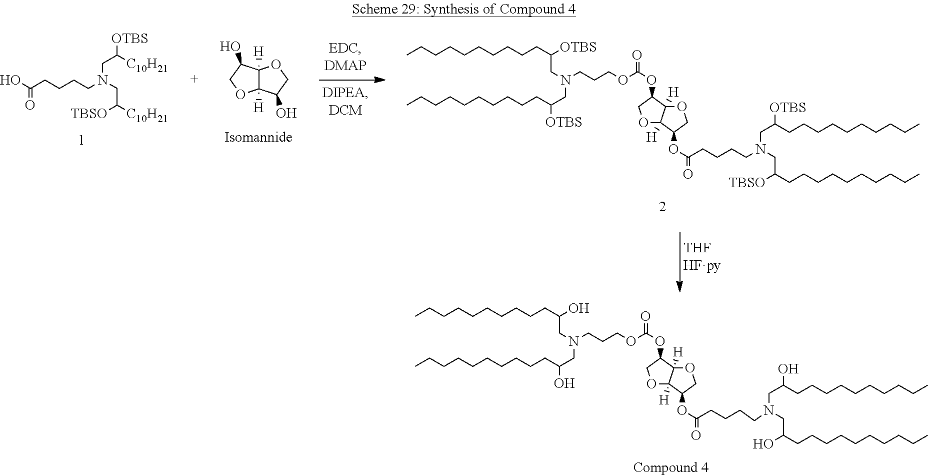

### Step 1: Synthesis of Intermediate (2)

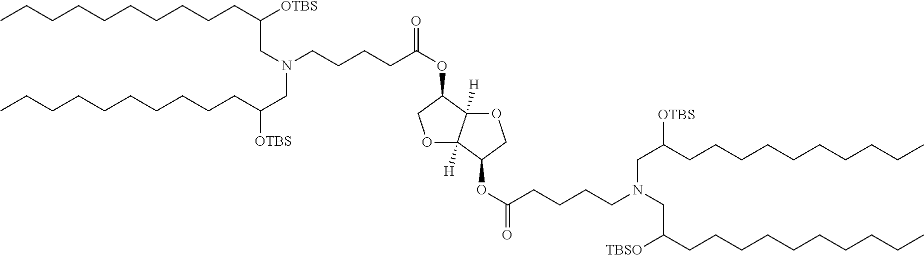 [SMILES: CCCCCCCCCCC(CN(CCCCC(=O)O[C@@H]1CO[C@H]2[C@@H]1OC[C@H]2OC(=O)CCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 29: To a solution of isomannide (51.2 mg, 0.350 mmol), acid (1) (750 mg, 1.05 mmol) in 7.5 mL of DCM was added DMAP (42.9 mg, 0.35 mmol), DIPEA (0.488 mL, 2.8 mmol) and EDC (268.4 mg, 1.4 mmol) were added, and the resulting mixture was stirred at room temperature overnight. After 22 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (24 g silica column, 4% ethyl acetate-hexane gradient) and obtain Intermediate (2) (344 mg, 64%).

### Results

ESI-MS: Calculated C₈₈H₁₈₀N₂O₁₀Si₄, [M+H⁺]=1538.28, Observed=769.7 [M/2+H⁺].

### Step 2: Synthesis of Compound 4

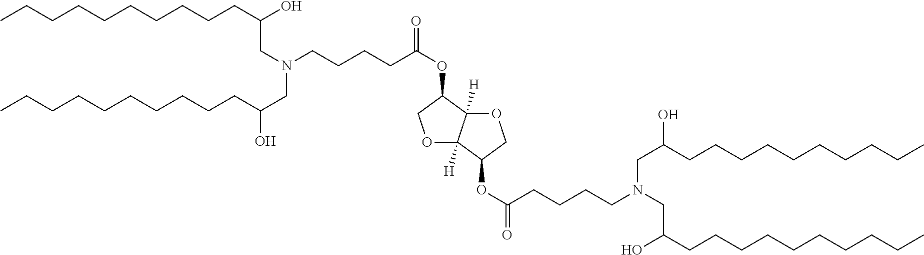 [SMILES: CCCCCCCCCCC(O)CN(CCCCC(=O)O[C@@H]1CO[C@H]2[C@@H]1OC[C@H]2OC(=O)CCCCN(CC(O)CCCCCCCCCC)CC(O)CCCCCCCCCC)CC(O)CCCCCCCCCC]

As depicted in Scheme 29: To a solution of Intermediate (2) (340.5 mg, 0.221 mmol) in 3 mL of anhydrous tetrahydrofuran at 0° C. was added hydrogen fluoride pyridine (70% HF.py complex, 1.5 mL). The reaction mixture was warmed to room temperature and stirred for 16 h. MS analysis indicated completion of the reaction. The reaction mixtures were combined, and diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (12 g, 38% ethyl acetate-hexane gradient) to obtain Compound 4 (124 mg, 52%).

### Results

ESI-MS: Calculated C₆₄H₁₂₄N₂O₁₀, [M+H⁺]=1081.94, Observed=1081.3 [M+H⁺], 541.4 [M/2+H⁺].

¹H NMR (400 MHz, CDCl₃) δ 5.20 (s, 4H), 4.97-4.42 (m, 2H), 4.33-4.17 (m, 2H), 4.10-3.82 (m, 4H), 3.36-3.19 (m, 4H), 3.15-3.04 (m, 4H), 2.59-2.22 (m, 12H), 2.14-1.75 (m, 4H), 1.78-1.45 (m, 12H), 1.43-0.98 (m, 64H), 0.88 (t, J=6.7 Hz, 12H).

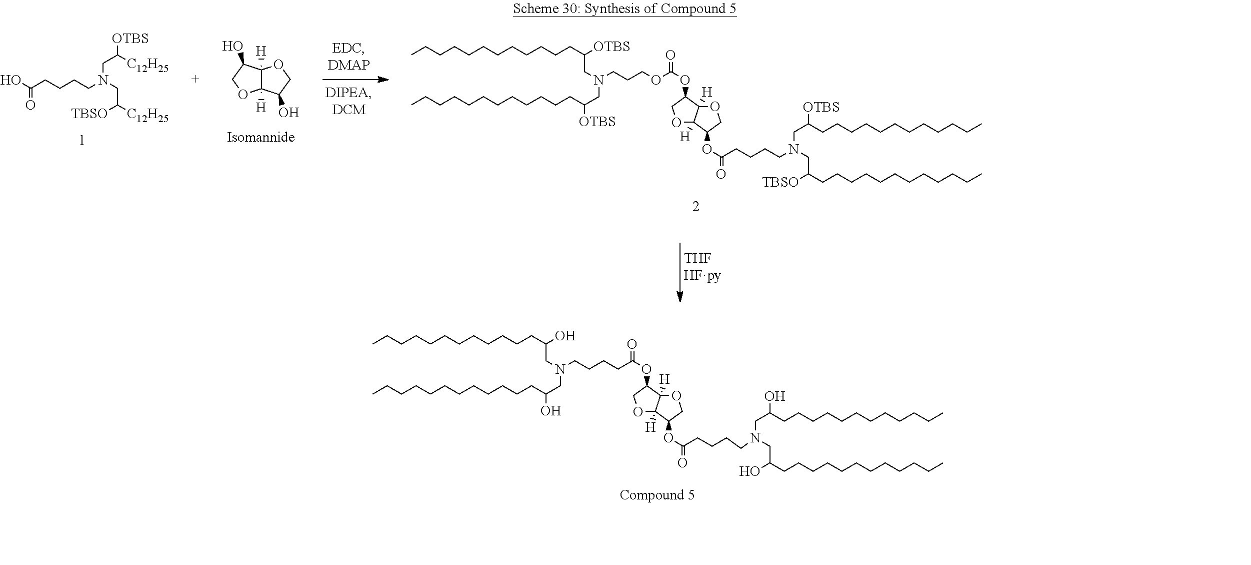

### Step 1: Synthesis of Intermediate (2)

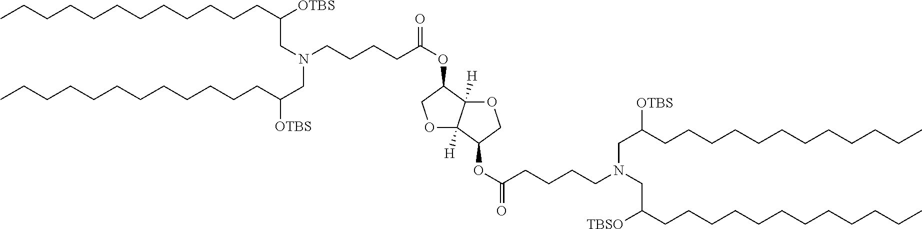 [SMILES: CCCCCCCCCCCCC(CN(CCCCC(=O)O[C@@H]1CO[C@H]2[C@@H]1OC[C@H]2OC(=O)CCCCN(CC(CCCCCCCCCCCC)O[Si](C)(C)C(C)(C)C)CC(CCCCCCCCCCCC)O[Si](C)(C)C(C)(C)C)CC(CCCCCCCCCCCC)O[Si](C)(C)C(C)(C)C)O[Si](C)(C)C(C)(C)C]

As depicted in Scheme 30: In a 20 mL vial was added isomannide (47.4 mg, 0.32 mmol), acid (1) (750 mg, 0.97 mmol) into 7.5 mL of DCM. To that solution was DMAP (39.8 mg, 0.32 mmol), DIPEA (0.45 mL, 2.6 mmol) and EDC (310 mg, 1.3 mmol), and the resulting mixture was stirred at room temperature overnight. After 22 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (24 g silica column, 4% ethyl acetate-hexane gradient) to obtain Intermediate (2) (352 mg, 66%).

### Results

ESI-MS: Calculated C₉₆H₁₉₈N₂O₁₀Si₄, [M+H⁺]=1650.41, Observed=825.3 [M/2+H⁺].

### Step 2: Synthesis of Compound 5

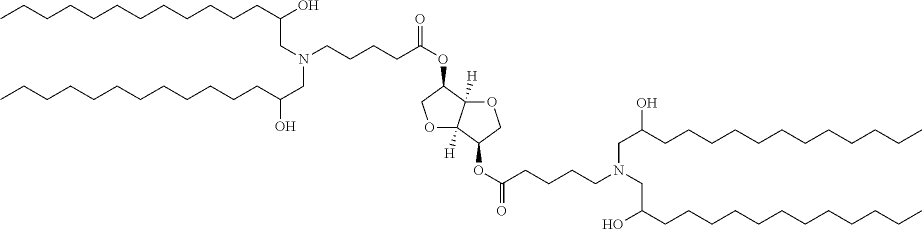 [SMILES: CCCCCCCCCCCCC(O)CN(CCCCC(=O)O[C@@H]1CO[C@H]2[C@@H]1OC[C@H]2OC(=O)CCCCN(CC(O)CCCCCCCCCCCC)CC(O)CCCCCCCCCCCC)CC(O)CCCCCCCCCCCC]

As depicted in Scheme 30: To a solution of Intermediate (2) (351.5 mg, 0.21 mmol) in 3 mL of anhydrous tetrahydrofuran at 0° C. was added hydrogen fluoride pyridine (70% HF.py complex, 0.13 mL, 1.49 mmol). The reaction mixture was warmed to room temperature and stirred for 20 h. MS analysis indicated completion of the reaction. The reaction mixtures were combined, and diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (12 g, 55% ethyl acetate-hexane gradient) to obtain Compound 5 (82 mg, 32%).

### Results

ESI-MS: Calculated C₇₂H₁₄₀N₂O₁₀, [M+H⁺]=1194.06, Observed=597.3 [M/2+H⁺].

¹H NMR (400 MHz, CDCl₃) δ 5.09 (s, 4H), 4.69 (m, 2H), 4.11-3.97 (m, 2H), 3.89-3.65 (m, 4H), 3.25-2.78 (m, 4H), 2.86-2.53 (m, 4H), 2.50-2.36 (m, 12H), 1.95-1.79 (m, 4H), 1.61-1.36 (m, 12H), 1.25 (m, 84H), 0.88 (t, J=6.7 Hz, 12H).

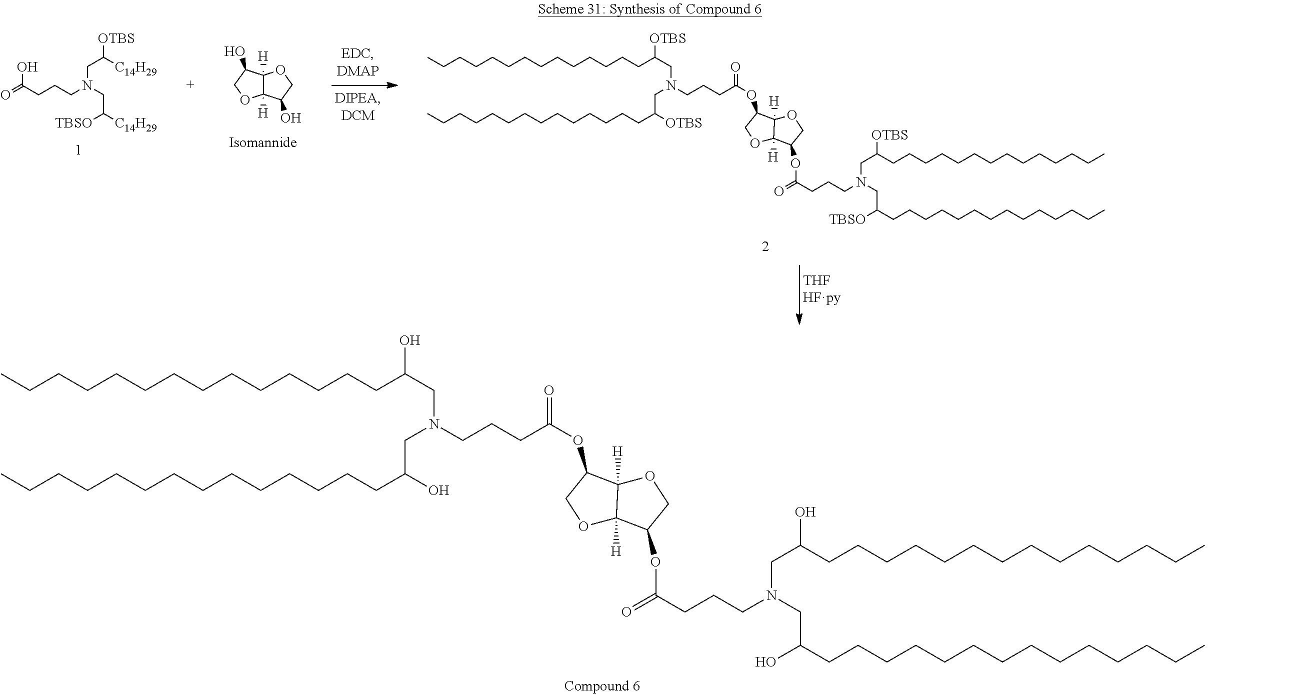

### Step 1: Synthesis of Intermediate (2)

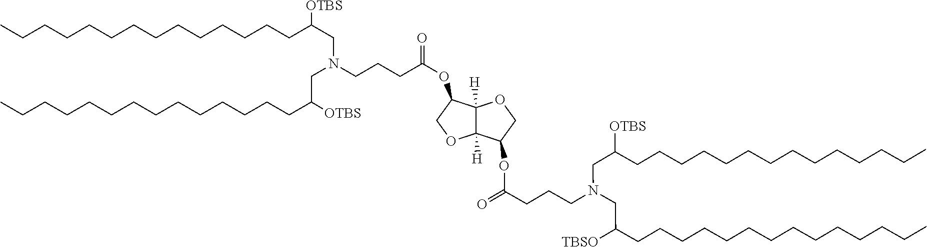 [SMILES: CCCCCCCCCCCCCCC(CN(CCCC(=O)O[C@@H]1CO[C@H]2[C@@H]1OC[C@H]2OC(=O)CCCN(CC(CCCCCCCCCCCCCC)O[Si](C)(C)C(C)(C)C)CC(CCCCCCCCCCCCCC)O[Si](C)(C)C(C)(C)C)CC(CCCCCCCCCCCCCC)O[Si](C)(C)C(C)(C)C)O[Si](C)(C)C(C)(C)C]

As depicted in Scheme 31: In a 20 mL vial was added isomannide (45 mg, 0.31 mmol), acid (1) (750 mg, 0.92 mmol) into 7.5 mL of DCM. To that solution was DMAP (56.6 mg, 0.46 mmol), DIPEA (0.64 mL, 3.69 mmol) and EDC (235.9 mg, 1.23 mmol) were added, and the resulting mixture was stirred at room temperature overnight. After 20 hr, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (24 g silica column, 5% ethyl acetate-hexane gradient) to obtain Intermediate (2) (230 mg, 44%).

### Results

ESI-MS: Calculated C₁₀₂H₂₀₈N₂O₁₀Si₄, [M+H⁺]=1734.50, Observed=868.4 [M/2+H⁺].

### Step 2: Synthesis of Compound 6

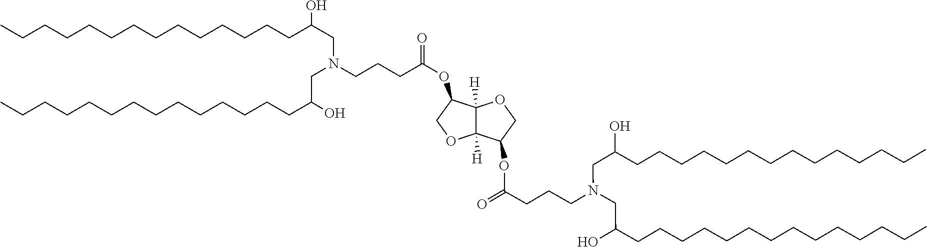 [SMILES: CCCCCCCCCCCCCCC(O)CN(CCCC(=O)O[C@@H]1CO[C@H]2[C@@H]1OC[C@H]2OC(=O)CCCN(CC(O)CCCCCCCCCCCCCC)CC(O)CCCCCCCCCCCCCC)CC(O)CCCCCCCCCCCCCC]

As depicted in Scheme 31: To a solution of Intermediate (2) (230 mg, 0.13 mmol) in 2 mL of anhydrous tetrahydrofuran at 0° C. was added hydrogen fluoride pyridine (70% HF.py complex, 0.5 mL). The reaction mixture was warmed to room temperature and stirred for 16 hr. MS analysis indicated completion of the reaction. Reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (12 g, 39% ethyl acetate-hexane gradient) to obtain Compound 6 (113 mg, 67%).

### Results

ESI-MS: Calculated C₇₈H₁₅₂N₂O₁, [M+H⁺]=1278.15, Observed=639.5 [M/2+H⁺].

¹H NMR (400 MHz, CDCl₃) δ 5.14-5.04 (m, 2H), 4.76-4.62 (m, 2H), 4.12-3.68 (m, 8H), 3.11-2.61 (m, 12H), 2.57-2.33 (m, 4H), 2.02 (d, J=14.0 Hz, 4H), 1.64-1.36 (m, 12H), 1.46-0.99 (m, 96H), 0.85 (d, J=6.7 Hz, 12H).

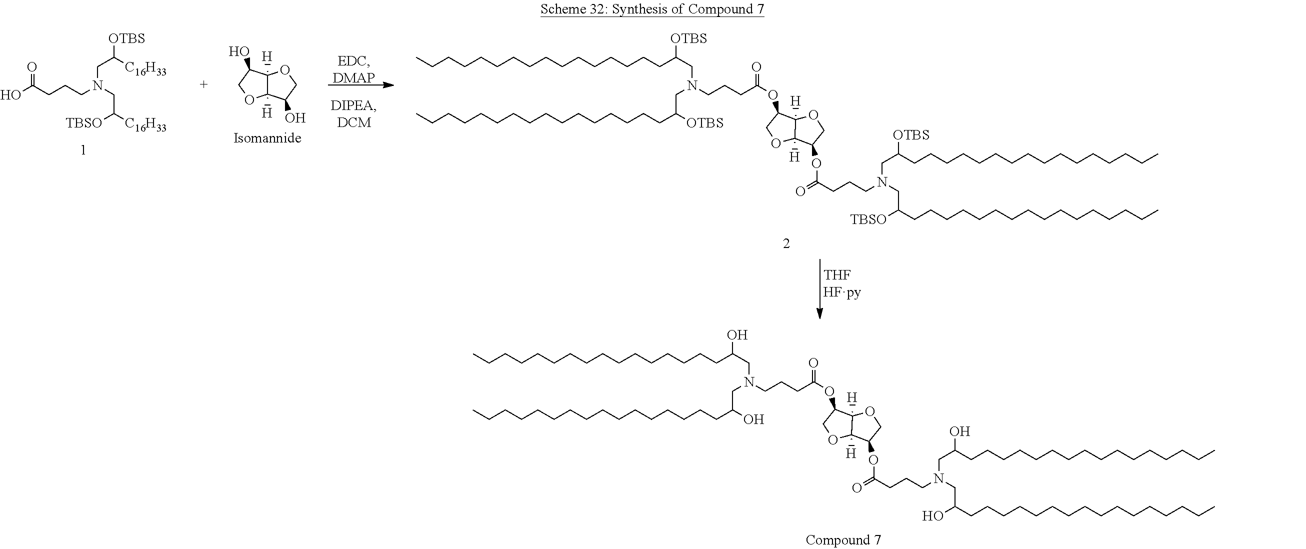

### Step 1: Synthesis of Intermediate (2)

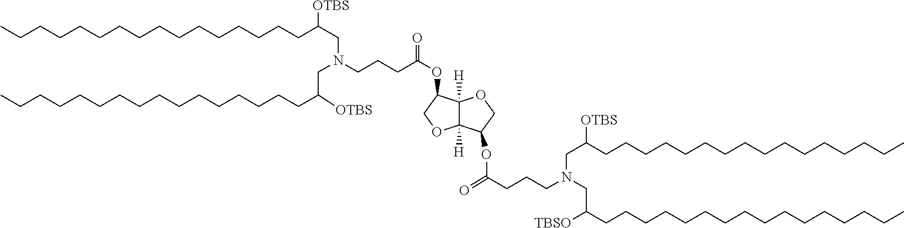 [SMILES: CCCCCCCCCCCCCCCCC(CN(CCCC(=O)O[C@@H]1CO[C@H]2[C@@H]1OC[C@H]2OC(=O)CCCN(CC(CCCCCCCCCCCCCCCC)O[Si](C)(C)C(C)(C)C)CC(CCCCCCCCCCCCCCCC)O[Si](C)(C)C(C)(C)C)CC(CCCCCCCCCCCCCCCC)O[Si](C)(C)C(C)(C)C)O[Si](C)(C)C(C)(C)C]

As depicted in Scheme 32: In a 20 mL vial was added isomannide (42.1 mg, 0.29 mmol), acid (1) (750 mg, 0.86 mmol) into 7.5 mL of DCM. To that solution was DMAP (35.3 mg, 0.29 mmol), DIPEA (0.4 mL, 2.3 mmol) and EDC (220.7 mg, 1.15 mmol) were added, and the resulting mixture was stirred at room temperature overnight. After 16 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (24 g silica column, 2% ethyl acetate-hexane gradient) to obtain Intermediate (2) (457 mg, 86%).

### Results

ESI-MS: Calculated C₁₁₀H₂₂₄N₂O₁₀Si₄, [M+H⁺]=1846.63, Observed=924.3 [M/2+H⁺].

### Step 2: Synthesis of Compound 7

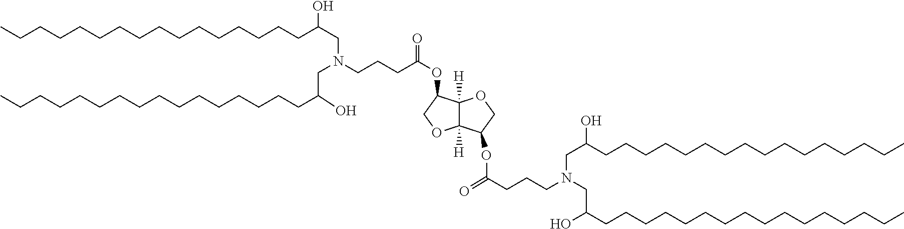 [SMILES: CCCCCCCCCCCCCCCCC(O)CN(CCCC(=O)O[C@@H]1CO[C@H]2[C@@H]1OC[C@H]2OC(=O)CCCN(CC(O)CCCCCCCCCCCCCCCC)CC(O)CCCCCCCCCCCCCCCC)CC(O)CCCCCCCCCCCCCCCC]

As depicted in Scheme 32: To a solution of Intermediate (2) (457 mg, 0.25 mmol) in 3 mL of anhydrous tetrahydrofuran at 0° C. was added hydrogen fluoride pyridine (70% HF.py complex, 1 mL). The reaction mixture was warmed to room temperature and stirred for 16 h. MS analysis indicated completion of the reaction. The reaction mixtures were combined, and diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (12 g, 85% ethyl acetate-hexane gradient) to obtain Compound 7 (186 mg, 54%).

### Results

ESI-MS: Calculated C₈₆H₁₆₈N₂O₁₀, [M+H⁺]=1390.28, Observed=695.3 [M/2+H⁺].

¹H NMR (400 MHz, CDCl₃) δ 5.15-4.96 (m, 2H), 4.80-4.61 (m, 2H), 4.24-3.67 (m, 8H), 3.35-2.56 (m, 12H), 2.59-2.34 (m, 4H), 2.24-1.82 (m, 4H), 1.65-1.35 (m, 12H), 1.67-1.00 (m, 112H), 0.88 (t, J=6.6 Hz, 12H).

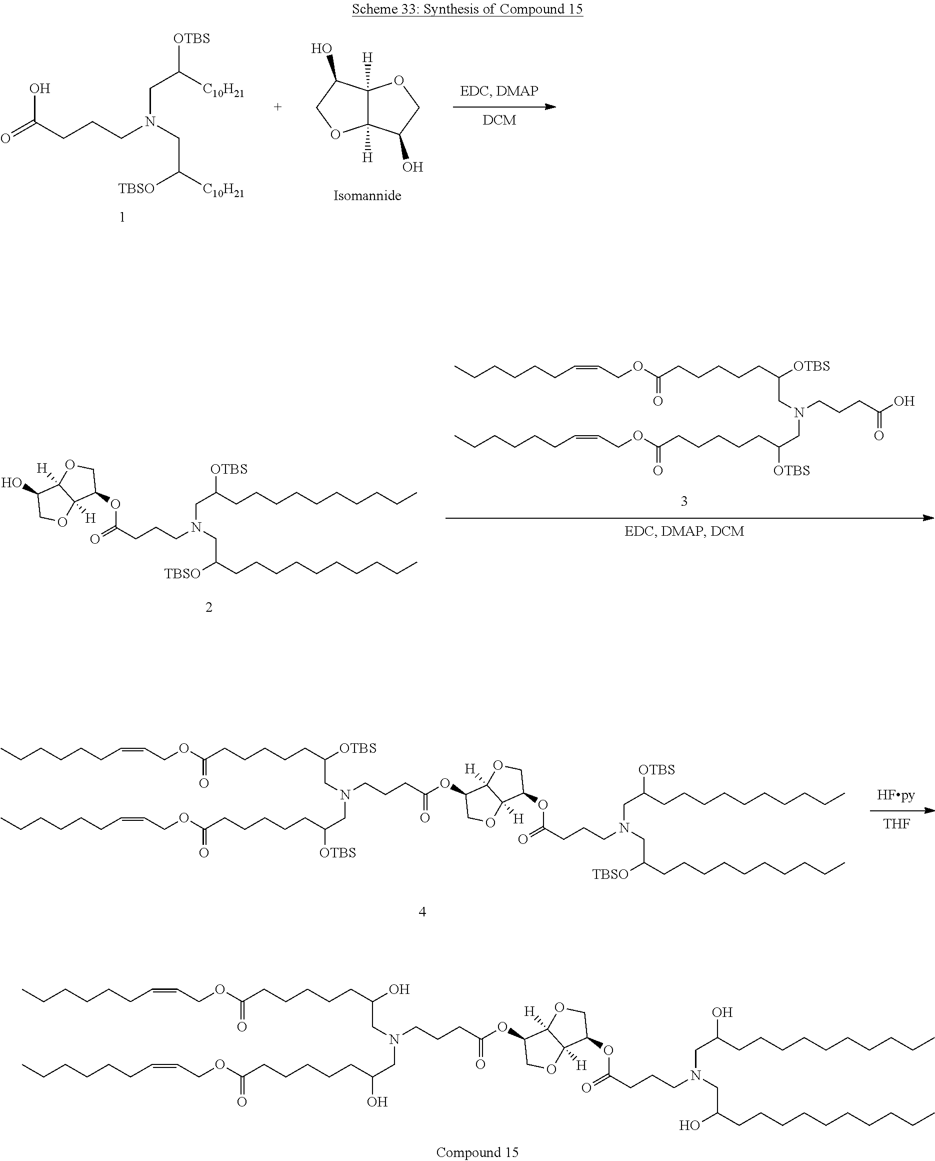

### Step 1: Synthesis of Intermediate (2)

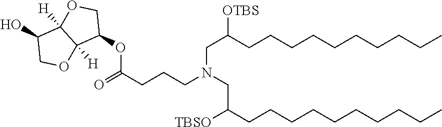 [SMILES: CCCCCCCCCCC(CN(CCCC(=O)O[C@@H]1CO[C@H]2[C@@H]1OC[C@H]2O)CC(CCCCCCCCCC)O[Si](C)(C)C(C)(C)C)O[Si](C)(C)C(C)(C)C]

As depicted in Scheme 33: In a 20 mL vial was added isomannide (142.3 mg, 0.97 mmol), acid (1) (750 mg, 1.07 mmol) into 7.5 mL of DCM. To that solution was DMAP (119.5 mg, 0.97 mmol) and EDC (242.6 mg, 1.27 mmol) were added, and the resulting mixture was stirred at room temperature overnight. After 16 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue (881.5 mg) was relatively pure and used for the next step without purification (esterification with acid (3)).

### Results

ESI-MS: Calculated C₄₆H₉₃NO₇Si₂, [M+H⁺]=828.66, Observed=828.6 [M+H⁺].

### Step 2: Synthesis of Intermediate (4)

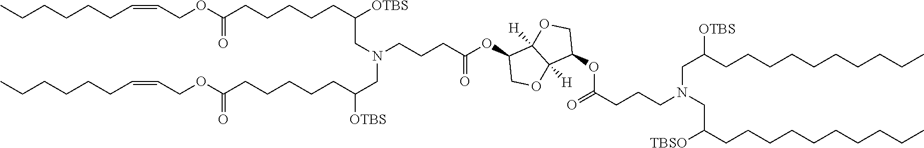 [SMILES: CCCCCC/C=C\COC(=O)CCCCCC(CN(CCCC(=O)O[C@@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCC(=O)OC/C=C\CCCCCC)O[Si](C)(C)C(C)(C)C)O[Si](C)(C)C(C)(C)C]

As depicted in Scheme 33: In a 20 mL vial was added Intermediate (2) (881.5 mg, 1.06 mmol), acid (3) (1049.3 mg, 1.17 mmol) into 9 mL of DCM. To that solution was DMAP (130.6 mg, 1.06 mmol) and EDC (265.2 mg, 1.38 mmol) were added, and the resulting mixture was stirred at room temperature overnight. After 16 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (40 g silica column, 4% ethyl acetate-hexane gradient) to obtain Intermediate (4) (652 mg, 36%).

### Results

ESI-MS: Calculated C₉₆H₁₈₈N₂O₁₄Si₄, [M+H⁺]=1706.32, Observed=854.0 [M/2+H⁺].

### Step 3: Synthesis of Compound 15

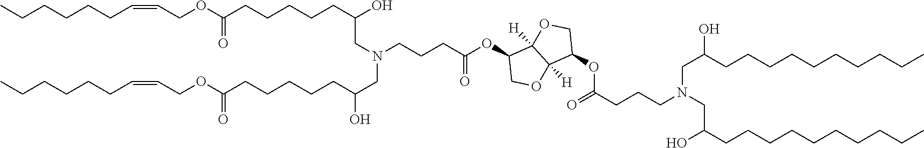 [SMILES: CCCCCC/C=C\COC(=O)CCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCC(=O)OC/C=C\CCCCCC]

As depicted in Scheme 33: To a solution of Intermediate (4) (652 mg, 0.38 mmol) in 4 mL of anhydrous tetrahydrofuran at 0° C. was added hydrogen fluoride pyridine (70% HF.py complex, 0.17 mL). The reaction mixture was warmed to room temperature and stirred for 20 h. MS analysis indicated completion of the reaction. Reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (12 g, 78% ethyl acetate-hexane gradient) to obtain Compound 15 (332 mg, 70%).

### Results

ESI-MS: Calculated C₇₂H₁₃₂N₂O₁₄, [M+H⁺]=1249.98, Observed=1249.8 [M+H⁺], 625.6 [M/2+H⁺].

¹H NMR (400 MHz, CDCl₃) δ 5.68-5.46 (m, 4H), 5.13-5.05 (m, 2H), 4.75-4.65 (m, 2H), 4.61 (d, J=6.8 Hz, 4H), 3.91 (dt, J=89.7, 8.6 Hz, 4H), 3.64 (s, 4H), 2.72-2.50 (m, 6H), 2.53-2.36 (m, 10H), 2.31 (t, J=7.5 Hz, 6H), 2.09 (q, J=7.3 Hz, 4H), 1.87-1.78 (m, 4H), 1.63 (p, J=7.5 Hz, 4H), 1.51-1.36 (m, 12H), 1.36-1.07 (m, 54H), 0.88 (t, J=6.6 Hz, 12H).

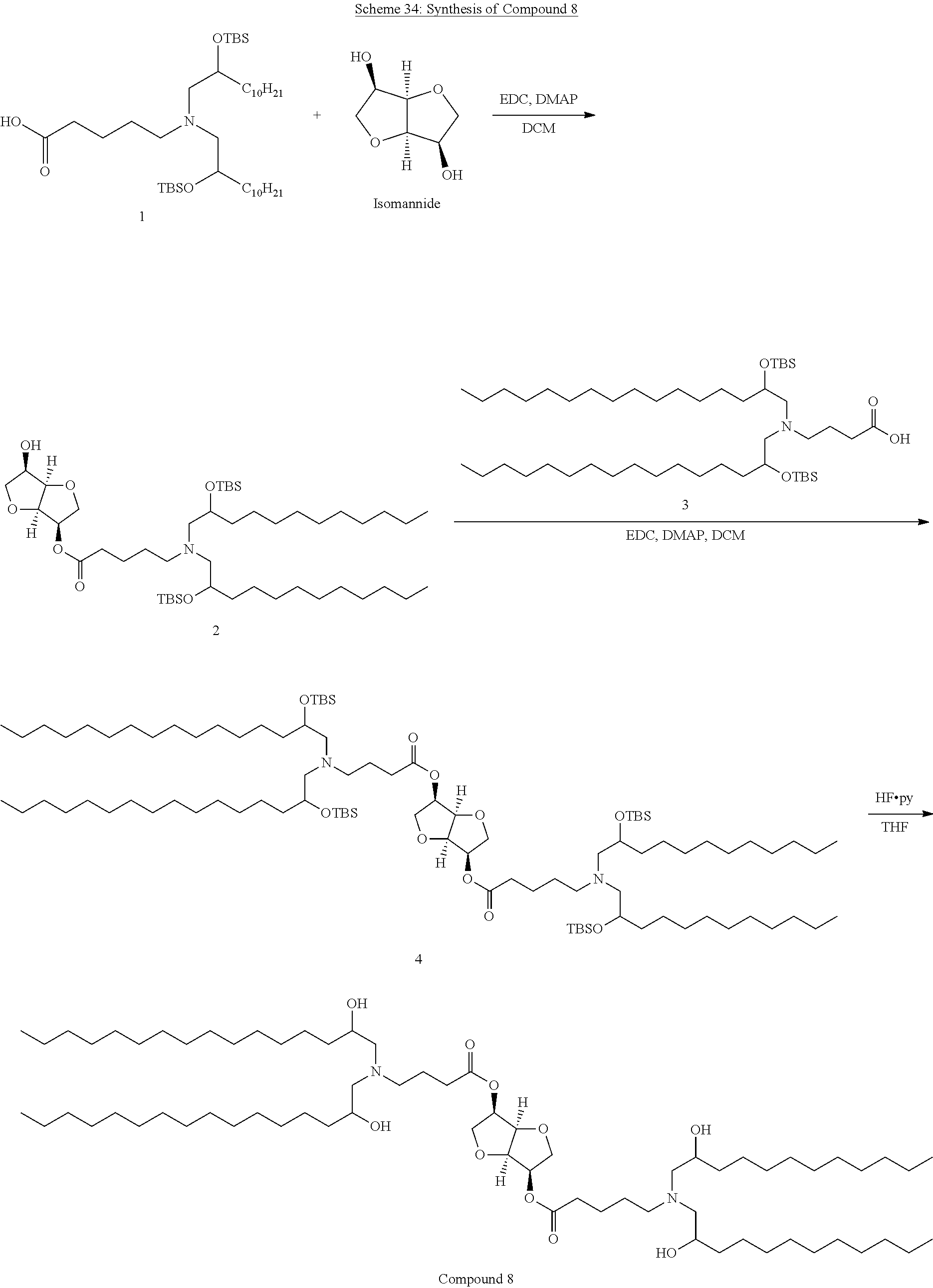

### Step 1: Synthesis of Intermediate (2)

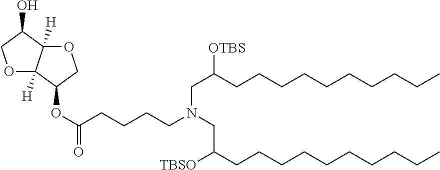 [SMILES: CCCCCCCCCCC(CN(CCCCC(=O)O[C@@H]1CO[C@H]2[C@@H]1OC[C@H]2O)CC(CCCCCCCCCC)O[Si](C)(C)C(C)(C)C)O[Si](C)(C)C(C)(C)C]

As depicted in Scheme 34: In a 20 mL vial was added isomannide (139.5 mg, 0.95 mmol), acid (1) (750 mg, 1.05 mmol) into 7.5 mL of DCM. To that solution was DMAP (117.1 mg, 0.95 mmol) and EDC (237.9 mg, 1.24 mmol) were added, and the resulting mixture was stirred at room temperature overnight. After 18 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue (884.9 mg) was relatively pure and used for the next step without purification (esterification with acid (3)).

### Results

ESI-MS: Calculated C₄₇H₉₅NO₇Si₂, [M+H⁺]=842.67, Observed=842.6 [M+H⁺].

### Step 2: Synthesis of Intermediate (4)

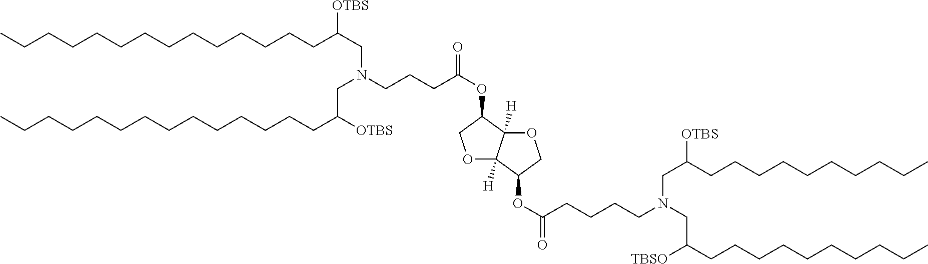 [SMILES: CCCCCCCCCCCCCCC(CN(CCCC(=O)O[C@@H]1CO[C@H]2[C@@H]1OC[C@H]2OC(=O)CCCCN(CC(CCCCCCCCCC)O[Si](C)(C)C(C)(C)C)CC(CCCCCCCCCC)O[Si](C)(C)C(C)(C)C)CC(CCCCCCCCCCCCCC)O[Si](C)(C)C(C)(C)C)O[Si](C)(C)C(C)(C)C]

As depicted in Scheme 34: To a solution of Intermediate (2) (884.9 mg, 1.05 mmol) and acid (3) (949.2 mg, 1.15 mmol) in 9 mL of DCM was added DMAP (128.9 mg, 1.05 mmol) and EDC (261.8 mg, 1.37 mmol), and the resulting mixture was stirred at room temperature overnight. After 18 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (40 g silica column, 4% ethyl acetate-hexane gradient) to obtain Intermediate (4) (900 mg, 52%).

### Results

ESI-MS: Calculated C₉₅H₁₉₄N₂O₁₀Si₄, [M+H⁺]=1636.39, Observed=818.9 [M/2+H⁺].

### Step 3: Synthesis of Compound 8

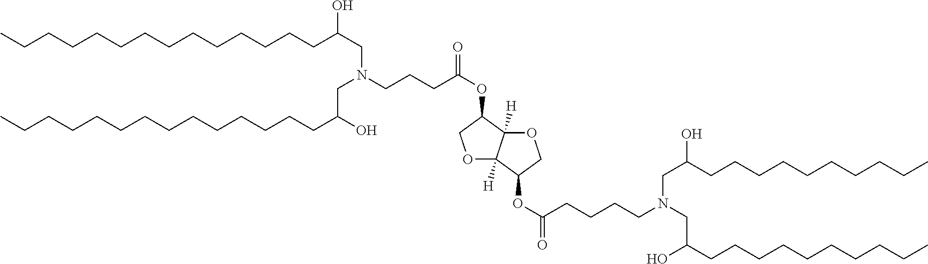 [SMILES: CCCCCCCCCCCCCCC(O)CN(CCCC(=O)O[C@@H]1CO[C@H]2[C@@H]1OC[C@H]2OC(=O)CCCCN(CC(O)CCCCCCCCCC)CC(O)CCCCCCCCCC)CC(O)CCCCCCCCCCCCCC]

As depicted in Scheme 34: To a solution of Intermediate (4) (900 mg, 0.55 mmol) in 4 mL of anhydrous tetrahydrofuran at 0° C. was added hydrogen fluoride pyridine (70% HF.py complex, 1 mL). The reaction mixture was warmed to room temperature and stirred for 18 h. MS analysis indicated completion of the reaction. Reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (24 g, 75% ethyl acetate-hexane gradient) to obtain Compound 8 (105 mg, 16%).

### Results

ESI-MS: Calculated C₇₁H₁₃₈N₂O₁₀Si₄, [M+H⁺]=1179.05, Observed=1179.9 [M+H⁺], 590.7 [M/2+H⁺].

¹H NMR (400 MHz, CDCl₃) δ 5.21-5.01 (m, 2H), 4.75-4.65 (m, 2H), 3.91 (dd, J=91.9, 8.4 Hz, 4H), 3.68-3.61 (m, 2H), 2.76-2.50 (m, 6H), 2.51-2.11 (m, 10H), 1.95-1.50 (m, 4H), 1.48-1.32 (m, 12H), 1.25 (s, 80H), 0.87 (t, J=7.1 Hz, 12H).

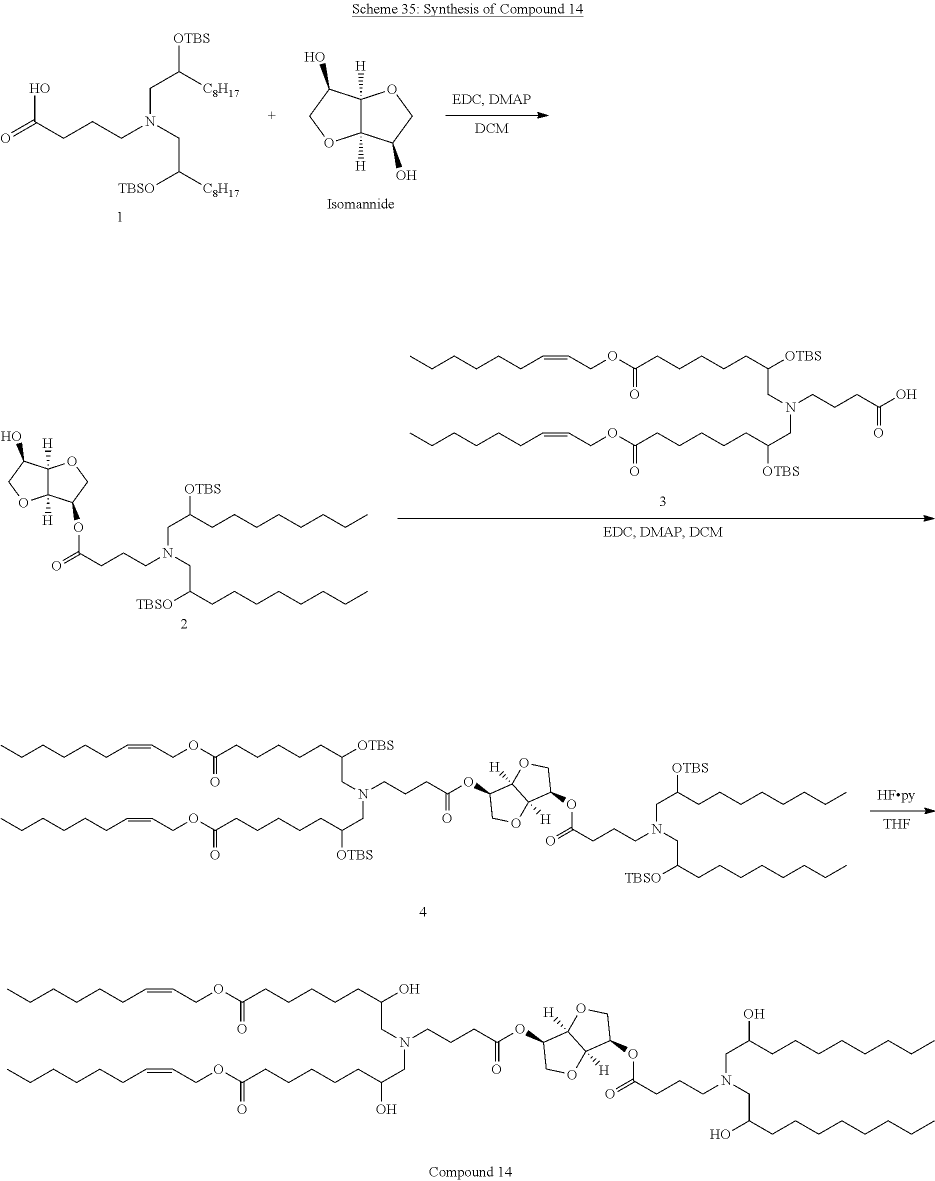

### Step 1: Synthesis of Intermediate (2)

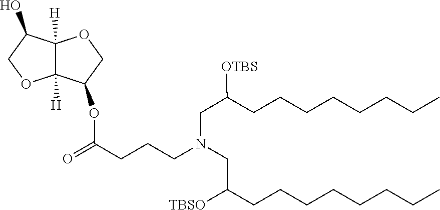 [SMILES: CCCCCCCCC(CN(CCCC(=O)O[C@@H]1CO[C@H]2[C@@H]1OC[C@H]2O)CC(CCCCCCCC)O[Si](C)(C)C(C)(C)C)O[Si](C)(C)C(C)(C)C]

As depicted in Scheme 35: In a 20 mL vial was added isomannide (154.7 mg, 1.06 mmol), acid (1) (750 mg, 1.16 mmol) into 7.5 mL of DCM. To that solution was DMAP (129.9 mg, 1.06 mmol) and EDC (263.8 mg, 1.38 mmol) were added, and the resulting mixture was stirred at room temperature overnight. After 18 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue (713.9 mg) was relatively pure and used for the next step without purification (esterification with acid (3)).

### Results

ESI-MS: Calculated C₄₂H₈₅NO₇Si₂, [M+H⁺]=772.60, Observed=772.5 [M+H⁺].

### Step 2: Synthesis of Intermediate (4)

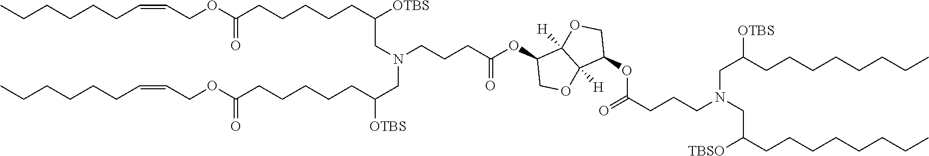 [SMILES: CCCCCC/C=C\COC(=O)CCCCCC(CN(CCCC(=O)O[C@@H]1CO[C@H]2[C@@H]1OC[C@H]2OC(=O)CCCN(CC(CCCCCCCC)O[Si](C)(C)C(C)(C)C)CC(CCCCCCCC)O[Si](C)(C)C(C)(C)C)CC(CCCCCC(=O)OC/C=C\CCCCCC)O[Si](C)(C)C(C)(C)C)O[Si](C)(C)C(C)(C)C]

As depicted in Scheme 35: In a 20 mL vial was added 2 (713.9 mg, 0.92 mmol), acid (3) (818 mg, 0.91 mmol) into 7 mL of DCM. To that solution was DMAP (113.4 mg, 0.92 mmol) and EDC (230.4 mg, 1.20 mmol) were added, and the resulting mixture was stirred at room temperature overnight. After 18 h, MS analysis indicated completion of the reaction. Then reaction mixture was diluted with DCM and washed sat. NaHCO₃ solution, water, and brine solution. The organic layer was dried over anhydrous Na₂SO₄ and concentrated. The crude residue was purified (40 g silica column, 6% ethyl acetate-hexane gradient) to obtain Intermediate (4)(470 mg, 31%).

### Results

ESI-MS: Calculated C₉₂H₁₈₀N₂O₁₄Si₄, [M+H⁺]=1650.26, Observed=826.1 [M/2+H⁺].

### Step 3: Synthesis of Compound 14

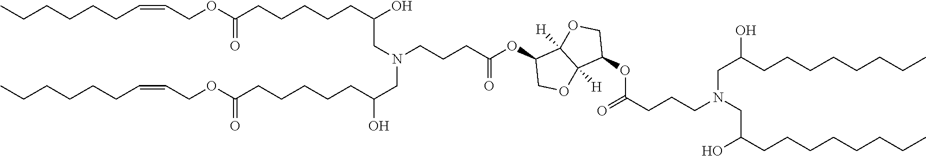 [SMILES: CCCCCC/C=C\COC(=O)CCCCCC(O)CN(CCCC(=O)O[C@@H]1CO[C@H]2[C@@H]1OC[C@H]2OC(=O)CCCN(CC(O)CCCCCCCC)CC(O)CCCCCCCC)CC(O)CCCCCC(=O)OC/C=C\CCCCCC]

As depicted in Scheme 35: To a solution of Intermediate (4) (470 mg, 0.27 mmol) in 3 mL of anhydrous tetrahydrofuran at 0° C. was added hydrogen fluoride pyridine (70% HF.py complex, 1 mL). The reaction mixture was warmed to room temperature and stirred for 18 h. MS analysis indicated completion of the reaction. Reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na₂SO₄ and concentrated. The crude was purified by flash chromatography on silica gel (12 g, 88% ethyl acetate-hexane gradient) to obtain Compound 14 (291 mg, 89%).

### Results

ESI-MS: Calculated C₆₈H₁₂₄N₂O₁₄Si₄, [M+H⁺]=1193.92, Observed=1193.8 [M+H⁺], 597.5 [M/2+H⁺].

¹H NMR (400 MHz, CDCl₃) δ 5.68-5.46 (m, 4H), 5.13-5.05 (m, 2H), 4.75-4.65 (m, 2H), 4.61 (d, J=6.8 Hz, 4H), 3.91 (dt, J=89.7, 8.6 Hz, 4H), 3.64 (s, 4H), 2.72-2.50 (m, 6H), 2.53-2.36 (m, 10H), 2.31 (t, J=7.5 Hz, 6H), 2.09 (q, J=7.3 Hz, 4H), 1.87-1.78 (m, 4H), 1.63 (p, J=7.5 Hz, 4H), 1.51-1.36 (m, 12H), 1.36-1.07 (m, 46H), 0.88 (t, J=6.6 Hz, 12H).

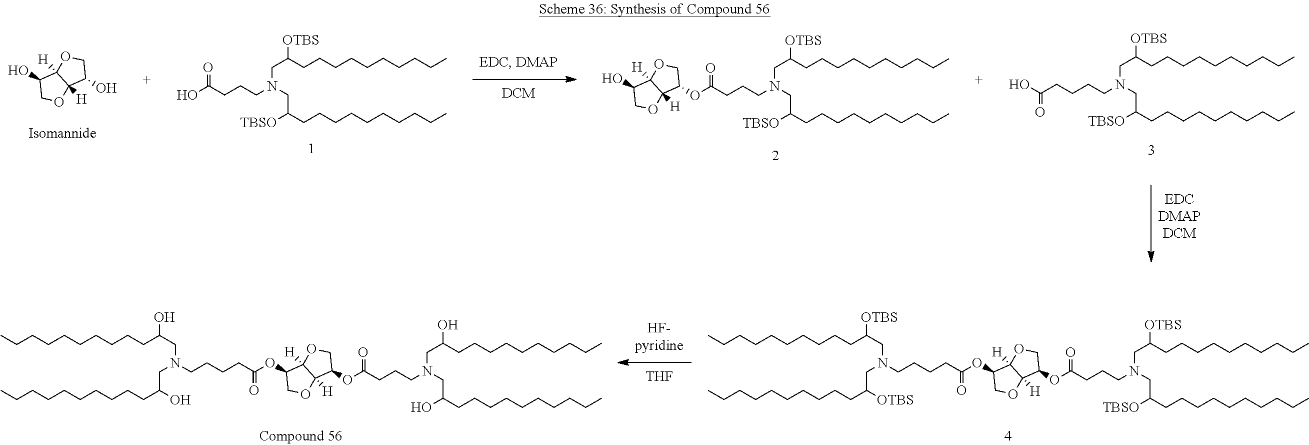

### Step 1: Synthesis of Intermediate (2)

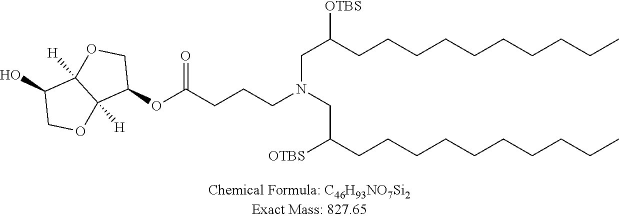 [SMILES: CCCCCCCCCCC(CN(CCCC(=O)O[C@@H]1CO[C@H]2[C@@H]1OC[C@H]2O)CC(CCCCCCCCCC)O[Si](C)(C)C(C)(C)C)O[Si](C)(C)C(C)(C)C]

As depicted in Scheme 36: To a solution of intermediate (1) (1.500 g, 2.142 mmol) in anhydrous DCM (15 mL), Isomannide (344 mg, 2.35 mmol) was added. DMAP (263 mg, 2.14 mmol) and EDC (534 mg, 2.79 mmol) were then added. The reaction mixture was stirred at room temperature overnight. MS analysis showed the presence of monoester. The reaction mixture was diluted with DCM and washed with a saturated NaHCO₃ solution, water, then brine solution. Compound extracted from aqueous layers at each step of the work up. Organic layer dried with anhydrous Na2SO4 and concentrated. Crude residue purified using a hexanes: ethyl acetate solvent system to obtain intermediate (2) (839 mg, 47% yield).

### Results:

ESI-MS: Calculated C46H93NO7Si2, [M+H+]=828.66, Observed=828.5

### Step 2: Synthesis of Intermediate (4)

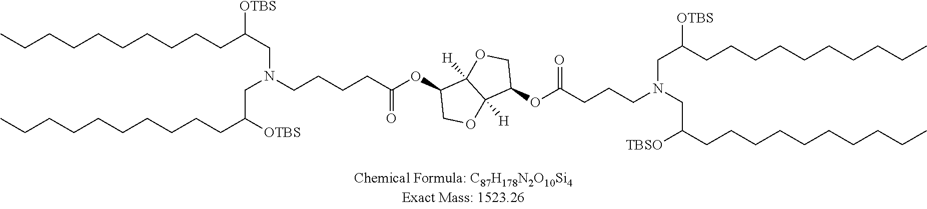 [SMILES: CCCCCCCCCCC(CN(CCCCC(=O)O[C@@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 36: To a solution of intermediate (3) (797 mg, 1.12 mmol) in anhydrous DCM (6.0 mL), intermediate (2) (839 mg, 1.01 mmol) was added. DMAP (125 mg, 1.02 mmol) and EDC (252 mg, 1.32 mmol) were then added. The reaction mixture was stirred at room temperature overnight. MS analysis showed the presence of the expected product. The reaction mixture was diluted with DCM and washed with a saturated NaHCO₃ solution, water, then brine. Compound extracted from aqueous layers at each step of the work up. Organic layer dried with anhydrous Na2SO4 and concentrated. The crude residue was purified using an ethyl acetate: hexanes solvent system to obtain intermediate (4) (1.070 g, 69% yield).

### Results:

ESI-MS: Calculated C87H178N2O10Si4, [M+H+]=1524.26, Observed=763.0 [M/2+H+]

### Step 3: Synthesis of Compound 56

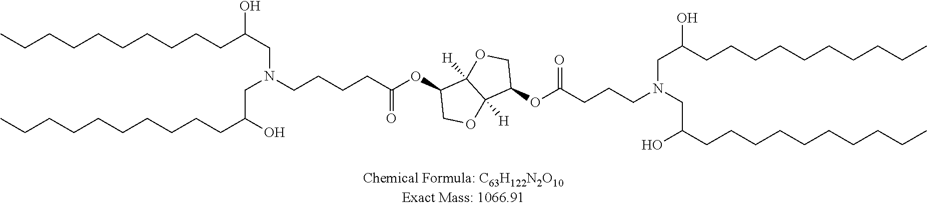 [SMILES: CCCCCCCCCCC(O)CN(CCCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

As depicted in Scheme 36: To a solution of intermediate (4) (1.070 g, 0.7018 mmol) in anhydrous THF (4.0 mL) at 0° C. was added HF-pyridine (1.8 mL, 14 mmol, 70 mass %). The reaction mixture was warmed to room temperature and stirred overnight. MS analysis indicated that the intended product had been produced. The reaction mixture was diluted with ethyl acetate and quenched by slow addition of solid NaHCO₃ followed by saturated NaHCO₃ solution. Reaction quenching conducted at 0° C. Next, the organic layer was washed with a saturated NaHCO₃ solution, then water and brine. Organic layer dried over anhydrous Na2SO4 and concentrated. Crude residue purified using a 12 g silica column and a hexanes: ethyl acetate solvent system. The purest fractions were collected and concentrated to obtain compound 56 (411 mg, 55% yield).

### Results:

1H NMR (400 MHz, CDCl3) δ 5.14-5.05 (m, 2H), 4.74-4.65 (m, 2H), 4.07-3.99 (m, 2H), 3.84-3.75 (m, 2H), 3.69-3.56 (m, 4H), 2.63-2.53 (m, 4H), 2.51-2.33 (m, 12H), 1.86-1.77 (m, 2H), 1.60-1.51 (m, 2H), 1.51-1.39 (m, 8H), 1.39-1.20 (m, 66H), 0.88 (t, 12H).

ESI-MS: Calculated C63H122N2O10, [M+H+]=1067.92, Observed=1067.8

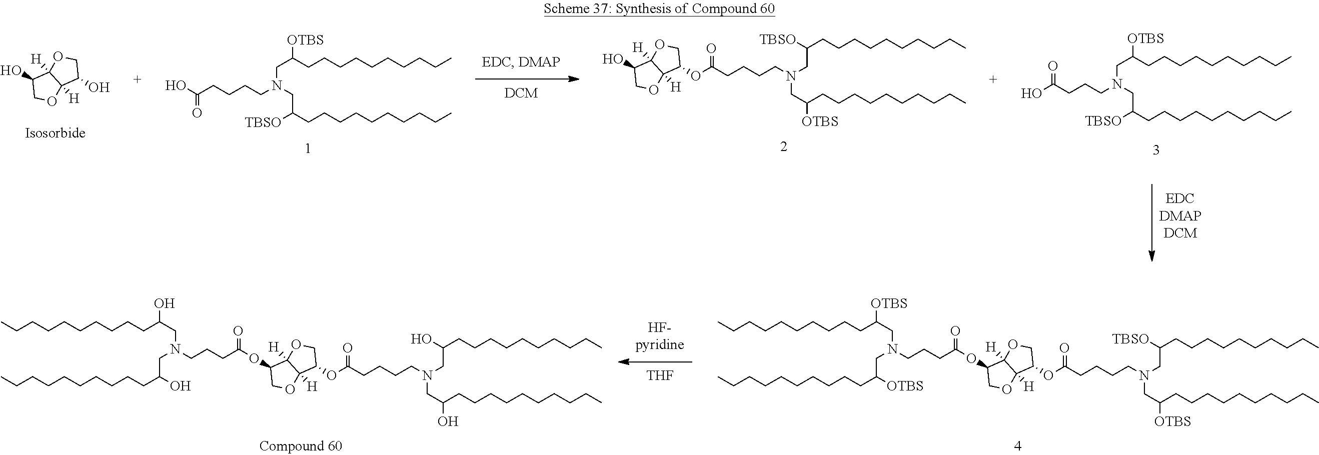

### Step 1: Synthesis of Intermediate (2)

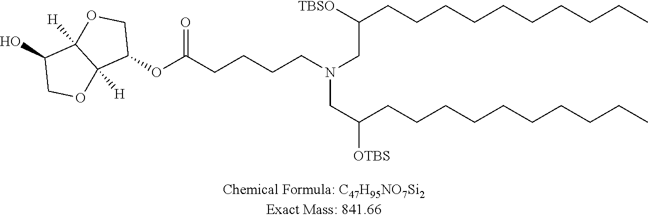 [SMILES: CCCCCCCCCCC(CN(CCCCC(=O)O[C@H]1CO[C@H]2[C@@H]1OC[C@H]2O)CC(CCCCCCCCCC)O[Si](C)(C)C(C)(C)C)O[Si](C)(C)C(C)(C)C]

As depicted in Scheme 37: To a solution of intermediate (1) (3.0 g, 4.2 mmol) in anhydrous DCM (30 mL), Isosorbide (1.20 g, 8.21 mmol) was added. DMAP (564 mg, 4.62 mmol) and EDC (1.047 g, 5.462 mmol) were then added. The reaction mixture was stirred at room temperature overnight. MS analysis showed the presence of monoester. The reaction mixture was diluted with DCM and washed with a saturated NaHCO₃ solution, water, then brine solution. Compound extracted from aqueous layers at each step of the work up. Organic layer dried with anhydrous Na2SO4 and concentrated. Crude residue purified using an hexanes: ethyl acetate solvent system to obtain intermediate (2) (2.348 g, 66% yield).

### Results:

ESI-MS: Calculated C47H95NO7Si2, [M+H+]=842.67, Observed=842.6

### Step 2: Synthesis of Intermediate (4)

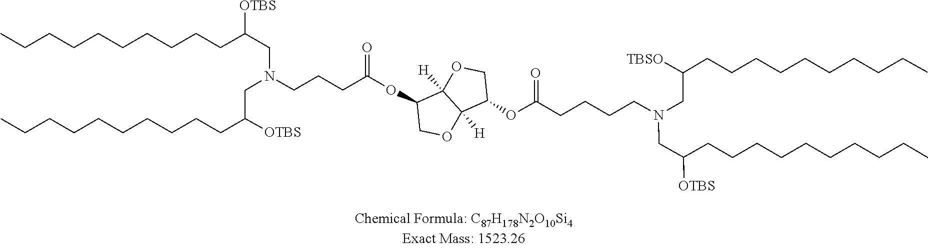 [SMILES: CCCCCCCCCCC(CN(CCCCC(=O)O[C@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 37: To a solution of intermediate (3) (366 mg, 0.523 mmol) in anhydrous DCM (6.0 mL), intermediate (2) (353 mg, 0.419 mmol) was added. DMAP (60 mg, 0.49 mmol) and EDC (114 mg, 0.595 mmol) were then added. The reaction mixture was stirred at room temperature overnight. MS analysis showed the presence of the expected product. The reaction mixture was diluted with DCM and washed with a saturated NaHCO₃ solution, water, then brine. Compound extracted from aqueous layers at each step of the work up. Organic layer dried with anhydrous Na2SO4 and concentrated. The crude residue was purified using an ethyl acetate: hexanes solvent system to obtain intermediate (4) (768 mg, quantitative yield).

### Results:

ESI-MS: Calculated C87H178N2O10Si4, [M+H+]=1524.26, Observed=763.0 [M/2+H+]

### Step 3: Synthesis of Compound 60

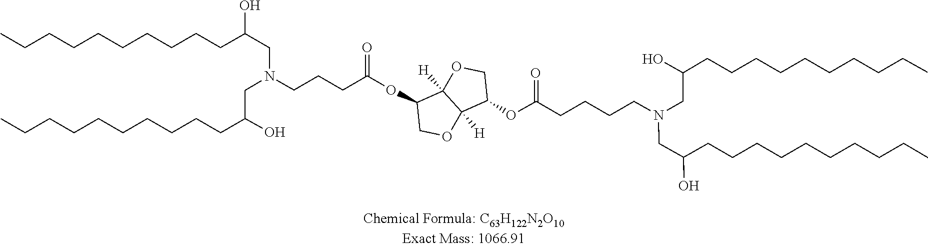 [SMILES: CCCCCCCCCCC(O)CN(CCCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

As depicted in Scheme 37: To a solution of intermediate (4) (768 mg, 0.504 mmol) in anhydrous THF (5.00 mL) at 0° C. was added HF-pyridine (2.50 mL, 19 mmol, 70 mass %). The reaction mixture was warmed to room temperature and stirred for 16 hours. MS analysis indicated completion of the reaction. The reaction mixture was diluted with ethyl acetate, quenched by slow addition of solid NaHCO₃ at 0° C., followed by saturated NaHCO₃ solution. The organic layer was washed with saturated NaHCO₃ solution, water, and brine. Then dried over anhydrous Na2SO4 and concentrated. The crude residue was purified using an ethyl acetate: hexanes solvent system to obtain compound 60 (284 mg, 53% yield).

### Results:

1H NMR (400 MHz, CDCl3) δ 5.22-5.12 (m, 3H), 4.87-4.79 (m, 1H), 4.52-4.44 (m, 1H), 4.01-3.90 (m, 3H), 3.83-3.75 (m, 1H), 3.68-3.55 (m, 4H), 2.62-2.52 (m, 4H), 2.50-2.29 (m, 12H), 1.84-1.76 (m, 2H), 1.48-1.45 (m, 2H), 1.46-1.37 (m, 8H), 1.38-1.19 (m, 66H), 0.88 (t, 12H).

ESI-MS: Calculated C63H122N2O10, [M+H+]=1067.92, Observed=1067.8

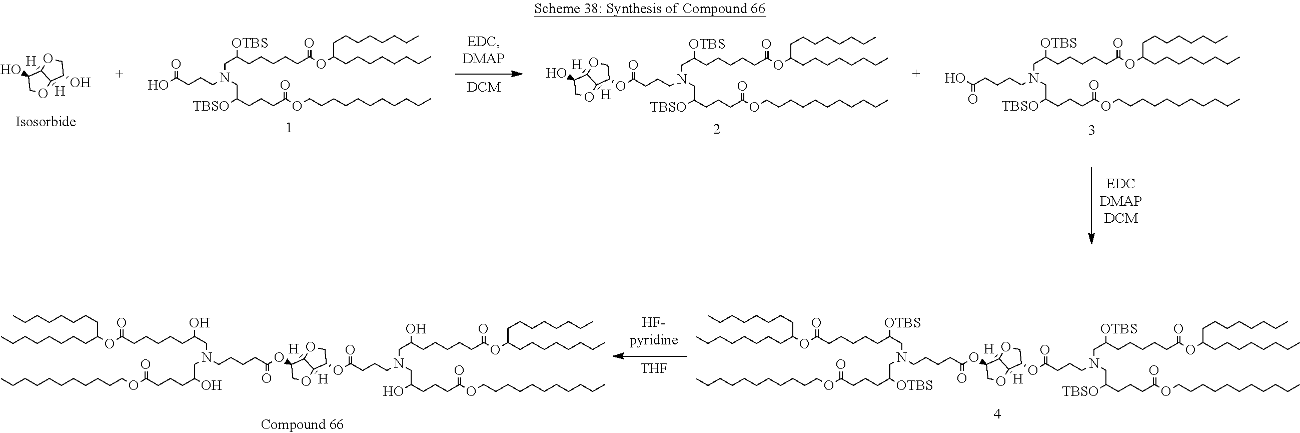

### Step 1: Synthesis of Intermediate (2)

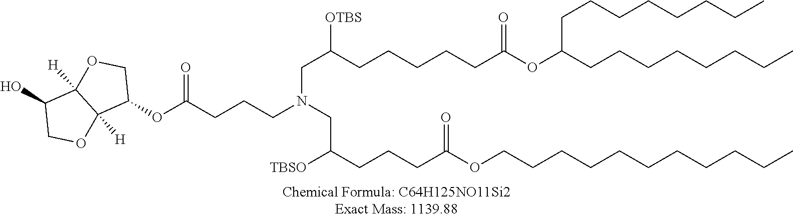 [SMILES: CCCCCCCCCCCOC(=O)CCCC(CN(CCCC(=O)O[C@H]1CO[C@H]2[C@@H]1OC[C@H]2O)CC(CCCCCC(=O)OC(CCCCCCCC)CCCCCCCC)O[Si](C)(C)C(C)(C)C)O[Si](C)(C)C(C)(C)C]

As depicted in Scheme 38: To a solution of intermediate (1) (1.001 g, 0.9884 mmol) in anhydrous DCM (10 mL), Isosorbide (216 mg, 1.48 mmol) was added. DMAP (133 mg, 1.09 mmol) and EDC (246 mg, 1.28 mmol) were then added. The reaction mixture was stirred at room temperature overnight. MS analysis showed the presence of monoester. The reaction mixture was diluted with DCM and washed with a saturated NaHCO₃ solution, water, then brine solution. Compound extracted from aqueous layers at each step of the work up. Organic layer dried with anhydrous Na2SO4 and concentrated. Crude residue purified using a hexanes: ethyl acetate solvent system to obtain intermediate (2) (664 mg, 59% yield).

### Results:

ESI-MS: Calculated C64H125NO11Si2, [M+H+]=1140.89, Observed=1140.7

### Step 2: Synthesis of Intermediate (4)

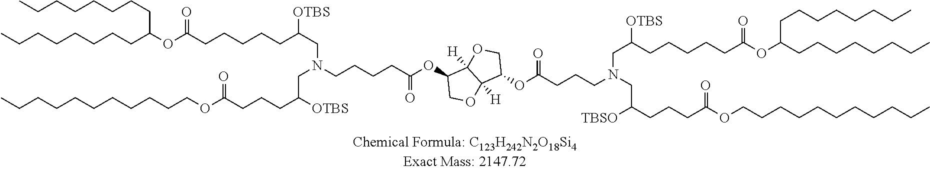 [SMILES: CCCCCCCCCCCOC(=O)CCCC(CN(CCCCC(=O)O[C@@H]1CO[C@H]2[C@@H]1OC[C@@H]2OC(=O)CCCN(CC(CCCCCC(=O)OC(CCCCCCCC)CCCCCCCC)O[Si](C)(C)C(C)(C)C)CC(CCCC(=O)OCCCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 38: To a solution of intermediate (3) (434 mg, 0.423 mmol) in anhydrous DCM (8 mL), IS-E3-001-Mono-TBS (352 mg, 0.309 mmol) was added. DMAP (41 mg, 0.34 mmol) and EDC (81 mg, 0.42 mmol) were then added. The reaction mixture was stirred at room temperature overnight. MS analysis showed the presence of the expected product. The reaction mixture was diluted with DCM and washed with a saturated NaHCO₃ solution, water, then brine. Compound extracted from aqueous layers at each step of the work up. Organic layer dried with anhydrous Na2SO4 and concentrated. The crude residue was purified using an ethyl acetate: hexanes solvent system to obtain intermediate (4) (459 mg, 69% yield).

### Results:

ESI-MS: Calculated C123H242N2O18Si4, [M+H+]=2148.72, Observed=1075.4 [M/2+H+]

### Step 3: Synthesis of Compound 66

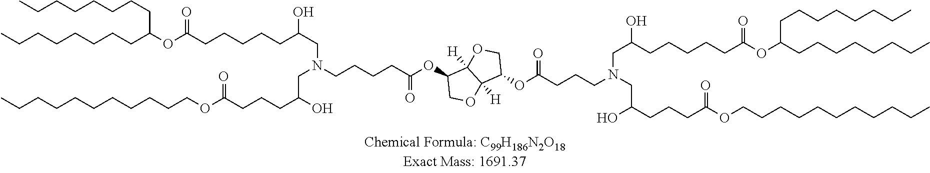 [SMILES: CCCCCCCCCCCOC(=O)CCCC(O)CN(CCCCC(=O)O[C@@H]1CO[C@H]2[C@@H]1OC[C@@H]2OC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC]

As depicted in Scheme 38: To a solution of intermediate (4) (459 mg, 0.214 mmol) in anhydrous THF (3.0 mL) at 0° C. was added HF-pyridine (0.17 mL, 1.3 mmol, 70 mass %). The reaction mixture was warmed to room temperature and stirred for 16 hours. MS analysis showed the presence of mono-TBS product. 0.17 mL of HF-pyridine was added and the reaction was stirred for an additional 5 hours. MS analysis indicated completion of the reaction. The reaction mixture was diluted with ethyl acetate, quenched by slow addition of solid NaHCO₃ at 0° C., followed by saturated NaHCO₃ solution. The organic layer was washed with saturated NaHCO₃ solution, water, and brine. Then dried over anhydrous Na2SO4 and concentrated. The crude residue was purified using an ethyl acetate: hexanes solvent system to obtain compound 66 (141 mg, 39% yield).

### Results:

1H NMR (400 MHz, CDCl3) δ 5.23-5.11 (m, 2H), 4.87-4.82 (m, 2H), 4.51-4.45 (m, 1H), 4.10-4.01 (m, 4H), 3.98-3.94 (m, 3H), 3.83-3.75 (m, 1H), 3.67-3.61 (m, 3H), 2.68-2.22 (m, 29H), 1.83-1.20 (m, 119H), 0.88 (t, 18H).

ESI-MS: Calculated C99H186N2O18, [M+H+]=1692.38, Observed=847.0 [M/2+H+]

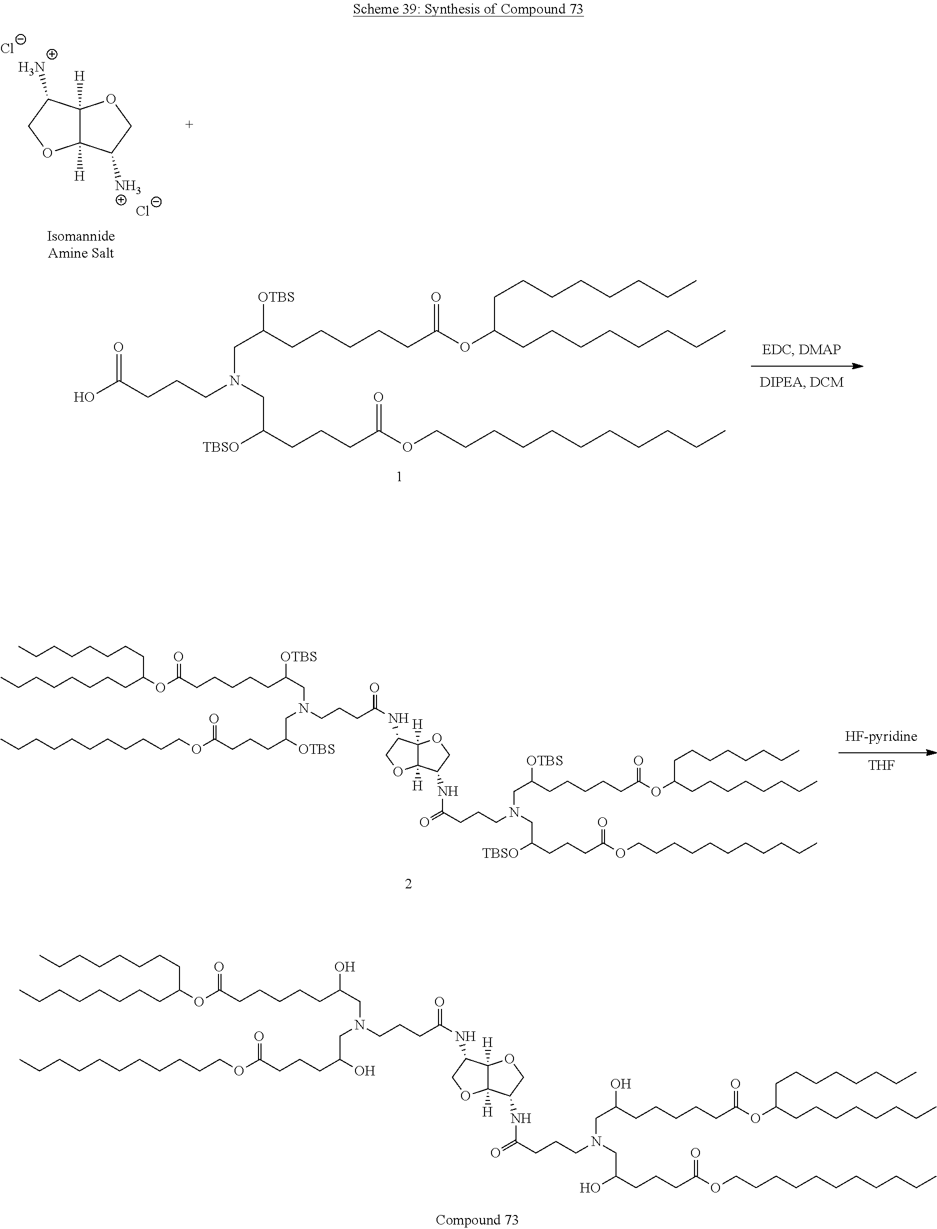

### Step 1: Synthesis of Intermediate (2)

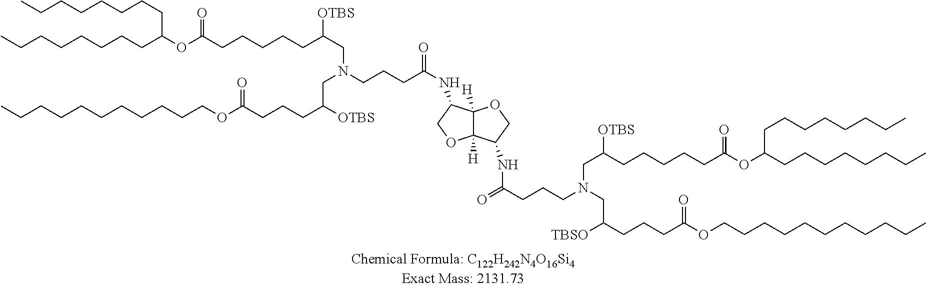 [SMILES: CCCCCCCCCCCOC(=O)CCCC(CN(CCCC(=O)N[C@H]1CO[C@H]2[C@@H]1OC[C@@H]2NC(=O)CCCN(CC(CCCCCC(=O)OC(CCCCCCCC)CCCCCCCC)O[Si](C)(C)C(C)(C)C)CC(CCCC(=O)OCCCCCCCCCCC)O[Si](C)(C)C(C)(C)C)CC(CCCCCC(=O)OC(CCCCCCCC)CCCCCCCC)O[Si](C)(C)C(C)(C)C)O[Si](C)(C)C(C)(C)C]

As depicted in Scheme 39: To a solution of intermediate (1) (5.498 g, 5.429 mmol) in anhydrous DCM (10 mL) was added Isomannide diamine salt (537 mg, 2.47 mmol), DMAP (315 mg, 2.57 mmol), DIPEA (3.5 mL, 20 mmol), and EDC (1.894 g, 9.880 mmol). The resulting mixture was stirred at room temperature overnight. MS analysis indicated completion of the reaction after 16 hours. The reaction mixture was diluted with DCM and washed with saturated NaHCO₃ solution, water, and brine solution. The organic layer was dried over anhydrous Na2SO4 and concentrated. The crude residue was purified using a hexanes: ethyl acetate solvent system to get intermediate (2) (3.12 g, 59% yield).

### Results:

ESI-MS: Calculated C122H242N4O16Si4, [M+H+]=2132.74, Observed=1067.3 [M/2+H+]

### Step 2: Synthesis of Compound 73

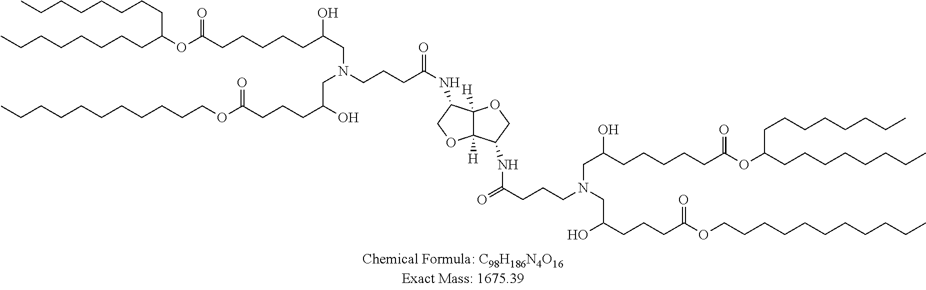 [SMILES: CCCCCCCCCCCOC(=O)CCCC(O)CN(CCCC(=O)N[C@H]1CO[C@H]2[C@@H]1OC[C@@H]2NC(=O)CCCN(CC(O)CCCCCC(=O)OC(CCCCCCCC)CCCCCCCC)CC(O)CCCC(=O)OCCCCCCCCCCC)CC(O)CCCCCC(=O)OC(CCCCCCCC)CCCCCCCC]

As depicted in Scheme 39: To a solution of intermediate (2) (3.12 g, 1.46 mmol) in anhydrous THF (12.5 mL) at 0° C. was added HF-pyridine (1.88 mL, 15 mmol, 70 mass %). The reaction mixture was warmed to room temperature and stirred for 16 hours. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water, and brine. Then dried over anhydrous Na2SO4 and concentrated. The crude residue was purified using a hexanes: dichloromethane solvent system on neutral alumina to obtain compound 73 (220 mg, 9% yield).

### Results:

1H NMR (400 MHz, CDCl3) δ 4.88-4.79 (m, 2H), 4.58-4.32 (m, 3H), 4.08-3.88 (m, 7H), 3.80-3.50 (m, 6H), 2.56-2.26 (m, 20H), 1.95-1.17 (m, 124H), 0.87 (t, 18H).

ESI-MS: Calculated C98H186N4O16, [M+H+]=1676.39, Observed=839 [M/2+H+]

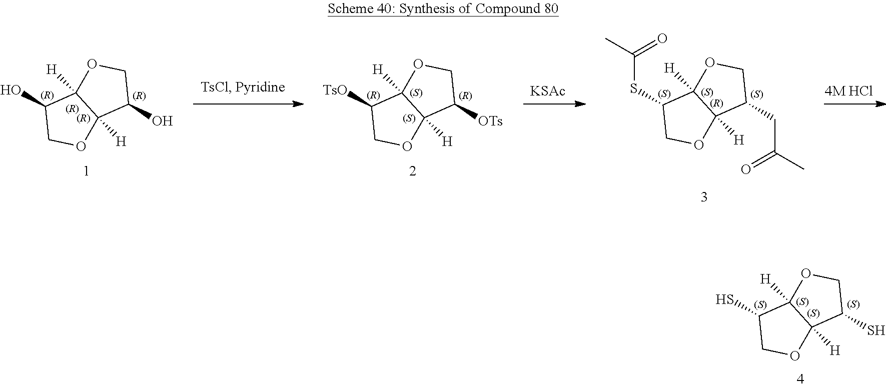

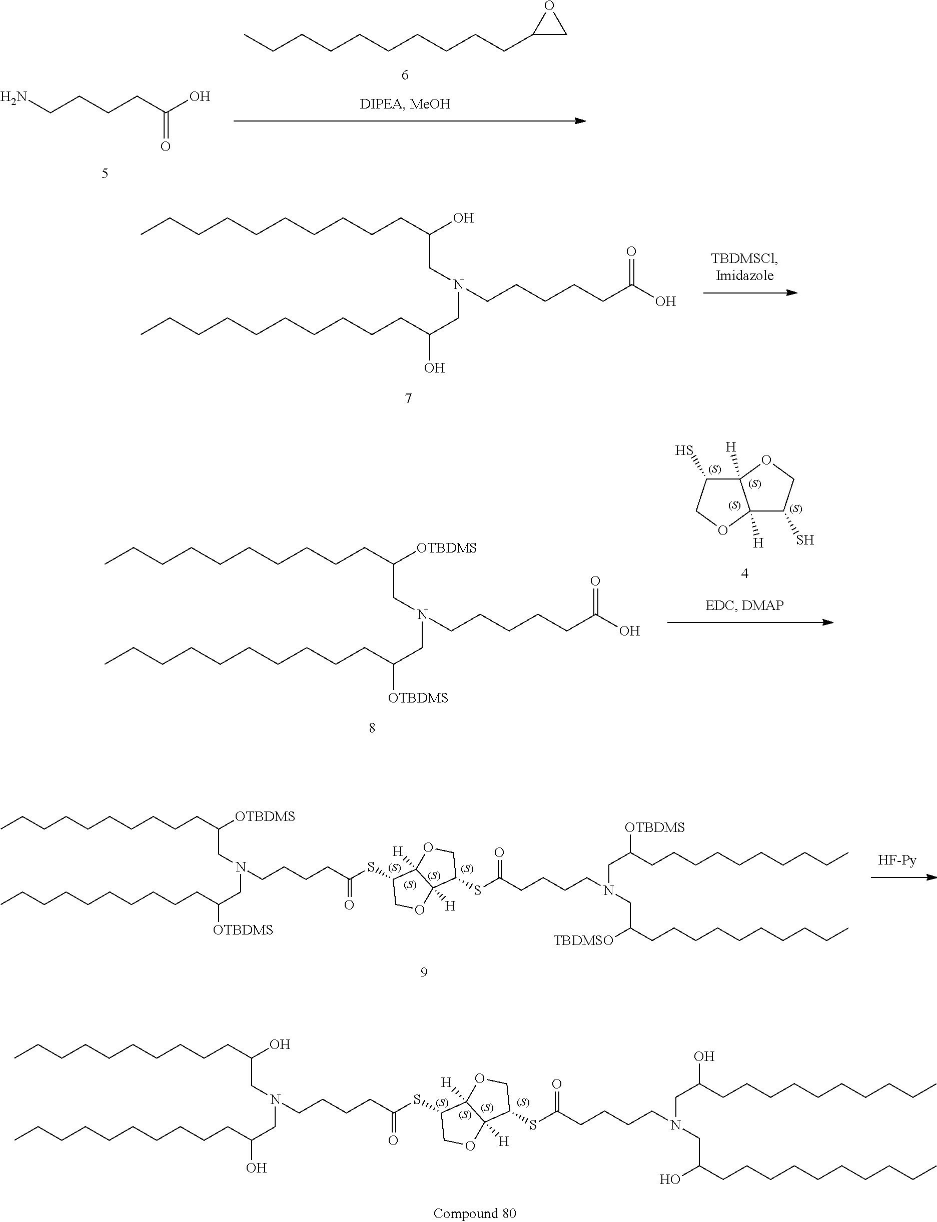

### Step 1: Synthesis of Intermediate (2)

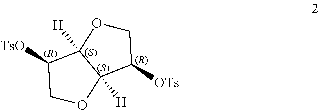

As depicted in Scheme 40: To a stirred solution of intermediate (1) (5.0 g, 34.2 mmol) in pyridine (10 mL) was added p-(chlorosulfonyl)toluene (26.1 g, 4 eq., 137 mmol) at room temperature. The reaction mixture stirred at RT for 18 h. The progress of reaction was monitored by TLC/LCMS data. After completion of the reaction, the reaction mixture was concentrated under reduced pressure. The crude was purified by flash column chromatography (using 0-60% ethyl acetate in n-hexane) to afford intermediate (2) (10.0 g, 64.3% yield) as a white crystalline solid.

### Results:

LCMS analysis: Purity 99.08%, Calculated C20H22O8S2=454.08, Observed=455.29 (m/z, M+H+).

### Step 2: Synthesis of Intermediate (3)

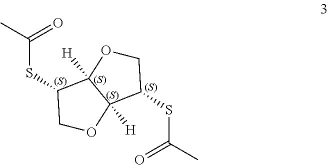

As depicted in Scheme 40: To a stirred solution of intermediate (2) (10 g, 22 mmol) in DMF (33.3 mL) was added potassium thioacetae (12.8 g, 5 eq., 110 mmol) under an inert atmosphere. The reaction mixture was stirred at 90° C. for 16 h. The progress of reaction was monitored by TLC (SM was consumed completely). After the completion of the reaction, mixture was quenched with ice cold water (20 ml) and extracted with ethyl acetate (60 ml). The organic layer was washed with brine solution 2 times, dried over anhydrous sodium sulphate and distilled under reduced pressure to get the crude compound. The crude was purified by flash chromatography (using 0-20% ethyl acetate in heptane) to afford intermediate (3) (3.5 g, 60.64% yield) as a reddish liquid.

### Results:

ELSD analysis: Purity 99.64%, Calculated C10H14O4S2=262.03, Observed=263.10 (m/z, M+H+).

### Step 3: Synthesis of Intermediate (4)

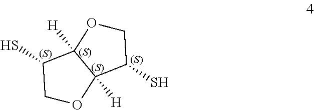

As depicted in Scheme 40: To a stirred solution of intermediate (3) (0.8 g, 3.05 mmol) in methanol (10 mL, 247 mmol) added 4N HCl in dioxane (0.5 mL) dropwise at 0° C. The reaction mixture was stirred at room temperature for 16 h. After completion of reaction, reaction mixture was neutralized with triethyl amine and evaporated under vacuum to afford crude compound intermediate (4) (0.50 g, 64% yield), which was used as such immediately without further purification.

### Results:

1H-NMR (400 MHz, CDCl3) −4.68 (s, 2H), 4.14-4.09 (dd, J=5.2 Hz, 9.6 Hz, 2H), 3.81-3.74 (m, 2H), 3.41-3.37 (m, 2H), 1.73-1.72 (d, J=8.0 Hz, 2H).

### Step 4: Synthesis of Intermediate (7)

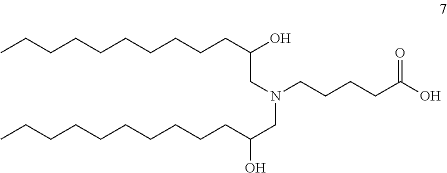 [SMILES: CCCCCCCCCCC(O)CN(CCCCC(=O)O)CC(O)CCCCCCCCCC]

As depicted in Scheme 40: To a stirred solution of intermediate (5) (15.0 g, 128 mmol) and intermediate (6) (51.9 g, 2.2 eq., 282 mmol) in methanol (0.5 L) was added ethylbis(propan-2-yl)amine (55.2 mL, 2.5 eq., 320 mmol). The resultant reaction mass was heated at 90° C. for 16 h. After 16 h reaction progress was monitor by TLC/ELSD. Reaction mass was cool to RT. Tetrahydrofuran (60 mL, 737 mmol), water (60 mL, 3.33 mol) and lithium(1+) hydrate hydroxide (2.44 g, 2 eq., 58.2 mmol) were added to RM. After 4 h, reaction progress monitored by TLC. Reaction mixture was evaporated under reduced pressure. Adjust the pH of mixture up to 2.0 by using 2N hydrochloric acid (200.0 mL) and extracted with DCM (2×250 mL). The organic layer separated, dried over sodium sulfate, distilled out under reduced pressure to get crude intermediate (7) (45.0 g, crude) as a colourless semisolid. Crude used as such for next step.

### Results:

ELSD analysis: Purity 99.93%, Calculated C29H59NO4=485.44, Observed=486.45 (m/z, M+H+).

### Step 5: Synthesis of Intermediate (8)

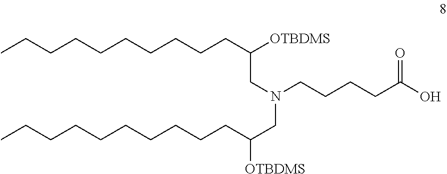 [SMILES: CCCCCCCCCCC(CN(CCCCC(=O)O)CC(CCCCCCCCCC)O[Si](C)(C)C(C)(C)C)O[Si](C)(C)C(C)(C)C]

As depicted in Scheme 40: To a stirred solution of intermediate (5) (15 g, 30.9 mmol) in dichloromethane (150.0 mL) were added 1H-imidazole (21 g, 10.0 eq., 309 mmol) and tert-butyl(chloro)dimethylsilane (18.6 g, 4 eq., 124 mmol). The reaction mixture was stirred at room temperature for 16 hours. The progress of reaction was monitored by TLC/ELSD. After completion, reaction mixture was diluted with DCM, water and extracted 3 times with DCM. The organic layer was collected, concentrated under reduced pressure to get crude and was purified by flash column chromatography (SiO₂: 0-30% ethyl acetate in hexane) to obtain intermediate (8) (10 g, 44.89% yield) as colourless liquid.

### Results:

ELSD analysis: Purity 99.35%, Calculated C41H87NO4Si2=713.62, Observed=714.60 (m/z, M+H+).

### Step 6: Synthesis of Intermediate (9)

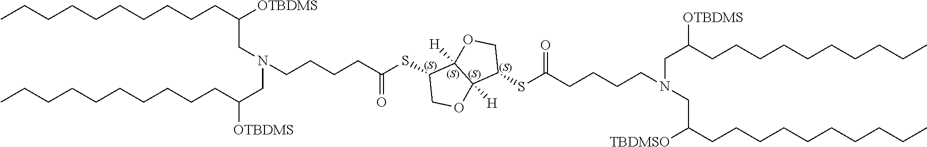

As depicted in Scheme 40: To a stirred solution of intermediate (8) (3.37 g, 2.1 eq., 4.71 mmol) in DCM (50 mL) was added N,N-dimethyl-4-pyridylamine (1.33 g, 4.8 eq., 10.8 mmol) and 2-methyl-2,6,8-triaza-6,7-decadiene hydrogen chloride (EDC·HCl) (1.03 g, 2.4 eq., 5.38 mmol). The reaction mixture was stirred at RT for 10 min and added intermediate (4) under inert atmosphere. The resultant reaction mass was stirred at RT for 16 h. Progress of reaction was monitor by ELSD. Reaction mass was concentrated under reduced pressure to afford crude, which was purified by column chromatography using 5-6% EtOAc in Heptane. The fraction was evaporated under reduced pressure to get intermediate (9) (0.25 g, 7.0% yield) as colourless liquid.

### Results:

ELSD analysis: Purity 97.31%, Calculated C88H180N2O8S2Si4=1569.23, Observed=1569.65 (m/z, M+H+).

### Step 7: Synthesis of Compound 80

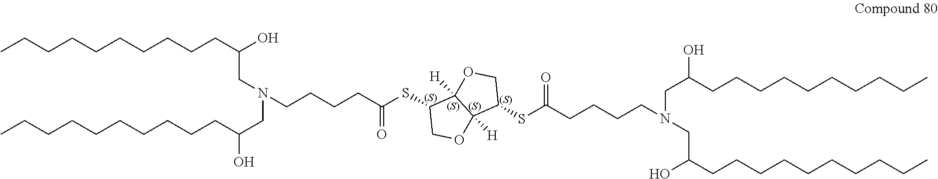

As depicted in Scheme 40: To stirred solution of intermediate (9) (250 mg, 159 μmol) in tetrahydrofuran (3 mL) was added hydrogen fluoride-pyridine complex (70% w/w (473 mg, 30 eq., 4.77 mmol). The reaction mixture was stirred at RT for 16 h. The progress of reaction was monitored by ELSD. After completion, reaction mixture was quenched by cold saturated sodium bicarbonate solution up to PH 8 and extracted with Pentane (3×10 ml). The organic layers were combine, dried over sodium sulphate anhydride, concentrate under reduced pressure to get compound 80 (0.90 g: 50.05% yield) as yellowish liquid.

### Results:

1H-NMR (400 MHz, CDCl3) −4.57-4.43 (br, 2H), 4.26-4.20 (m, 2H), 4.14-3.95 (m, 2H), 3.80-3.74 (m, 2H), 3.72-2.60 (m, 4H), 2.60-2.56 (m, 8H), 2.55-2.40 (m, 8H), 1.75-1.60 (m, 4H), 1.48-1.38 (m, 14H), 1.35-1.22 (m, 62H), 0.87 (t, J=6.4 Hz, 12H).

ELSD analysis: Purity 96.60%, Calculated C64H124N2O8S2=1112.88, Observed=1113.95 (m/z, M+H+).

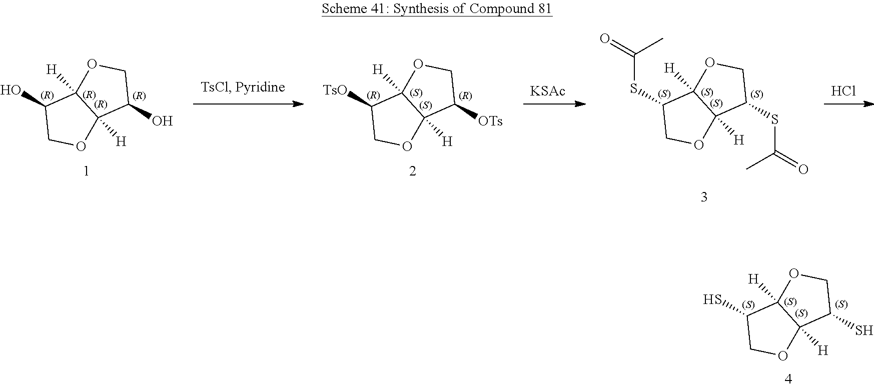

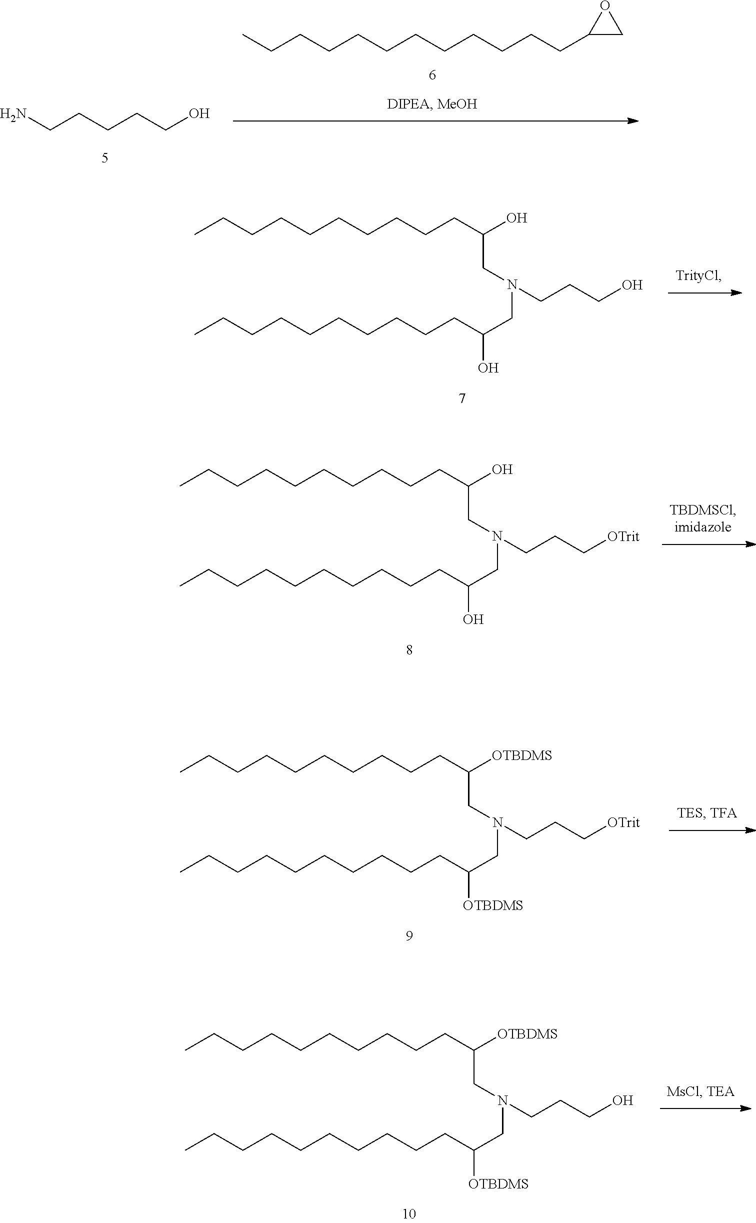

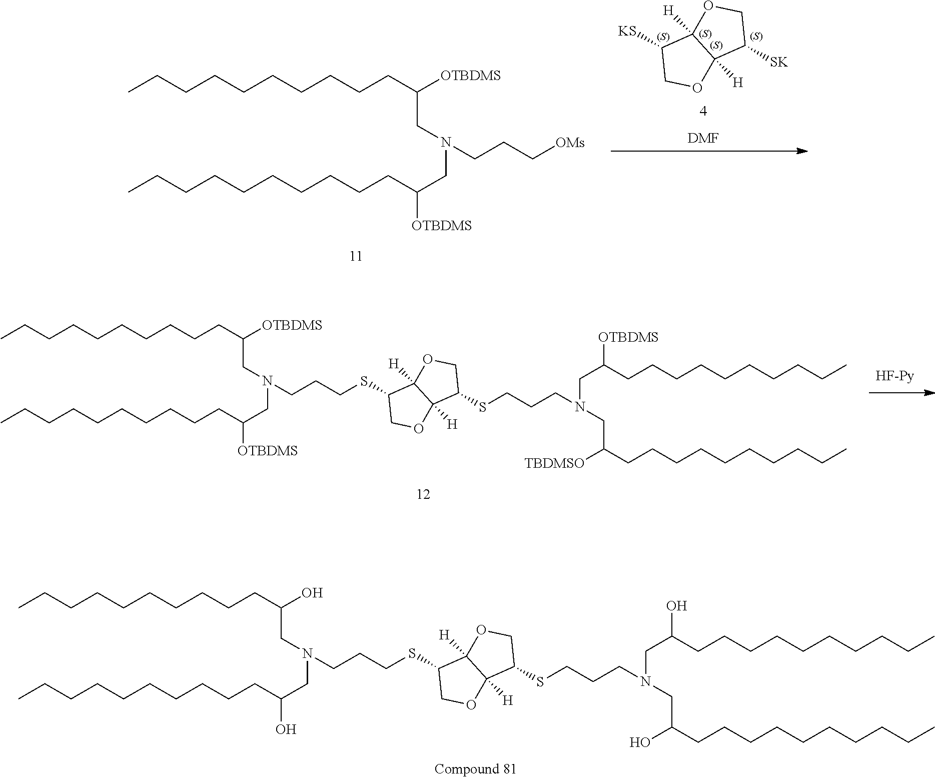

### Step 1: Synthesis of Intermediate (2)

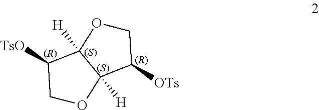

As depicted in Scheme 41: To a stirred solution of intermediate (1) (5 g, 34.2 mmol) in pyridine (10 mL) was added p-(chlorosulfonyl)toluene (26.1 g, 4 eq., 137 mmol) at room temperature. The reaction mixture was stirred at RT for 18 h. The progress of reaction was monitored by TLC/LCMS data. After completion of the reaction, the reaction mixture was concentrated under reduced pressure. The solid residue was purified by flash column chromatography (using 0-60% ethyl acetate in n-hexane) to afford intermediate (2) (10 g, 64.3% yield) as a white crystalline solid.

### Results:

LCMS analysis: Purity 99.08%, Calculated C20H22O8S2=454.08, Observed=455.29 (m/z, M+H+).

### Step 2: Synthesis of Intermediate (3)

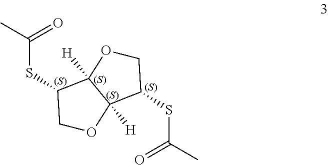

As depicted in Scheme 41: To a stirred solution of intermediate (2) (10 g, 22 mmol) in DMF (33.3 mL) under an inert atmosphere was added potassium thioacetae (12.8 g, 5 eq., 110 mmol). The reaction mixture was stirred at 90° C. for 16 h. The progress of reaction was monitored by TLC (SM was consumed completely). After the completion of the reaction, mixture was quenched with ice cold water (20 ml) and extracted with ethyl acetate (60 ml). The organic layer was washed with brine solution 2 times, dried over anhydrous sodium sulphate and distilled under reduced pressure to get the crude compound. The crude was purified by flash chromatography (using 0-20% ethyl acetate in heptane) to afford intermediate (3) (3.5 g, 60.64% yield) as a reddish liquid.

### Results:

ELSD analysis: Purity 99.64%, Calculated C10H14O4S2=262.03, Observed=263.10 (m/z, M+H+).

### Step 3: Synthesis of Intermediate (4)

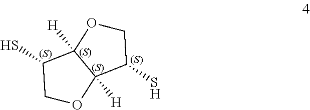

As depicted in Scheme 41: To a stirred solution of intermediate (3) (0.8 g, 3.05 mmol) in methanol (10 mL, 247 mmol) added 4N HCl in dioxane (0.5 mL) dropwise at 0° C. The reaction mixture was stirred at room temperature for 16 h. After completion of reaction, reaction mixture was neutralized with triethyl amine and evaporated under vacuum to afford crude compound intermediate (4) (0.50 g, 64% yield), which was used as such immediately without further purification.

### Results:

1H-NMR (400 MHz, CDCl3) −4.68 (s, 2H), 4.12-4.09 (dd, J=5.2 Hz, 9.6 Hz, 2H), 3.81-3.74 (m, 2H), 3.41-3.37 (m, 2H), 1.73-1.72 (d, J=8.0 Hz, 2H).

### Step 4: Synthesis of Intermediate (7)

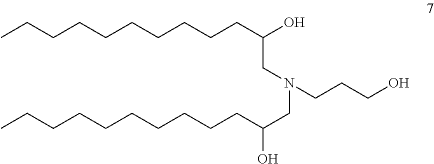 [SMILES: CCCCCCCCCCC(O)CN(CCCO)CC(O)CCCCCCCCCC]

As depicted in Scheme 41: To a stirred solution of intermediate (5) (15.0 g, 0.2 mol) in isopropanol (200 ml, 2.62 mol), was added intermediate (6) (81 g, 2.2 eq., 439 mmol) at RT. Reaction mass was heated to reflux for 16 h. The progress of reaction mass was monitored by ELSD/TLC (Sm was consumed). Reaction mass was concentrated under reduced pressure. The resulting crude was purified over silica using 5-10% Methanol in DCM to give intermediate (7) (88.6 g, 78% yield) as white crystalline solid.

### Results

ELSD analysis: Purity 98.49%, Calculated C27H57NO3=443.43, Observed=444.00 (m/z, M+H+).

### Step 5: Synthesis of Intermediate (8)

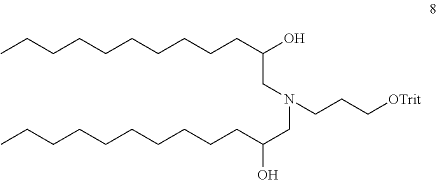

As depicted in Scheme 41: To a stirred solution of intermediate (7) (88 g, 198 mmol) in dry dichloromethane (0.5 L, 7.81 mol) was added chlorotriphenylmethane (60.8 g, 1.1 eq., 218 mmol) and pyridine (48 mL, 3 eq., 595 mmol) under argon atmosphere at 0° C. After being stirred for 2 h at 0° C., the reaction mixture was stirred for 16 h at RT. Progress of reaction was monitored by ELSD/TLC (Sm was consumed). Reaction mixture was quenched in ice cold water (1000 mL) and extracted with DCM (100 ml). The combined organic layer was washed with brine, dried over Na2SO4, filtered, and concentrated in vacuum to give intermediate (8) (128.0 g, crude) which was forwarded to next step without further purification.

### Results:

ELSD analysis: Purity 92.39, Calculated C46H71NO3=685.54, Observed=686.30 (m/z, M+H+).

### Step 6: Synthesis of Intermediate (9)

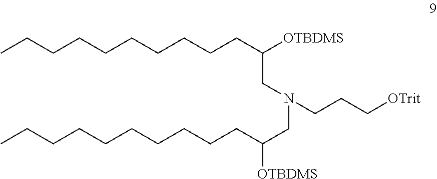

As depicted in Scheme 41: To a stirred solution of intermediate (8) (128 g, 187 mmol) (15 g, 30.9 mmol) in dichloromethane (400.0 mL) were added imidazole (152 g, 12 eq., 2.24 mol) and (tert-butyl)(chloro)bis(methyl)silane (169 g, 6 eq., 1.12 mol). The reaction mixture was stirred at room temperature for 16 hours. The progress of reaction was monitored by TLC/ELSD. After completion, reaction mixture was diluted with DCM, water and extracted 3 times with DCM. The organic layer was collected, concentrated under reduced pressure to get crude. The crude was purified by flash column chromatography (SiO₂: 0-30% ethyl acetate in hexane) to obtain intermediate (9) (83 g, 48.6% Yield) as colourless liquid.

### Results:

ELSD analysis: Purity 99.69%, Calculated C58H99NO3Si2=913.72, Observed=914.40 (m/z, M+H+).

### Step 7: Synthesis of Intermediate (10)

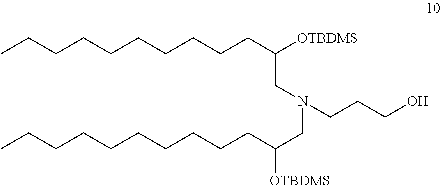 [SMILES: CCCCCCCCCCC(CN(CCCO)CC(CCCCCCCCCC)O[Si](C)(C)C(C)(C)C)O[Si](C)(C)C(C)(C)C]

As depicted in Scheme 41: To a stirred solution of intermediate (9) (20.0 g, 21.9 mmol) in dichloromethane (0.2 L, 3.12 mol) was added triethylsilane (3.9 mL, 1.2 eq., 26.2 mmol) and trifluoroacetic acid (5.02 mL, 3 eq., 65.6 mmol) simultaneously at 0° C. The resulting reaction mixture was stirred at room temperature for 15 min. The progress of reaction was monitored by TLC (SM was consumed). After completion, reaction mass was quenched by saturated solution of sodium bicarbonate (make pH 8-9) and extracted with DCM (2×200 ml). The combined organic layer was dried over anhydrous sodium sulphate and concentrated under reduced pressure to get crude. The crude was purified over silica using (0-20% EtOAC/Hexane) product intermediate (10) (6.0 g, 40.8% yield) as a colourless liquid.

### Results:

ELSD analysis: Purity 98.92%, Calculated: C39H85NO3Si2=671.61, Observed=672.48 (m/z, M+H+).

### Step 8: Synthesis of Intermediate (11)

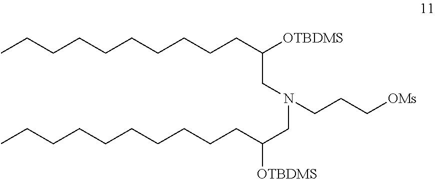 [SMILES: CCCCCCCCCCC(CN(CCCOS(C)(=O)=O)CC(CCCCCCCCCC)O[Si](C)(C)C(C)(C)C)O[Si](C)(C)C(C)(C)C]

As depicted in Scheme 41: To a stirred solution of intermediate (10) (3 g, 4.46 mmol) in dichloromethane (20 mL, 312 mmol) was added triethylamine (6.22 mL, 10 eq., 44.6 mmol). The reaction mixture was cooled to 0° C. and added (chlorosulfonyl)methane (414 μL, 1.2 eq., 5.35 mmol) dropwise. The reaction mixture was stirred for 2 h at room temperature. The progress of reaction mass was monitored by ELSD/TLC (SM was consumed). The reaction mixture was evaporated under reduced pressure. To the crude compound was added diethyl ether and decanted (3 times) the ether layer. The residue was concentrated under reduced pressure to get intermediate (11) (3 g, 89.5% yield) as greenish liquid.

### Results:

ELSD analysis: Purity 92.31%, Calculated C40H87NO5SSi2=671.61, Observed=672.48 (m/z, M+H+).

### Step 9: Synthesis of Intermediate (12)

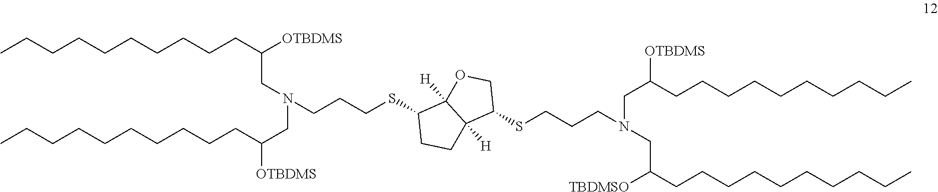 [SMILES: CCCCCCCCCCC(CN(CCCS[C@H]1CO[C@H]2[C@@H]1CC[C@@H]2SCCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 41: To a stirred solution of intermediate (4) (1.02 g, 4 mmol) in dimethylformamide (60 mL, 775 mmol) purged with nitrogen for 15 minutes then added intermediate (11) (6 g, 2 eq., 8 mmol). The reaction mixture was stirred at 90° C. for 16 h. After completion of reaction, reaction mixture was quenched with cold water 60 ml and extracted with ethyl acetate (2×30 ml). The combined organic layer was dried over anhydrous sodium sulphate and concentrated under reduced pressure. The crude was purified in flash chromatography 0-5% Ethyl acetate/Hexane to afford as intermediate (12) (1.0 g, 16.8% yield) as colourless liquid.

### Results:

ELSD analysis: Purity 97.62%, Calculated C84H176N2O6S2Si4=1485.20=, Observed=1486.65 (m/z, M+H+).

### Step 10: Synthesis of Compound 81

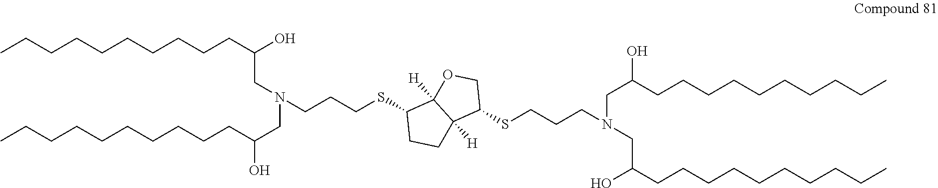 [SMILES: CCCCCCCCCCC(O)CN(CCCS[C@H]1CO[C@H]2[C@@H]1CC[C@@H]2SCCCN(CC(O)CCCCCCCCCC)CC(O)CCCCCCCCCC)CC(O)CCCCCCCCCC]

As depicted in Scheme 41: To stirred solution of intermediate (12) (0.5 g, 336 μmol)) in dicloromethane (5 ml) was added 2M HCl in ether dropwise at 0° C. The reaction was stirred at room temperature for 16 h. The progress of reaction was monitored by ELSD. After completion, reaction mixture was concentrate under reduced pressure and crude was purified by flash chromatography 0 to 5% MeOH/DCM to get compound 81(0.2 g, 57.75% yield) as a light yellowish liquid.

### Results:

1H-NMR (400 MHz, CDCl3) −4.70-1.65 (m, 2H), 4.21-4.14 (m, 2H), 4.05-3.85 (m, 4H), 3.78-3.67 (m, 2H), 3.34-3.28 (m, 2H), 3.27-2.68 (m, 18H), 2.10-1.90 (m, 4H), 1.52-1.36 (m, 12H), 1.35-1.20 (m, 62H), 0.87 (t, J=6.4 Hz, 12H).

ELSD analysis: Purity 99.59%, Calculated C60H120N2O6S2=1028.86, Observed=1029.80 (m/z, M+H+).

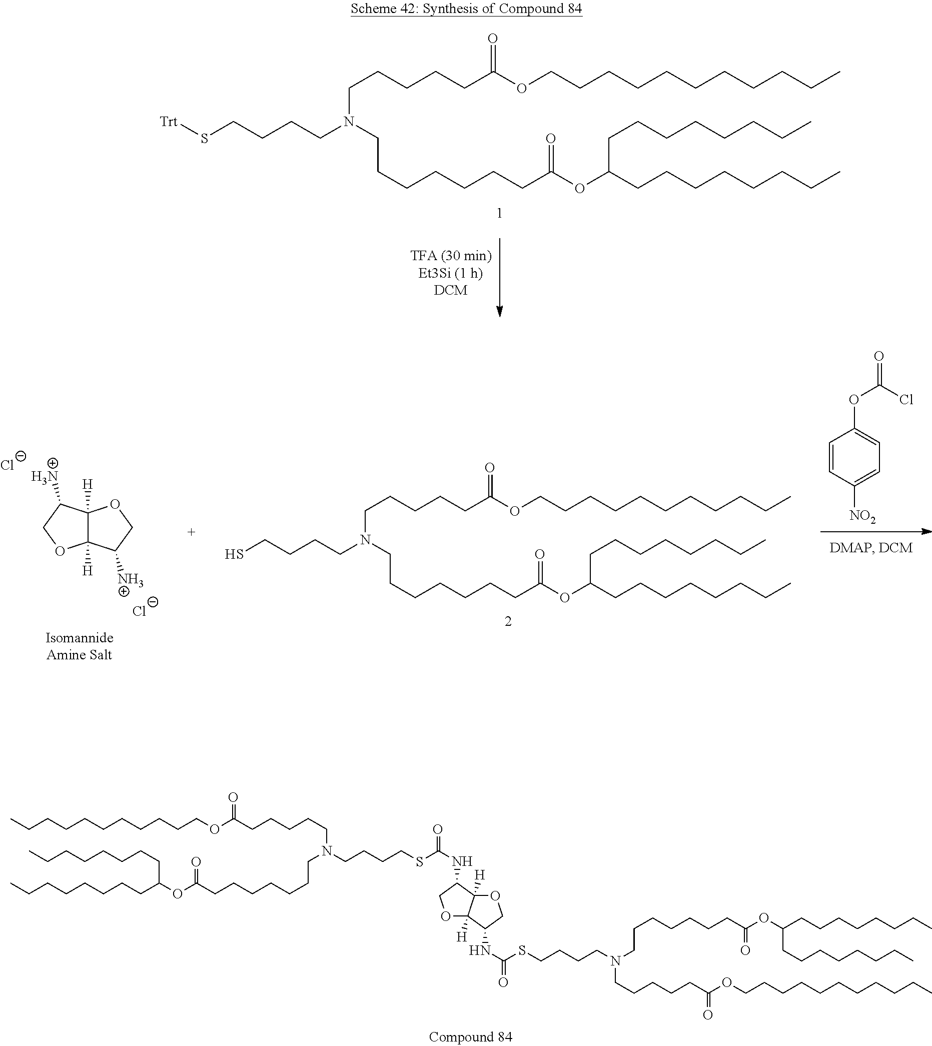

### Step 1: Synthesis of Intermediate (2)

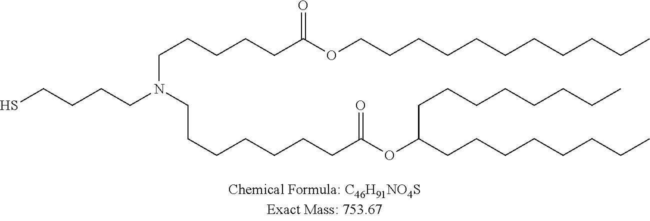 [SMILES: CCCCCCCCCCCOC(=O)CCCCCN(CCCCS)CCCCCCCC(=O)OC(CCCCCCCC)CCCCCCCC]

As depicted in Scheme 42: To a solution of intermediate (1) (8.112 g, 8.140 mmol) in anhydrous DCM (28 mL) under nitrogen, TFA (31 mL, 405 mmol) was added. The reaction mixture was stirred at room temperature for 30 minutes. Triethylsilane (1.6 mL, 10 mmol) was added, and the reaction mixture was stirred for an additional hour. Once removal of the Trityl group was confirmed using MS analysis, DCM and TFA were removed using the rotary evaporator to obtain intermediate (2) (6.0 g, 98% yield).

### Results:

ESI-MS: Calculated C46H91NO4S, [M+H+]=754.67, Observed=754.6

### Step 2: Synthesis of Compound 84

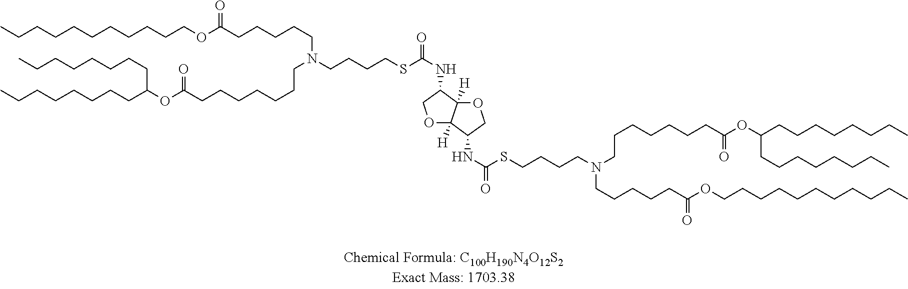 [SMILES: CCCCCCCCCCCOC(=O)CCCCCN(CCCCCCCC(=O)OC(CCCCCCCC)CCCCCCCC)CCCCSC(=O)N[C@H]1CO[C@H]2[C@@H]1OC[C@@H]2NC(=O)SCCCCN(CCCCCCCC(=O)OC(CCCCCCCC)CCCCCCCC)CCCCCC(=O)OCCCCCCCCCCC]

As depicted in Scheme 42: To a solution of Isomannide diamine salt (81 mg, 0.37 mmol) in anhydrous DCM (8 mL) was added DMAP (10 mg, 0.082 mmol) and TEA (0.6 mL, 4 mmol). To the resultant mixture, (4-nitrophenyl) carbonochloridate (193 mg, 0.958 mmol) was added and stirred for 20 minutes. Intermediate (2) (640 mg, 0.849 mmol) was added and the reaction mixture was stirred overnight. MS analysis confirmed the formation of desired product. The reaction mixture was diluted with DCM and washed with saturated NaHCO₃ solution, water and brine solution. The organic layer was dried over anhydrous Na2SO4 and concentrated. The crude residue was purified using a dichloromethane: methanol solvent system to obtain compound 84 (26 mg, 4% yield).

### Results:

1H NMR (400 MHz, CDCl3) δ 4.88-4.76 (m, 2H), 4.57-4.52 (m, 2H), 4.37-4.32 (m, 2H), 4.07-3.99 (m, 4H), 3.96-3.87 (m, 21H), 3.78-3.70 (m, 2H), 2.94-2.85 (m, 4H), 2.44-2.35 (m, 8H), 2.32-2.21 (m, 8H), 1.77-1.17 (m, 136H), 0.85 (t, J=6.5 Hz, 18H).

ESI-MS: Calculated C100H190N4O12S2, [M+H+]=1704.39, Observed=854.0 [M/2+H+] Table 0 gives the corresponding yields, calculated masses, and observed masses for the some of the above syntheses.

TABLE 0
Yield, Calculated Mass and Observed
Mass Data for Selected Syntheses

| Compound | Yield | |
|---|---|---|
| Number | (%) | Mass [M + H⁺] |
| 12 | 22% | Calculated Mass:1678.36 |
| | | Observed Mass: 840.0 [M/2 + H⁺] |
| 18 | 31% | Calculated Mass: 1670.44 |
| | | Observed Mass: 836.0 [M/2 + H+] |
| 45 | 3% | Calculated Mass: 1092.80 |
| | | Observed Mass: 1093.50 |
| 46 | 2% | Calculated Mass: 1056.89 |
| | | Observed Mass: 1057.80 |
| 48 | 36% | Calculated Mass: 1083.92 |
| | | Observed Mass: 1083.8, 542.5 [M/2 + H+] |
| 56 | 18% | Calculated Mass: 1067.92 |
| | | Observed Mass: 1067.8 |
| 57 | 8% | Calculated Mass: 1734.42 |
| | | Observed Mass: 868.0 [M/2 + H+] |
| 58 | 23% | Calculated Mass: 1706.39 |
| | | Observed Mass: 854.0 [M/2 + H⁺] |
| 59 | 29% | Calculated Mass: 1614.38 |
| | | Observed Mass: 808.0 [M/2 + H+] |
| 60 | 35% | Calculated Mass: 1067.92 |
| | | Observed Mass: 1067.8 |
| 61 | 22% | Calculated Mass: 1011.85 |
| | | Observed Mass: 1011.8 |
| 62 | 6% | Calculated Mass: 1025.87 |
| | | Observed Mass: 1025.7 |
| 63 | 5% | Calculated Mass: 1180.04 |
| | | Observed Mass: 1179.9 |
| 64 | 20% | Calculated Mass: 1678.36 |
| | | Observed Mass: 839.9 [M/2 + H⁺] |
| 65 | 8% | Calculated Mass: 1081.93 |
| | | Observed Mass: 1081.8 |
| 66 | 16% | Calculated Mass: 1692.38 |
| | | Observed Mass: 847.0 [M/2 + H+] |
| 67 | 19% | Calculated Mass: 1095.95 |
| | | Observed Mass: 1095.8 |
| 68 | 12% | Calculated Mass: 1706.39 |
| | | Observed Mass: 854.0 [M/2 + H+] |
| 69 | 11% | Calculated Mass: 1734.42 |
| | | Observed Mass: 868.0 [M/2 + H+] |
| 72 | 25% | Calculated Mass: 1612.41 |
| | | Observed Mass: 807.0 [M/2 + H+] |
| 73 | 5% | Calculated Mass: 1676.39 |
| | | Observed Mass: 839.0 [M/2 + H+] |
| 74 | 7% | Calculated Mass: 1444.08 |
| | | Observed Mass: 1445.0, 722.8 [M/2 + H+] |
| 75 | 67% | Calculated Mass: 1380.10 |
| | | Observed Mass: 1379.9, 690.8 [M/2 + H+] |
| 76 | 16% | Calculated Mass: 1668.48 |
| | | Observed Mass: 835.0 [M/2 + H+] |
| 77 | 29% | Calculated Mass: 1732.46 |
| | | Observed Mass: 867.0 [M/2 + H+] |
| 78 | 17% | Calculated Mass: 1704.42 |
| | | Observed Mass: 853.0 [M/2 + H+] |
| 80 | 12% | Calculated Mass: 1112.88 |
| | | Observed Mass: 1113.95 |
| 81 | 1% | Calculated Mass: 1028.86 |
| | | Observed Mass: 1029.80 |
| 84 | 4% | Calculated Mass: 1704.39 |
| | | Observed Mass: 854.0 [M/2 + H+] |
| 85 | 14% | Calculated Mass: 1472.08 |
| | | Observed Mass: 1472.8, 736.7[M/2 + H+] |
| 86 | 2% | Calculated Mass: 1120.83 |
| | | Observed Mass: 1121.75 |

### Example 2: Lipid Nanoparticle Formulation

Cationic lipids described herein can be used in the preparation of lipid nanoparticles according to methods known in the art. For example, suitable methods include methods described in International Publication No. WO 2018/089801, which is hereby incorporated by reference in its entirety.

The lipid nanoparticles in the examples of the present invention were formulated using Process A of WO 2018/089801 (see, e.g., Example 1 and FIG. 1 of WO 2018/089801). Process A (“A”) relates to a conventional method of encapsulating mRNA by mixing mRNA with a mixture of lipids, without first pre-forming the lipids into lipid nanoparticles. In an exemplary process, an ethanolic solution of a mixture of lipids (cationic lipid, phosphatidylethanolamine, cholesterol, and polyethylene glycol-lipid) at a fixed lipid to mRNA ratio were combined with an aqueous buffered solution of target mRNA at an acidic pH under controlled conditions to yield a suspension of uniform LNPs. After ultrafiltration and diafiltration into a suitable diluent system, the resulting nanoparticle suspensions were diluted to final concentration, filtered, and stored frozen at −80° C. until use.

Lipid nanoparticle formulations of Table 1 were prepared by Process A. All of the lipid nanoparticle formulations comprised hEPO mRNA and the different lipids (Cationic Lipid: DMG-PEG2000: Cholesterol: DOPE) in mol % ratios of 40:1.5:28.5:30 (Cationic Lipid: DMG-PEG2000: Cholesterol: DOPE).

The Polydispersity Index (Pdl) of lipid nanoparticles can be determined by diluting the formulation in 10% trehalose at about 0.1 mg/ml mRNA concentration and then measuring the size on Malvern zetasizer. The lipid nanoparticle size can be obtained with Malvern Zetasizer Nano-ZS.

Dynamic light scattering (DLS) measurements were performed using a Malvern Instruments Zetasizer with a backscattering detector angle of 173° and a 4-mW, 633-nm He—Ne laser (Worcestershire, UK). The samples were analyzed by diluting in 10% Trehalose and measuring the size and Poly dispersity Index (PDI) in an optical grade polystyrene cuvette.

TABLE 1
Characterization data for exemplary lipid nanoparticles
comprising cationic lipids of the present invention

| | | | | Poly- | Encapsulation | |
|---|---|---|---|---|---|---|
| | | | | dispersity | efficiency | |
| Compound | | | Size | Index | (EE) | |
| # | mRNA | Process | (nm) | (PDI) | % | N/P* |
| 1 | hEPO | A | 93 | 0.14 | 85 | 4 |
| 2 | hEPO | A | 77 | 0.16 | 68 | 4 |
| 3 | hEPO | A | 95 | 0.11 | 86 | 4 |
| 4 | hEPO | A | 78 | 0.16 | 94 | 4 |
| 5 | hEPO | A | 107 | 0.11 | 75 | 4 |
| 6 | hEPO | A | 114 | 0.16 | 99 | 4 |
| 8 | hEPO | A | 91 | 0.13 | 95 | 4 |
| 9 | hEPO | A | 114 | 0.1 | 91 | 4 |
| 11 | hEPO | A | 120 | 0.1 | 94 | 4 |
| 14 | hEPO | A | 83 | 0.19 | 77 | 4 |
| 15 | hEPO | A | 88 | 0.11 | 91 | 4 |
| 16 | hEPO | A | 84 | 0.11 | 77 | 4 |
| 19 | hEPO | A | 94 | 0.13 | 74 | 4 |
| 20 | hEPO | A | 95 | 0.13 | 89 | 4 |
| 21 | hEPO | A | 78 | 0.139 | 96 | 4 |
| 22 | hEPO | A | 75 | 0.1 | 91 | 4 |
| 24 | hEPO | A | 88 | 0.11 | 83 | 4 |
| 25 | hEPO | A | 81 | 0.13 | 96 | 4 |
| 26 | hEPO | A | 96 | 0.089 | 95 | 4 |
| 27 | hEPO | A | 89 | 0.14 | 95 | 4 |
| 28 | hEPO | A | 91 | 0.103 | 94 | 4 |
| 29 | hEPO | A | 126 | 0.13 | 96 | 4 |
| 30 | hEPO | A | 106 | 0.16 | 94 | 4 |
| 31 | hEPO | A | 98 | 0.12 | 97 | 4 |
| 32 | hEPO | A | 94 | 0.2 | 94 | 4 |
| 33 | hEPO | A | 109 | 0.11 | 99 | 4 |
| 34 | hEPO | A | 84 | 0.1 | 92 | 4 |
| 35 | hEPO | A | 92 | 0.08 | 82 | 4 |
| 36 | hEPO | A | 94 | 0.2 | 88 | 4 |
| 37 | hEPO | A | 65 | 0.25 | 95 | 4 |
| 38 | hEPO | A | 69 | 0.12 | 72 | 4 |
| 40 | hEPO | A | 84 | 0.11 | 66 | 4 |
| 51 | hEPO | A | 71 | 0.12 | 81 | 4 |
| 52 | hEPO | A | 74 | 0.14 | 95 | 4 |
| 53 | hEPO | A | 97 | 0.2 | 96 | 4 |
| 54 | hEPO | A | 79 | 1.6 | 87 | 4 |
| 55 | hEPO | A | 101 | 0.15 | 99 | 4 |

*The N/P ratio is defined as the ratio of the number of nitrogen in cationic lipid to the number of phosphate in nucleic acid.
ND indicates that the value was not determined.

### Example 3: Delivery of hEPO mRNA by Intramuscular Administration

### Mouse Studies

In summary, lipid screening studies were conducted with female BALB/cJ mice 6-8 weeks of age. Mice were dosed with 0.1 μg in 30 μL of LNPs by a single intramuscular (IM) injection into the gastrocnemius leg muscle. Blood samples were taken 6 and 24 hours post injection and hEPO levels were measured in the blood serum of the mice using an ELISA assay according to the manufacture's protocol. WO2022/099003 A1 also describes an in vivo assay for intramuscular administration (e.g. on page 46, paragraph [00206]).

Further details of the intramuscular experiment performed in this application are provided below.

### Study Design Table

| | | | Dose | | Dose | Dosing | Terminal |
|---|---|---|---|---|---|---|---|
| Group | No. of | Test | Levels | Conc. | Volume | Regimen | Time |
| No. | Animals | Article | (μg/An.) | (μg/mL) | (μL/An.) | ROA | Point |
| 1 | 4 | MC3 | 0.1 | 3.33 | 30 | Once on Day 1 | At 24 hours |
| 2 | 3 | 20 | 0.1 | 3.33 | | via IM injections | post dose on |
| 3 | 4 | 21 | 0.1 | 3.33 | | into the right | Day 2 |
| 4 | 4 | 22 | 0.1 | 3.33 | | gastrocenemius | |
| 5 | 4 | 24 | 0.1 | 3.33 | | muscle. | |
| 6 | 4 | 25 | 0.1 | 3.33 | | | |
| 7 | 4 | 26 | 0.1 | 3.33 | | | |
| 8 | 4 | 27 | 0.1 | 3.33 | | | |
| 9 | 3 | 28 | 0.1 | 3.33 | | | |
| 10 | 4 | 29 | 0.1 | 3.33 | | | |
| 11 | 4 | 30 | 0.1 | 3.33 | | | |
| 12 | 4 | 32 | 0.1 | 3.33 | | | |
| 13 | 4 | 33 | 0.1 | 3.33 | | | |
| 14 | 4 | 34 | 0.1 | 3.33 | | | |
| 15 | 4 | 35 | 0.1 | 3.33 | | | |
| 16 | 4 | 40 | 0.1 | 3.33 | | | |
| 17 | 4 | MC3 | 0.1 | 3.33 | | | |
| 18 | 4 | 1 | 0.1 | 3.33 | | | |
| 19 | 4 | 2 | 0.1 | 3.33 | | | |
| 20 | 4 | 3 | 0.1 | 3.33 | | | |
| 21 | 4 | 4 | 0.1 | 3.33 | | | |
| 22 | 4 | 5 | 0.1 | 3.33 | | | |
| 23 | 4 | 6 | 0.1 | 3.33 | | | |
| 24 | 4 | 8 | 0.1 | 3.33 | | | |
| 25 | 4 | 9 | 0.1 | 3.33 | | | |
| 26 | 4 | 11 | 0.1 | 3.33 | | | |
| 27 | 4 | 14 | 0.1 | 3.33 | | | |
| 28 | 4 | 15 | 0.1 | 3.33 | | | |
| 29 | 4 | 16 | 0.1 | 3.33 | | | |
| 30 | 4 | 19 | 0.1 | 3.33 | | | |
| 31 | 4 | 37 | 0.1 | 3.33 | | | |
| 32 | 4 | 38 | 0.1 | 3.33 | | | |
| 33 | 4 | 31 | 0.1 | 3.33 | | | |

An. = animal;
TA = test article;
Conc. = concentration;
ROA = route of administration;
IM = intramuscular.

### Test Materials and Treatment Regimen

Test materials remained RNase free during loading into the syringe (as applicable).

### Test Article Class of Compound: Oligonucleotides

### ABSL-1

Treatment Regimen: On Day 1, animals from Groups 1-33 were dosed via intramuscular injection while under light isoflurane anesthesia according to the study design table above. Animals in Groups 1-33 were injected with EPO mRNA LNPs in the right leg only. Groups 1 and 17 animals received MC3 control. The cationic lipid MC3 is currently investigated for in vivo delivery of e.g. siRNA (see WO2010/144740).

### Study Animals

### Animals:

| Species/Sex | Mouse/Female |
|---|---|
| Strain | BALB/cJ (Jax #000651) |
| Number | N = 130 |
| Age | 6-8 Weeks |

Acclimation: Animals were acclimatised to the Test Facility for at least 24 hours.

Housing: All animals were socially housed in polycarbonate cages with contact bedding in an animal housing room.

Food and Water: Food (Envigo irradiated 2918 diet) and filtered tap water was provided to animals ad libitum.

### In-Life Observations and Measures

Animal Health Checks: At least once daily animals received a cage side health check observation.

Clinical Observations: Clinical observations were performed for all animals on Day 1 prior to dose administration and prior to euthanasia. Clinical observations were performed more often if abnormal clinical signs were exhibited by animals on study.

Body Weights: Body weights were recorded prior to test material administration. Body weights were rounded to the nearest 0.1 g.

Interim Sample Collections: Interim whole blood (^{~}50 μL) was collected by tail snip or saphenous vein at 6 and 24 hours post dose administration (±5%). Blood samples were collected into serum separator tubes, allowed to clot at room temperature for at least 10 minutes, centrifuged at ambient temperature at minimum 1000 g for 10 minutes and the serum was extracted. All serum samples were stored at nominally −70° C. until analysis hEPO by the Testing Facility. The results of the EPO analysis were included in the Data Submission.

### In-Life Sample Collection Table

| | In Life Sample Collection Time Points |
|---|---|
| Group No. | Whole Blood |
| 1-33 | T = 6 Hours Post Dose |
| Volume | ~50 μL |
| Additive | None |

No. = number

### Terminal Procedures

Euthanasia: On Day 2, 24 hours post dose, all animals were euthanized by CO₂ asphyxiation followed by thoracotomy and terminal blood collection.

Terminal Blood Collections: Whole blood was collected via cardiac puncture into serum separator tube, allowed to clot at room temperature for at least 10 minutes, centrifuged at ambient temperature at minimum 1000 g for 10 minutes and the serum was extracted. Serum samples were stored at nominally −70° C. until analyzed for hEPO by the Test Facility.

### Terminal Sample Collection Table

| | Terminal Sample Collection Time Points |
|---|---|
| Group No. | Whole Blood |
| 1-33 | T = 24 Hours Post Dose |
| Volume | MOV |
| Additive | None |

No. = number;
MOV = maximum obtainable volume.

### In-Vitro Assays:

ELISA Assay: Human erythropoietin (hEPO) levels in sera samples were determined by ELISA kit (R&D systems, Cat #DEP-00) according to the manufactory instruction and the results were included in the Data Submission. The “shaker” protocol was used. The serum samples were diluted between 1:40 and 1:100.

### Reporting and Data Retention

Data Submission: A tabulated data summary of animal assignment, individual and group means (as applicable) for times of dose administration and euthanasia, body weights, clinical observations in-vitro analysis and mortality (as applicable) were delivered for this study.

### Results

The results of this example are shown in Tables 2, 3 and 4 below. Lipid nanoparticles comprising the cationic lipids of the present invention exhibited improved hEPO expression levels compared to lipid nanoparticles comprising MO, which is the currently investigated for in vivo delivery of e.g. siRNA (see WO2010/144740).

TABLE 2
Results of hEPO mRNA delivery studies for lipids-intramuscular administration of hEPO
mRNA lipid formulations comprising the claimed isosorbide-derived cationic lipids.

| | | hEPO 6 Hr | | hEPO 24 Hr | |
|---|---|---|---|---|---|
| | | (ng/ml) | | (ng/mL) | |
| | hEPO 6 Hr | Standard | hEPO 24 Hr | Standard | # of |
| Compound # | (ng/ml) Mean | Deviation (SD) | (ng/ml) Mean | Deviation (SD) | mice |
| MC3 | 7.1 | 0.9 | NT | NT | 4 |
| 20 | 25.2 | 4.0 | NT | NT | 3 |
| 21 | 31.6 | 4.7 | NT | NT | 4 |
| 22 | 34.1 | 12.6 | NT | NT | 4 |
| 24 | 30.2 | 6.0 | 19.4 | 3.6 | 4 |
| 25 | 42.7 | 13.6 | NT | NT | 4 |
| 26 | 42.2 | 11.5 | NT | NT | 4 |
| 27 | 28.3 | 5.2 | NT | NT | 4 |
| 28 | 40.2 | 1.75 | NT | NT | 3 |
| 29 | 38.4 | 10.6 | NT | NT | 4 |
| 30 | 25.2 | 4.0 | NT | NT | 4 |
| 31 | 14.1 | 4.1 | NT | NT | 4 |
| 32 | 33.2 | 5.0 | NT | NT | 4 |
| 33 | 17.6 | 4.3 | NT | NT | 4 |
| 34 | 33.8 | 6.9 | NT | NT | 4 |
| 35 | 28.5 | 11.4 | NT | NT | 4 |
| 40 | 11.9 | 1.1 | 7 | 0.7 | 4 |

NT indicates not tested.

TABLE 3
Results of hEPO mRNA delivery studies-intramuscular administration of hEPO mRNA lipid
formulations comprising the claimed isomannide-derived cationic lipids.

| | | hEPO 6 Hr | | hEPO 24 Hr | |
|---|---|---|---|---|---|
| | | (ng/mL) | | (ng/ml) | |
| | hEPO 6 Hr | Standard | hEPO 24 Hr | Standard | # of |
| Compound # | (ng/mL) Mean | Deviation (SD) | (ng/ml) Mean | Deviation (SD) | mice |
| MC3 | 5.6 | 1.2 | NT | NT | 4 |
| 1 | 42.5 | 13.6 | NT | NT | 4 |
| 2 | 36.3 | 10.2 | NT | NT | 4 |
| 3 | 37.4 | 4.0 | 18.9 | 4.4 | 4 |
| 4 | 43.2 | 8.9 | NT | NT | 4 |
| 5 | 26.3 | 4.4 | NT | NT | 4 |
| 6 | 40.9 | 7.1 | NT | NT | 4 |
| 8 | 25.7 | 8.6 | NT | NT | 4 |
| 9 | 30.7 | 5.0 | 19.8 | 4.8 | 4 |
| 11 | 36 | 3.9 | NT | NT | 4 |
| 14 | 19.3 | 8.0 | NT | NT | 4 |
| 15 | 22.8 | 2.7 | NT | NT | 4 |
| 16 | 9.4 | 5.1 | NT | NT | 4 |
| 19 | 10.7 | 4.3 | 5.35 | 2.2 | 4 |
| 37 | 27.4 | 10.5 | NT | NT | 4 |
| 38 | 22.6 | 9.5 | NT | NT | 4 |

NT indicates not tested.

Further lipids of the invention were tested in accordance with the experiments described in example 3 above and the results of those experiments are set out in Table 4 below.

TABLE 4
Results of hEPO mRNA delivery studies for lipids-intramuscular administration of hEPO
mRNA lipid formulations comprising the claimed isosorbide-derived cationic lipids.

| | hEPO 6 Hr | hEPO 6 Hr (ng/ml) |
|---|---|---|
| Compound # | (ng/ml) Mean | Standard Deviation (SD) |
| 12 | 34.4 | 2.3 |
| 36 | 31.7 | 11.2 |
| 44 | 2.0 | 1.1 |
| 45 | 2.2 | 0.0 |
| 46 | 2.1 | 0.0 |
| 48 | 81.3 | 25.3 |
| 51 | 47.6 | 17.5 |
| 52 | 72.7 | 11.7 |
| 53 | 72.0 | 25.9 |
| 54 | 12.0 | 2.9 |
| 55 | 30.9 | 13.4 |
| 56 | 51.9 | 19.5 |
| 63 | 60.5 | 16.7 |
| 64 | 36.2 | 15.3 |
| 65 | 28.0 | 3.5 |
| 66 | 23.2 | 3.6 |
| 67 | 34.4 | 9.9 |
| 70 | 47.6 | 7.8 |
| 71 | 72.0 | 25.9 |
| 72 | 6.2 | 2.4 |
| 73 | 23.4 | 13.6 |
| 74 | 2.1 | 0.0 |
| 75 | 2.1 | 0.0 |
| 76 | 18.0 | 6.4 |
| 77 | 10.8 | 2.8 |
| 79 | 21.2 | 4.0 |
| 80 | 0.9 | 0.3 |
| 81 | 2.3 | 1.0 |
| 82 | 3.1 | 1.6 |
| 83 | 8.0 | 1.7 |
| 84 | 22.1 | 6.0 |
| 85 | 23.8 | 2.4 |

Comparative ether compounds not falling within the scope of the claims in accordance with the experiments described in example 3 above and the results of those experiments are set out in Table 5 below.

TABLE 5
Results of hEPO mRNA delivery studies for lipids - intramuscular administration of hEPO
mRNA lipid formulations comprising the claimed isosorbide-derived cationic lipids.

| | | hEPO 6 Hr (ng/ml) |
|---|---|---|
| | hEPO 6 Hr (ng/ml) | Standard Deviation |
| Compound | Mean | (SD) |
| 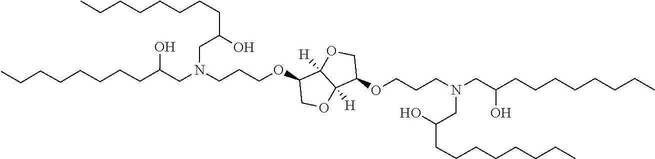 [SMILES: CCCCCCCCC(O)CN(CCCO[C@@H]1CO[C@H]2[C@@H]1OC[C@H]2OCCCN(CC(O)CCCCCCCC)CC(O)CCCCCCCC)CC(O)CCCCCCCC] | 11.3 | 1.6 |
| 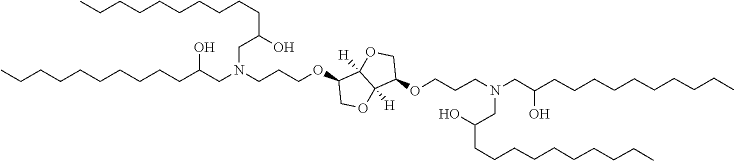 [SMILES: CCCCCCCCCCC(O)CN(CCCO[C@@H]1CO[C@H]2[C@@H]1OC[C@H]2OCCCN(CC(O)CCCCCCCCCC)CC(O)CCCCCCCCCC)CC(O)CCCCCCCCCC] | 33.8 | 10.0 |
| 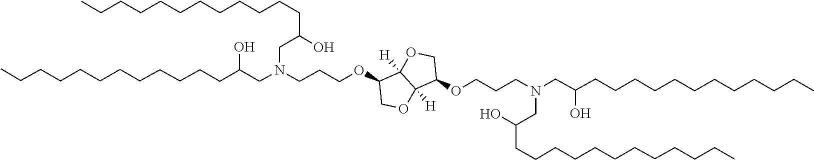 [SMILES: CCCCCCCCCCCCC(O)CN(CCCO[C@@H]1CO[C@H]2[C@@H]1OC[C@H]2OCCCN(CC(O)CCCCCCCCCCCC)CC(O)CCCCCCCCCCCC)CC(O)CCCCCCCCCCCC] | 13.9 | 4.2 |

### Example 4: In Vitro Degradation Study

Lipid degradability by MOUSE/HUMAN lung S9 in vitro

Assay format—4 or 5 time points in triplicate.

I. Assay procedure:
1) Plan experiment, compounds, and reagents.
2) Dissolve each lipid in DMSO or IPA to make 5 mM stock, then dilute by IPA to 200 μM work solution.
3) Thaw mouse and human lung S9.
4) Prepare pooled incubation mixture as in the reaction formulas below on ice.
5) Aliquot 495 μL incubation mixture prepared in step #4 to each well of a 2 mL 96-well plate.
6) Add 5 μL compound to each well to initiate the reaction. Take t0 samples (as in step #8).
7) Cover the plate with 2 layers of breathable seals and incubate the plate on an orbital shaker at 150 rpm in a 37° C. CO₂ incubator.
8) At each time point, pipette to mix the incubation mixture 5 times, then take 70 μL of incubation mixture to a fresh plate. Store in −20° C. freezer immediately.
9) Add 210 μL (3× volume) of the cold stop solution to each well of the sample plates collected. Mix at 600 rpm on an orbital shaker for 15 min.
10) Centrifuge the quenched plates at 3800 rpm for 10 min at 4° C. and transfer supernatant to fresh plates.
11) Load the supernatant on filter plates and centrifuge again at 3800 rpm for 5 min at 4° C. Collect final samples in fresh plates for LC/MS.

### II. Time Course and Stop Solutions:

4-5 Time points (hour): e.g. 0, 4, 8, 24.48 hr
stop solution: 1:1:1 ACN/MeOH/IPA (v/v/v) with propranolol & MC3 as internal standard. Store at 4° C.

### III. Reaction Components and Formulas:

MOUSE/HUMAN lung S9

| Final conc. | | Reaction component | Volume (μL) |
|---|---|---|---|
| | | H₂O | 155.0 |
| 100 | mM | 5x potassium phosphate | 100.0 |
| | | buffer pH 7.4 | |
| 2.4 | mg/ml | each species lung S9 | 240.0 |
| | | (5 mg/ml) | |
| 2 | μM | lipid (200 μM) | 5.0 |
| | | Total | 500 |

### Example 5: RiboGreen Assay

The RiboGreen Assay is a fluorescence-based method for the determination of mRNA concentration (Total and Free) and % encapsulation using Quant-iT™ RiboGreen® RNA reagent in mRNA containing lipid nanoparticles.

### Materials/Reagents

Triton-X, 98%, for molecular biology, DNAse, RNAse and Protease free, Acros Organics, Cat. AC327371000
UltraPure DNase/RNase-free Distilled Water Life Technologies, Cat. 10977-023
RNaseZap® RNase Decontamination Solution Life Technologies, Cat. AM9784
Quant-iT™ RiboGreen® RNA Reagent Life Technologies, Cat. R11491 or Quant-IT™ RiboGreen® RNA Assay Kit Life Technologies, Cat. R11490
RNase free 20× TE Buffer Life Technologies, Cat. T11493
RNaseZap® RNase Decontamination Solution Life Technologies, Cat. AM9784

### Equipment

Molecular Devices Gemini EM Microplate Reader
RNase Free Microcentrifuge Tubes (2.0 mL)
RNase Free Flacon Tubes (15 and 50 mL)
Vortex mixer
Corning® 96 Well Special Optics Microplate with Clear Background (Cat #3615)
   Preparation of mRNA Standards

mRNA Dilution

| | 50X | 2X |
|---|---|---|
| 10X TE buffer | 950 μL | 1920 μL |
| mRNA | 50 μL | 80 μL |

| | 0 | 0.02 ug/mL | 0.05 ug/mL | 0.2 ug/mL | 0.4 ug/mL | 0.6 ug/mL |
|---|---|---|---|---|---|---|
| Standards | blank | mRNA-1 | mRNA-2 | mRNA-3 | mRNA-4 | mRNA-5 |
| 10× TE buffer | 950 μL | 930 μL | 900 μL | 750 μL | 550 μL | 350 μL |
| 4% Triton | 50 μL | 50 μL | 50 μL | 50 μL | 50 μL | 50 μL |
| 2× mRNA dil. | 0 μL | 20 μL | 50 μL | 200 μL | 400 μL | 600 μL |
| 200-Fold RG | 1000 μL | 1000 μL | 1000 μL | 1000 μL | 1000 μL | 1000 μL |

### Sample Preparation

Sample Dilution

| | 100X |
|---|---|
| 10X TE buffer | 990 μL |
| LNP sample | 10 μL |

Samples

| | Total mRNA | Free mRNA |
|---|---|---|
| 10X TE buffer | 900 μL | 750 μL |
| 4% Triton | 50 μL | 0 μL |
| LNP sample dil. (100x) | 50 μL | 250 μL |
| 200-Fold RG | 1000 μL | 1000 μL |

### 200-Fold RiboGreen

200-Fold RiboGreen

| | |
|---|---|
| # tubes*5 uL = Amount | Ex. (10 tubes + 2 extra) *5 uL = |
| of RG in (#tube)mL TE | 60 μL RG in 12 mL TE |

### Procedure

To each of the standards (Blank, mRNA-1, mRNA-2. mRNA-3, mRNA-4, mRNA-5) and Samples (free mRNA and total mRNA), add 1.0 mL of 200-fold Ribogreen Reagent Solution and gently mix by inversion. This is a 2× Dilution.
Add 200 μL of each standard and sample in triplicate using the reverse pipetting technique in a 96-well Costar Black with Clear Background Plate. Ensure no bubbles are present in the plate before the fluorescence reading.
Read the fluorescence signal using the below instrument parameters:
Read Type: Fluorescence, Bottom Read
Excitation: 485 nm; Cut-off: 515 nm; Emission: 530 nm
Plate Type: 96-well Costar Black with Clear Background

### Data Analysis

The average fluorescence from each calibration standard is plotted against the concentration to generate a linear calibration curve using the MS Excel software. The coefficient of determination (R²) of calibration curve must be R²>0.99.

The linear equation generated can be interpreted as follows:

$$y = mx + c$$

Where,
Y=average fluorescence value
m: slope
x: concentration (pg/mL)
c: y-intercept
   Using the linear equation, calculate the concentration of free and total mRNA concentration in the test sample by replacing the y value in the equation with the average fluorescence value of each respective sample
   Once the concentration is determined, the actual concentration in the sample can be back-calculated by multiplying the concentration in the test sample with the dilution factor (DF) as follows:

$$Free m R N A Conc . = Conc . of Free m R N A in Test Sample X 800 ( D F )$$

$$Total m R N A Conc . = Conc . of Total m R N A in Test Sample X 4000 ( D F )$$

Concentration of encapsulated mRNA can be determined by subtracting the concentration of free mRNA from the total mRNA.
% Encapsulation can then be calculated by taking the ratio of encapsulated mRNA over total mRNA and multiplying the result with 100.

### Example 6: Influenza Titer

Groups of Balb/c mice (Mus musculus) as per the treatment group were immunized under isoflurane anesthesia with a dose of 0.4 ug per mouse in 0.05 mL of Compound 24/or Compound 3/Modified Tasmania H3 mRNA-lipid nanoparticles via the IM route in the quadriceps, on day 0 in one hind leg and day 21 in the contralateral leg. Mice were evaluated for a minimum of 3 days post-administration and any animal that lost displayed severe clinical signs after the veterinarian's assessment was euthanized by administration of 5 mg/kg of meloxicam by subcutaneous injection.

Blood was collected via submandibular or orbital sinus bleeds (in-life bleeds were performed on day −1 and on day 20) and cardiac puncture (terminal bleed, day 35) from all animals under sedation. Mice were bled on pre-study to obtain a base-line pre-immune serum sample and for pre-screening purposes.

HAI assays were performed using the A/Tasmania/503/2020 (H3N2) virus stocks (BIOQUAL, Inc.). Sera were treated with receptor-destroying enzyme (RDE) by diluting one-part serum with three parts enzyme and incubated overnight in a 37° C. water bath. Enzyme was inactivated by a 30-minute incubation period at 56° C. followed by addition of six parts PBS for a final dilution of 1/10. HAI assays were performed in V-bottom 96-well plates using four hemagglutinating units (HAU) of virus and 0.5% turkey RBC. The reference serum for each strain was included as a positive control on every assay plate. Each plate also included a back-titration to confirm the antigen dose (4 HAU/25 pl) as well as a negative control sample (PBS or naive control serum). The HAI titer was determined as the highest dilution of serum resulting in complete inhibition of hemagglutination. Results were only valid for plates with the appropriate back-titration result (verifying 4 HAU/25 ul added) and a reference serum titer within 2-fold of the expected titer. Compound 3 was shown to induce an HAI GMT of 320 and Compound 24 was shown to induce an HAI GMT of 190, demonstrating induction of immunogenicity with both LNPs.

From the foregoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

### Numbered Embodiments

1. A compound having a structure according to Formula (I):

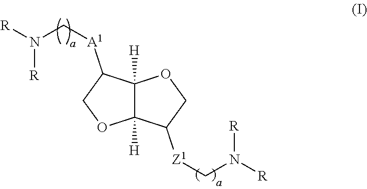

or a pharmaceutically acceptable salt thereof wherein:
A¹ is selected from —C(═O)O—, —C(═O)S—, —C(═O)NH—, —OC(═O)O—, —OC(═O)NH—, —NHC(═O)O—, —SC(═O)NH—, —OCH₂CH₂O—, —OCH₂O—, —OCH(CH₃)O—, —S— and —S—S—, wherein the left hand side of each recited structure is bound to the —(CH₂)ₐ—;
Z¹ is selected from —OC(═O)—, —SC(═O)—, —NHC(═O)—, —OC(═O)O—, —NHC(═O)O—, —OC(═O)NH—, —NHC(═O)S—, —OCH₂CH₂O—, —OCH₂O—, —OCH(CH₃)O—, —S— and —S—S—, wherein the right hand side of each recited structure is bound to the —(CH₂)ₐ—;
each R is independently selected from:

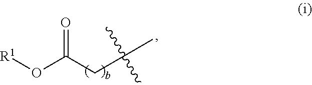

wherein each R¹ is independently selected from optionally substituted alkyl, optionally substituted alkenyl, and optionally substituted alkynyl;

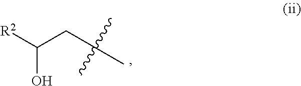

wherein each R² is independently selected from optionally substituted alkyl, optionally substituted alkenyl, optionally substituted alkynyl, and —W¹—X¹,
   wherein each W¹ is independently selected from optionally substituted alkylene and optionally substituted alkenylene, and
   each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;

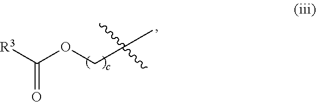

wherein each R³ is independently selected from optionally substituted alkyl, optionally substituted alkenyl, and optionally substituted alkynyl; and

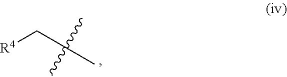

wherein each R⁴ is independently selected from optionally substituted cycloalkyl or optionally substituted heterocycloalkyl;
wherein at least three R are independently selected from

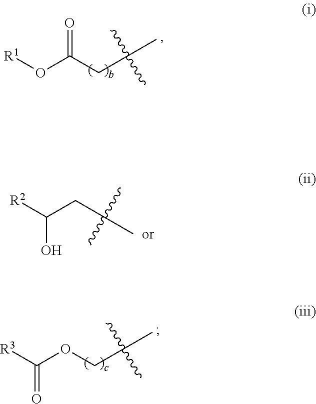

each a is independently selected from 2, 3, 4, and 5;
each b is independently selected from 2, 3, 4, 5, 6, 7, 8, 9 and 10; and
each c is independently selected from 2, 3, 4, 5, 6, 7, 8, 9 and 10.

2. The compound of numbered embodiment 1, wherein the compound has a structure according to Formula (I′):

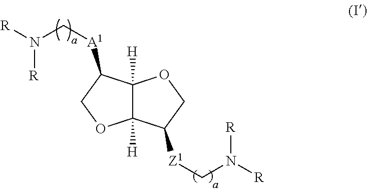

or a pharmaceutically acceptable salt thereof.

3. The compound of numbered embodiment 1, wherein the compound has a structure according to Formula (IA):

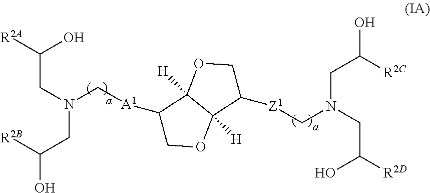

or a pharmaceutically acceptable salt thereof, wherein each R^{2A}, R^{2′}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

4. The compound of numbered embodiment 1 or 3, wherein the compound has a structure according to Formula (IE):

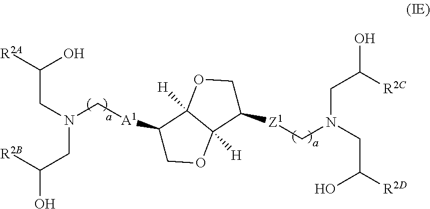

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

5. The compound of any one of numbered embodiments 1-4, wherein the compound has a structure according to Formula (IB1a):

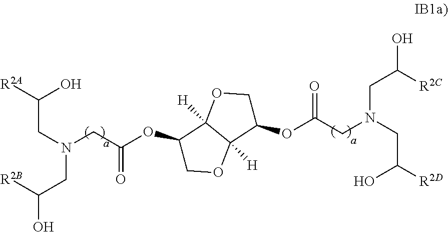

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is independently selected from 3 or 4.

6. The compound of numbered embodiment 1 or 2, wherein the compound has a structure according to Formula (IB1b):

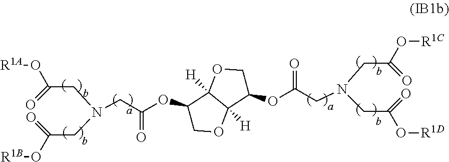

or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl;
optionally wherein i) each a is 2, and/or ii) each b is independently selected from 5 or 7.

7. The compound of numbered embodiment 1 or 2, wherein the compound has a structure according to Formula (IB1c):

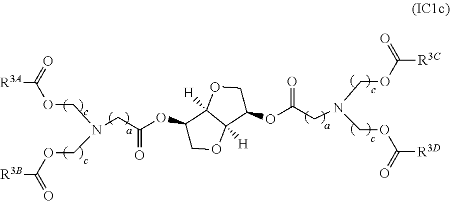

or a pharmaceutically acceptable salt thereof, wherein each R^{3A}, R^{3B}, R^{3C} and R^{3D} is independently selected from optionally substituted alkyl, optionally substituted alkenyl, and optionally substituted alkynyl;
optionally wherein i) each a is 3, and/or ii) each c is 6.

8. The compound of numbered embodiment 1 or 2, wherein the compound has a structure according to Formula (IB1d):

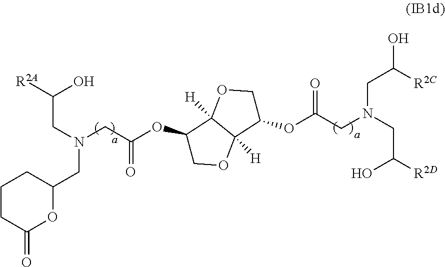

or a pharmaceutically acceptable salt thereof, wherein each R^{2A}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is 3.

9. The compound of numbered embodiment 1, wherein the compound has a structure according to Formula (I″):

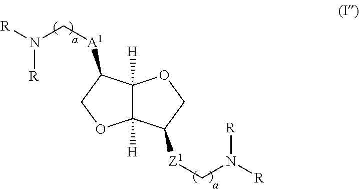

or a pharmaceutically acceptable salt thereof.

10. The compound of numbered embodiment 1 or 9, wherein the compound has a structure according to Formula (IB2a):

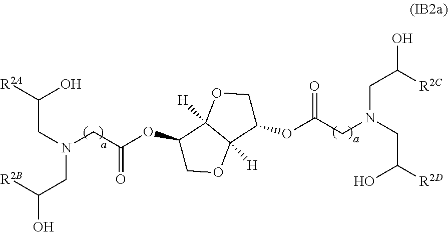

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is independently selected from 3 or 4.

11. The compound of any one of numbered embodiments 1-4, wherein the compound has a structure according to Formula (IC1a):

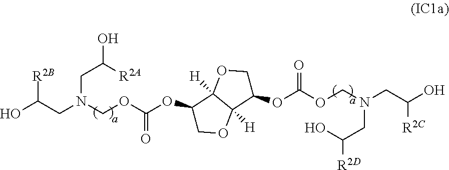

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is independently selected from 3 or 4.

12. The compound of numbered embodiment 1 or 2, wherein the compound has a structure according to Formula (IC1b):

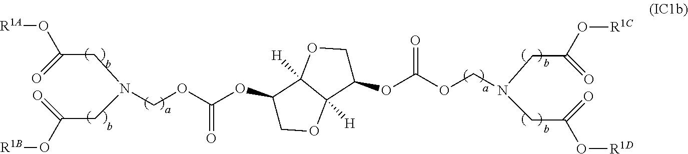

or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl;
optionally wherein each a is 2, and/or ii) each b is independently selected from 5 or 7.

13. The compound of numbered embodiment 1 or 9, wherein the compound has a structure according to Formula (IC2a):

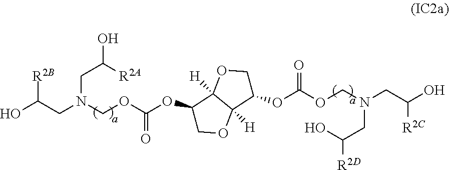

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is 3.

14. The compound of numbered embodiment 1 or 3, wherein the compound has a structure according to Formula (ID):

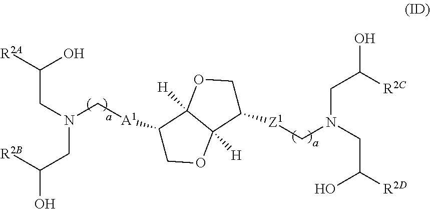

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

15. The compound of any one of numbered embodiments 1-3, 9, or 14, wherein A¹ is —C(═O)NH—, wherein the left hand side of the recited structure is bound to the —(CH₂)ₐ—, and Z¹ is —NHC(═O)—, wherein the right hand side of the recited structure is bound to the —(CH₂)ₐ—;
optionally wherein each a is 3.

16. The compound of any one of numbered embodiments 1-3, 9, or 14, wherein A¹ is —OC(═O)NH—, wherein the left hand side of the recited structure is bound to the —(CH₂)ₐ—, and Z¹ is —NHC(═O)O—, wherein the right hand side of the recited structure is bound to the —(CH₂)ₐ—;
optionally wherein each a is 3.

17. The compound of any one of numbered embodiments 1-3, 9, or 14, wherein A¹ is —SC(═O)NH—, wherein the left hand side of the recited structure is bound to the —(CH₂)ₐ—, and Z¹ is —NHC(═O)S—, wherein the right hand side of the recited structure is bound to the —(CH₂)ₐ—;
optionally wherein each a is 3.

18. The compound of any one of numbered embodiments 1-3, 9, or 14, wherein A¹ is —C(═O)S—, wherein the left hand side of the recited structure is bound to the —(CH₂)ₐ—, and Z¹ is —SC(═O)—, wherein the right hand side of the recited structure is bound to the —(CH₂)ₐ—;
optionally wherein each a is 3.

19. The compound of any one of numbered embodiments 1-3, 9, or 14, wherein A¹ is —S—S—, and Z¹ is —S—S—;
optionally wherein each a is 3.

20. The compound of any one of numbered embodiments 1-3, 9, or 14, wherein A¹ is —S—, and Z¹ is —S—;
optionally wherein each a is 4.

21. The compound of any one of numbered embodiments 1-3, 9, or 14, wherein A¹ is —S—S—, and Z¹ is —SC(═O)—, wherein the right hand side of the recited structure is bound to the —(CH₂)ₐ—;
optionally wherein each a is 3.

22. The compound of any one of numbered embodiments 1-4 or 9, wherein A¹ is —NHC(═O)O—, wherein the left hand side of the recited structure is bound to the —(CH₂)ₐ—, and Z¹ is —OC(═O)NH—, wherein the right hand side of the recited structure is bound to the —(CH₂)ₐ—;
optionally wherein each a is 3.

23. A compound having a structure according to Formula (II):

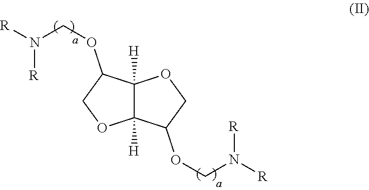

or a pharmaceutically acceptable salt thereof wherein:
each R is independently selected from:

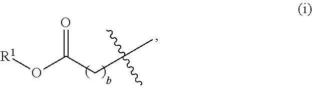

wherein each R¹ is independently selected from optionally substituted alkyl, optionally substituted alkenyl, and optionally substituted alkynyl; and

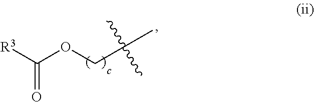

wherein each R³ is independently selected from optionally substituted alkyl, optionally substituted alkenyl, and optionally substituted alkynyl;
each a is independently selected from 2, 3, 4, and 5;
each b is independently selected from 2, 3, 4, 5, 6, and 7; and
each c is independently selected from 2, 3, 4, 5, 6, and 7.

24. The compound of numbered embodiment 23, wherein the compound has a structure according to Formula (II′):

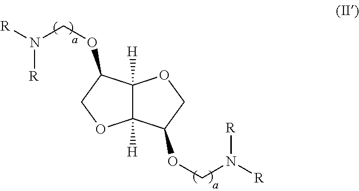

or a pharmaceutically acceptable salt thereof.

25. The compound of numbered embodiment 23 or 24, wherein the compound has a structure according to Formula (IIA):

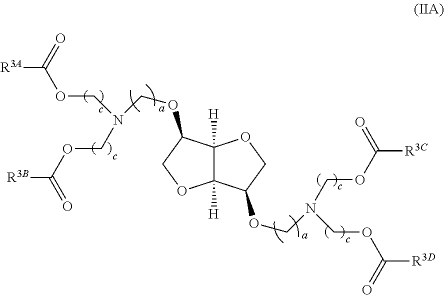

or a pharmaceutically acceptable salt thereof, wherein each R^{3A}, R^{3B}, R^{3C} and R^{3D} is independently selected from optionally substituted alkyl, optionally substituted alkenyl, and optionally substituted alkynyl.

26. The compound of numbered embodiment 23 or 24, wherein the compound has a structure according to Formula (IIB):

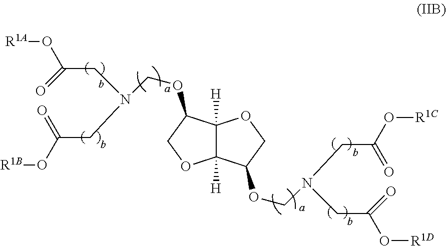

or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl.

27. The compound of any one of numbered embodiments 1-4, 9, or 14 or a pharmaceutically acceptable salt thereof, wherein A¹ and Z¹ are the same.

28. The compound of any one of numbered embodiments 1-4, 9, or 14 or a pharmaceutically acceptable salt thereof, wherein A¹ and Z¹ are different.

29. The compound of any one of numbered embodiments 1-4, 9, 14, 27 or 28 or a pharmaceutically acceptable salt thereof, wherein A¹ is —C(═O)O—, wherein the left hand side of the recited structure is bound to the —(CH₂)ₐ—.

30. The compound of any one of numbered embodiments 1-4, 9, 14, 27 or 28 or a pharmaceutically acceptable salt thereof, wherein A¹ is —OC(═O)O—, wherein the left hand side of the recited structure is bound to the —(CH₂)ₐ—.

31. The compound of any one of numbered embodiments 1-4, 9, 14 or 27-30 or a pharmaceutically acceptable salt thereof, wherein Z¹ is —OC(═O)—, wherein the right hand side of the recited structure is bound to the —(CH₂)ₐ—.

32. The compound of any one of numbered embodiments 1-4, 9, 14 or 27-30 or a pharmaceutically acceptable salt thereof, wherein Z¹ is —OC(═O)O—, wherein the right hand side of the recited structure is bound to the —(CH₂)ₐ—.

33. The compound of any one of numbered embodiments 1-32 or a pharmaceutically acceptable salt thereof, wherein each a is independently selected from 3 and 4.

34. The compound of any one of numbered embodiments 1-33 or a pharmaceutically acceptable salt thereof, wherein each a is 3.

35. The compound of any one of numbered embodiments 1-33 or a pharmaceutically acceptable salt thereof, wherein each a is 4.

36. The compound of any one of numbered embodiments 1-33 or a pharmaceutically acceptable salt thereof, wherein the value for the a on the left hand side of the depicted Formula is 3 and the value for the a on the right hand side of the depicted Formula is 4.

37. The compound of any one of numbered embodiments 1-33 or a pharmaceutically acceptable salt thereof, wherein the value for the a on the left hand side of the depicted Formula is 4 and the value for the a on the right hand side of the depicted Formula is 3.

38. The compound of any one of numbered embodiments 1, 2, 6, 9, 12, 15-24 or 26-37 or a pharmaceutically acceptable salt thereof, wherein the compound has a structure according to Formula (I), Formula (I′), Formula (IB1b), Formula (I″), Formula (IC1b), Formula (II), Formula (II′), or Formula (IIB), wherein each b is independently selected from 5, 6, and 7.

39. The compound of any one of numbered embodiments 1, 2, 6, 9, 12, 15-24 or 26-38 or a pharmaceutically acceptable salt thereof, wherein the compound has a structure according to Formula (I), Formula (I′), Formula (IB1b), Formula (I″), Formula (IC1b), Formula (II), Formula (II′), or Formula (IIB), wherein each b is independently selected from 5 and 7.

40. The compound of any one of numbered embodiments 1, 2, 6, 9, 12, 15-24 or 26-39 or a pharmaceutically acceptable salt thereof, wherein the compound has a structure according to Formula (I), Formula (I′), Formula (IB1b), Formula (I″), Formula (IC1b), Formula (II), Formula (II′), or Formula (IIB), wherein each b is 5.

41. The compound of any one of numbered embodiments 1, 2, 6, 9, 12, 15-24 or 26-39 or a pharmaceutically acceptable salt thereof, wherein the compound has a structure according to Formula (I), Formula (I′), Formula (IB1b), Formula (I″), Formula (IC1b), Formula (II), Formula (II′), or Formula (IIB), wherein each b is 7.

42. The compound of any one of numbered embodiments 1, 2, 7, 9, 15-25, or 27-41 or a pharmaceutically acceptable salt thereof, wherein the compound has a structure according to Formula (I), Formula (I′), Formula (IB1c), Formula (I″), Formula (II), Formula (II′), or Formula (IIA), wherein each c is 6.

43. The compound of any one of numbered embodiments 1, 2, 9, 15-22 or 27-42, wherein the compound has a structure according to Formula (I), Formula (I′) or Formula (I″), wherein R⁴ is optionally substituted heterocycloalkyl.

44. The compound of any one of numbered embodiments 1, 2, 9, 15-22 or 27-43, wherein the compound has a structure according to Formula (I), Formula (I′) or Formula (I″), wherein R⁴ is

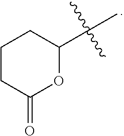 [SMILES: *C1CCCC(=O)O1]

45. The compound of any one of numbered embodiments 1, 2, 9, 15-22, or 27-44 or a pharmaceutically acceptable salt thereof, wherein the compound has a structure according to Formula (I), Formula (I′), or Formula (I″), wherein each R is independently selected from:

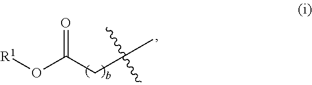

wherein each R¹ is independently selected from optionally substituted alkyl, optionally substituted alkenyl, and optionally substituted alkynyl;

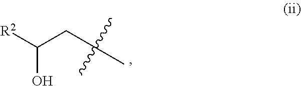

wherein each R² is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹; and

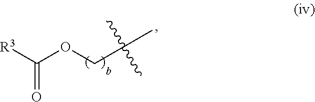

wherein each R³ is independently selected from optionally substituted alkyl, optionally substituted alkenyl, and optionally substituted alkynyl.

46. The compound of any one of numbered embodiments 1, 2, 9, 15-22, or 27-45 a pharmaceutically acceptable salt thereof, wherein the compound has a structure according to Formula (I), Formula (I′), Formula (I″), Formula (II), or Formula (II′), wherein each R is independently selected from

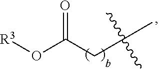

wherein each R¹ is independently selected from optionally substituted alkyl, optionally substituted alkenyl, and optionally substituted alkynyl.

47. The compound of any one of numbered embodiments 1, 2, 6, 9, 12, 15-24, or 26-46 or a pharmaceutically acceptable salt thereof, wherein the compound has a structure according to Formula (I), Formula (I′), Formula (IB1b), Formula (I″), Formula (IC1b), Formula (II), Formula (II′), or Formula (IIB), wherein each R¹ or each R^{1A}, R^{1B}, R^{1C} and R^{1D}, when present, is independently selected from optionally substituted (C₅-C₂₅) alkyl, optionally substituted (C₅-C₂₅) alkenyl, and optionally substituted (C₅-C₂₅) alkynyl.

48. The compound of any one of numbered embodiments 1, 2, 6, 9, 12, 15-24, or 26-46 or a pharmaceutically acceptable salt thereof, wherein the compound has a structure according to Formula (I), Formula (I′), Formula (IB1b), Formula (I″), Formula (IC1b), Formula (II), Formula (II′), or Formula (IIB), wherein each R¹ or each R^{1A}, R^{1B}, R^{1C} and R^{1D}, when present, is independently selected from optionally substituted alkyl.

49. The compound of any one of numbered embodiments 1, 2, 6, 9, 12, 15-24, or 26-48 or a pharmaceutically acceptable salt thereof, wherein the compound has a structure according to Formula (I), Formula (I′), Formula (IB1b), Formula (I″), Formula (IC1b), Formula (II), Formula (II′), or Formula (IIB), wherein each R¹ or each R^{1A}, R^{1B}, R^{1C} and R^{1D}, when present, is independently selected from optionally substituted (C₅-C₂₅) alkyl, for example optionally substituted (C₁₀-C₂₀) alkyl.

50. The compound of any one of numbered embodiments 1, 2, 6, 9, 12, 15-24, or 26-49 or a pharmaceutically acceptable salt thereof, wherein the compound has a structure according to Formula (I), Formula (I′), Formula (IB1b), Formula (I″), Formula (IC1b), Formula (II), Formula (II′), or Formula (IIB), wherein each R¹ or each R^{1A}, R^{1B}, R^{1C} and R^{1D}, when present, is independently selected from:

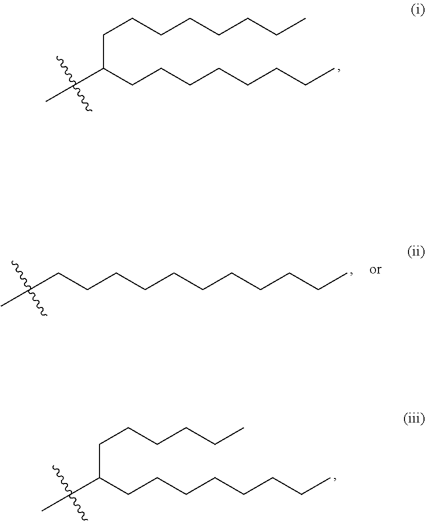 [SMILES: *C(CCCCCCCC)CCCCCCCC ; *CCCCCCCCCCC ; *C(CCCCCC)CCCCCCCC]

optionally wherein each R¹ or each R^{1A}, R^{1B}, R^{1C} and R^{1D}, when present, is independently selected from options (i) and (ii).

51. The compound of any one of numbered embodiments 1, 2, 9, 15-22, or 27-45 or a pharmaceutically acceptable salt thereof, wherein the compound has a structure according to Formula (I), Formula (I′), or Formula (I″), wherein each R is independently selected from

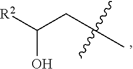

wherein each R² is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

52. The compound of any one of numbered embodiments 1-5, 8-11, 13-22, 27-45, or 47-51 or a pharmaceutically acceptable salt thereof, wherein the compound has a structure according to Formula (I), Formula (I′), Formula (IA), Formula (IE), Formula (IB1a), Formula (IB1d), Formula (I″), Formula (IB2a), Formula (IC1a), Formula (IC2a), or Formula (ID), wherein each R² or each R^{2A}, R^{2B}, R^{2C} and R^{2D}, when present, is independently selected from optionally substituted (C₅-C₂₅) alkyl, optionally substituted (C₅-C₂₅) alkenyl, optionally substituted (C₅-C₂₅) alkynyl, and —W¹—X¹,
wherein each W¹ is independently selected from optionally substituted (C₁-C₁₀) alkylene and optionally substituted (C₂-C₁₀) alkenylene, and
each X¹ is independently selected from —*O—(C═O)-optionally substituted (C₅-C₂₅) alkyl, —(*C═O)—O-optionally substituted (C₅-C₂₅) alkyl, —*O—(C═O)-optionally substituted (C₅-C₂₅) alkenyl, and —(*C═O)—O-optionally substituted (C₅-C₂₅) alkenyl, wherein the atom marked with a * is connected to W¹.

53. The compound of any one of numbered embodiments 1-5, 8-11, 13-22, 27-45, or 47-52 or a pharmaceutically acceptable salt thereof, wherein the compound has a structure according to Formula (I), Formula (I′), Formula (IA), Formula (IE), Formula (IB1a), Formula (IB1d), Formula (I″), Formula (IB2a), Formula (IC1a), Formula (IC2a), or Formula (ID), wherein each R² or each R^{2A}, R^{2B}, R^{2C} and R^{2D}, when present, is independently selected from optionally substituted (C₅-C₂₅) alkyl, for example optionally substituted (C₅-C₂₀) alkyl, and —W¹—X¹,
wherein each W¹ is independently selected from optionally substituted (C₁-C₁₀) alkylene, for example optionally substituted (C₂-C₆) alkylene, and optionally substituted (C₂-C₁₀) alkenylene, for example optionally substituted (C₂-C₆) alkenylene, and
each X¹ is independently selected from —*O—(C═O)-optionally substituted (C₅-C₂₅) alkyl, for example —*O—(C═O)-optionally substituted (C₈-C₂₀) alkyl, —(*C═O)—O-optionally substituted (C₅-C₂₅) alkyl, for example —(*C═O)—O-optionally substituted (C₈-C₂₀) alkyl, —*O—(C═O)-optionally substituted (C₅-C₂₅) alkenyl, for example —*O—(C═O)-optionally substituted (C₈-C₂₀) alkenyl, and —(*C═O)—O-optionally substituted (C₅-C₂₅) alkenyl, for example —(*C═O)—O-optionally substituted (C₈-C₂₀) alkenyl, wherein the atom marked with a * is connected to W¹.

54. The compound of any one of numbered embodiments 1-5, 8-11, 13-22, 27-45, or 47-51 or a pharmaceutically acceptable salt thereof, wherein the compound has a structure according to Formula (I), Formula (I′), Formula (IA), Formula (IE), Formula (IB1a), Formula (IB1d), Formula (I″), Formula (IB2a), Formula (IC1a), Formula (IC2a), or Formula (ID), wherein each R² or each R^{2A}, R^{2B}, R^{2C} and R^{2D}, when present, is independently selected from optionally substituted alkyl and —W¹—X¹, optionally wherein
each W¹ is independently selected from optionally substituted alkylene, and
each X¹ is independently selected from —*O—(C═O)-optionally substituted alkyl, —(*C═O)—O-optionally substituted alkyl, and —(*C═O)—O-optionally substituted alkenyl, wherein the atom marked with a * is connected to W¹.

55. The compound of any one of numbered embodiments 1-5, 8-11, 13-22, 27-45, 47-51, or 54 or a pharmaceutically acceptable salt thereof, wherein the compound has a structure according to Formula (I), Formula (I′), Formula (IA), Formula (IE), Formula (IB1a), Formula (IB1d), Formula (I″), Formula (IB2a), Formula (IC1a), Formula (IC2a), or Formula (ID), wherein each R² or each R^{2A}, R^{2B}, R^{2C} and R^{2D}, when present, is independently selected from optionally substituted alkyl.

56. The compound of any one of numbered embodiments 1-5, 8-11, 13-22, 27-45, 47-52, 54 or 55 or a pharmaceutically acceptable salt thereof, wherein the compound has a structure according to Formula (I), Formula (I′), Formula (IA), Formula (IE), Formula (IB1a), Formula (IB1d), Formula (I″), Formula (IB2a), Formula (IC1a), Formula (IC2a), or Formula (ID), wherein each R² or each R^{2A}, R^{2B}, R^{2C} and R^{2D}, when present, is independently selected from optionally substituted (C₅-C₂₅) alkyl, for example optionally substituted (C₅-C₂₀) alkyl.

57. The compound of any one of numbered embodiments 1-5, 8-11, 13-22, 27-45, 47-51, or 54 or a pharmaceutically acceptable salt thereof, wherein the compound has a structure according to Formula (I), Formula (I′), Formula (IA), Formula (IE), Formula (IB1a), Formula (IB1d), Formula (I″), Formula (IB2a), Formula (IC1a), Formula (IC2a), or Formula (ID), wherein each R² or each R^{2A}, R^{2B}, R^{2C} and R^{2D}, when present, is independently selected from —W¹—X¹, optionally wherein
each W¹ is independently selected from optionally substituted alkylene, and
each X¹ is independently selected from —*O—(C═O)-optionally substituted alkyl, —(*C═O)—O-optionally substituted alkyl, and —(*C═O)—O-optionally substituted alkenyl, wherein the atom marked with a * is connected to W¹

58. The compound of any one of numbered embodiments 1-5, 8-11, 13-22, 27-45, 47-52, 54, or 57 or a pharmaceutically acceptable salt thereof, wherein the compound has a structure according to Formula (I), Formula (I′), Formula (IA), Formula (IE), Formula (IB1a), Formula (IB1d), Formula (I″), Formula (IB2a), Formula (IC1a), Formula (IC2a), or Formula (ID), wherein each R² or each R^{2A}, R^{2B}, R^{2C} and R^{2D}, when present, is independently selected from —W¹—X¹,
wherein each W¹ is independently selected from optionally substituted (C₁-C₁₀) alkylene, for example optionally substituted (C₂-C₆) alkylene, and optionally substituted (C₂-C₁₀) alkenylene, for example optionally substituted (C₂-C₆) alkenylene, and
   each X¹ is independently selected from —*O—(C═O)-optionally substituted (C₅-C₂₅) alkyl, for example —*O—(C═O)-optionally substituted (C₈-C₂₀) alkyl, —(*C═O)—O-optionally substituted (C₅-C₂₅) alkyl, for example —(*C═O)—O-optionally substituted (C₈-C₂₀) alkyl, —*O—(C═O)-optionally substituted (C₅-C₂₅) alkenyl, for example —*O—(C═O)-optionally substituted (C₈-C₂₀) alkenyl, and —(*C═O)—O-optionally substituted (C₅-C₂₅) alkenyl, for example —(*C═O)—O-optionally substituted (C₈-C₂₀) alkenyl, wherein the atom marked with a * is connected to W¹.

59. The compound of any one of numbered embodiments 1-5, 8-11, 13-22, 27-45, or 47-58 or a pharmaceutically acceptable salt thereof, wherein the compound has a structure according to Formula (I), Formula (I′), Formula (IA), Formula (IE), Formula (IB1a), Formula (IB1d), Formula (I″), Formula (IB2a), Formula (IC1a), Formula (IC2a), or Formula (ID), wherein each R² or each R^{2A}, R^{2B}, R^{2C} and R^{2D}, when present, is independently selected from:

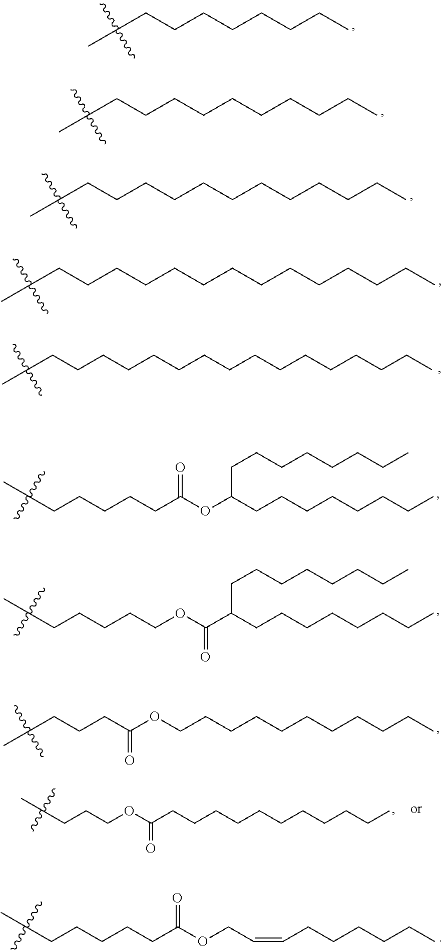

60. The compound of any one of numbered embodiments 1, 2, 9, 15-24, or 27-45 or a pharmaceutically acceptable salt thereof, wherein the compound has a structure according to Formula (I), Formula (I′). Formula (I″). Formula (II), or Formula (II′), wherein each R is independently selected from,

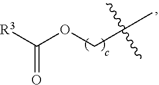

wherein each R³ is independently selected from optionally substituted alkyl, optionally substituted alkenyl, and optionally substituted alkynyl.

61. The compound of any one of numbered embodiments 1, 2, 7, 9, 15-25, 27-45, 47-50, or 52-60 or a pharmaceutically acceptable salt thereof, wherein the compound has a structure according to Formula (I), Formula (I′), Formula (IB1c), Formula (I″), Formula (II), Formula (II′), or Formula (IIA), wherein each R³ or each R^{3A}, R^{3B}, R^{3C} and R^{3D}, when present, is independently selected from optionally substituted (C₅-C₂₅) alkyl, optionally substituted (C₅-C₂₅) alkenyl, and optionally substituted (C₅-C₂₅) alkynyl.

62. The compound of any one of numbered embodiments 1, 2, 7, 9, 15-25, 27-45, 47-50, or 52-60 or a pharmaceutically acceptable salt thereof, wherein the compound has a structure according to Formula (I), Formula (I′), Formula (IB1c), Formula (I″), Formula (II), Formula (II′), or Formula (IIA), wherein each R³ or each R^{3A}, R^{3B}, R^{3C} and R^{3D}, when present, is independently selected from optionally substituted alkyl.

63. The compound of any one of numbered embodiments 1, 2, 7, 9, 15-25, 27-45, 47-50, 52-60 or 62 or a pharmaceutically acceptable salt thereof, wherein the compound has a structure according to Formula (I), Formula (I′), Formula (IB1c), Formula (I″), Formula (II), Formula (II′), or Formula (IIA), wherein each R³ or each R^{3A}, R^{3B}, R^{3C} and R^{3D}, when present, is independently selected from optionally substituted (C₅-C₂₅) alkyl, for example optionally substituted (C₁₀-C₂₀) alkyl.

64. The compound of any one of numbered embodiments 1, 2, 7, 9, 15-25, 27-45, 47-50, 52-60, 62 or 63 or a pharmaceutically acceptable salt thereof, wherein the compound has a structure according to Formula (I), Formula (I′), Formula (IB1c), Formula (I″), Formula (II), Formula (II′), or Formula (IIA), wherein each R³ or each R^{3A}, R^{3B}, R^{3C} and R^{3D}, when present, is independently selected from:

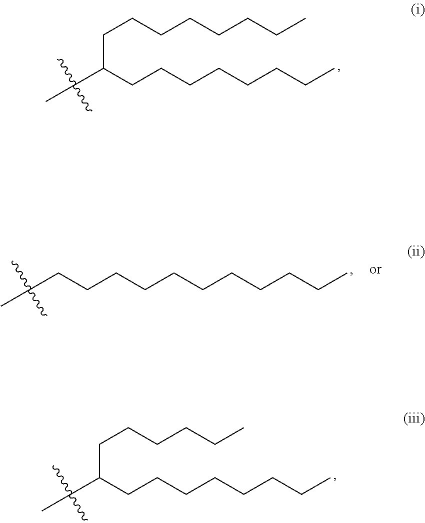 [SMILES: *C(CCCCCCCC)CCCCCCCC ; *CCCCCCCCCCC ; *C(CCCCCC)CCCCCCCC]

optionally wherein each R³ or each R^{3A}, R^{3B}, R^{3C} and R^{3D}, when present, is option (iii).

65. The compound of any of the preceding embodiments or a pharmaceutically acceptable salt thereof, wherein the compound of Formula (I) has a structure according to Formula (ID1):

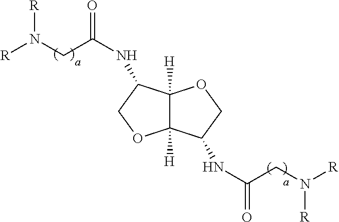

or a pharmaceutically acceptable salt thereof, wherein each R is independently selected from:

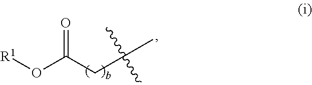

wherein each R¹ is independently selected from optionally substituted alkyl, optionally substituted alkenyl, and optionally substituted alkynyl;

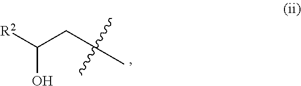

wherein each R² is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;

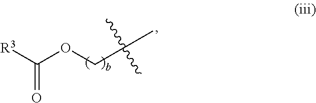

wherein each R³ is independently selected from optionally substituted alkyl, optionally substituted alkenyl, and optionally substituted alkynyl;
optionally wherein (i) each a is 3 or 4 and/or (ii) each b is 5, 6, or 7.

66. The compound of any of the preceding embodiments or a pharmaceutically acceptable salt thereof, wherein the compound of Formula (I) has a structure according to Formula (ID2):

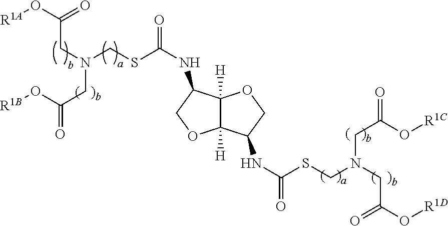

or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl;
optionally wherein (i) each a is 4, and/or (ii) each b is independently selected from 5 or 7.

67. The compound of any of the preceding embodiments or a pharmaceutically acceptable salt thereof, wherein the compound of Formula (I) has a structure according to Formula (IE1):

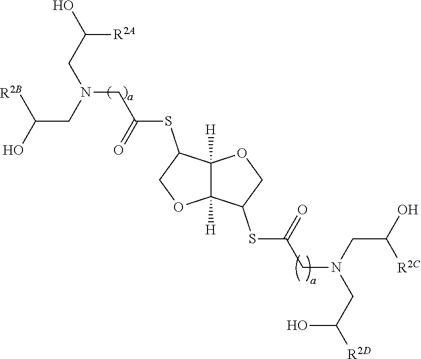

or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is 3 or 4.

68. The compound of any of the preceding embodiments or a pharmaceutically acceptable salt thereof, wherein the compound of Formula (I) has a structure according to Formula (IE2):

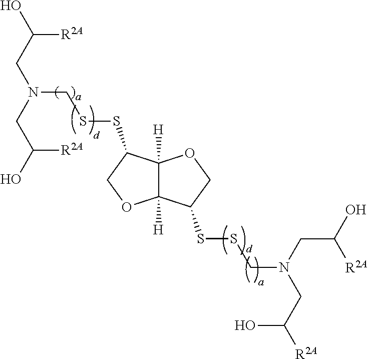

or a pharmaceutically acceptable salt thereof, wherein d is 0 or 1, and
wherein each R², R^{2B}, R^{2C} and R^{2U} is independently selected from optionally substituted alkyl, optionally substituted alkenyl, optionally substituted alkynyl, and —W¹—X¹,
   wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹;
optionally wherein each a is 3 or 4.

69. A compound selected from those listed in Table A or Table B, or a pharmaceutically acceptable salt thereof.

70. A composition comprising the cationic lipid of any one of numbered embodiments 1-69, and further comprising:
(i) one or more non-cationic lipids,
(ii) one or more cholesterol-based lipids, and
(iii) one or more PEG-modified lipids.

71. The composition of numbered embodiment 70, wherein the composition is a lipid nanoparticle, optionally a liposome.

72. The composition of numbered embodiment 71, wherein the one or more cationic lipid(s) constitute(s) about 30 mol %-60 mol % of the lipid nanoparticle.

73. The composition of numbered embodiment 71 or 72, wherein the one or more non-cationic lipid(s) constitute(s) about 10 mol %-50 mol % of the lipid nanoparticle.

74. The composition of any one of numbered embodiments 71-73, wherein the one or more PEG-modified lipid(s) constitute(s) about 1 mol %-10 mol % of the lipid nanoparticle.

75. The composition of any one of numbered embodiments 71-74, wherein the cholesterol-based lipid constitutes about 10 mol %-50 mol % of the lipid nanoparticle.

76. The composition of any one of numbered embodiments 71-75, wherein the lipid nanoparticle encapsulates a nucleic acid, optionally an mRNA encoding a peptide or protein.

77. The composition of any one of numbered embodiments 71-76, wherein the lipid nanoparticle encapsulates an mRNA encoding a peptide or protein, optionally for use in a vaccine.

78. The composition of numbered embodiment 77, wherein the lipid nanoparticles have an encapsulation percentage for mRNA of
(i) at least 50%;
(ii) at least 55%;
(iii) at least 60%;
(iv) at least 65%;
(v) at least 70%;
(vi) at least 75%;
(vii) at least 80%;
(viii) at least 85%;
(ix) at least 90%; or
(x) at least 95%.

79. The composition of numbered embodiment 77 or 78 for use in therapy.

80. The composition of numbered embodiment 77 or 78 for use in a method of treating or preventing a disease amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

81. The composition for use according to numbered embodiment 79 or 80, wherein the composition is administered intravenously, intrathecally, intramuscularly, intranasally, sublingually, or by pulmonary delivery, optionally through nebulization.

82. The composition for use according to any one of numbered embodiments 79-81, wherein the composition is administered intramuscularly.

83. A method for treating or preventing a disease wherein said method comprises administering to a subject in need thereof the composition of numbered embodiment 77 or 78 and wherein the disease is amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

84. The method of numbered embodiment 83, wherein the composition is administered intravenously, intrathecally, intramuscularly, intranasally, sublingually, or by pulmonary delivery, optionally through nebulization.

85. The method of numbered embodiment 83 or 84, wherein the composition is administered intramuscularly.

1. A compound having a structure according to Formula (I): 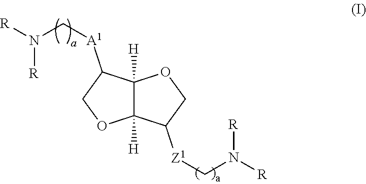
or a pharmaceutically acceptable salt thereof wherein:
A¹ is selected from —C(═O)O—, —C(═O)S—, —C(═O)NH—, —OC(═O)O—, —OC(═O)NH—, —NHC(═O)O—, —SC(═O)NH—, —OCH₂CH₂O—, —OCH₂O—, —OCH(CH₃)O—, —S— and —S—S—, wherein the left hand side of each recited structure is bound to the —(CH₂)ₐ—;
Z¹ is selected from —OC(═O)—, —SC(═O)—, —NHC(═O)—, —OC(═O)O—, —NHC(═O)O—, —OC(═O)NH—, —NHC(═O)S—, —OCH₂CH₂O—, —OCH₂O—, —OCH(CH₃)O—, —S— and —S—S—, wherein the right hand side of each recited structure is bound to the —(CH₂)ₐ—;
each R is independently selected from: 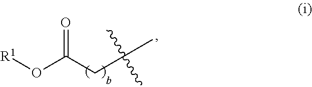
wherein each R¹ is independently selected from optionally substituted alkyl, optionally substituted alkenyl, and optionally substituted alkynyl; 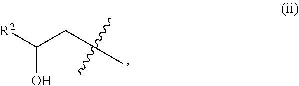
wherein each R² is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹; 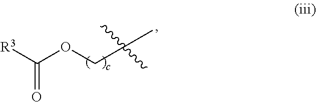
wherein each R³ is independently selected from optionally substituted alkyl, optionally substituted alkenyl, and optionally substituted alkynyl; and 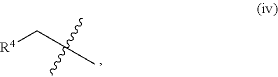
wherein each R⁴ is independently selected from optionally substituted cycloalkyl or optionally substituted heterocycloalkyl;
wherein at least three R are independently selected from 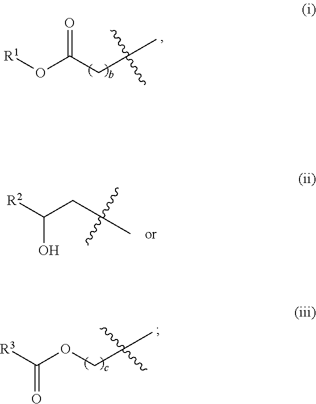
each a is independently selected from 2, 3, 4, and 5;
each b is independently selected from 2, 3, 4, 5, 6, 7, 8, 9 and 10; and
each c is independently selected from 2, 3, 4, 5, 6, 7, 8, 9 and 10.

2. The compound of claim 1, wherein the compound has a structure according to Formula (IB1a): 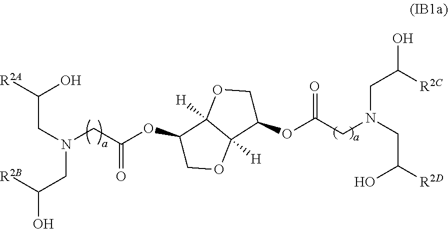
or a pharmaceutically acceptable salt thereof, wherein each R^{2A}, R^{2B}, R2c and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

3. The compound of claim 1, wherein the compound has a structure according to Formula (IB1b): 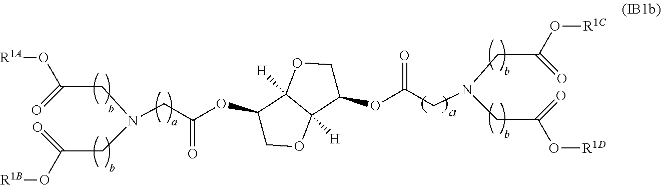
or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl.

4. The compound of claim 1, wherein the compound has a structure according to Formula (IB1c): 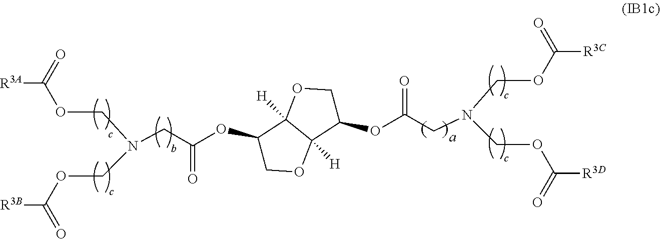
or a pharmaceutically acceptable salt thereof, wherein each R^{3A}, R^{3B}, R^{3C} and R^{3D} is independently selected from optionally substituted alkyl, optionally substituted alkenyl, and optionally substituted alkynyl.

5. The compound of claim 1, wherein the compound has a structure according to Formula (IB2a): 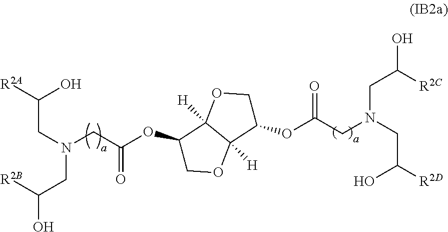
or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

6. The compound of claim 1, wherein the compound has a structure according to Formula (IC1a): 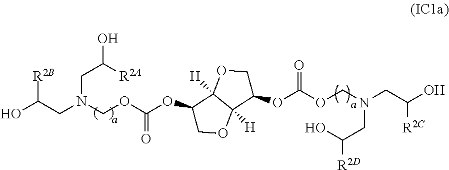
or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

7. The compound of claim 1, wherein the compound has a structure according to Formula (IC1b): 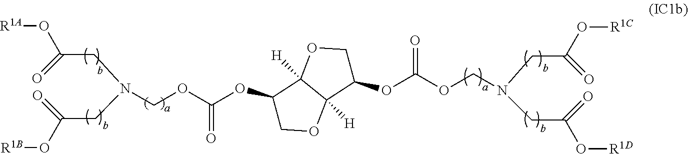
or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl.

8. The compound of claim 1, wherein the compound has a structure according to Formula (IC2a): 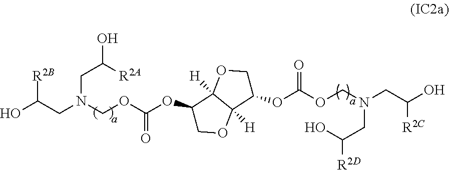
or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

9. The compound of claim 1, wherein the compound of Formula (I) has a structure according to:
(a) Formula (ID1): 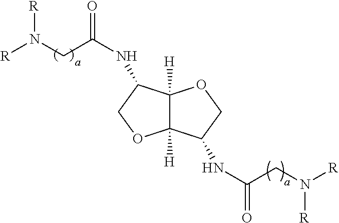
or a pharmaceutically acceptable salt thereof, wherein each R is independently selected from: 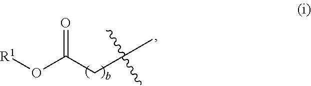
wherein each R¹ is independently selected from optionally substituted alkyl, optionally substituted alkenyl, and optionally substituted alkynyl; 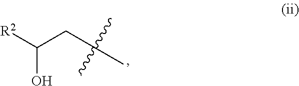
wherein each R² is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹; 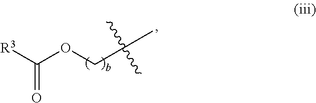
wherein each R³ is independently selected from optionally substituted alkyl, optionally substituted alkenyl, and optionally substituted alkynyl;
(b) Formula (ID2): 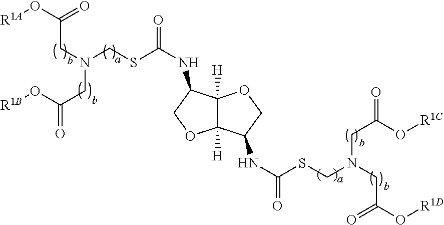
or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl;
(c) Formula (IE1): 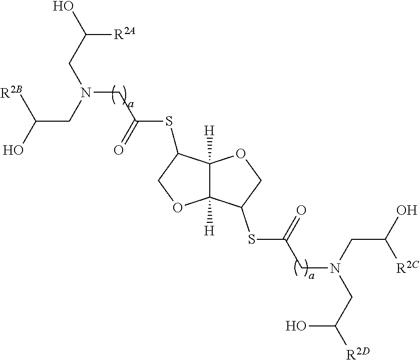
or a pharmaceutically acceptable salt thereof, wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹; or
(d) Formula (IE2): 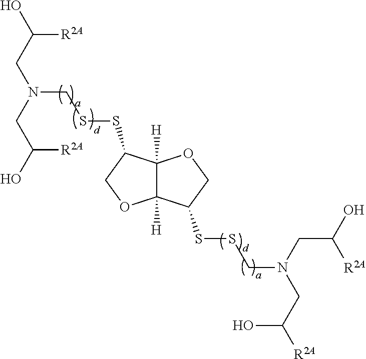
or a pharmaceutically acceptable salt thereof, wherein d is 0 or 1, and
wherein each R^{2A}, R^{2B}, R^{2C} and R^{2D} is independently selected from optionally substituted alkyl, optionally substituted alkenyl, optionally substituted alkynyl, and —W¹—X¹,
wherein each W¹ is independently selected from optionally substituted alkylene and optionally substituted alkenylene, and
each X¹ is independently selected from —*O—(C═O)-optionally substituted alkyl, —(*C═O)—O-optionally substituted alkyl, —*O—(C═O)-optionally substituted alkenyl, and —(*C═O)—O-optionally substituted alkenyl, wherein the atom marked with a * is connected to W¹.

10. A compound having a structure according to Formula (II): 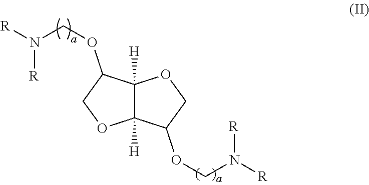
or a pharmaceutically acceptable salt thereof wherein:
each R is independently selected from: 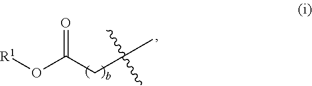
wherein each R¹ is independently selected from optionally substituted alkyl, optionally substituted alkenyl, and optionally substituted alkynyl; and 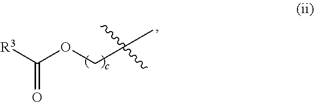
wherein each R³ is independently selected from optionally substituted alkyl, optionally substituted alkenyl, and optionally substituted alkynyl;
each a is independently selected from 2, 3, 4, and 5;
each b is independently selected from 2, 3, 4, 5, 6, and 7; and
each c is independently selected from 2, 3, 4, 5, 6, and 7.

11. The compound of claim 10, wherein the compound has a structure according to Formula (IIA): 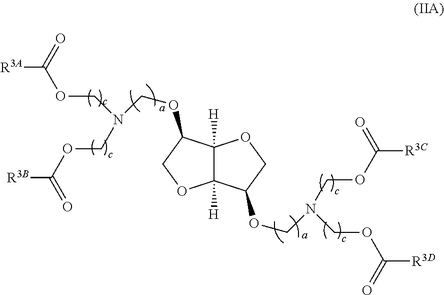
or a pharmaceutically acceptable salt thereof, wherein each R^{3A}, R^{3B}, R^{3C} and R^{3D} is independently selected from optionally substituted alkyl, optionally substituted alkenyl, and optionally substituted alkynyl.

12. The compound of claim 10, wherein the compound has a structure according to Formula (IIB): 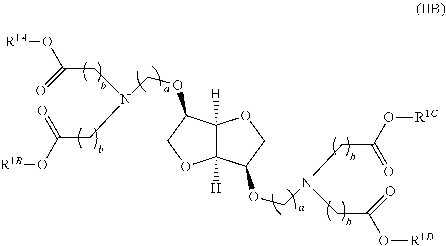
or a pharmaceutically acceptable salt thereof, wherein each R^{1A}, R^{1B}, R^{1C} and R^{1D} is independently selected from optionally substituted alkyl, optionally substituted alkenyl, and optionally substituted alkynyl.

13. A composition comprising the cationic lipid of claim 1, and further comprising:
(i) one or more non-cationic lipids,
(ii) one or more cholesterol-based lipids, and
(iii) one or more PEG-modified lipids.

14. The composition of claim 13, wherein the composition is a lipid nanoparticle.

15. (canceled)

16. A method of treating or preventing a disease, said method comprising administering to a subject in need thereof a therapeutically effective amount of the composition of claim 14, wherein the disease is amenable to treatment or prevention by the peptide or protein encoded by the mRNA.

17. The lipid nanoparticle of claim 14, wherein the lipid nanoparticle encapsulates an mRNA encoding a peptide or protein.

18. A composition comprising the cationic lipid claim 10, and further comprising:
(i) one or more non-cationic lipids,
(ii) one or more cholesterol-based lipids, and
(iii) one or more PEG-modified lipids.

19. The composition of claim 19, wherein the composition is a lipid nanoparticle.

20. A method of treating or preventing a disease, said method comprising administering to a subject in need thereof a therapeutically effective amount of the composition of claim 18, wherein the disease is amenable to treatment or prevention by the peptide or protein encoded by the mRNA.

21. The lipid nanoparticle of claim 19, wherein the lipid nanoparticle encapsulates an mRNA encoding a peptide or protein.
